SEQUENCE LISTING (312 sequences)

SEQ ID NO: 1 - DNA, 714 bp, Treponema pallidum
  source 1..714: mol_type = genomic DNA
gtgaatagacctttactgagtgtggccggatcgttgttcgtcgctgcgtgggctctatatattttctcctgctttcagcacggtcacgttcctccacgcagaattcccccgcatgataccttcggcgctctacccactgctgcacttcccagcaacgcgcgggacaccgccgcacacccgtctgacacggcagacaatacgtccggctcgtccaccacgacagacccacggtcacatggaaacgccccgccggcccctgtaggcggagcagcacagacacacacacaaccgcctgtacaaacagcaatgcgcatagccctttggaaccgtgcaacacatggtgaacagggagcactccagcacctcttggcaggactgtggatacaaactgaaatctccccgaactcaggcgatatccatcctctgctgttttttgaccgagaacacgcggagatcacattctcacgcgcatcagtccaagaaatcttcctggtagatagcgcgcacacacaccgcaagacggtgtcatttctcacgcgcaacaccgcaatttccagcatccgccgccgccttgaggtaacatttgaatcccacgaggtgatacacgtaagggcggttgaagacgtagcacggctcaaaattggcagcacgtcgatgtgggacggtcaatacaccagatatcacgccggtccggctagtgctccttcgccctga

SEQ ID NO: 2 - protein, 237 aa, Treponema pallidum
MNRPLLSVAGSLFVAAWALYIFSCFQHGHVPPRRIPPHDTFGALPTAALPSNARDTAAHPSDTADNTSGSSTTTDPRSHGNAPPAPVGGAAQTHTQPPVQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 3 - protein, 139 aa, Treponema pallidum
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 4 - protein, 11 aa, Treponema pallidum
GEQGALQHLLA

SEQ ID NO: 5 - protein, 13 aa, Treponema pallidum
QTEISPNSGDIHP

SEQ ID NO: 6 - protein, 7 aa, Treponema pallidum
NTAISSI

SEQ ID NO: 7 - protein, 14 aa, Treponema pallidum
DVARLKIGSTSMWD

SEQ ID NO: 8 - protein, 12 aa, Treponema pallidum
RYHAGPASAPSP

SEQ ID NO: 9 - protein, 525 aa, Treponema pallidum
MIDPSATSRYGSPRLVSNGFRHRRKVVYQRVGHRRFSLIFFFVVVLGRSPRLWAQVSFTPDIEGYAELAWGIASETGGAGALKHGFKTTTDFKIVFPIVAKKDFKYRGEGNVYAEINVKALKLSLESNGGAKFDTKGSAKTIEATLHCYGAYLTIGKNPDFKSTFAVLWEPWTANGDYKSKGDKPVYEPGFEGAGGKLGYKQTDIAGTGLTFDIAFKFASNTDWEGKDSQGKAPAAGVTHSKYGLGGDILFGWERTREDGVQEYIKVELTGNSTLSGDYARAGAAVPAAADDILWDVGAKVSMKLWGLCALAATDVGHKKENAANVNGTVGADALLTLGYRWFSAGGYFASKASNVFKDVFLTNAMDMQTHDCAAYIKLETKGSDPDTSFLEGLDLGVDVCTYMPVHWKALALPAAAAAAAANINFPVYGKVWGSYRHDMGEYGWVKVYANLYGGTNKKNDAAALTKWSKEYCGYYECGVVVSPLEKVEIRLSWEQGKLQENSNVVIEKNVTERWQFVGACRLIW

SEQ ID NO: 10 - DNA, 420 bp, Treponema pallidum
  source 1..420: mol_type = genomic DNA
gtacaaacagcaatgcgcatagccctttggaaccgtgcaacacatggtgaacagggagcactccagcacctcttggcaggactgtggatacaaactgaaatctccccgaactcaggcgatatccatcctctgctgttttttgaccgagaacacgcggagatcacattctcacgcgcatcagtccaagaaatcttcctggtagatagcgcgcacacacaccgcaagacggtgtcatttctcacgcgcaacaccgcaatttccagcatccgccgccgccttgaggtaacatttgaatcccacgaggtgatacacgtaagggcggttgaagacgtagcacggctcaaaattggcagcacgtcgatgtgggacggtcaatacaccagatatcacgccggtccggctagtgctccttcgccctga

SEQ ID NO: 11 - DNA, 417 bp, Synthetic construct
  source 1..417: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtctgaaaattggtagcaccagcatgtgggatggccaatacacccgttatcacgcgggtccggcgagcgcgccgagcccg

SEQ ID NO: 12 - protein, 167 aa, Synthetic construct
MGSTSGSGHHHHHHSSGRENLYFQGSMAVQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 13 - protein, 143 aa, Synthetic construct
GSMAVQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 14 - protein, 7 aa, Neisseria meningitidis
GDDLHMG

SEQ ID NO: 15 - protein, 8 aa, Neisseria meningitidis
KTVNGIRH

SEQ ID NO: 16 - protein, 10 aa, Neisseria meningitidis
VLYNQAEKGS

SEQ ID NO: 17 - protein, 11 aa, Treponema pallidum
RAYSEKDTRYA

SEQ ID NO: 18 - protein, 17 aa, Treponema pallidum
GKLVQNLPNIMMPPGIT

SEQ ID NO: 19 - protein, 9 aa, Treponema pallidum
AKTIEATLH

SEQ ID NO: 20 - protein, 7 aa, Treponema pallidum
ESNGGAK

SEQ ID NO: 21 - protein, 12 aa, Treponema pallidum
DYKSKGDKPVYE

SEQ ID NO: 22 - protein, 13 aa, Treponema pallidum
LQENSNVVIEKNV

SEQ ID NO: 23 - protein, 10 aa, Treponema pallidum
DNNQPFDLTV

SEQ ID NO: 24 - protein, 20 aa, Treponema pallidum
FQKNPRTGPGKHTHGFRTTN

SEQ ID NO: 25 - protein, 22 aa, Treponema pallidum
LAVELASSKSSTALSFTKPTAS

SEQ ID NO: 26 - protein, 17 aa, Treponema pallidum
GTTNRFNIINAAGNLLN

SEQ ID NO: 27 - protein, 16 aa, Treponema pallidum
NGDYKSKGDKPVYEPG

SEQ ID NO: 28 - protein, 9 aa, Treponema pallidum
DYKSKGDKP

SEQ ID NO: 29 - protein, 9 aa, Treponema pallidum
SKGDKPVYE

SEQ ID NO: 30 - protein, 9 aa, Treponema pallidum
KSKGDKPVY

SEQ ID NO: 31 - protein, 17 aa, Treponema pallidum
GTTNRFNIINPAGNLLN

SEQ ID NO: 32 - protein, 17 aa, Treponema pallidum
QGVLADAPYMGIAESIW

SEQ ID NO: 33 - protein, 10 aa, Treponema pallidum
KYRGEGNVYA

SEQ ID NO: 34 - protein, 10 aa, Treponema pallidum
GKLQENSNVV

SEQ ID NO: 35 - protein, 9 aa, Treponema pallidum
LVQNLPNIM

SEQ ID NO: 36 - protein, 9 aa, Treponema pallidum
NLPNIMMPP

SEQ ID NO: 37 - protein, 9 aa, Treponema pallidum
NIMMPPGIT

SEQ ID NO: 38 - protein, 142 aa, Synthetic construct
VQTAMRIALWNRATHGEQGALQHLLAGLWIQTEKTVNGIRHGDIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 39 - protein, 144 aa, Synthetic construct
VQTAMRIALWNRATHGEQGALQHLLAGLWIQTEVLYNQAEKGSGDIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 40 - protein, 144 aa, Synthetic construct
VQTAMRIALWNRATHGEQGALQHLLAGLWIQTEISPNSGDIHPLLFFDRGDDLHMGAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 41 - protein, 147 aa, Synthetic construct
VQTAMRIALWNRATHGEQGALQHLLAGLWIQTEISPNSGDIHPLLFFDRVLYNQAEKGSAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 42 - protein, 144 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAGDDLHMGSIRRRLEVTFESHEVIHVRAVEDVARLKIGSTSMWDGQYTRYHAGPASAPSP

SEQ ID NO: 43 - protein, 145 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAKTVNGIRHSIRRRLEVTFESHEVIHVRAVEDVARLKIGSTSMWDGQYTRYHAGPASAPSP

SEQ ID NO: 44 - protein, 147 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAVLYNQAEKGSSIRRRLEVTFESHEVIHVRAVEDVARLKIGSTSMWDGQYTRYHAGPASAPSP

SEQ ID NO: 45 - protein, 139 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARGDDLHMGMWDGQYTRYHAGPASAPSP

SEQ ID NO: 46 - protein, 140 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARKTVNGIRHMWDGQYTRYHAGPASAPSP

SEQ ID NO: 47 - protein, 142 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARVLYNQAEKGSMWDGQYTRYHAGPASAPSP

SEQ ID NO: 48 - protein, 141 aa, Synthetic construct
VQTAMRIALWNRATHGEQGALQHLLAGLWIQTRAYSEKDTRYAHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 49 - protein, 147 aa, Synthetic construct
VQTAMRIALWNRATHGEQGALQHLLAGLWIQTGKLVQNLPNIMMPPGITHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 50 - protein, 143 aa, Synthetic construct
VQTAMRIALWNRATHGEQGALQHLLAGLWIQTEAKTIEATLHGDIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 51 - protein, 139 aa, Synthetic construct
VQTAMRIALWNRATHGEQGALQHLLAGLWIQTAKTIEATLHHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 52 - protein, 141 aa, Synthetic construct
VQTAMRIALWNRATHGEQGALQHLLAGLWIQTEESNGGAKGDIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 53 - protein, 144 aa, Synthetic construct
VQTAMRIALWNRATHGEQGALQHLLAGLWIQTEDYKSKGDKPVYEIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 54 - protein, 145 aa, Synthetic construct
VQTAMRIALWNRATHGEQGALQHLLAGLWIQTELQENSNVVIEKNVIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 55 - protein, 145 aa, Synthetic construct
VQTAMRIALWNRATHGEQGALQHLLAGLWIQTEISPNSGDIHPLLFFDDNNQPFDLTVEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 56 - protein, 142 aa, Synthetic construct
VQTAMRIALWNRATHGEQGALQHLLAGLWIQTEISPNSGDIHPLLFFDESNGGAKEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 57 - protein, 148 aa, Synthetic construct
VQTAMRIALWNRATHGEQGALQHLLAGLWIQTEISPNSGDIHPLLFFDLQENSNVVIEKNVEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 58 - protein, 144 aa, Synthetic construct
VQTAMRIALWNRATHGEQGALQHLLAGLWIQTEISPNSGDIHPLLFFDREHAEITFSRASVQEIFLVDSAHTHRKTVSFLTRNDNNQPFDLTVIRRRLEVTFESHEVIHVRAVEDVARLKIGSTSMWDGQYTRYHAGPASAPSP

SEQ ID NO: 59 - protein, 145 aa, Synthetic construct
VQTAMRIALWNRATHGEQGALQHLLAGLWIQTEISPNSGDIHPLLFFDREHAEITFSRASVQEIFLVDSAHTHRKTVSFLTRNRAYSEKDTRYAIRRRLEVTFESHEVIHVRAVEDVARLKIGSTSMWDGQYTRYHAGPASAPSP

SEQ ID NO: 60 - protein, 141 aa, Synthetic construct
VQTAMRIALWNRATHGEQGALQHLLAGLWIQTEISPNSGDIHPLLFFDREHAEITFSRASVQEIFLVDSAHTHRKTVSFLTRNESNGGAKIRRRLEVTFESHEVIHVRAVEDVARLKIGSTSMWDGQYTRYHAGPASAPSP

SEQ ID NO: 61 - protein, 147 aa, Synthetic construct
VQTAMRIALWNRATHGEQGALQHLLAGLWIQTEISPNSGDIHPLLFFDREHAEITFSRASVQEIFLVDSAHTHRKTVSFLTRNLQENSNVVIEKNVIRRRLEVTFESHEVIHVRAVEDVARLKIGSTSMWDGQYTRYHAGPASAPSP

SEQ ID NO: 62 - protein, 140 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVADNNQPFDLTVWDGQYTRYHAGPASAPSP

SEQ ID NO: 63 - protein, 151 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARFQKNPRTGPGKHTHGFRTTNWDGQYTRYHAGPASAPSP

SEQ ID NO: 64 - protein, 153 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARLAVELASSKSSTALSFTKPTASWDGQYTRYHAGPASAPSP

SEQ ID NO: 65 - protein, 141 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARAYSEKDTRYAWDGQYTRYHAGPASAPSP

SEQ ID NO: 66 - protein, 147 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVAGTTNRFNIINAAGNLLNWDGQYTRYHAGPASAPSP

SEQ ID NO: 67 - protein, 147 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARNGDYKSKGDKPVYEPGWDGQYTRYHAGPASAPSP

SEQ ID NO: 68 - protein, 143 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSP

SEQ ID NO: 69 - protein, 142 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVADYKSKGDKPVYEWDGQYTRYHAGPASAPSP

SEQ ID NO: 70 - protein, 140 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARDYKSKGDKPWDGQYTRYHAGPASAPSP

SEQ ID NO: 71 - protein, 140 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARSKGDKPVYEWDGQYTRYHAGPASAPSP

SEQ ID NO: 72 - protein, 140 aa, Synthetic construct
VQTAMRIALWNRATHGEQGALQHLLAGLWIQTEISPNSGDIHPLLFFDREHAEITFSRASVQEIFLVDSAHTHRKTVSFLTRNTAISSIRRRLEVTFESHEVIHVRAVEDVARKSKGDKPVYWDGQYTRYHAGPASAPSP

SEQ ID NO: 73 - protein, 149 aa, Synthetic construct
VQTAMRIALWNRATHGEQGALQHLLAGLWIQTERAYSEKDTRYAGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSP

SEQ ID NO: 74 - protein, 155 aa, Synthetic construct
VQTAMRIALWNRATHGEQGALQHLLAGLWIQTEGKLVQNLPNIMMPPGITGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSP

SEQ ID NO: 75 - protein, 155 aa, Synthetic construct
VQTAMRIALWNRATHGEQGALQHLLAGLWIQTEGTTNRFNIINAAGNLLNGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSP

SEQ ID NO: 76 - protein, 155 aa, Synthetic construct
VQTAMRIALWNRATHGEQGALQHLLAGLWIQTEGTTNRFNIINPAGNLLNGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSP

SEQ ID NO: 77 - protein, 155 aa, Synthetic construct
VQTAMRIALWNRATHGEQGALQHLLAGLWIQTEQGVLADAPYMGIAESIWGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSP

SEQ ID NO: 78 - protein, 148 aa, Synthetic construct
VQTAMRIALWNRATHGEQGALQHLLAGLWIQTEKYRGEGNVYAGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSP

SEQ ID NO: 79 - protein, 147 aa, Synthetic construct
VQTAMRIALWNRATHGEQGALQHLLAGLWIQTEAKTIEATLHGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSP

SEQ ID NO: 80 - protein, 148 aa, Synthetic construct
VQTAMRIALWNRATHGEQGALQHLLAGLWIQTEGKLQENSNVVGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSP

SEQ ID NO: 81 - protein, 164 aa, Synthetic construct
VQTAMRIALWNRATHGEQGALQHLLAGLWIQTEAKTIEATLHGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHAGPASAPSPGKLVQNLPNIMMPPGIT

SEQ ID NO: 82 - protein, 155 aa, Synthetic construct
VQTAMRIALWNRATHGEQGALQHLLAGLWIQTEAKTIEATLHGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHGKLVQNLPNIMMPPGIT

SEQ ID NO: 83 - protein, 147 aa, Synthetic construct
VQTAMRIALWNRATHGEQGALQHLLAGLWIQTEAKTIEATLHGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHLVQNLPNIM

SEQ ID NO: 84 - protein, 147 aa, Synthetic construct
VQTAMRIALWNRATHGEQGALQHLLAGLWIQTEAKTIEATLHGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHNLPNIMMPP

SEQ ID NO: 85 - protein, 147 aa, Synthetic construct
VQTAMRIALWNRATHGEQGALQHLLAGLWIQTEAKTIEATLHGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHNIMMPPGIT

SEQ ID NO: 86 - protein, 153 aa, Synthetic construct
VQTAMRIALWNRATHGEQGALQHLLAGLWIQTEESNGGAKGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHGKLVQNLPNIMMPPGIT

SEQ ID NO: 87 - protein, 156 aa, Synthetic construct
VQTAMRIALWNRATHGEQGALQHLLAGLWIQTEGKLQENSNVVGDIHPLLFFDREHAEITFSRASVQEIFLVDSAHTHRKTVSFLTRNTAISSIRRRLEVTFESHEVIHVRAVEDVARDYKSKGDKPVYEWDGQYTRYHGKLVQNLPNIMMPPGIT

SEQ ID NO: 88 - protein, 157 aa, Synthetic construct
VQTAMRIALWNRATHGEQGALQHLLAGLWIQTEDYKSKGDKPVYEIHPLLFFDESNGGAKEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 89 - protein, 163 aa, Synthetic construct
VQTAMRIALWNRATHGEQGALQHLLAGLWIQTEDYKSKGDKPVYEIHPLLFFDLQENSNVVIEKNVEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 90 - protein, 159 aa, Synthetic construct
VQTAMRIALWNRATHGEQGALQHLLAGLWIQTELQENSNVVIEKNVIHPLLFFDESNGGAKEITFSRASVQEIFLVDSAHTHRKTVSFLTRNTAISSIRRRLEVTFESHEVIHVRAVEDVADYKSKGDKPVYEWDGQYTRYHGKLVQNLPNIMMPPGIT

SEQ ID NO: 91 - protein, 141 aa, Synthetic construct
VQTAMRIALWNRATHGEQGALQHLLAGLWIQTEGDDLHMGGDIHPLLFFDREHAEITFSRASVQEIFLVDSAHTHRKTVSFLTRNTAISSIRRRLEVTFESHEVIHVRAVEDVARLKIGSTSMWDGQYTRYHAGPASAPSP

SEQ ID NO: 92 - protein, 274 aa, Neisseria meningitidis
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 93 - protein, 427 aa, Neisseria meningitidis
MFERSVIAMACIFALSACGGGGGGSPDVKSADTLSKPAAPVVAEKETEVKEDAPQAGSQGQGAPSTQGSQDMAAVSAENTGNGGAATTDKPKNEDEGPQNDMLQNSAESANQTGNNQPADSSDSAPASNPAPANGGSNFGRVDLANGVLIDGPSQNITLTHCKGDSCNGDNLLDEEAPSKSEFENLNESERIEKYKKDGKSDKFTNLVATAVQANGTNKYVIIYKDKSASSSFARFRRSARSRRSLPAEMPLIPVNQADTLIVDGEAVSLTGHSGNIFAPEGNYRYLTYGAEKLPGGSYALRVQGEPAKGEMLAGTAVYNGEVLHFHTENGRPYPTRGRFAAKVDFGSKSVDGIIDSGDDLHMGTQKFKAAIDGNGFKGTWTENGGGDVSGRFYGPAGEEVAGKYSYRPTDAEKGGFGVFAGKKEQD

SEQ ID NO: 94 - protein, 598 aa, Treponema pallidum
MGRQVMQAGVLAGMVCAASGYAGVLTPQVSGTAQLQWGIAFQKNPRTGPGKHTHGFRTTNSLTISLPLVSKHTHTRRGEARSGVWAQLQLKDLAVELASSKSSTALSFTKPTASFQATLHCYGAYLTVGTSPSCVVNFAQLWKPFVTRAYSEKDTRYAPGFSGSGAKLGYQAHNVGNSGVDVDIGFLSFLSNGAWDSTDTTHSKYGFGADATLSYGVDRQRLLTLELAGNATLDQNYVKGTEDSKNENKTALLWGVGGRLTLEPGAGFRFSFALDAGNQHQSNAHAQTQERAILKAREVFRRVEGKLVQNLPNIMMPPGITEQTTLIEMVGLAALIAEGTLGSAIQTVLAAGALAALVSQLVPNIEQGVRDVFRSSDPRVVTAKLLAFLERAPMNALNIDALLRMQWKWLSSGIYFATAGTNIFGKRVFATTRAHYFDFAGFLKLETKSGDPYTHLLTGLNAGVEARVYIPLTYIRYRNNGGYELNGAVPPGTINMPILGKAWCSYRIPLGSHAWLAPHTSVLGTTNRFNIINPAGNLLNERALQYQVGLTFSPFEKVELSAQWEQGVLADAPYMGIAESIWSERHFGTLVCGMKVTW

SEQ ID NO: 95 - DNA, 465 bp, Synthetic construct
  source 1..465: mol_type = other DNA
gtacaaacagctatgaggatagcactatggaaccgtgcgacccacggtgagcagggtgctttgcagcatctgttagcaggcctgtggattcagaccgaggcgaaaaccattgaagcaaccctgcatggcgacatccacccgctcctgtttttcgatcgtgagcacgccgaaatcaccttcagccgtgcttcggtgcaagagatcttcttggtcgacagcgcgcatacccaccgcaagacggtttcctttctgacccgtaataccgcgattagctctatcagacgtcgcctggaggtgacgtttgagagccatgaagtaatccacgtgcgcgccgtggaagacgttgcgcgtgattacaaatccaagggcgacaaaccggtttatgaatgggatggtcaatatactcgctaccacggcaagttggttcaaaacctgccgaacatcatgatgccaccgggtattact

SEQ ID NO: 96 - DNA, 471 bp, Synthetic construct
  source 1..471: mol_type = other DNA
gtacaaacagcaatgaggatagctctatggaaccgtgctacccacggcgaacaaggtgcgctgcagcacctgctggctggcctgtggattcagaccgaagactacaagtccaaaggtgataagccggtgtatgagatccacccgctgttgtttttcgacgaatctaatggtggtgcgaaagaaatcacgttcagcagagcgtccgtacaagagatctttttggttgatagcgcacatacccatcgtaaaaccgtgagctttttgacccgcaacaccgcaatttctagcattcgtcgccggctggaggtgaccttcgagtcgcacgaggtcatccacgttcgtgccgttgaggacgtggcccgtgcgtatagcgaaaaggacacgcgttacgcgtgggatggtcagtatacgcgctaccacggtaagctcgttcaaaacctgccgaatattatgatgccaccgggcatcact

SEQ ID NO: 97 - protein, 158 aa, Synthetic construct
VQTAMRIALWNRATHGEQGALQHLLAGLWIQTEDYKSKGDKPVYEIHPLLFFDVKPSFTGVEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 98 - protein, 157 aa, Synthetic construct
VQTAMRIALWNRATHGEQGALQHLLAGLWIQTEDYKSKGDKPVYEIHPLLFFDESGFNFSEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 99 - protein, 157 aa, Synthetic construct
VQTAMRIALWNRATHGEQGALQHLLAGLWIQTEDYKSKGDKPVYEIHPLLFFDGNEIKDSEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 100 - protein, 158 aa, Synthetic construct
APVYRPQDHAATINGVSDQGALQHLLAGLWIQTEDYKSKGDKPVYEIHPLLFFDESNGGAKEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 101 - protein, 154 aa, Synthetic construct
VNVSNLQVDHLWKRQGALQHLLAGLWIQTEDYKSKGDKPVYEIHPLLFFDESNGGAKEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 102 - protein, 160 aa, Synthetic construct
VDFPLNPNTRFLAGKLPAETQGALQHLLAGLWIQTEDYKSKGDKPVYEIHPLLFFDESNGGAKEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 103 - protein, 158 aa, Synthetic construct
KNGAGSTDLKWEQFLQKYQGALQHLLAGLWIQTEDYKSKGDKPVYEIHPLLFFDESNGGAKEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 104 - protein, 156 aa, Synthetic construct
ANRWISGPHWWNSLSSQGALQHLLAGLWIQTEDYKSKGDKPVYEIHPLLFFDESNGGAKEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 105 - protein, 157 aa, Synthetic construct
VQTAMRIALWNRATHGSSEAAAKTLAGLWIQTEDYKSKGDKPVYEIHPLLFFDESNGGAKEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 106 - protein, 157 aa, Synthetic construct
VQTAMRIALWNRATHGVKPSFTGVLAGLWIQTEDYKSKGDKPVYEIHPLLFFDESNGGAKEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 107 - protein, 158 aa, Synthetic construct
VQTAMRIALWNRATHGPESGFNFSPLAGLWIQTEDYKSKGDKPVYEIHPLLFFDESNGGAKEITFSRASVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 108 - protein, 162 aa, Synthetic construct
VQTAMRIALWNRATHGEQGALQHLLAGLWIQTEDYKSKGDKPVYEIHPLLFFDESNGGAKEITFSRGNEIKDSVQEIFLVDSAHTHRKTVSFLTRNTAISSIRRRLEVTFESHEVIHVRAVEDVARAYSEKDTRYAWDGQYTRYHGKLVQNLPNIMMPPGIT

SEQ ID NO: 109 - protein, 162 aa, Synthetic construct
VQTAMRIALWNRATHGEQGALQHLLAGLWIQTEDYKSKGDKPVYEIHPLLFFDESNGGAKEITFSRASVQEIFLVDSAHTHGNEIKDSTVSFLTRNTAISSIRRRLEVTFESHEVIHVRAVEDVARAYSEKDTRYAWDGQYTRYHGKLVQNLPNIMMPPGIT

SEQ ID NO: 110 - protein, 161 aa, Synthetic construct
VQTAMRIALWNRATHGEQGALQHLLAGLWIQTEDYKSKGDKPVYEIHPLLFFDESNGGAKEITFSRASVQEIFLVDSAHTHRKTVSFLTRNSTGDEQQGTIRRRLEVTFESHEVIHVRAVEDVARAYSEKDTRYAWDGQYTRYHGKLVQNLPNIMMPPGIT

SEQ ID NO: 111 - protein, 161 aa, Synthetic construct
VQTAMRIALWNRATHGEQGALQHLLAGLWIQTEDYKSKGDKPVYEIHPLLFFDESNGGAKEITFSRASVQEIFLVDSAHTHRKTVSFLTRNAVGKDIEGQIRRRLEVTFESHEVIHVRAVEDVARAYSEKDTRYAWDGQYTRYHGKLVQNLPNIMMPPGIT

SEQ ID NO: 112 - protein, 159 aa, Synthetic construct
VQTAMRIALWNRATHGEQGALQHLLAGLWIQTEDYKSKGDKPVYEIHPLLFFDESNGGAKEITFSRASVQEIFLVDSAHTHRKTVSFLTRNFGQRKEDIRRRLEVTFESHEVIHVRAVEDVARAYSEKDTRYAWDGQYTRYHGKLVQNLPNIMMPPGIT

SEQ ID NO: 113 - protein, 160 aa, Synthetic construct
VQTAMRIALWNRATHGEQGALQHLLAGLWIQTEDYKSKGDKPVYEIHPLLFFDESNGGAKEITFSRASVQEIFLVDSAHTHRKTVSFLTRNVKPSFTGVIRRRLEVTFESHEVIHVRAVEDVARAYSEKDTRYAWDGQYTRYHGKLVQNLPNIMMPPGIT

SEQ ID NO: 114 - protein, 159 aa, Synthetic construct
VQTAMRIALWNRATHGEQGALQHLLAGLWIQTEDYKSKGDKPVYEIHPLLFFDESNGGAKEITFSRASVQEIFLVDSAHTHRKTVSFLTRNGNEIKDSIRRRLEVTFESHEVIHVRAVEDVARAYSEKDTRYAWDGQYTRYHGKLVQNLPNIMMPPGIT

SEQ ID NO: 115 - protein, 16 aa, Synthetic construct
KQTVVSRIQFKGNKAF

SEQ ID NO: 116 - protein, 18 aa, Synthetic construct
APVYRPQDHAATINGVSD

SEQ ID NO: 117 - protein, 8 aa, Synthetic construct
PFGQRKED

SEQ ID NO: 118 - protein, 8 aa, Synthetic construct
VKPSFTGV

SEQ ID NO: 119 - protein, 9 aa, Synthetic construct
PESGFNFSP

SEQ ID NO: 120 - protein, 14 aa, Synthetic construct
VNVSNLQVDHLWKR

SEQ ID NO: 121 - protein, 10 aa, Synthetic construct
STGDEQQGTP

SEQ ID NO: 122 - protein, 10 aa, Synthetic construct
SSEAAAKTRS

SEQ ID NO: 123 - protein, 9 aa, Synthetic construct
AVGKDIEGQ

SEQ ID NO: 124 - protein, 20 aa, Synthetic construct
VDFPLNPNTRFLAGKLPAET

SEQ ID NO: 125 - protein, 8 aa, Synthetic construct
YGNEIKDS

SEQ ID NO: 126 - protein, 16 aa, Synthetic construct
ANRWISGPHWWNSLSS

SEQ ID NO: 127 - protein, 18 aa, Synthetic construct
KGANGEGGTQGTKRTDIL

SEQ ID NO: 128 - protein, 18 aa, Synthetic construct
KNGAGSTDLKWEQFLQKY

SEQ ID NO: 129 - DNA, 48 bp, Synthetic construct
  source 1..48: mol_type = other DNA
aagcagactgttgtctcgcggatacagtttaagggaaataaggcgttt

SEQ ID NO: 130 - DNA, 54 bp, Synthetic construct
  source 1..54: mol_type = other DNA
gcgccggtgtatcggccgcaggaccacgccgcgacaatcaacggtgtttctgac

SEQ ID NO: 131 - DNA, 24 bp, Synthetic construct
  source 1..24: mol_type = other DNA
ccattcggacagcgcaaggaagac

SEQ ID NO: 132 - DNA, 24 bp, Synthetic construct
  source 1..24: mol_type = other DNA
gtaaagccgagtttcacaggagtg

SEQ ID NO: 133 - DNA, 27 bp, Synthetic construct
  source 1..27: mol_type = other DNA
cccgaatccgggtttaacttttctccc

SEQ ID NO: 134 - DNA, 42 bp, Synthetic construct
  source 1..42: mol_type = other DNA
gtgaacgtaagcaacctgcaggtggaccacctctggaagcgc

SEQ ID NO: 135 - DNA, 30 bp, Synthetic construct
  source 1..30: mol_type = other DNA
tccacaggcgatgagcaacagggcacgccg

SEQ ID NO: 136 - DNA, 30 bp, Synthetic construct
  source 1..30: mol_type = other DNA
tcttcagaagctgcggcgaagactcgctca

SEQ ID NO: 137 - DNA, 27 bp, Synthetic construct
  source 1..27: mol_type = other DNA
gccgtggggaaagacattgaggggcag

SEQ ID NO: 138 - DNA, 60 bp, Synthetic construct
  source 1..60: mol_type = other DNA
gtggactttccgctcaaccctaacacccgctttttagcaggtaagctgcctgcagaaacg

SEQ ID NO: 139 - DNA, 24 bp, Synthetic construct
  source 1..24: mol_type = other DNA
tatgggaacgagataaaggactcc

SEQ ID NO: 140 - DNA, 48 bp, Synthetic construct
  source 1..48: mol_type = other DNA
gctaatcgctggatatcaggtccccactggtggaattctctttcttcg

SEQ ID NO: 141 - DNA, 54 bp, Synthetic construct
  source 1..54: mol_type = other DNA
aagggcgcgaacggtgagggcggaacgcaaggtacgaaacgcaccgatatactg

SEQ ID NO: 142 - DNA, 54 bp, Synthetic construct
  source 1..54: mol_type = other DNA
aagaacggcgcaggctccactgatttgaaatgggaacagtttttgcaaaagtat

SEQ ID NO: 143 - protein, 7 aa, Treponema pallidum
ESGFNFS

SEQ ID NO: 144 - protein, 7 aa, Treponema pallidum
GNEIKDS

SEQ ID NO: 145 - protein, 9 aa, Treponema pallidum
STGDEQQGT

SEQ ID NO: 146 - protein, 7 aa, Treponema pallidum
FGQRKED

SEQ ID NO: 147 - DNA, 431 bp, Synthetic construct
  source 1..431: mol_type = other DNA
gtgcagaccgcgatgcgtatcgcgctgtggaaccgtgcgacccacggtgaacaaggtgcgctgcaacacctgctggcgggtctgtggattcagaccgaaggcgatgatctgcacatgggcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtctgaaaattggtagcaccagcatgtgggatggccaatacacccgttatcacgcgggtccggcgagcgcgccgagcccggcggccgc

SEQ ID NO: 148 - DNA, 434 bp, Synthetic construct
  source 1..434: mol_type = other DNA
gtgcagaccgcgatgcgtatcgcgctgtggaaccgtgcgacccacggtgaacaaggtgcgctgcaacacctgctggcgggtctgtggattcagaccgaaaaaaccgtgaacggcattcgtcatggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtctgaaaattggtagcaccagcatgtgggatggccaatacacccgttatcacgcgggtccggcgagcgcgccgagcccggcggccgc

SEQ ID NO: 149 - DNA, 440 bp, Synthetic construct
  source 1..440: mol_type = other DNA
gtgcagaccgcgatgcgtatcgcgctgtggaaccgtgcgacccacggtgaacaaggtgcgctgcaacacctgctggcgggtctgtggattcagaccgaagtgctgtataaccaggcggaaaaaggcagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtctgaaaattggtagcaccagcatgtgggatggccaatacacccgttatcacgcgggtccggcgagcgcgccgagcccggcggccgc

SEQ ID NO: 150 - DNA, 440 bp, Synthetic construct
  source 1..440: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtggcgatgatctgcacatgggcgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtctgaaaattggtagcaccagcatgtgggatggccaatacacccgttatcacgcgggtccggcgagcgcgccgagcccggcggccgc

SEQ ID NO: 151 - DNA, 441 bp, Synthetic construct
  source 1..441: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgtgctgtataaccaggcggaaaaaggtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgtgctgtataaccaggcggaaaaagacgttgcgcgtctgaaaattggtagcaccagcatgtgggatggccaatacacccgttatcacgcgggtccggcgagcgcgccgagcccggcggccgc

SEQ ID NO: 152 - DNA, 440 bp, Synthetic construct
  source 1..440: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgggcgatgatctgcacatgggcagcatccgtcgtcgtctggaagtgacctttgagagccacgaagtgatccacgttcgtgcggtggaagacgttgcgcgtctgaaaattggtagcaccagcatgtgggatggccaatacacccgttatcacgcgggtccggcgagcgcgccgagcccggcggccgc

SEQ ID NO: 153 - DNA, 443 bp, Synthetic construct
  source 1..443: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgaaaaccgtgaacggcattcgtcatagcatccgtcgtcgtctggaagtgacctttgagagccacgaagtgatccacgttcgtgcggtggaagacgttgcgcgtctgaaaattggtagcaccagcatgtgggatggccaatacacccgttatcacgcgggtccggcgagcgcgccgagcccggcggccgc

SEQ ID NO: 154 - DNA, 449 bp, Synthetic construct
  source 1..449: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcggtgctgtataaccaggcggaaaaaggcagcagcatccgtcgtcgtctggaagtgacctttgagagccacgaagtgatccacgttcgtgcggtggaagacgttgcgcgtctgaaaattggtagcaccagcatgtgggatggccaatacacccgttatcacgcgggtccggcgagcgcgccgagcccggcggccgc

SEQ ID NO: 155 - DNA, 425 bp, Synthetic construct
  source 1..425: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtggcgatgatctgcacatgggtatgtgggatggccaatacacccgttatcacgcgggtccggcgagcgcgccgagcccggcggccgc

SEQ ID NO: 156 - DNA, 428 bp, Synthetic construct
  source 1..428: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtaaaaccgtgaacggtattcgtcacatgtgggatggccaatacacccgttatcacgcgggtccggcgagcgcgccgagcccggcggccgc

SEQ ID NO: 157 - DNA, 434 bp, Synthetic construct
  source 1..434: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgtgctgtataaccaggcggaaaaaggcagcatgtgggatggccaatacacccgttatcacgcgggtccggcgagcgcgccgagcccggcggccgc

SEQ ID NO: 158 - DNA, 423 bp, Synthetic construct
  source 1..423: mol_type = other DNA
gtacaaacagcaatgaggatagctctatggaaccgtgctacccacggcgaacagggtgcgctgcagcacctgttagcaggcctttggattcaaacccgtgcgtatagcgaaaaagatacccgttacgcgcacccgctgttgtttttcgatcgtgagcacgcggaaattaccttcagccgcgcgtctgttcaagagatctttctggtggacagcgcccatacgcatcgtaagaccgttagctttctgaccagaaatactgccattagctcgatccgtcgtcgcttggaggttaccttcgagtcccatgaagtaatccacgtgcgcgctgtggaggacgtcgcgcgtctgaaaatcggcagcacgtccatgtgggacggtcagtatacgcgctaccatgctggtccggcatccgcgccatctccg

SEQ ID NO: 159 - DNA, 441 bp, Synthetic construct
  source 1..441: mol_type = other DNA
gtacaaacagctatgaggatagcactatggaaccgtgctacccacggtgagcagggtgcgctgcagcacctgttggcgggcctgtggattcaaacgggtaaacttgttcagaatctgccgaacatcatgatgccgccaggcatcacccatccgctgttgtttttcgaccgtgagcacgccgaaatcaccttctctcgtgcttccgtgcaagagatttttctggtggacagcgcgcacacccaccgtaagacggtttcgtttttaacgcgtaacaccgcgattagcagcatccgtcgccgtttggaagtcactttcgagagccatgaagtgattcacgtgcgcgcggttgaagatgttgcacgcctgaaaatcggctccaccagcatgtgggatggccaatatacccgctaccatgcgggtccggcaagcgccccgtctccg

SEQ ID NO: 160 - DNA, 429 bp, Synthetic construct
  source 1..429: mol_type = other DNA
gtacaaacagctatgaggatagcactatggaatcgtgccacgcacggcgaacagggtgctctgcaacatctgctggcaggtttatggattcagaccgaggctaaaaccattgaagcgactctgcatggcgacatccacccgctgttgttttttgatcgtgagcacgcggaaatcaccttcagccgtgcgtcggtgcaagagatcttcctggtggacagcgcgcacacccaccgcaaaaccgttagctttctcacgcgcaacactgcgattagctctatccgccgtcgtttggaggtcaccttcgagagccacgaagttattcatgttcgtgcagtagaagacgtggcccgtctgaagatcggctccacctccatgtgggatggtcagtataccagataccatgcgggtccagcatctgctccgagcccg

SEQ ID NO: 161 - DNA, 417 bp, Synthetic construct
  source 1..417: mol_type = other DNA
gtacaaacagcaatgaggatagctctatggaatcgtgctacccacggtgaacaaggtgcgctgcagcacttactggcaggcctgtggattcagaccgcgaaaacgatcgaagcaacgctgcatcatccgctgttgttttttgatcgtgagcacgcggaaatcaccttcagccgtgcctctgttcaagagatcttcctggtggacagcgcgcacacccaccgcaaaacggtgtcgttcctgactcgcaacaccgcgatttccagcattcgtcgccgtttggaggtcacctttgagagccacgaggtgattcatgttagagccgttgaagatgtagcgcgtcttaagatcggctccaccagcatgtgggacggccagtacacccgttatcatgctggtccggcatccgcgccaagcccg

SEQ ID NO: 162 - DNA, 423 bp, Synthetic construct
  source 1..423: mol_type = other DNA
gtacaaacagcaatgaggatagctctatggaaccgcgctacccacggcgaacagggtgcgctgcaacatctgttggcaggcctgtggattcagactgaggagtctaacggtggtgccaagggtgatattcacccactgttgtttttcgaccgcgagcacgccgaaatcaccttcagccgtgcgtccgtgcaggagatctttcttgtagacagcgcgcacacccatcgtaaaaccgttagctttttgacccgtaataccgcaattagctccattcgtcgtcgcctggaagttacgttcgagtcccatgaagtcatccacgtgcgtgcggtggaagatgttgcgcgtctgaaaatcggcagcacctctatgtgggacggccaatatacgagataccatgcgggtccggcaagcgctccgtcgccg

SEQ ID NO: 163 - DNA, 432 bp, Synthetic construct
  source 1..432: mol_type = other DNA
gtacaaacagctatgaggatagcactatggaaccgcgctacccacggtgaacagggtgcgctgcagcatctgctggcaggcctgtggattcagacggaggactacaaaagcaaaggtgataaaccggtttatgaaattcacccgctgttattttttgatcgtgagcacgccgagatcaccttcagccgtgcttctgttcaagagatctttttggttgatagcgcgcacacccaccgcaagaccgtttcgttcttgacccgtaataccgcaattagcagcattcgtagacgcctggaagtgacgttcgagtcccatgaagtcatccacgtgcgtgcggtggaagacgtggcccgtctcaagatcggcagcacttcaatgtgggacggccaatatacccgctaccatgcgggtccggcgtctgcgccatccccg

SEQ ID NO: 164 - DNA, 435 bp, Synthetic construct
  source 1..435: mol_type = other DNA
gtacaaacagctatgaggatagcactatggaaccgtgccacccacggtgagcagggcgcgctgcaacacctgctggcgggtctgtggattcagaccgaactgcaagagaatagcaatgttgttattgaaaaaaacgtgatccacccactgttgtttttcgaccgtgagcacgccgaaatcacgttcagccgtgcgtccgtgcaagagatcttcctcgtcgatagcgcgcacacccaccgcaaaaccgttagctttctgacccgcaacaccgcaatttctagcattcgtcgtcgcttggaggtgacgtttgaatcccatgaagtcatccatgttcgtgctgtggaagatgttgcgcgtcttaagatcggcagcacttcaatgtgggacggccagtataccagataccatgcgggtccggcatctgctccgtcgccg

SEQ ID NO: 165 - DNA, 435 bp, Synthetic construct
  source 1..435: mol_type = other DNA
gtacaaacagctatgaggatagcactatggaaccgtgcgacccacggcgaacagggcgcactgcaacacctgttagcgggtctgtggattcaaaccgaaatctccccgaatagcggtgacatccacccgctgctgttcttcgacgacaacaaccaaccgtttgacctgacggtggagatcactttcagccgtgcgtctgttcaggagatctttctggttgatagcgcgcatacccaccgcaaaaccgtgagctttttgacgcgtaataccgcaatttcgagcattcgtcgccgcttggaagtgactttcgagtcccatgaagttattcacgtgcgtgctgtcgaggatgttgcgcgtcttaagatcggcagcaccagcatgtgggatggtcagtataccagataccatgcaggtccggcttctgccccatccccg

SEQ ID NO: 166 - DNA, 426 bp, Synthetic construct
  source 1..426: mol_type = other DNA
gtacaaacagcaatgaggatagctctatggaaccgcgcgacccacggcgagcagggtgcgctgcagcatctgctggcaggcctgtggattcaaacggaaatctcaccgaacagcggtgacatccacccgctgttgtttttcgatgaatccaacggcggtgccaaggagatcaccttcagccgtgcctccgtgcaagagatctttcttgtggacagcgcgcacacccaccgtaaaaccgtttcttttctcacgcgtaataccgcaatttcttccattcgcagacgtctggaagtcaccttcgagagccatgaagtgattcacgttcgtgctgttgaggacgttgcgcgtttgaagatcggcagcaccagcatgtgggatggtcagtacactcgctatcatgcgggtccagcttcggctccgagcccg

SEQ ID NO: 167 - DNA, 444 bp, Synthetic construct
  source 1..444: mol_type = other DNA
gtacaaacagcaatgaggatagctctatggaaccgtgcgacgcatggtgaacagggcgcgctgcaacacctgttggctggcctgtggattcagaccgaaatctccccgaattctggtgatatccacccgctgctcttctttgacctgcaagaaaattccaacgtcgtgattgaaaaaaacgtggagatcaccttcagccgtgcgagcgttcaagagatctttttagttgattctgcccacacgcaccgtaaaaccgttagctttctgactcgcaacaccgcaatttcgagcattcgtcgccgtttggaggtcaccttcgagagccatgaagtgatccacgttagagctgtggaggacgttgcgcgtctgaagatcggttccaccagcatgtgggacggccagtatacccgctaccatgccggtccggcaagcgcgccatctccg

SEQ ID NO: 168 - DNA, 432 bp, Synthetic construct
  source 1..432: mol_type = other DNA
gtacaaacagctatgaggatagcactatggaaccgtgctacgcatggtgaacagggcgcgctgcaacacctgttagcgggcctgtggattcagaccgaaatcagcccaaattctggtgacatccacccgctgctctttttcgaccgtgagcacgccgagattaccttcagccgtgcgtccgtgcaagagatctttctggttgatagcgcgcatacccaccgcaagaccgtgagctttttgactcgcaatgacaacaaccaaccgttcgacctgacggttattcgtagacgcttggaagtcaccttcgagtcgcacgaggtgattcatgttcgtgctgtggaagatgttgcgcgtctgaaaatcggcagcaccagcatgtgggatggtcagtatacccgttaccatgccggtccggcatctgcaccgtccccg

SEQ ID NO: 169 - DNA, 435 bp, Synthetic construct
  source 1..435: mol_type = other DNA
gtacaaacagcaatgaggatagctctatggaatcgtgccacgcatggtgagcagggtgctctgcagcacttgctggcaggcctgtggattcaaaccgaaatcagcccgaacagcggtgatattcacccgctgcttttttttgatcgtgaacatgcggaaattacgttcagccgtgcttccgtgcaggagatcttcttggttgactccgcgcacacccatcgtaaaaccgttagctttctgactcgcaaccgcgcgtattccgaaaaagataccagatacgcaattcgtcgtcgcctggaagttaccttcgagagccacgaggtcatccacgttcgtgcggtggaggacgtggcgcgtctcaagatcggctcgaccagcatgtgggacggccaatatacccgctaccatgccggtccggcgtctgcaccgtctcca

SEQ ID NO: 170 - DNA, 423 bp, Synthetic construct
  source 1..423: mol_type = other DNA
gtacaaacagctatgaggatagcactatggaaccgcgctactcacggcgaacagggtgcgctgcaacacttgctggcaggcctgtggattcagaccgagatctccccgaatagcggtgacattcatccgctgttgtttttcgaccgcgagcacgccgagatcacgttcagccgtgcgagcgttcaagagatttttctggtggacagcgcgcatacccacagaaagaccgttagctttctcacgcgtaacgaatctaacggcggtgccaagattcgtcgtcgcctggaagttaccttcgagtcccatgaagtcatccacgtgcgtgctgtggaagatgtagcacgtttaaaaatcggctcgaccagcatgtgggatggtcagtacacccgttatcatgcgggtccggcgtctgcgccatccccg

SEQ ID NO: 171 - DNA, 441 bp, Synthetic construct
  source 1..441: mol_type = other DNA
gtacaaacagcaatgaggatagctctatggaaccgtgcaacgcacggcgaacagggtgctttgcaacatctgttggcgggtctgtggattcagaccgaaatctcgccgaattctggtgacatccacccgctgctgttttttgatcgtgagcacgccgaaattaccttcagccgtgcgtccgtgcaggagatcttcctggttgacagcgcgcatacccaccgtaagaccgtttcctttctgactcgtaatctgcaagagaactctaacgtagttattgagaaaaacgtgattcgccgtcgcttagaggttaccttcgagagccatgaagtcatccacgtgcgcgctgtggaagatgtcgcccgtctcaagatcggcagcacgagcatgtgggacggccaatatacccgctaccatgcgggtccggcatccgcgccaagcccg

SEQ ID NO: 172 - DNA, 420 bp, Synthetic construct
  source 1..420: mol_type = other DNA
gtacaaacagctatgaggatagcactatggaaccgcgccacccacggcgagcagggcgcgctgcaacatctcttggctggcctgtggattcagaccgaaatctctccgaatagcggtgacatccacccgctgttgtttttcgaccgtgaacacgcggagatcacctttagccgtgcgtccgtgcaagagatcttcttagttgacagcgcgcatacccatagaaagaccgtgtcgttcctgacgcgtaataccgcaatttctagcattcgtcgccgtctggaagtgaccttcgagagccatgaggttattcacgtgcgtgccgttgaagatgtcgcagataacaaccagccatttgacctgacggtttgggatggtcaatatactcgctaccacgcgggtccggcgtccgctccgagcccg

SEQ ID NO: 173 - DNA, 1008 bp, Synthetic construct
  source 1..1008: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtttccagaagaacccgcgtaccggtccgggcaaacacacccacggttttcgtaccaccaactgggatggccaatacacccgttatcacgcgggtccggcgagcgcgccgagcccggcggccgcctaagtaactaaactcgagcaccaccaccaccaccactaatgttaattaagttgggcgttcctaggctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctgagcaataactagcataaccccttggggcctctaaacgggtcttgaggggttttttgctgaaaggaggaactatatccgga

SEQ ID NO: 174 - DNA, 1014 bp, Synthetic construct
  source 1..1014: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtctggcggtggagctggcgagcagcaagagcagcaccgcgctgagcttcaccaaaccgaccgcgagctgggatggccaatacacccgttatcacgcgggtccggcgagcgcgccgagcccggcggccgcctaagtaactaaactcgagcaccaccaccaccaccactaatgttaattaagttgggcgttcctaggctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctgagcaataactagcataaccccttggggcctctaaacgggtcttgaggggttttttgctgaaaggaggaactatatccgga

SEQ ID NO: 175 - DNA, 423 bp, Synthetic construct
  source 1..423: mol_type = other DNA
gtacaaacagctatgaggatagcactatggaaccgtgcgacccacggtgaacagggtgcactccaacacctgctggctggcctgtggattcaaaccgaaatctccccgaacagcggcgacatccacccgctgttatttttcgatcgtgagcacgccgagatcacgttcagccgtgcttcggtgcaggagatctttttggttgatagcgcgcatacccacagaaagaccgtaagctttttgacccgtaatacggcgatttcttccattcgtcgccgcctggaagtgaccttcgagtcccatgaagttattcacgtgcgtgcggtcgaggacgttgcgcgtgcgtatagcgaaaaagacactcgctacgcctgggatggtcagtatacccgctaccatgcgggcccagcaagcgcaccgtctccg

SEQ ID NO: 176 - DNA, 441 bp, Synthetic construct
  source 1..441: mol_type = other DNA
gtacaaacagcaatgaggatagctctatggaaccgtgctacccacggcgagcagggtgcgctgcaacacctgcttgccggcctgtggattcagacggaaatttctccgaacagcggtgacatccacccgctgttgtttttcgaccgcgagcacgccgaaatcacgttctctagagcgagcgttcaagagatctttttggtggacagcgcgcatacccatcgtaaaaccgtaagctttctgacccgtaataccgcaatttcgagcattcgtcgtcgcctggaagtcaccttcgagtcccatgaggtgattcacgttcgtgctgtggaagatgttgcaggcactacaaatcgcttcaacatcatcaacgcggcgggaaacctgttgaattgggatggtcagtacacccgttatcatgcgggtccggcaagcgcgccgtcccca

SEQ ID NO: 177 - DNA, 996 bp, Synthetic construct
  source 1..996: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtaacggtgactacaagagcaaaggcgataagccggtgtatgagccgggttgggatggccaatacacccgttatcacgcgggtccggcgagcgcgccgagcccggcggccgcctaagtaactaaactcgagcaccaccaccaccaccactaatgttaattaagttgggcgttcctaggctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctgagcaataactagcataaccccttggggcctctaaacgggtcttgaggggttttttgctgaaaggaggaactatatccgga

SEQ ID NO: 178 - DNA, 984 bp, Synthetic construct
  source 1..984: mol_type = other DNA
gtgcagaccgcgatgcgtatcgcgctgtggaaccgtgcgacccacggtgaacaaggtgcgctgcaacacctgctggcgggtctgtggattcagaccgaaattagcccgaacagcggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgactacaagagcaaaggtgataagccggtgtatgagtgggatggccaatacacccgttatcacgcgggtccggcgagcgcgccgagcccggcggccgcctaagtaactaaactcgagcaccaccaccaccaccactaatgttaattaagttgggcgttcctaggctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctgagcaataactagcataaccccttggggcctctaaacgggtcttgaggggttttttgctgaaaggaggaactatatccgga

SEQ ID NO: 179 - DNA, 426 bp, Synthetic construct
  source 1..426: mol_type = other DNA
gtacaaacagcaatgaggatagctctatggaaccgtgcgactcatggtgaacaaggtgccttacagcacctgttggctggcctgtggattcagacggaaatctccccgaacagcggcgacatccacccgctgttgttctttgatcgtgagcacgcggagatcaccttttctcgtgcgtcggttcaggagatcttccttgttgacagcgcccacacgcatcgtaagaccgttagctttctgacccgcaataccgcgatttctagcattagacgtcgcctggaagtcaccttcgagagccatgaagtgattcacgtgcgtgctgtggaagatgtagcagactacaaatccaagggtgataaaccagtttatgagtgggacggccaatatacccgctaccatgcgggtccggcatccgcgccgagcccg

SEQ ID NO: 180 - DNA, 420 bp, Synthetic construct
  source 1..420: mol_type = other DNA
gtacaaacagcaatgaggatagctctatggaacagagctactcacggcgaacaaggtgcgctgcaacatctgctggctggcctgtggattcagaccgaaatctccccgaacagcggtgacatccacccgctcttgtttttcgatcgtgagcacgcggaaatcacgttcagccgtgcgagcgtacaggagatctttttagttgattctgcccacacccatcgtaagacggtgagctttttgacccgtaataccgcgatttctagcattcgtcgccgcctggaggtcaccttcgagagccatgaagttattcacgtgcgcgcggttgaggacgtggcacgtgattataaatccaaaggcgacaagccgtgggacggtcagtacacccgttaccatgccggtccggcatccgcgccatcgccg

SEQ ID NO: 181 - DNA, 420 bp, Synthetic construct
  source 1..420: mol_type = other DNA
gtacaaacagcaatgaggatagctctatggaacagagccacccacggcgagcagggtgcactgcagcatctgctcgctggcctgtggattcagacggaaatctctccgaatagcggtgacatccacccgctgttgtttttcgatcgtgagcacgcggaaatcacgttcagccgtgcgtccgtgcaagagatctttttggttgatagcgcgcatacccaccgcaagaccgttagctttctgactcgcaacaccgcaatttctagcattcgtcgtcgcctggaagtcaccttcgagagccatgaagtgattcatgttcgtgctgtggaagatgttgcgcgttccaaaggtgacaaaccggtgtacgagtgggacggccaatacacccgttatcacgccggtccggcatcggcgccaagtccg

SEQ ID NO: 182 - DNA, 420 bp, Synthetic construct
  source 1..420: mol_type = other DNA
gtacaaacagctatgaggatagcactatggaaccgcgcgacccacggcgaacagggtgcactgcaacatctgttagccggtctgtggattcagaccgagattagcccaaacagcggcgacatccatccgctgctgtttttcgaccgcgagcacgccgaaatcacctttagccgcgcgtccgtgcaagagatcttcctggtcgatagcgcgcacacgcaccgtaagacggtttccttcttgacccgtaataccgcaatttctagcattagacgccgtttggaagtgacctttgagagccatgaggtgatccacgttcgtgctgtggaagatgttgcgcgtaagtcgaaaggtgataaaccggtttactgggacggccagtatactcgttaccatgcgggtccggcatctgctccgtccccg

SEQ ID NO: 183 - DNA, 455 bp, Synthetic construct
  source 1..455: mol_type = other DNA
gtgcagaccgcgatgcgtatcgcgctgtggaaccgtgcgacccacggtgaacaaggtgcgctgcaacacctgctggcgggtctgtggattcagaccgaacgtgcgtatagcgaaaaagatacccgttatgcgggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgattataaaagcaaaggtgataaaccggtgtatgaatgggatggccaatacacccgttatcacgcgggtccggcgagcgcgccgagcccggcggccgc

SEQ ID NO: 184 - DNA, 473 bp, Synthetic construct
  source 1..473: mol_type = other DNA
gtgcagaccgcgatgcgtatcgcgctgtggaaccgtgcgacccacggtgaacaaggtgcgctgcaacacctgctggcgggtctgtggattcagaccgaaggcaaactggtgcagaacctgccgaacattatgatgccgccgggcattaccggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgattataaaagcaaaggtgataaaccggtgtatgaatgggatggccaatacacccgttatcacgcgggtccggcgagcgcgccgagcccggcggccgc

SEQ ID NO: 185 - DNA, 473 bp, Synthetic construct
  source 1..473: mol_type = other DNA
gtgcagaccgcgatgcgtatcgcgctgtggaaccgtgcgacccacggtgaacaaggtgcgctgcaacacctgctggcgggtctgtggattcagaccgaaggcaccaccaaccgttttaacattattaacgcggcgggcaacctgctgaacggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgattataaaagcaaaggtgataaaccggtgtatgaatgggatggccaatacacccgttatcacgcgggtccggcgagcgcgccgagcccggcggccgc

SEQ ID NO: 186 - DNA, 473 bp, Synthetic construct
  source 1..473: mol_type = other DNA
gtgcagaccgcgatgcgtatcgcgctgtggaaccgtgcgacccacggtgaacaaggtgcgctgcaacacctgctggcgggtctgtggattcagaccgaaggcaccaccaaccgttttaacattattaacccggcgggcaacctgctgaacggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgattataaaagcaaaggtgataaaccggtgtatgaatgggatggccaatacacccgttatcacgcgggtccggcgagcgcgccgagcccggcggccgc

SEQ ID NO: 187 - DNA, 481 bp, Synthetic construct
  source 1..481: mol_type = other DNA
ccatgggagtgcagaccgcgatgcgtatcgcgctgtggaaccgtgcgacccacggtgaacaaggtgcgctgcaacacctgctggcgggtctgtggattcagaccgaacagggcgtgctggcggatgcgccgtatatgggcattgcggaaagcatttggggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgattataaaagcaaaggtgataaaccggtgtatgaatgggatggccaatacacccgttatcacgcgggtccggcgagcgcgccgagcccggcggccgc

SEQ ID NO: 188 - DNA, 452 bp, Synthetic construct
  source 1..452: mol_type = other DNA
gtgcagaccgcgatgcgtatcgcgctgtggaaccgtgcgacccacggtgaacaaggtgcgctgcaacacctgctggcgggtctgtggattcagaccgaaaaatatcgtggcgaaggcaacgtgtatgcgggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgattataaaagcaaaggtgataaaccggtgtatgaatgggatggccaatacacccgttatcacgcgggtccggcgagcgcgccgagcccggcggccgc

SEQ ID NO: 189 - DNA, 449 bp, Synthetic construct
  source 1..449: mol_type = other DNA
gtgcagaccgcgatgcgtatcgcgctgtggaaccgtgcgacccacggtgaacaaggtgcgctgcaacacctgctggcgggtctgtggattcagaccgaagcgaaaaccattgaagcgaccctgcatggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgattataaaagcaaaggtgataaaccggtgtatgaatgggatggccaatacacccgttatcacgcgggtccggcgagcgcgccgagcccggcggccgc

SEQ ID NO: 190 - DNA, 452 bp, Synthetic construct
  source 1..452: mol_type = other DNA
gtgcagaccgcgatgcgtatcgcgctgtggaaccgtgcgacccacggtgaacaaggtgcgctgcaacacctgctggcgggtctgtggattcagaccgaaggcaaactgcaggaaaacagcaacgtggtgggcgacattcacccgctgctgttctttgatcgtgagcacgcggaaatcaccttcagccgtgcgagcgtgcaggagatttttctggttgacagcgcgcacacccaccgtaagaccgtgagcttcctgacccgtaacaccgcgatcagcagcatccgtcgtcgtctggaagtgacctttgagagccacgaagtgatccacgttcgtgcggtggaagacgttgcgcgtgattataaaagcaaaggtgataaaccggtgtatgaatgggatggccaatacacccgttatcacgcgggtccggcgagcgcgccgagcccggcggccgc

SEQ ID NO: 191 - DNA, 492 bp, Synthetic construct
  source 1..492: mol_type = other DNA
gtacaaacagcaatgaggatagctctatggaaccgtgcgacccacggcgaacagggtgcgctgcaacatctgttggctggcctgtggattcagaccgaagccaaaacgattgaggccacgctgcatggtgacatccatccgctgttgtttttcgaccgtgagcacgcggaaatcactttcagccgtgcgtccgtgcaagagatctttttagttgatagcgcgcacacccaccgcaagacggtgagctttttgacccgtaataccgcgatctccagcattcgtcgtcgcctggaagtcaccttcgagagccacgaggtcatccacgttcgtgcagttgaagacgttgcgagagattataaaagtaaaggcgacaagccggtgtacgagtgggatggccagtatacccgctaccatgctggtccggcatctgctccgtcgccaggtaagctggtgcaaaacctgccgaacatcatgatgccgcctggtattacc

SEQ ID NO: 192 - DNA, 441 bp, Synthetic construct
  source 1..441: mol_type = other DNA
gtacaaacagctatgaggatagcactatggaaccgcgccacgcacggcgagcagggtgcgctgcaacatttgctggcaggcctgtggatccagaccgaagcgaaaaccattgaggcgactctgcacggcgacatccatccgctcctgttctttgatcgtgaacacgccgaaatcaccttcagccgtgcgagcgttcaagagatctttcttgtggacagcgctcacacccaccgcaaaacggtgagctttttgaccagaaacaccgcaatttcttcgattcgtcgtcgcctggaggtgaccttcgagtcccatgaagttatccacgttcgtgctgtggaagatgtagcgcgtgactacaagtccaaaggtgacaagccggtttatgagtgggatggtcagtacacccgttatcatttggtccaaaatctgccgaacattatg

SEQ ID NO: 193 - DNA, 441 bp, Synthetic construct
  source 1..441: mol_type = other DNA
gtacaaacagctatgaggatagcactatggaaccgtgcgacgcacggcgaacagggtgcgctgcaacacttgctggcgggtctgtggattcagaccgaagcaaaaaccattgaggctaccctgcatggcgacatccacccgctgctcttctttgaccgcgaacatgcggaaattaccttttctcgtgctagcgttcaagagatcttcctggttgatagcgcacacacccaccgcaagacggtcagctttttgactcgcaacaccgccatctcgagcattcgtagacgtttggaggttaccttcgagtcccatgaagtgatccacgtgcgtgcggtggaagatgtggcccgtgactataaatccaaaggcgacaagccggtttacgagtgggatggtcagtatacccgttaccataacctgccgaatatcatgatgccgcca

SEQ ID NO: 194 - DNA, 441 bp, Synthetic construct
  source 1..441: mol_type = other DNA
gtacaaacagctatgaggatagcactatggaatcgcgcgacccacggcgagcaaggtgcgctgcaacacctgctcgcgggcctgtggattcagaccgaggccaaaaccattgaagcgacgctgcatggcgacatccacccgctgttgtttttcgatcgtgagcacgctgaaatcaccttctctcgtgctagcgttcaggagatctttttggtggatagcgcacatacccatcgtaaaacggttagctttctgacccgcaacaccgcaatttcgagcatccgtcgtcgcctggaagtcaccttcgagtcccatgaagtgatccacgttcgtgccgttgaagacgtggcgcgtgactacaagtccaagggtgacaaaccggtgtacgagtgggatggtcagtatactcgctatcacaacatcatgatgccgccaggtattact

SEQ ID NO: 195 - DNA, 459 bp, Synthetic construct
  source 1..459: mol_type = other DNA
gtacaaacagctatgaggatagcactatggaaccgtgctacccacggcgaacaaggtgcgctgcagcacctgctcgcgggtctgtggattcagaccgaggaatccaatggtggtgcgaaaggcgacatccatccgctgttgttttttgatcgtgaacatgcagaaatcacgttcagccgtgcgagcgttcaagagatcttcttagtggacagcgcacacacccaccgcaagaccgtgagctttttgactcgcaacaccgcgatttcgagcattcgtcgccgtctggaggttaccttcgagtcccatgaagtcatccacgtccgtgctgtggaggacgtggcccgtgattacaaatctaagggcgacaaaccggtttatgagtgggatggccaatatacccgctaccacggtaagctggttcagaatctgccgaacatcatgatgccgccaggtattacg

SEQ ID NO: 196 - DNA, 468 bp, Synthetic construct
  source 1..468: mol_type = other DNA
gtacaaacagcaatgaggatagctctatggaaccgcgcgacccacggcgaacagggtgcgctgcagcacctgcttgccggtctgtggatccagaccgaaggtaagctgcaagagaactccaatgtcgtgggtgacatccacccgctcttgtttttcgaccgtgaacatgccgagatcacgttcagccgtgctagcgttcaagagatctttctggtggacagcgcacatactcaccgtaaaaccgtcagctttttgacccgcaacaccgcaatttcttcgattagacgtcgcttagaggtgaccttcgagagccatgaagttattcatgttcgtgcggtggaagatgttgcgcgtgattataaatccaagggcgacaaaccggtttatgagtgggatggccagtacacccgttaccacggcaagctggtgcaaaacctgccgaatatcatgatgccaccgggtattacg

SEQ ID NO: 197 - DNA, 489 bp, Synthetic construct
  source 1..489: mol_type = other DNA
gtacaaacagctatgaggatagcactatggaaccgtgcaacgcacggcgagcagggtgcgctgcaacatttgctggcgggtctgtggattcaaaccgaagactataaatcgaagggtgataagccggtctatgagatccatccgctgttattctttgacctccaagagaattctaacgttgtgattgaaaagaacgtggaaatcacctttagccgtgcgagcgttcaggagatctttttggttgatagcgcgcacacccatcgtaaaaccgtttccttcttgacgcgtaacactgcgatttcttccatccgtagacgcctggaggtgaccttcgaaagccacgaagtcatccacgtgcgtgccgttgaggacgtagctcgtgcatacagcgaaaaggacacccgctacgcttgggatggtcagtatacgcgctaccacggcaaactggtgcagaatctgccgaatatcatgatgccgccaggcattacc

SEQ ID NO: 198 - DNA, 477 bp, Synthetic construct
  source 1..477: mol_type = other DNA
gtacaaacagctatgaggatagcactatggaaccgtgcgacccacggcgaacagggtgcgctgcaacacctgctcgccggtctgtggattcagacggaactgcaagagaacagcaacgtcgtgatcgagaagaacgtgatccacccgttgttgtttttcgacgagtccaatggcggtgcgaaagaaatcaccttcagccgtgctagcgttcaagagatctttcttgtggactccgcgcacacgcatcgtaagaccgtatcttttttgacccgcaacaccgcaattagcagcatccgtcgccgtctggaagtgaccttcgagtcacatgaagtcatccacgtgcgtgccgttgaagatgttgcagactacaagtcgaaaggtgataagccggtttatgagtgggatggccagtatacccgctaccacggtaaactggttcagaatctgccgaatattatgatgccgccaggtattact

SEQ ID NO: 199 - DNA, 474 bp, Synthetic construct
  source 1..474: mol_type = other DNA
gtacaaacagcaatgaggatagctctatggaaccgcgctacccacggcgaacagggcgcactgcagcacctcttagccggtctgtggattcaaaccgaagactacaagtcgaaaggcgataagccggtttatgagatccacccgctgctgttttttgacgttaaaccgtccttcaccggtgtcgagattacgttctctcgtgcgagcgtgcaagaaatcttcttggtcgatagcgcgcacacccatcgtaagacggttagctttttgacccgcaacaccgcgatttccagcatccgtcgtcgcctggaagttactttcgagtctcacgaggtgatccatgttcgtgctgtggaggacgtggcgcgtgcgtatagcgaaaaggacacccgttacgcatgggatggtcagtataccagataccacggtaaactggtgcaaaacctgccaaatatcatgatgccgccgggtattacg

SEQ ID NO: 200 - DNA, 471 bp, Synthetic construct
  source 1..471: mol_type = other DNA
gtacaaacagctatgaggatagcactatggaaccgtgccacccacggcgaacagggcgctctgcagcatctcctggcaggcctgtggattcaaaccgaagactacaaaagcaagggtgataaaccggtttatgaaatccacccgctgttgtttttcgacgagtctggttttaacttctcggagatcacgttctctcgtgctagcgttcaagagatctttttagtcgacagcgcacatacccaccgtaaaaccgttagctttttgacccgtaataccgcgattagcagcattcgtcgccgtctggaagtaacgttcgagtcccatgaagtgatccacgtgagagcggtggaagatgttgcgcgtgcgtattccgagaaggacactcgctacgcgtgggatggtcagtatacccgctaccacggcaagctggtgcaaaacctgccgaatatcatgatgccgccaggtattacc

SEQ ID NO: 201 - DNA, 471 bp, Synthetic construct
  source 1..471: mol_type = other DNA
gtacaaacagctatgaggatagcactatggaaccgtgcgacgcacggcgaacaaggtgctctgcagcacctgctggcaggcctctggatccagaccgaagactacaaatcgaagggtgataaaccggtttacgagatccacccgctgttatttttcgacggcaacgaaatcaaggactctgagatcactttcagccgtgcctctgtgcaagagatctttttggtcgatagcgcacatacgcatcgtaagaccgtgtcctttttgacccgtaataccgctatcagctccattcgtcgtcgcctggaggtgaccttcgagagccatgaggttattcacgttcgtgcggttgaagacgtcgcgagagcgtatagcgaaaaagatacccgctacgcgtgggatggccagtatacccgctatcacggtaaactggtgcaaaacctgccgaatattatgatgccgccaggtattacc

SEQ ID NO: 202 - DNA, 474 bp, Synthetic construct
  source 1..474: mol_type = other DNA
gctccagtatataggccccaagatcacgcagcgaccattaacggcgtttcggaccagggtgcgctgcaacacctgctggcaggcttatggattcaaacggaagactacaagtccaaaggtgataaaccggtttatgaaatccacccgctgctgtttttcgacgagtctaacggcggcgctaaagagatcacgttcagccgtgcgagcgttcaagagatctttttggtagatagcgcccacacccatcgtaagaccgtgtcctttttgacccgcaacaccgcgatctcttccattcgtagacgcctggaagtcaccttcgagagccatgaagtgatccacgttcgtgctgtggaagatgtcgcacgtgcgtatagcgagaaggacactcgctacgcctgggatggtcagtatacccgttaccacggtaaactcgtgcagaatctgccgaatattatgatgccaccgggtattact

SEQ ID NO: 203 - DNA, 462 bp, Synthetic construct
  source 1..462: mol_type = other DNA
gtaaatgtttcaaacctacaagtcgatcacctgtggaaacgtcagggtgctctgcaacacctgctcgcaggtctgtggattcaaaccgaagactacaagtcgaaaggcgacaagccggtttatgagatccacccgctgttgtttttcgatgagtctaatggcggtgcgaaagaaatcacgttcagccgtgccagtgttcaggagatctttttagttgatagcgcgcatacccatcgtaagaccgtctcctttttgactcgtaacaccgcaattagcagcattcgtcgccgtctggaggtgaccttcgagtcccatgaagtgatccacgtacgcgcggtggaagacgtggcgcgtgcgtatagcgaaaaggacacccgctacgcttgggatggtcagtataccagataccacggcaaattggttcaaaacctgccgaacattatgatgccgccaggcatcacg

SEQ ID NO: 204 - DNA, 480 bp, Synthetic construct
  source 1..480: mol_type = other DNA
gtagattttccactaaatcccaacacaaggttcctggcaggcaagctgccggctgaaacgcagggtgcgctgcagcacctgctggcgggcctctggattcagaccgaagactacaaatctaagggtgataagccggtttatgagatccacccgctgttgtttttcgacgagagcaatggcggtgcgaaagaaatcactttcagccgtgcttccgtgcaagaaatctttttagtagactctgcgcatacccatcgtaaaaccgttagctttttgactagaaacaccgcaattagctccatccgtcgccgtttggaggtcaccttcgagtcgcacgaagtgattcacgtgcgcgcagttgaggacgtggcccgtgcgtatagcgagaaagatacccgctacgcgtgggatggtcaatatacgcgttaccacggtaagctggttcaaaacctgccgaacatcatgatgccaccgggcattacc

SEQ ID NO: 205 - DNA, 474 bp, Synthetic construct
  source 1..474: mol_type = other DNA
aaaaatggggctggatcaacagatctaaagtgggaacagttcttacagaaataccagggtgcgctgcaacacctgctcgcgggtctgtggattcagaccgaagactacaaatccaaaggcgataagccggtttatgagatccacccgctgttgtttttcgatgaatccaacggcggcgcgaaagagatcacgttcagcagagcctcggtgcaagagatctttctggtggatagcgcacacacccatcgtaagaccgtgagcttcttgacccgcaacaccgctatttctagcattcgtcgtcgcttggaggtcacctttgaaagtcatgaagtcatccacgttcgtgctgtggaggacgttgcgcgtgcgtatagcgaaaaggacacccgttacgcatgggacggccagtatacgcgctaccacggtaagctggttcaaaacctgccgaatatcatgatgccgccaggtattact

SEQ ID NO: 206 - DNA, 468 bp, Synthetic construct
  source 1..468: mol_type = other DNA
gctaataggtggataagtggaccccactggtggaacagcctgagctctcagggcgcgctgcaacacctgctggcaggcttatggattcagaccgaagactacaaaagcaaaggtgataaaccggtttatgagatccacccgctgttgtttttcgatgaaagtaatggcggtgcgaaagagatcacgttcagccgtgcgtccgtgcaagaaatctttctcgtggatagcgctcacacccatcgtaagactgtaagctttttgacccgcaacaccgcaattagctccattcgtcgccgtctggaagttaccttcgagtcccatgaggtgatccacgtgcgtgccgttgaagacgttgcgcgtgcgtattctgagaaggacacccgctacgcttgggacggccagtataccagataccacggtaagctggtccaaaacctgccgaatatcatgatgccgccaggtattacg

SEQ ID NO: 207 - DNA, 471 bp, Synthetic construct
  source 1..471: mol_type = other DNA
gtacaaacagctatgaggatagcactatggaaccgtgctacccacggcagctcggaggcggcggctaagacgctggcgggtctgtggattcagaccgaagactacaagtccaaaggtgataaaccggtttatgaaatccacccgctgttgtttttcgacgaaagcaatggtggcgcaaaggaaatcacgttctctcgtgcgtccgtgcaagagatcttcttagtggactccgcacacacccatcgtaaaaccgttagctttttgacccgtaataccgcaattagcagcatcagacgtcgcctggaggtcacctttgaatctcatgaggttattcacgtgcgtgccgtcgaggacgtggcgcgtgcgtatagcgagaaagatacgcgctacgcctgggatggccagtatactcgctaccacggtaagctggttcaaaacctgccgaacatcatgatgccaccgggtattacc

SEQ ID NO: 208 - DNA, 471 bp, Synthetic construct
  source 1..471: mol_type = other DNA
gtacaaacagctatgaggatagcactatggaaccgcgctacccacggcgtgaagccgagcttcaccggtgttctggcgggtctgtggattcagaccgaggactataagtctaaaggtgataaaccggtgtatgaaatccacccgctgctgttttttgacgaatctaatggcggcgcgaaagagatcacgttcagccgtgcgagcgttcaagagatctttttggttgatagcgcacatactcaccgtaagactgtatcgttcttgaccagaaacaccgcaatttccagcattcgtcgccgtctggaggtgaccttcgagtcccatgaagtcatccacgtgcgtgctgtggaagacgttgcgcgtgcctacagcgaaaaggacacccgttacgcgtgggatggtcagtatacccgctaccacggtaaactggttcaaaacttaccgaatatcatgatgccaccgggtattacg

SEQ ID NO: 209 - DNA, 474 bp, Synthetic construct
  source 1..474: mol_type = other DNA
gtacaaacagctatgaggatagcactatggaaccgtgctacccacggcccggaatccggttttaacttctctccgctggcaggcctgtggattcagaccgaggactacaaatccaagggtgataaaccggtttatgaaatccacccgctcttgtttttcgatgagtctaatggtggcgcaaaagagatcacgttcagccgtgcgagcgttcaagagatctttctggtcgacagcgcgcatacccatcgtaaaaccgttagctttttgacccgcaacaccgcgattagctcgatccgtcgtagactggaagtcaccttcgagtcccacgaggtgatccacgtgcgtgcggttgaagacgtggcccgtgcgtatagcgaaaaggacacgcgctacgcttgggatggtcagtatacccgctaccacggtaagctggtgcaaaacctgccgaatattatgatgccaccgggtattact

SEQ ID NO: 210 - DNA, 486 bp, Synthetic construct
  source 1..486: mol_type = other DNA
gtacaaacagcaatgaggatagctctatggaaccgcgctacgcacggcgagcagggtgctctgcaacacctgttggcgggtctgtggattcaaaccgaagattataaatctaagggtgataaaccggtttatgaaatccacccgttgttgtttttcgacgaaagcaacggcggcgcgaaagagatcacgttcagccgtggtaacgaaatcaaggactctgtacaagagatcttcctggtggattccgcgcacacccaccgtaagacggtgagctttctgacccgtaataccgcaattagctccattcgtagacgtctcgaggtgacctttgagagccatgaagtgattcatgttcgtgccgtcgaggacgttgcacgtgcgtattcggaaaaggacacccgctacgcgtgggatggtcagtacacccgctaccacggtaaactggttcagaatctgccgaacatcatgatgccaccgggtattact

SEQ ID NO: 211 - DNA, 486 bp, Synthetic construct
  source 1..486: mol_type = other DNA
gtacaaacagcaatgaggatagctctatggaaccgtgctacccacggcgaacagggtgctctgcagcacctgcttgccggtctgtggatccaaaccgaggactacaaaagcaaaggtgacaaaccggtttatgaaatccacccgctgttgtttttcgacgagtctaacggcggtgcgaaagagatcacgttcagccgtgcgtctgttcaagagatcttcttggtggatagcgcgcatactcacggcaatgaaatcaaggacagcaccgtttcctttttgacccgcaacaccgcaattagctccattcgtcgccgcctggaggtcacctttgaaagtcatgaagtgatccacgtgcgtgccgtcgaggatgttgcacgtgcgtattcggaaaaggataccagatacgcgtgggatggtcagtatacccgttaccacggtaagctggtgcaaaacctgccgaatattatgatgccgccaggtattacg

SEQ ID NO: 212 - DNA, 483 bp, Synthetic construct
  source 1..483: mol_type = other DNA
gtacaaacagctatgaggatagcactatggaaccgcgcgacccacggcgaacagggcgcgctgcagcacctgctggcaggtctgtggattcagaccgaagactacaagtcgaaaggtgacaaaccggtttatgaaatccacccgctcttatttttcgatgagtccaacggcggtgcgaaagagatcactttctctcgtgcgagcgttcaagagatctttttggtcgatagcgcacatacccaccgtaagaccgtgagctttttgacccgcaacagcacgggtgacgagcaacaaggtacaattcgtagacgtctggaggtgaccttcgagtcccatgaagtcatccacgttcgtgccgttgaagacgtggctcgtgcgtatagcgaaaaagatacccgctacgcttgggatggccagtatacccgttaccacggtaagctggtgcagaatctgccgaatattatgatgccgccaggtattacg

SEQ ID NO: 213 - DNA, 483 bp, Synthetic construct
  source 1..483: mol_type = other DNA
gtacaaacagcaatgaggatagctctatggaaccgtgcgacccacggcgaacaaggtgcgctgcagcatctgctggcaggtctgtggattcagaccgaagactacaagagtaaaggcgataagccggtgtatgaaatccacccgttgctcttttttgatgaatccaacggcggtgctaaagagatcacgttcagccgtgcgagcgttcaagagatcttcttagtcgatagcgcacacacccatcgtaaaactgtaagctttttgacccgcaacgctgtcggtaaggacatcgagggtcagattcgtcgccgtctggaagttaccttcgagtctcacgaggttatccatgttcgtgcggtggaggacgtggcccgtgcgtattcggaaaaagacacccgctacgcctgggatggccaatataccagataccacggcaagctggtgcagaatctgccgaatattatgatgccgccaggtattacg

SEQ ID NO: 214 - DNA, 477 bp, Synthetic construct
  source 1..477: mol_type = other DNA
gtacaaacagctatgaggatagcactatggaaccgtgctacccacggcgaacagggtgcgctgcagcatctgctggcgggtctgtggattcagaccgaagactacaagtccaagggcgataaaccggtttatgaaatccacccgctgttgtttttcgacgagtcgaacggcggtgcgaaagagatcacgttcagccgtgcttctgttcaagagatctttcttgtggatagcgcacatacccaccgcaagacggtcagctttttgacccgtaatttcggccaacgtaaggaggacatccgtcgccgtctggaagttaccttcgagagccacgaagtgattcacgtgcgtgccgttgaagacgtcgcgagagcgtattccgagaaagatacccgctatgcatgggatggtcagtacacccgctaccacggtaaactggtgcaaaacctcccgaatattatgatgccgccaggtattact

SEQ ID NO: 215 - DNA, 480 bp, Synthetic construct
  source 1..480: mol_type = other DNA
gtacaaacagcaatgaggatagctctatggaacagagctacccacggcgaacagggcgcgctgcaacatctgcttgccggtctgtggattcaaacggaggactacaagtccaaaggtgataaaccggtttatgaaatccacccgctgctgtttttcgatgaaagcaatggcggtgcaaaagagatcacgtttagccgtgcgtctgttcaagagatctttctcgtggacagcgcacacacccaccgtaaaaccgttagcttcttgacccgcaacgtgaagccgtcgttcaccggtgttattcgtcgtcgcttggaggtgaccttcgagtcccatgaagtgatccacgttcgtgctgtggaagacgtcgcgcgtgcgtatagcgaaaaggacactcgctacgcgtgggatggtcagtatacccgctaccacggtaagctggtccagaatctgccgaacattatgatgccgccaggtattacc

SEQ ID NO: 216 - DNA, 477 bp, Synthetic construct
  source 1..477: mol_type = other DNA
gtacaaacagctatgaggatagcactatggaacagagctacccacggcgaacaaggtgcgctgcaacatctgttggcgggtctgtggatccagaccgaggactacaaaagcaaaggtgataagccggtctatgaaatccacccgctgctcttctttgatgaatccaacggtggcgcgaaagagatcacgttcagccgtgcgtcggttcaagagatctttttggtggacagcgcacacacccaccgtaagaccgttagctttttgacccgtaacggcaacgagatcaaagattctattcgtcgtcgcctggaagtcaccttcgagtcccatgaggtgattcatgttcgtgctgtggaagacgtggcccgtgcgtatagcgaaaaggacactcgctacgcatgggatggccagtacacccgctatcacggcaagctggttcagaatctgccgaatattatgatgccgccaggtattacg

SEQ ID NO: 217 - protein, 14 aa, Synthetic construct
GANGPHYYKSLESP

SEQ ID NO: 218 - protein, 16 aa, Synthetic construct
PVYGFPGPTYLPKLAG

SEQ ID NO: 219 - protein, 9 aa, Synthetic construct
PDFSSPKHL

SEQ ID NO: 220 - protein, 10 aa, Synthetic construct
QLAETQSATT

SEQ ID NO: 221 - protein, 12 aa, Synthetic construct
PTMPDTENQAPP

SEQ ID NO: 222 - protein, 16 aa, Synthetic construct
SSSYSAGNWHRPRSLL

SEQ ID NO: 223 - protein, 8 aa, Synthetic construct
TKLAKFRI

SEQ ID NO: 224 - protein, 16 aa, Synthetic construct
GNTYRDTNSARARVVE

SEQ ID NO: 225 - protein, 15 aa, Synthetic construct
SVFGSGPFVKSTFQY

SEQ ID NO: 226 - protein, 20 aa, Synthetic construct
NFVARAAGLPADAIATGAAS

SEQ ID NO: 227 - protein, 15 aa, Synthetic construct
RFNFNGFKGTRNLKG

SEQ ID NO: 228 - protein, 8 aa, Synthetic construct
NWSWPGYT

SEQ ID NO: 229 - protein, 15 aa, Synthetic construct
GLVQYRPRGSGGVAA

SEQ ID NO: 230 - protein, 11 aa, Synthetic construct
SYPFYYEKERL

SEQ ID NO: 231 - protein, 14 aa, Synthetic construct
EKVVEEEREQTNTR

SEQ ID NO: 232 - protein, 10 aa, Synthetic construct
DNNQPFDLTV

SEQ ID NO: 233 - DNA, 42 bp, Synthetic construct
  source 1..42: mol_type = other DNA
ggcgcaaacggaccacattattacaagagccttgaaagcccc

SEQ ID NO: 234 - DNA, 48 bp, Synthetic construct
  source 1..48: mol_type = other DNA
ccagtgtacggctttcccggtcctacgtatttacccaagctcgcaggg

SEQ ID NO: 235 - DNA, 27 bp, Synthetic construct
  source 1..27: mol_type = other DNA
ccagacttttcctcacccaaacacctg

SEQ ID NO: 236 - DNA, 30 bp, Synthetic construct
  source 1..30: mol_type = other DNA
caacttgcagagacgcagagcgccacaacc

SEQ ID NO: 237 - DNA, 36 bp, Synthetic construct
  source 1..36: mol_type = other DNA
cccacaatgccagatacagagaaccaggcgcctccg

SEQ ID NO: 238 - DNA, 51 bp, Synthetic construct
  source 1..51: mol_type = other DNA
agcagctcgtcttattcagccggtaactggcaccgcccacgttcattgctt

SEQ ID NO: 239 - DNA, 24 bp, Synthetic construct
  source 1..24: mol_type = other DNA
accaagctggctaagttccgcatc

SEQ ID NO: 240 - DNA, 47 bp, Synthetic construct
  source 1..47: mol_type = other DNA
ggaaatacctaccgggatcgaacagtgcgagagcacgtgtcgtcgaa

SEQ ID NO: 241 - DNA, 45 bp, Synthetic construct
  source 1..45: mol_type = other DNA
tctgtttttgggagtggtccctttgtaaagtcaacttttcaatac

SEQ ID NO: 242 - DNA, 60 bp, Synthetic construct
  source 1..60: mol_type = other DNA
aactttgtagcacgcgcggcagggttaccggcagacgcgatagcaacaggcgcagcttca

SEQ ID NO: 243 - DNA, 45 bp, Synthetic construct
  source 1..45: mol_type = other DNA
cgattcaatttcaatgggttcaaaggaacgcgcaatttaaaggga

SEQ ID NO: 244 - DNA, 24 bp, Synthetic construct
  source 1..24: mol_type = other DNA
aactggagctggcctggatacacc

SEQ ID NO: 245 - DNA, 45 bp, Synthetic construct
  source 1..45: mol_type = other DNA
ggactggtgcaataccgcccgcggggatctgggggcgttgcagcc

SEQ ID NO: 246 - DNA, 33 bp, Synthetic construct
  source 1..33: mol_type = other DNA
agttatccgttctattacgagaaggagcggctg

SEQ ID NO: 247 - DNA, 42 bp, Synthetic construct
  source 1..42: mol_type = other DNA
gaaaaagtggttgaagaagagagagagcagactaacacgcgc

SEQ ID NO: 248 - DNA, 30 bp, Synthetic construct
  source 1..30: mol_type = other DNA
gataacaatcagcccttcgacctgaccgta

SEQ ID NO: 249 - protein, 15 aa, Synthetic construct
AEHPVYGFPGPTYLP

SEQ ID NO: 250 - protein, 15 aa, Synthetic construct
AGFVARLIANYLLKG

SEQ ID NO: 251 - protein, 15 aa, Synthetic construct
KVAGFVARLIANYLL

SEQ ID NO: 252 - protein, 15 aa, Synthetic construct
LSSWVGNTAGKVAGF

SEQ ID NO: 253 - protein, 15 aa, Synthetic construct
PELAKILITHTVTHG

SEQ ID NO: 254 - protein, 15 aa, Synthetic construct
LAKILITHTVTHGLA

SEQ ID NO: 255 - protein, 15 aa, Synthetic construct
FVARLIANYLLKGSQ

SEQ ID NO: 256 - protein, 15 aa, Synthetic construct
NSNLRYLMRVGTPEL

SEQ ID NO: 257 - protein, 15 aa, Synthetic construct
RYLMRVGTPELAKIL

SEQ ID NO: 258 - protein, 15 aa, Synthetic construct
AKILITHTVTHGLAN

SEQ ID NO: 259 - protein, 15 aa, Synthetic construct
RLIANYLLKGSQYSM

SEQ ID NO: 260 - protein, 15 aa, Synthetic construct
WISGPHWWNSLSSWV

SEQ ID NO: 261 - protein, 15 aa, Synthetic construct
ANYLLKGSQYSMLVP

SEQ ID NO: 262 - protein, 15 aa, Synthetic construct
LRYLMRVGTPELAKI

SEQ ID NO: 263 - protein, 15 aa, Synthetic construct
NSLSSWVGNTAGKVA

SEQ ID NO: 264 - protein, 15 aa, Synthetic construct
WVGNTAGKVAGFVAR

SEQ ID NO: 265 - protein, 15 aa, Synthetic construct
GKVAGFVARLIANYL

SEQ ID NO: 266 - protein, 15 aa, Synthetic construct
GNTAGKVAGFVARLI

SEQ ID NO: 267 - protein, 15 aa, Synthetic construct
TPELAKILITHTVTH

SEQ ID NO: 268 - protein, 15 aa, Synthetic construct
GVDVPVNSNLRYLMR

SEQ ID NO: 269 - protein, 15 aa, Synthetic construct
AGKVAGFVARLIANY

SEQ ID NO: 270 - protein, 15 aa, Synthetic construct
IANYLLKGSQYSMLV

SEQ ID NO: 271 - protein, 15 aa, Synthetic construct
VAGFVARLIANYLLK

SEQ ID NO: 272 - protein, 15 aa, Synthetic construct
SNLRYLMRVGTPELA

SEQ ID NO: 273 - protein, 15 aa, Synthetic construct
RPQDHAATINGVSDF

SEQ ID NO: 274 - protein, 15 aa, Synthetic construct
INGVSDFFKICSPIG

SEQ ID NO: 275 - protein, 15 aa, Synthetic construct
RGGSDLWGICIQAPT

SEQ ID NO: 276 - protein, 15 aa, Synthetic construct
SDLWGICIQAPTMPD

SEQ ID NO: 277 - protein, 15 aa, Synthetic construct
DSQHFLSVLGQGLLQ

SEQ ID NO: 278 - protein, 15 aa, Synthetic construct
LGQGLLQPSSSSYSA

SEQ ID NO: 279 - protein, 15 aa, Synthetic construct
GQGLLQPSSSSYSAG

SEQ ID NO: 280 - protein, 15 aa, Synthetic construct
QHFLSVLGQGLLQPS

SEQ ID NO: 281 - protein, 15 aa, Synthetic construct
YDSQHFLSVLGQGLL

SEQ ID NO: 282 - protein, 15 aa, Synthetic construct
SQHFLSVLGQGLLQP

SEQ ID NO: 283 - protein, 15 aa, Synthetic construct
AGNWHRPRSLLGVLT

SEQ ID NO: 284 - protein, 15 aa, Synthetic construct
LTCTAKEVGAIHEES

SEQ ID NO: 285 - protein, 15 aa, Synthetic construct
VDFPLNPNTRFLAGK

SEQ ID NO: 286 - protein, 15 aa, Synthetic construct
AETVQRVALVALRQK

SEQ ID NO: 287 - protein, 15 aa, Synthetic construct
QRVALVALRQKLISE

SEQ ID NO: 288 - protein, 15 aa, Synthetic construct
VALVALRQKLISEGI

SEQ ID NO: 289 - protein, 15 aa, Synthetic construct
VQRVALVALRQKLIS

SEQ ID NO: 290 - protein, 15 aa, Synthetic construct
PNTRFLAGKLPAETV

SEQ ID NO: 291 - protein, 15 aa, Synthetic construct
NTRFLAGKLPAETVQ

SEQ ID NO: 292 - protein, 15 aa, Synthetic construct
LRQKLISEGIIKALD

SEQ ID NO: 293 - protein, 15 aa, Synthetic construct
STDLKWEQFLQKYCL

SEQ ID NO: 294 - protein, 15 aa, Synthetic construct
LKWEQFLQKYCLECP

SEQ ID NO: 295 - protein, 15 aa, Synthetic construct
LVQYRPRGSGGVAAN

SEQ ID NO: 296 - protein, 15 aa, Synthetic construct
RGSGGVAANGNKEVY

SEQ ID NO: 297 - protein, 15 aa, Synthetic construct
RSCEKVVEEEREQTN

SEQ ID NO: 298 - protein, 15 aa, Synthetic construct
LRTVRRIFSWMVQHV

SEQ ID NO: 299 - protein, 15 aa, Synthetic construct
YPSLRTVRRIFSWMV

SEQ ID NO: 300 - protein, 15 aa, Synthetic construct
VRRIFSWMVQHVDSL

SEQ ID NO: 301 - protein, 15 aa, Synthetic construct
PSLRTVRRIFSWMVQ

SEQ ID NO: 302 - protein, 15 aa, Synthetic construct
GNTYRDTNSARARVV

SEQ ID NO: 303 - protein, 15 aa, Synthetic construct
NSARARVVEQALELA

SEQ ID NO: 304 - protein, 15 aa, Synthetic construct
RTVRRIFSWMVQHVD

SEQ ID NO: 305 - protein, 15 aa, Synthetic construct
ARARVVEQALELAEK

SEQ ID NO: 306 - protein, 15 aa, Synthetic construct
SARARVVEQALELAE

SEQ ID NO: 307 - protein, 15 aa, Synthetic construct
RIFSWMVQHVDSLGI

SEQ ID NO: 308 - protein, 15 aa, Synthetic construct
EKTYPSLRTVRRIFS

SEQ ID NO: 309 - protein, 15 aa, Synthetic construct
YRDTNSARARVVEQA

SEQ ID NO: 310 - protein, 15 aa, Synthetic construct
GAASVFGSGPFVKST

SEQ ID NO: 311 - protein, 15 aa, Synthetic construct
SVFGSGPFVKSTFQY

SEQ ID NO: 312 - protein, 15 aa, Synthetic construct
AASVFGSGPFVKSTF

(19) United States
(12) Patent Application Publication
(10) Pub. No.: US 2026/0297141 A1
(43) Pub. Date: Oct. 1, 2026

(54) TP0751 CHIMERA-CONTAINING VACCINE

(71) Applicants: UVIC INDUSTRY PARTNERSHIPS INC., Victoria (CA); UNIVERSITY OF WASHINGTON, Seattle, WA (US)

(72) Inventors: Caroline E. Cameron, Victoria (CA); Martin J. Boulanger, Victoria (CA); Alloysius Martin Gomez Thomas, Victoria (CA); Simon Houston, Victoria (CA); Raghavendran Ramaswamy, Victoria (CA); Lexie Thompson, Victoria (CA); Sheila A. Lukehart, Seattle, WA (US); Lorenzo Giacani, Seattle, WA (US); Barbara Molini, Seattle, WA (US); Anna Wald, Seattle, WA (US)

(73) Assignees: UVIC INDUSTRY PARTNERSHIPS INC., Victoria, BC (CA); UNIVERSITY OF WASHINGTON, Seattle, WA (US)

(21) Appl. No.: 19/479,497

(22) PCT Filed: Nov. 15, 2023
(86) PCT No.: PCT/US2023/079879

§ 371 Date: Oct. 28, 2025

Related U.S. Application Data
(60) Provisional application No. 63/499,007, filed on Apr. 28, 2023.

(51) Int. Cl. C07K 14/20; A61K 31/43; A61K 31/546; A61K 31/65; A61K 39/00; A61K 39/02; A61P 31/04; A61P 37/04

(52) U.S. Cl. CPC C07K 14/20; A61K 31/43; A61K 31/546; A61K 31/65; A61K 39/0225; A61P 31/04; A61P 37/04; A61K 2039/575; A61K 2039/70

(57) ABSTRACT

Provided are chimeric Tp0751 (99-237) peptides containing one or more non-native epitopes that replace all or of part of one or more loops 1-8 of the native Tp0751 (99-237) peptide. The non-native epitopes can be from one or more sexually transmitted infections (STIs), such as Treponema pallidum, Chlamydia, or Neisseria gonorrhoeae. Also provided are nucleic acid molecules that encode the chimeric Tp0751 (99-237) peptides. Methods of using the chimeric Tp0751 (99-237) peptides to stimulate an immune response, for example to treat syphilis or other sexually STIs are provided.

### CROSS REFERENCE TO RELATED APPLICATIONS

This application claims priority to U.S. 63/499,007 filed Apr. 28, 2023, herein incorporated by reference in its entirety.

### ACKNOWLEDGMENT OF GOVERNMENT SUPPORT

This invention was made with government support under Contract No. U19AI144133 awarded by the National Institutes of Health. The government has certain rights in the invention.

### INCORPORATION OF ELECTRONIC SEQUENCE LISTING

The electronic sequence listing, submitted herewith as an xml file named “Sequence.xml” (321,428 bytes), created on Nov. 10, 2023, is herein incorporated by reference in its entirety.

### FIELD

The present application provides chimeric Tp0751 (99-237) peptides containing one or more non-native epitopes that replace all or of part of one or more loops 1-8 of the native Tp0751 (99-237) peptide, as well as methods of using the chimeric Tp0751 (99-237) peptides to stimulate an immune response, for example to treat syphilis or other sexually transmitted infections (STI), or other infectious diseases.

### BACKGROUND

Syphilis is a chronic, multistage disease caused by Treponema pallidum subsp. pallidum. Infectious syphilis has an estimated global burden of 56 million prevalent cases and 38 million new infections per year (1). Targeted public health control initiatives, pioneered by the Centers for Disease Control-National Plan to Eliminate Syphilis from the United States (14, 15) and the World Health Organization-Initiative for the Global Elimination of Congenital Syphilis (16), have raised awareness of syphilis prevalence and have undoubtedly averted an even greater spike in disease incidence, but have not achieved the goal of syphilis elimination.

Early infection is typified by a primary stage ulcerative lesion, called a chancre, at the site of infection, followed by a disseminated rash and mucosal lesions during the secondary stage of infection (17). Because syphilis transmission occurs by contact with the infectious primary chancre or secondary lesions, prevention or attenuation of these lesions would eliminate or reduce person-to-person syphilis transmission. In addition, reducing or eliminating the dissemination of T. pallidum within the infected host could reduce or eliminate serious sequelae of infectious and congenital syphilis.

Thus, new biomedical strategies, including vaccine development, are needed.

### SUMMARY

Provided herein is a protein engineering strategy that leverages the core structural features of the lipocalin-like domain of Tp0751 to develop a highly stable, protein-based vaccine platform that can accommodate multiple epitopes from various different proteins (FIGS. 2A-2B). Using structure-guided principles, regions of Tp0751 (99-237) were identified that are amenable to substitution. Chimeric constructs were generated and tested in expression trials followed by higher resolution approaches to evaluate various biochemical and biophysical parameters including expression levels, protein folding and stability relative to wild type Tp0751 (99-237).

Using a new protein engineering methodology, new chimeric Tp0751 (99-237) peptides were designed and tested. Provided herein are chimeric Tp0751 (99-237) peptides, which include at least one non-native epitope peptide that replaces all of or part of one or more loops of a native Tp0751 (99-237) peptide. An exemplary native Tp0751 (99-237) peptide sequence is provided in SEQ ID NO: 3, and the one or more loops of the native Tp0751 (99-237) peptide that can be partially or completely replaced are loop 1 GEQGALQHLLA (SEQ ID NO: 4), loop 2 QTEISPNSGDIHP (SEQ ID NO: 5), loop 3 REHA (aa 147-150 of SEQ ID NO: 2), loop 4 AS (aa 157-158 of SEQ ID NO: 2), loop 5 RK (aa 172-173 of SEQ ID NO: 2), loop 6 NTAISSI (SEQ ID NO: 6), loop 7 HE (aa 198-199 of SEQ ID NO: 2), and loop 8 aa DVARLKIGSTSMWD (SEQ ID NO: 7). This is also shown in FIG. 1C. In some examples, the chimeric Tp0751 peptide further includes at least one non-native epitope peptide that replaces all or of part of the native C-terminus of the native Tp0751 (99-237) peptide, wherein the native C-terminus of the native Tp0751 (99-237) peptide is SEQ ID NO: 8. In some examples, 2, 3, 4, 5, 6, 7, or 8 of the loops of the native Tp0751 (99-237) peptide are partially or completely replaced with the at least one non-native epitope peptide.

In some examples, the non-native epitope peptide that replaces all or of part of one or more loops of a native Tp0751 (99-237) peptide are from a pathogen (such as a bacteria) that causes an STI, such as epitopes from one or more of Treponema pallidum, Chlamydia, or Neisseria gonorrhoeae. In one example, the one or more non-native epitope peptides (e.g., antigenic sequences) are from T. pallidum, such as from a TprC protein, such as one or more of SEQ ID NOs: 17, 18, 24, 25, 26, 31, 32, 35, 36, or 37. In one example, the one or more antigenic sequences are from T. pallidum, such as from a TprK protein, such as one or more of SEQ ID NOs: 19, 20, 21, 22, 27, 28, 29, 30, 33, or 34. In one example, the one or more antigenic sequences are from T. pallidum, such as from a Tp0326 protein, such as SEQ ID NO: 23. In one example, the one or more antigenic sequences are from T. pallidum, such as from a Tp0856 protein, such as SEQ ID NO: 143 or 145. In one example, the one or more antigenic sequences are from T. pallidum, such as from a Tp0346 protein, such as SEQ ID NO: 144. In one example, the one or more antigenic sequences are from T. pallidum, such as from a Tp0483 protein, such as SEQ ID NO: 146. In one example, the one or more antigenic sequences include a T. pallidum B cell epitope, such as one or more of SEQ ID NOs: 115, 116, 117, 118, 119, 120, 121, 122, 123, 124, 125, 126, 127, 128, 217, 218, 219, 220, 221, 222, 223, 224, 225, 226, 227, 228, 229, 230, 231, or 232. In one example, the one or more antigenic sequences include a T. pallidum T cell epitope, such as one or more of SEQ ID NOs: 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, or 312

In some examples, the non-native epitope peptide includes one or more TprC epitopes, and one or more other B cell epitopes, one or more other T cell epitopes, or one or more other T cell epitopes and B cell epitopes. In some examples, the non-native epitope peptide includes one or more TprK epitopes, and one or more other B cell epitopes, one or more other T cell epitopes, or one or more other T cell epitopes and B cell epitopes. In some examples, the non-native epitope peptide includes epitopes from TprC, TprK, and one or more other B cell epitopes, one or more other T cell epitopes, or one or more other T cell epitopes and B cell epitopes. In one example, the one or more antigenic sequences are from T. pallidum, such as from Tp0326 protein (e.g., SEQ ID NO: 23), Tp0856 protein (e.g., SEQ ID NO: 143 or 145), Tp0346 protein (e.g., SEQ ID NO: 144), Tp0483 protein, (e.g., SEQ ID NO: 146), alone or in combination, such as further including one or more other B cell epitopes, one or more other T cell epitopes, or one or more other T cell epitopes and B cell epitopes.

In some examples, the non-native epitope peptide that replaces all or of part of one or more loops of a native Tp0751 (99-237) peptide are from a pathogen, such as from a bacterial surface protein, such as outer surface protein A (OspA) and/or outer surface protein C (OspC) of B. burgdorferi (such a peptide could be used to treat or prevent Borrelia/Lyme disease).

In some examples, the chimeric Tp0751 (99-237) peptide has at least 80%, at least 85%, at least 90%, at least 95%, at least 96%, at least 97%, at least 98%, at least 99%, or 100% sequence identity to any one of SEQ ID NOS: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114.

Also provided are isolated nucleic acid molecules that encode the chimeric Tp0751 (99-237) peptides provided herein. In some examples, such nucleic acid coding sequences are optimized for expression in bacterial cells, such as E. coli. Such molecules can be DNA or RNA, such as mRNA. Vectors and cells including such nucleic acid molecules are provided. In one example, the disclosed isolated nucleic acid molecules encoding a chimeric Tp0751 (99-237) peptide have at least 80%, at least 85%, at least 90%, at least 95%, at least 96%, at least 97%, at least 98%, at least 99%, or 100% sequence identity to SEQ ID NO: 95, 96, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 201, 203, 204, 205, 206, 307, 208, 209, 210, 211, 212, 213, 214, 215, or 216.

Also provided are liposomes and compositions that include the chimeric Tp0751 (99-237) peptides, or nucleic acid molecules or vectors encoding such. In some examples, such a composition further includes a pharmaceutically acceptable carrier and/or an adjuvant.

Also provided are methods of stimulating an immune response in a subject, and methods of treating or preventing Treponema pallidum subsp. pallidum infection in a subject. Such methods include administering to the subject a therapeutically effective amount of a chimeric Tp0751 (99-237) peptide provided herein, or an isolated nucleic acid molecule encoding a chimeric Tp0751 (99-237) peptide provided herein (or a vector including such a nucleic acid). In some examples, such methods reduce one or more symptoms of syphilis in the subject by at least 20%, at least 25%, at least 50%, at least 60%, at least 70%, at least 80%, at least 90%, at least 95%, at least 99% or 100%. In some examples, such methods can further include administration of one or more antibiotics, such as penicillin, doxycycline, tetracycline and/or ceftriaxone.

The foregoing and other objects and features of the disclosure will become more apparent from the following detailed description, which proceeds with reference to the accompanying figures.

### BRIEF DESCRIPTION OF DRAWINGS

FIGS. 1A-1C. A) Ribbon structure of WT Tp0751 lipocalin domain (PDB ID 5JK2), SEQ ID NO: 3. B) Ribbon structure depicting the inherent flexibility in regions of the Tp0751 (99-237) lipocalin domain, represented by the thermal motion parameter (B-factor) distribution. B-factor is represented by thickness, where thin lines depict structural rigidity, and thicker lines depict structural flexibility. C) Protein sequence of Tp0751 (99-237) (SEQ ID NO: 3) depicting secondary structures and regions targeted for engraftment (depicted with boxes). Loop 1 of Tp0751 (99-237) is aa 114-124 of SEQ ID NO: 2 (GEQGALQHLLA; SEQ ID NO: 4), Loop 2 of Tp0751 (99-237) is aa 129-141 of SEQ ID NO: 2 (QTEISPNSGDIHP; SEQ ID NO: 5), Loop 3 of Tp0751 (99-237) is aa 147-150 of SEQ ID NO: 2 (REHA), Loop 4 of Tp0751 (99-237) is aa 157-158 of SEQ ID NO: 2 (AS), Loop 5 of Tp0751 (99-237) is aa 172-173 of SEQ ID NO: 2 (RK), Loop 6 of Tp0751 (99-237) is aa 181-187 of SEQ ID NO: 2 (NTAISSI; SEQ ID NO: 6), Loop 7 of Tp0751 (99-237) is aa 198-199 of SEQ ID NO: 2 (HE), Loop 8 aa 208-221 of SEQ ID NO: 2 (DVARLKIGSTSMWD; SEQ ID NO: 7). The C-terminus of Tp0751 (99-237) is aa 226-237 of SEQ ID NO: 2 (RYHAGPASAPSP; SEQ ID NO: 8).

FIGS. 2A-2B A) exemplary chimera engineering workflow and the criteria for success of each step. B) exemplary pipeline for identification of additional epitopes for engraftment onto the Tp0751 (99-237) scaffold.

FIG. 3. Small-scale expression of Tp0751 chimeras. Samples taken from multiple stages of purification (insoluble pellet, lysis supernatant, purified sample) from E. coli cultures were evaluated on a 12% Bolt Bis-Tris Plus gel at 200 V for 30 minutes, and expression was assessed by the presence or absence of a strong band at the expected molecular weight (designated by arrows). Only nickel-enriched samples are shown above. Chimeric constructs that showed expression equivalent to wild type Tp0751 (99-237) and that could be enriched by nickel pulldown indicated correctly folded protein and were advanced to large scale expression trials. Loading volumes were normalized to expression volumes and benchmarked to WT to allow for direct comparison.

FIG. 4. Purity gels from large-scale Tp0751 chimera preparations following nickel affinity and size exclusion chromatography (SEC) purification. Each chimera was purified from large scale E. coli cultures with minimal protein aggregation or precipitation and was successfully on either an ENrich™ SEC 70 or a Superdex™ 75 column in 20 mM HEPES pH 8.0, 150 mM NaCl+1% glycerol. Purified proteins were visualized on a 12% Bolt Bis-Tris Plus gel at 200 V for 30 minutes. Loading volumes were normalized to expression volumes and benchmarked to WT to allow for direct comparison.

FIGS. 5A-5E. Purification data set for Tp0751 chimera 1.21 (see Table 3 for sequence descriptions) A) Structural schematic showing targeted Tp0751 loop 8 (dark grey). B) Small-scale soluble expression of Tp0751 WT and 1.21, showing that 1.21 has similar expression to WT. C) SEC trace and corresponding purity gel indicating successful purification of 1.21. D) Melting temperature (Tₘ) for both WT and 1.21, showing that 1.21 is equivalent to WT. E) Proteolytic stability profiles of WT and 1.21 following trypsin digestions at various timepoints, showing that 1.21 is equivalent to WT.

FIGS. 6A-6E. Purification data set for Tp0751 chimera 2.7 (see Table 3 for sequence descriptions) A) Structural schematic showing targeted Tp0751 loops 8 and 2 (dark grey). B) Small-scale soluble expression of Tp0751 WT and 2.7, showing that 2.7 has similar expression to WT. C) SEC trace and corresponding purity gel indicating successful purification of 2.7. D) Melting temperature (Tₘ) for both WT and 2.7, showing that 2.7 is equivalent to WT. E) Proteolytic stability profiles of WT and 2.7 following trypsin digestions at various timepoints, showing that 2.7 is equivalent to WT.

FIGS. 7A-7E. Purification data set for Tp0751 chimera 3.2 (SEQ ID NO: 82; see Table 3 for sequence descriptions) A) Structural schematic showing targeted Tp0751 loops 8 and 2 and the C-term (dark grey). B) Small-scale soluble expression of Tp0751 WT and 3.2, showing that 3.2 has similar expression to WT. C) SEC trace and corresponding purity gel indicating successful purification of 3.2. D) Melting temperature (Tₘ) for both WT and 3.2, showing that 3.2 is equivalent to WT. E) Proteolytic stability profiles of WT and 3.2 following trypsin digestions at various timepoints, showing that 3.2 is equivalent to WT.

FIGS. 8A-8E. Purification data set for Tp0751 chimera 4.1 (SEQ ID NO: 88; see Table 3 for sequence descriptions) A) Structural schematic showing targeted Tp0751 loops 8, 2 and 3 and the C-term (dark grey). B) Small-scale soluble expression of Tp0751 WT and 4.1, showing that 4.1 has similar expression to WT. C) SEC trace and corresponding purity gel indicating successful purification of 4.1. D) Melting temperature (Tₘ) for both WT and 4.1, showing that 4.1 is equivalent to WT. E) Proteolytic stability profiles of WT and 4.1 following trypsin digestions at various timepoints, showing that 4.1 is equivalent to WT.

FIGS. 9A-9B. Immunization with chimera construct 3.2 (SEQ ID NO: 82) significantly attenuates both lesion volume (FIG. 1a) and ulceration (FIG. 1b) over 22 days (immunized rabbits, n=8, unimmunized control rabbits, n=8). A) Lesion volume in the chimera immunized rabbits was smaller compared to the unimmunized control rabbits (P=0.01). B) Proportion of lesions ulcerating was also significantly reduced in the chimera immunized animals, compared to the unimmunized animals (P=0.02). Data shown are mean+/−SEM. Significance was assessed using two-way ANOVA.

FIG. 10. Immunized animals demonstrated lower T. pallidum burden at local infection site. Total number of T. pallidum in pooled lesion aspirates [day 19 post-challenge (PC)] was assessed via qPCR. Local sites of chimera immunized animals had significantly lower total Tp burden on day 19 PC (P=0.01). Data presented as mean+/−SEM and significance was assessed using an unpaired t-test.

FIGS. 11A-11B. Immunization with the chimera induced high antibody titers against Tp0751, TprC and TprK. A) Engineering Tp0751 by removing intrinsic loops did not affect its immunogenicity. B) The Tpr epitopes grafted onto Tp0751 were also able to induce a B-cell response.

FIGS. 12A-12B. Inhibition of dissemination in the immunized animals was observed using the rabbit infectivity test (RIT). Popliteal lymph nodes extract of challenged rabbits (day 22 PC) were injected into naïve rabbits to assess dissemination; time to development of orchitis (swelling of testicles) is inversely related to the number of viable T. pallidum inoculated. A) At day 29 post-inoculation, 75% of the animals in the unimmunized control group developed orchitis, while only 25% of the animals in the chimera immunized group developed orchitis (P=0.045). B) The RIT rabbits were monitored for 180 days; 100% of the animals in the unimmunized control group developed orchitis and 75% of the animals in the chimera immunized group developed orchitis. Although the trend was non-significant, the median days to orchitis for the immunized group was 3 days later than the unimmunized control group. Using the standard curve described in (Lukehart, S. A., et al. 2022. Vaccine), there was approximately an 85% reduction in dissemination to the popliteal lymph nodes in the chimera immunized animals. Comparisons were conducted by chi-squared test (A) and Mann-Whitney test (B). Bar observed in (B) is the median.

FIG. 13. Chimera specific serum significantly obstructs attachment of in vitro T. pallidum to human brain endothelial cells. In vitro serum inhibition assays followed by qPCR demonstrated that the chimera immunized pre-challenge sera significantly inhibits attachment of in vitro T. pallidum to endothelial cells (P=0.005). Results were normalized to human GAPDH amount and presented as median with 95% CI. Significance was assessed using Mann-Whitney test.

FIG. 14. Immunization with chimera construct 3.2 (SEQ ID NO: 82) inhibits dissemination of T. pallidum to testis. Multiple samples from 5 different testis sections were collected from three random rabbits per group on day 22 post-challenge and T. pallidum burden was evaluated using digital PCR (dPCR). The chimera immunized rabbits (n=3) had significantly lower T. pallidum burden in the testicles (P=0.0008), compared to the unimmunized control rabbits (n=3). Data presented as median with 95% CI. Significance assessed using Mann-Whitney test.

FIGS. 15A-15B. Immunization of mice with new chimera constructs emulsified in human-track adjuvants induced an antibody response. Groups of mice were immunized twice subcutaneously (4-week intervals) with 20 μg/immunization of Construct 3.2 (SEQ ID NO: 82), 3.7 (SEQ ID NO: 87), or 4.1 (SEQ ID NO: 88), emulsified in PAI-RIBI Natural, LiT4Q or PAI-sRI-T½ adjuvants [n=3 per construct+adjuvant or n=3 adjuvant only (sham-immunized control)]. The latter two adjuvants are human-track adjuvants. Antibody titers (all isotypes) were determined using serum collected 4 weeks after the primary immunization (immunized animals, W4) and 2 weeks after the final immunization/terminal bleed (immunized and sham-immunized animals, W8). Mice immunized with Construct 4.1 had the highest antibody titers compared to mice immunized with Constructs 3.2 and 3.7 (A). Sham-immunized (adjuvant only) mice did not produce antibodies that recognized all 3 constructs (B). Each data point represents a single mouse (1:100 serum dilution). Results are presented as median.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is an exemplary native Tp0751 coding sequence.

SEQ ID NO: 2 is an exemplary native full-length Tp0751 amino acid sequence (lipocalin containing domain aa 99-237, underlined).

SEQ ID NO: 3 is a native lipocalin containing domain of Tp0751 (aa 99-237).

SEQ ID NO: 4 is a native loop 1 sequence of Tp0751 (aa 99-237).

SEQ ID NO: 5 is a native loop 2 sequence of Tp0751 (aa 99-237).

SEQ ID NO: 6 is a native loop 6 sequence of Tp0751 (aa 99-237).

SEQ ID NO: 7 is a native loop 8 sequence of Tp0751 (aa 99-237).

SEQ ID NO: 8 is a native C-terminal sequence of Tp0751 (aa 99-237).

SEQ ID NO: 9: is an exemplary full-length T. pallidum TprK protein, which can be
used to generate epitopes and generate Tp0751 (99-237) chimera protein sequences.
MIDPSATSRYGSPRLVSNGFRHRRKVVYQRVGHRRFSLIFFFVVVLGRSPRLWAQVSFTPDI
EGYAELAWGIASETGGAGALKHGFKTTTDFKIVFPIVAKKDFKYRGEGNVYAEINVKALK
LSLESNGGAKFDTKGSAKTIEATLHCYGAYLTIGKNPDFKSTFAVLWEPWTANGDYKSKG
DKPVYEPGFEGAGGKLGYKQTDIAGTGLTFDIAFKFASNTDWEGKDSQGKAPAAGVTHSK
YGLGGDILFGWERTREDGVQEYIKVELTGNSTLSGDYARAGAAVPAAADDILWDVGAKV
SMKLWGLCALAATDVGHKKENAANVNGTVGADALLTLGYRWFSAGGYFASKASNVFK
DVFLTNAMDMQTHDCAAYIKLETKGSDPDTSFLEGLDLGVDVCTYMPVHWKALALPAA
AAAAAANINFPVYGKVWGSYRHDMGEYGWVKVYANLYGGTNKKNDAAALTKWSKEY
CGYYECGVVVSPLEKVEIRLSWEQGKLQENSNVVIEKNVTERWQFVGACRLIW

SEQ ID NO: 10 is an exemplary native lipocalin containing domain of Tp0751 (aa 99-237) coding sequence.

SEQ ID NO: 11 is an exemplary lipocalin containing domain of Tp0751 (aa 99-237) coding sequence optimized for expression in E. coli.

SEQ ID NO: 12 is an exemplary lipocalin containing domain of Tp0751 (aa 99-237) protein sequence optimized for expression in E. coli.

SEQ ID NO: 13 is an exemplary lipocalin containing domain of Tp0751 (aa 99-237) protein sequence for E. coli post-TEV cleavage (removal of purification tag).

SEQ ID NO: 14-16 are exemplary epitopes from Neisseria meningitidis NHBA (SEQ ID NO: 14) and NmfHbp (SEQ ID NO: 15 and 16).

SEQ ID NO: 17-22 and 24-37 are exemplary epitopes from T. pallidum Tpr protein C (TprC) and Tpr protein K (TprK).

SEQ ID NO: 23 is an exemplary epitope from T. pallidum Tp0326.

SEQ ID NOs: 38-91 are exemplary Tp0751 (99-237) chimera protein sequences.

SEQ ID NO: 92 is an exemplary full-length Neisseria meningitidis fHbp (factor H
binding protein) protein, which can be used to generate epitopes and generate Tp0751
(99-237) chimera protein sequences.
MNRTAFCCLSLTTALILTACSSGGGGVAADIGAGLADALTAPLDHKDKGLQSLTLDQSVR
KNEKLKLAAQGAEKTYGNGDSLNTGKLKNDKVSRFDFIRQIEVDGQLITLESGEFQVYKQ
SHSALTAFQTEQIQDSEHSGKMVAKRQFRIGDIAGEHTSFDKLPEGGRATYRGTAFGSDDA
GGKLTYTIDFAAKQGNGKIEHLKSPELNVDLAAADIKPDGKRHAVISGSVLYNQAEKGSYS
LGIFGGKAQEVAGSAEVKTVNGIRHIGLAAKQ
SEQ ID NO: 93 is an exemplary full-length Neisseria meningitidis NHBA (Neisserial
heparin binding antigen) protein, which can be used to generate epitopes and
generate Tp0751 (99-237) chimera protein sequences.
MFERSVIAMACIFALSACGGGGGGSPDVKSADTLSKPAAPVVAEKETEVKEDAPQAGSQG
QGAPSTQGSQDMAAVSAENTGNGGAATTDKPKNEDEGPQNDMLQNSAESANQTGNNQP
ADSSDSAPASNPAPANGGSNFGRVDLANGVLIDGPSQNITLTHCKGDSCNGDNLLDEEAPS
KSEFENLNESERIEKYKKDGKSDKFTNLVATAVQANGTNKYVIIYKDKSASSSFARFRRSA
RSRRSLPAEMPLIPVNQADTLIVDGEAVSLTGHSGNIFAPEGNYRYLTYGAEKLPGGSYAL
RVQGEPAKGEMLAGTAVYNGEVLHFHTENGRPYPTRGRFAAKVDFGSKSVDGIIDSGDDL
HMGTQKFKAAIDGNGFKGTWTENGGGDVSGRFYGPAGEEVAGKYSYRPTDAEKGGFGV
FAGKKEQD
SEQ ID NO: 94 is an exemplary full-length T. pallidum TprC protein, which can be
used to generate epitopes and generate Tp0751 (99-237) chimera protein sequences.
MGRQVMQAGVLAGMVCAASGYAGVLTPQVSGTAQLQWGIAFQKNPRTGPGKHTHGFRT
TNSLTISLPLVSKHTHTRRGEARSGVWAQLQLKDLAVELASSKSSTALSFTKPTASFQATLH
CYGAYLTVGTSPSCVVNFAQLWKPFVTRAYSEKDTRYAPGFSGSGAKLGYQAHNVGNSG
VDVDIGFLSFLSNGAWDSTDTTHSKYGFGADATLSYGVDRQRLLTLELAGNATLDQNYV
KGTEDSKNENKTALLWGVGGRLTLEPGAGFRFSFALDAGNQHQSNAHAQTQERAILKAR
EVFRRVEGKLVQNLPNIMMPPGITEQTTLIEMVGLAALIAEGTLGSAIQTVLAAGALAALV
SQLVPNIEQGVRDVFRSSDPRVVTAKLLAFLERAPMNALNIDALLRMQWKWLSSGIYFAT
AGTNIFGKRVFATTRAHYFDFAGFLKLETKSGDPYTHLLTGLNAGVEARVYIPLTYIRYRN
NGGYELNGAVPPGTINMPILGKAWCSYRIPLGSHAWLAPHTSVLGTTNRFNIINPAGNLLN
ERALQYQVGLTFSPFEKVELSAQWEQGVLADAPYMGIAESIWSERHFGTLVCGMKVTW
SEQ ID NO: 95 is an exemplary Tp0751 (99-237) chimera coding sequence (SEQ ID
NO: 82).
GTACAAACAGCTATGAGGATAGCACTATGGAACCGTGCGACCCACGGTGAGCAGGGT
GCTTTGCAGCATCTGTTAGCAGGCCTGTGGATTCAGACCGAGGCGAAAACCATTGAAG
CAACCCTGCATGGCGACATCCACCCGCTCCTGTTTTTCGATCGTGAGCACGCCGAAATC
ACCTTCAGCCGTGCTTCGGTGCAAGAGATCTTCTTGGTCGACAGCGCGCATACCCACC
GCAAGACGGTTTCCTTTCTGACCCGTAATACCGCGATTAGCTCTATCAGACGTCGCCTG
GAGGTGACGTTTGAGAGCCATGAAGTAATCCACGTGCGCGCCGTGGAAGACGTTGCGC
GTGATTACAAATCCAAGGGCGACAAACCGGTTTATGAATGGGATGGTCAATATACTCG
CTACCACGGCAAGTTGGTTCAAAACCTGCCGAACATCATGATGCCACCGGGTATTACT
SEQ ID NO: 96 is an exemplary Tp0751 (99-237) chimera coding sequence (SEQ ID
NO: 88)
GTACAAACAGCAATGAGGATAGCTCTATGGAACCGTGCTACCCACGGCGAACAAGGT
GCGCTGCAGCACCTGCTGGCTGGCCTGTGGATTCAGACCGAAGACTACAAGTCCAAAG
GTGATAAGCCGGTGTATGAGATCCACCCGCTGTTGTTTTTCGACGAATCTAATGGTGGT
GCGAAAGAAATCACGTTCAGCAGAGCGTCCGTACAAGAGATCTTTTTGGTTGATAGCG
CACATACCCATCGTAAAACCGTGAGCTTTTTGACCCGCAACACCGCAATTTCTAGCATT
CGTCGCCGGCTGGAGGTGACCTTCGAGTCGCACGAGGTCATCCACGTTCGTGCCGTTG
AGGACGTGGCCCGTGCGTATAGCGAAAAGGACACGCGTTACGCGTGGGATGGTCAGT
ATACGCGCTACCACGGTAAGCTCGTTCAAAACCTGCCGAATATTATGATGCCACCGGG
CATCACT

SEQ ID NOs: 97-114 are exemplary Tp0751 (99-237) chimera protein sequences.

SEQ ID NOs: 115-128 are bioinformatically predicted T. pallidum B cell epitopes that can be used in the disclosed chimera constructs.

SEQ ID Nos: 129-142 are nucleotide sequences of peptides of SEQ ID NOs: 115-128.

SEQ ID NO: 143 is a T. pallidum epitope from Tp0856 that can be used in the disclosed chimera constructs.

SEQ ID NO: 144 is a T. pallidum epitope from Tp0346 that can be used in the disclosed chimera constructs.

SEQ ID NO: 145 is a T. pallidum epitope from Tp0856 that can be used in the disclosed chimera constructs.

SEQ ID NO: 146 is a T. pallidum epitope from Tp0483 that can be used in the disclosed chimera constructs.

SEQ ID NOs: 147-216 are exemplary nucleotide sequences of peptides of SEQ ID NOs: 38-81, 83-87, and 89-114.

SEQ ID NOs: 217-232 are bioinformatically predicted T. pallidum B cell epitopes that can be used in the disclosed chimera constructs.

SEQ ID NOs: 233-248 are nucleotide sequences of peptides of SEQ ID NOs: 217-232.

SEQ ID NOs: 249-312 are bioinformatically predicted T. pallidum T cell epitopes that can be used in the disclosed chimera constructs.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a Tp0751 (99-237) loop” includes single or plural Tp0751 (99-237) loops and is considered equivalent to the phrase “comprising at least one Tp0751 (99-237) loop.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. It is further to be understood that any and all base sizes or amino acid sizes, and all molecular weight or molecular mass values, given for nucleic acids or polypeptides are approximate, and are provided for descriptive purposes, unless otherwise indicated.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. All GenBank accession numbers and provided herein are incorporated by reference.

Adjuvant: A vehicle used to enhance antigenicity. Adjuvants which can be used in combination with the chimeric Tp0751 (99-237) proteins disclosed herein include, but are not limited to, a suspension of minerals (e.g., alum, aluminum hydroxide, or phosphate) on which antigen is adsorbed; or water-in-oil emulsion in which antigen solution is emulsified in mineral oil (e.g., Freund incomplete adjuvant), sometimes with the inclusion of killed mycobacteria (e.g., Freund's complete adjuvant) to further enhance antigenicity. Immunostimulatory oligonucleotides (such as those including a CpG motif) can also be used as adjuvants (for example see U.S. Pat. Nos. 6,194,388; 6,207,646; 6,214,806; 6,218,371; 6,239,116; 6,339,068; 6,406,705; and 6,429,199). Adjuvants include biological molecules (a “biological adjuvant”), such as costimulatory molecules. Other exemplary adjuvants include IL-2, RANTES, GM-CSF, TNF-α, IFN-γ, G-CSF, LFA-3, CD72, B7-1, B7-2, OX-40L, 4-1 BBL, immune stimulating complex (ISCOM) matrix, and toll-like receptor (TLR) agonists, such as TLR-9 agonists, Poly I:C, or PolyICLC. Additional adjuvants include monophosphoryl lipid A (MPL) and Alhydroxiquim-II.

Other exemplary adjuvants include chitosan, Bacillus-Calmette-Guerin adjuvant and RIBI adjuvant. In one example, the adjuvant is a human track adjuvant (i.e., adjuvant whose components are suitable for use in humans), LiT4Q, PAI-sRI-T1/2 alum, or AS04. In one example the adjuvant used is RIBI Natural. RIBI Natural includes monophosphoryl lipid A [MPL]+trehalose 6,6′-dimycolate [TDM]+cell wall skeleton from Mycobacterium phlei [CWS]. In one example the adjuvant used is RIBI synthetic. RIBI Synthetic contains MPL, synthetic trehalose dicorynomycolate (TDcM (a low toxicity analogue of TDM that is considered safe for human use), and muramyl dipeptide (MDP). In one example the adjuvant used is LiT4Q. In one example the adjuvant used is PAI-sRI-T1/2. In one example the adjuvant used is alum. In one example the adjuvant used is AS04. Thus, one or more of these adjuvants can be included in the pharmaceutical compositions provided herein that contain one or more chimeric Tp0751 proteins (such as one or more chimeric Tp0751 (99-237) proteins, for example to enhance their antigenicity).

Administration: The introduction of a composition, such as the chimeric Tp0751 (99-237) proteins provided herein, into a subject by a chosen route. Administration can be local or systemic. For example, if the chosen route is intravenous, the composition is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of an amino acid in a polypeptide with one or more different amino acids. In some examples herein, a native amino acid in Tp0751 (99-237) protein (such as SEQ ID NO: 3), such as in any one of loops 1-8, is replaced with a different non-native amino acid.

Antibody: An immunoglobulin, antigen-binding fragment, or derivative thereof, which specifically binds and recognizes an analyte (antigen), such as the disclosed chimeric Tp0751 (99-237) proteins. The term “antibody” as used herein encompasses various antibody structures, including but not limited to monoclonal antibodies, polyclonal antibodies, multispecific antibodies (e.g., bispecific antibodies), and antibody fragments, so long as they exhibit the desired antigen-binding activity.

Specific, non-limiting examples of binding fragments encompassed within the term antibody include (i) a Fab fragment consisting of the V_{L}, V_{H}, C_{L} and C_{H1} domains; (ii) an F_{d} fragment consisting of the V_{H} and C_{H1} domains; (iii) an Fv fragment consisting of the VL and VH domains of a single arm of an antibody, (iv) a dAb fragment (Ward et al., Nature 341:544-546, 1989) which consists of a V_{H} domain; (v) an isolated complementarity determining region (CDR); and (vi) a F(ab′)₂ fragment, a bivalent fragment comprising two Fab fragments linked by a disulfide bridge at the hinge region.

Antigen: A compound, composition, or substance that can stimulate the production of antibodies, a T cell response, or both in an animal, including compositions that are injected or absorbed into an animal. An exemplary antigen is a chimeric Tp0751 (99-237) peptide, such as any of SEQ ID NOS: 38-91 and 97-114. An antigen reacts with the products of specific humoral or cellular immunity, including those induced by heterologous immunogens. The term “antigen” includes all related antigenic epitopes, such as any of SEQ ID NOS: 19, 20, 21, 22, 23, 27, 28, 29, 30, 33, 34, 143-146, 115-128, 217-232, and 249-312.

Chimera: A biological molecule, such as a protein or nucleic acid molecule, comprising at least a portion of two different (heterologous) proteins or nucleic acid molecules. A chimeric Tp0751 (99-237) peptide is one having one or more of its loops 1-8 replaced (all or in part) by a non-native epitope peptide, such one from a bacterium that causes an STI or other infectious disease.

Conservative variant: Conservative amino acid substitutions are those substitutions that do not substantially affect or decrease the biological activity of a protein, such as the antigenic activity of a chimeric Tp0751 (99-237) protein provided herein. As one example, a chimeric Tp0751 (99-237) protein provided herein can include 1-20 conservative substitutions and retain the ability to generate an immune response. The term “conservative variant” also includes the use of a substituted amino acid in place of an unsubstituted parent amino acid, provided that the variant retains the ability to generate an immune response.

Conservative amino acid substitution tables providing functionally similar amino acids are known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some aspects herein, the chimeric Tp0751 (99-237) protein sequences provided herein (such as any one of SEQ ID NOS: 38-91 and 97-114) include no more than 20, no more than 15, no more than 10, no more than 9, no more than 8, no more than 7, no more than 6, no more than 5, no more than 4, no more than 3, no more than 2 or no more than 1 amino acid substitutions relative to any amino acid sequence disclosed herein.

Consists Essentially Of/Consists Of: With regard to a protein (such as a chimeric Tp0751 (99-237) protein provided herein), a protein that consists essentially of a specified amino acid sequence if it does not include any additional amino acid residues. However, the protein can include additional non-peptide components, such as labels (for example, fluorescent, radioactive, or solid particle labels), PEG, sugars or lipids. A protein that consists of a specified amino acid sequence does not include any additional amino acid residues, nor does it include additional non-peptide components, such as lipids, PEG, sugars or labels.

Degenerate variant: A polynucleotide encoding a protein (such as a chimeric Tp0751 (99-237) protein provided herein) that includes a sequence that is degenerate as a result of the genetic code. There are 20 natural amino acids, most of which are specified by more than one codon. Therefore, all degenerate nucleotide sequences are included in this disclosure as long as the amino acid sequence of the chimeric Tp0751 (99-237) protein encoded by the nucleotide sequence is unchanged.

Epitope: An antigenic determinant. Epitopes are particular chemical groups or peptide sequences on a molecule that are antigenic (that elicit a specific immune response). An antibody specifically binds a particular antigenic epitope on a polypeptide, such as a chimeric Tp0751 (99-237) protein provided herein. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. In some examples an epitope includes at least 2, at least 3, at least 4, at least 5, at least 6, at least 7, at least 8, at least 9, at least 10, at least 11, at least 12, at least 15, or at least 20 amino acids (such as 2, 3, 4, 5, 6, 7, 8, 9, 10, 11, 12, 13, 14, 15, 16, 17, 18, 19, 20, 21, 22, 23, 24 or 25 aa, such as 2-10, 2-7, 5-10, 5-12, 5-13, 5-15, 7-20, 7-22 or 8-10 aa) in a unique spatial conformation. Exemplary methods of determining spatial conformation of epitopes include, for example, x-ray crystallography and 2-dimensional nuclear magnetic resonance. Exemplary epitopes that can be used in the chimeras and methods provided herein include those provided in SEQ ID NOS: 19, 20, 21, 22, 23, 27, 28, 29, 30, 33, 34, 143-146, 115-128, 217-232, and 249-312.

Heterologous: Originating from a separate genetic source or species.

Immune response: A response of a cell of the immune system, such as a B cell, T cell, or monocyte, to a stimulus. In one aspect, the response is specific for a particular antigen (an “antigen-specific response”), such as a chimeric Tp0751 (99-237) protein provided herein. In one aspect, an immune response is a T cell response, such as a CD4+ response or a CD8+ response. In another aspect, the response is a B cell response, and results in the production of specific antibodies (such as T. pallidum- or Tp0751-specific antibodies). In some examples, an immune response is both a T cell and B cell response.

Immunize: To render a subject (such as a mammal) partially or fully protected from an infectious disease (for example, T. pallidum), such as by vaccination. The term “immunize” does not require complete (100%) protection against an infectious disease. In some aspects, immunization of a subject decreases the subject's risk of infection or disease by at least 30%, at least 40%, at least 50%, at least 60%, at least 70%, at least 80% or at least 90%, compared to in the absence of immunization.

Immunogen: A compound, composition, or substance (for example, a chimeric Tp0751 (99-237) protein provided herein or nucleic acid encoding such) that can elicit an immune response in an animal, including compositions that are injected or absorbed into an animal. Administration of an immunogen to a subject can lead to protective immunity against a pathogen (such as an STI or other infection, such as T. pallidum).

Immunogenic composition: A composition, such as a composition containing a chimeric Tp0751 (99-237) protein provided herein or a nucleic acid encoding the chimeric Tp0751 (99-237) protein, that induces a measurable T cell response against cells expressing the chimeric Tp0751 (99-237) protein, induces a measurable B cell response (such as production of antibodies that specifically bind to epitopes of the a chimeric Tp0751 (99-237) protein, or both. For in vitro use, the immunogenic composition can consist of the isolated nucleic acid, vector including the nucleic acid/or immunogenic protein. For in vivo use, the immunogenic composition will typically include the nucleic acid, vector including the nucleic acid, and or immunogenic protein, in one or more pharmaceutically acceptable carriers, and/or other agents. An immunogenic composition can optionally include an adjuvant, a costimulatory molecule, a nucleic acid encoding a costimulatory molecule, or combinations thereof.

Immunogenic Protein: A protein which includes an allele-specific motif or other sequence such that the peptide will bind an MHC molecule and induce a T cell response, a B cell response (e.g., antibody production), or both, against the antigen. Exemplary immunogenic proteins are the chimeric Tp0751 (99-237) protein provided herein (such as any one of SEQ ID NOS: 38-91 and 97-114).

Immunogenic fragments (e.g., epitopes) of a protein cause induction of an immune response, as measured by clinical response (for example an increase in a population of immune cells, increased cytolytic activity against cells that express a chimeric Tp0751 (99-237) protein provided herein, increased production of chimeric Tp0751 (99-237) protein antibodies, inhibition of dissemination, or measurable reduction of chancre volume or ulcerations, or combinations thereof). Immunogenic proteins can also be made from nucleic acids. Examples of a nucleic acid based therapeutically active molecule is a nucleic acid sequence that encodes a chimeric Tp0751 (99-237) protein, wherein the nucleic acid sequence is operably linked to a control element such as a promoter.

Isolated or Purified: An “isolated” or “purified” biological component (e.g., proteins, nucleic acids, cellular components, or cells) is one that has been substantially separated from other biological components in the environment in which the component occurs (e.g., separated from other proteins, nucleic acids, cellular components, or cells). Proteins and nucleic acids can be “isolated” from a sample using standard purification/isolation methods.

Absolute purity or isolation is not required, it is intended as a relative term. Thus, for example, isolated nucleic acids and proteins are those in which the nucleic acids or proteins are more enriched than it was in its initial environment. In one example, a preparation is purified/isolated such that the nucleic acid, protein, or cellular component at least 50% of the total content of the preparation, for example, at least 60%, at least 70%, at least 80%, at least 90%, at least 95%, at least 98%, or at least 99% pure. In a specific, non-limiting example, a purified nucleic acid, protein, or cellular component is at least 70% free of other components. In a specific example, an isolated or “purified” chimeric Tp0751 (99-237) protein is substantially free of other proteins, lipids, carbohydrates or other materials with which it was associated. In one embodiment, a chimeric Tp0751 (99-237) protein is at least 50%, for example at least 80%, at least 90%, or at least 95% free of other proteins, lipids, carbohydrates or other materials with which it was associated (such as a cell lysate).

Liposome and lipid nanoparticle (LNP): A lipid bilayer vesicle, can be used as a delivery system. Thus, liposomes and LNPs can be used to deliver the chimeric Tp0751 (99-237) protein proteins disclosed herein (or nucleic acids and vectors encoding the chimeric Tp0751 (99-237) proteins).

Modification: A change in a protein or nucleic acid sequence. For example, amino acid sequence modifications include, for example, substitutions, insertions and deletions, or combinations thereof. Insertions include amino and/or carboxyl terminal fusions as well as intrasequence insertions of single or multiple amino acid residues. Deletions are characterized by the removal of one or more amino acid residues from the protein sequence. Substitutional modifications are those in which at least one residue has been removed and a different residue (or residues) inserted in its place. Amino acid substitutions are typically of single residues, but can occur at a number of different locations at once. In some aspects herein, a chimeric Tp0751 (99-237) protein is modified by substitution or replacement of an amino acid at one or more positions of the native Tp0751 (99-237) protein, specifically two or more amino acids in one or more of the 8 loops and/or the C-terminus of Tp0751 (99-237).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 15th Edition (1975), describes compositions and formulations suitable for pharmaceutical delivery of a chimeric Tp0751 (99-237) protein herein disclosed (or a nucleic acid encoding such).

The nature of the carrier can depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymers as well as in which one or more amino acid residue is a non-natural amino acid, for example an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues. Amino acids in a polypeptide generally are chemically bound together via amide linkages (CONH).

Preventing, treating or ameliorating a disease: Inhibiting the development or progression of a disease or condition, for example, in a subject who is at risk of or has a T. pallidum infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease for the purpose of reducing the risk of developing pathology.

Protein tag: Small peptides fused to a protein of interest. In some aspects, the protein tag is located at the N-terminus or C-terminus of a protein (such as a chimeric Tp0751 (99-237) protein). Types of protein tags include, but are not limited to, affinity tags (e.g., HiBiT, glutathione S-transferase (GST), or His tag), epitope tags (e.g., HA, V5, FLAG, or Myc), and fluorescent tags (e.g., GFP, or a variant thereof).

Recombinant: A recombinant protein or nucleic acid is one that has a sequence that is not naturally occurring or has a sequence that is made by an artificial combination of two otherwise separated segments of sequence. This artificial combination can accomplished, for example, by chemical synthesis or genetic engineering techniques.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are well known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, 90%, 95%, 96%, 97%, 98% or 99% sequence identity counted over the full-length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Sexually transmitted infection/disease (STI/STD): An infection/disease transmitted through sexual contact. Infections can be caused by bacteria, viruses, or parasites. Specific examples include Treponema pallidum subsp. pallidum infection which causes syphilis, Chlamydia infection which causes chlamydia, and Neisseria gonorrhoeae which causes gonorrhea.

Subject: Living multi-cellular vertebrate organisms, a category that includes human and non-human mammals, for example mammals that get syphilis (such as humans and rabbits).

Therapeutically effective amount: A quantity of a composition to achieve a desired effect in a subject being treated. For instance, this can be the amount of a chimeric Tp0751 (99-237) protein or a vector encoding such a protein, necessary to induce an immune response (such as a B-cell and T cell response), such as increase production of Th1 cytokines (such as interferon-γ (IFN-γ), interleukin-2 (IL-2), and/or interleukin-12 (IL-12) (e.g., relative to no administration of the chimeric Tp0751 (99-237) protein or nucleic acid), such as an increase of at least 20%, at least 30%, at least 40%, at least 50%, at least 75%, at least 80%, at least 90%, at least 95%, at least 100%, at least 200%, at least 300%, at least 400%, or at least 500%), increase antibody production against T. pallidum (e.g., relative to no administration of the chimeric Tp0751 (99-237) protein or nucleic acid), such as an increase of at least 20%, at least 30%, at least 40%, at least 50%, at least 75%, at least 80%, at least 90%, at least 95%, at least 100%, at least 200%, at least 300%, at least 400%, or at least 500%), increase phagocytosis of opsonized T. pallidum (e.g., relative to no administration of the chimeric Tp0751 (99-237) protein or nucleic acid), such as an increase of at least 20%, at least 30%, at least 40%, at least 50%, at least 75%, at least 80%, at least 90%, at least 95%, at least 100%, at least 200%, at least 300%, at least 400%, or at least 500%), reduce chancre volume (e.g., relative to no administration of the chimeric Tp0751 (99-237) protein or nucleic acid), such as a reduction of at least 10%, at least 20%, at least 30%, at least 40%, at least 50%, at least 75%, at least 80%, at least 90%, at least 95%, or even 100%), reduce the % of chancres that ulcerate (e.g., relative to no administration of the chimeric Tp0751 (99-237) protein or nucleic acid), such as a reduction of at least 10%, at least 20%, at least 30%, at least 40%, at least 50%, at least 75%, at least 80%, at least 90%, at least 95%, or even 100%), reduced dissemination of T. pallidum to tissues (e.g., relative to no administration of the chimeric Tp0751 (99-237) protein or nucleic acid), such as a reduction of at least 10%, at least 20%, at least 30%, at least 40%, at least 50%, at least 75%, at least 80%, at least 90%, at least 95%, or even 100%), reduce dissemination of T. pallidum to lymph nodes and other tissues and organs (e.g., relative to no administration of the chimeric Tp0751 (99-237) protein or nucleic acid), such as a reduction of at least 10%, at least 20%, at least 30%, at least 40%, at least 50%, at least 75%, at least 80%, at least 90%, at least 95%, or even 100%), improve outward symptoms of a T. pallidum infection (e.g., relative to no administration of the chimeric Tp0751 (99-237) protein or nucleic acid, such as a reduction of at least 10%, at least 20%, at least 30%, at least 40%, at least 50%, at least 75%, at least 80%, at least 90%, at least 95%, or even 100%), or combinations thereof. When administered to a subject, a dosage will generally be used that will achieve target tissue concentrations (for example, in lymphocytes) that has been shown to achieve an in vitro effect.

To obtain a protective immune response against a pathogen, such as T. pallidum, multiple administrations of a disclosed chimeric Tp0751 (99-237) protein or nucleic acid, and/or administration of a disclosed chimeric Tp0751 (99-237) protein or nucleic acid as the “prime” in a prime boost protocol, wherein the boost immunogen can be different from the prime immunogen, may be required. Accordingly, a therapeutically effective amount of a disclosed chimeric Tp0751 (99-237) protein or nucleic acid can be the amount of the chimeric Tp0751 (99-237) protein or nucleic acid sufficient to elicit a priming immune response in a subject that can be subsequently boosted with the same or a different immunogen to generate a protective immune response.

Tp0751 (also known as pallilysin): A lipid anchored outer membrane protein of T. pallidum, which binds to laminin. Tp0751 promotes adhesion of the bacterium to endothelial cells and enables dissemination within the host. Antibodies against this adhesin are present in serum from both natural and experimental T. pallidum infections.

The term Tp0751 includes a Tp0751 gene, cDNA, mRNA, or protein. Nucleic acid and protein sequences for Tp0751 are publicly available. SEQ ID NOS: 1 and 2 provide exemplary coding and protein sequences. Tp0751 begins as pre-protein, from which a mature protein found in T. pallidum is generated by cleaving off the signal sequence (amino acids 1-23).

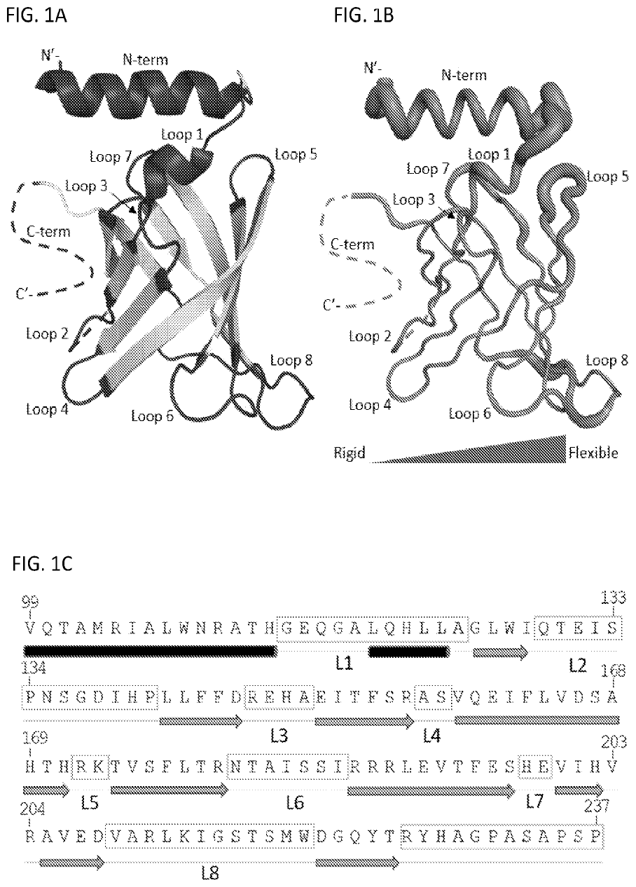

Tp0751 includes a compact lipocalin-like fold (Tp0751 (99-237)) that incorporates a highly stable core of eight beta strands connected by a network of loops (FIG. 1C) that are shown herein to be amenable to substitution with B cell and/or T cell epitopes. An exemplary native Tp0751 (aa 99-237) protein sequence is provided in SEQ ID NO: 3. A chimeric Tp0751 (aa 99-237) protein is one wherein at least one non-native epitope peptide replaces all or of part of one or more of the 8 loops of a native Tp0751 (99-237) peptide, and optionally all or part of the C-terminus of a native Tp0751 (99-237) (see FIG. 1C).

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a pathogen that causes an STI or other infection, or to a cellular constituent correlated with a pathological condition. A vaccine may include a polynucleotide (such as a nucleic acid encoding a disclosed chimeric Tp0751 (99-237) protein), a peptide or polypeptide (such as a disclosed chimeric Tp0751 (99-237) protein), a virus, a cell or one or more cellular constituents. In one specific, non-limiting example, a vaccine reduces the severity of the symptoms associated with an STI, such as T. pallium, compared to a control. In another non-limiting example, a vaccine reduces infection, such as T. pallium, compared to a control.

Methods of administration can vary according to the vaccine, but can include inoculation, ingestion, inhalation or other forms of administration. Vaccines may be administered with an adjuvant to boost the immune response.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. In some aspects, the vector is a virus vector, such as a lentivirus vector, adenovirus vector, or an adeno-associated viral vector (AAV). In another example, a vector is a plasmid vector.

### Overview

Syphilis is a chronic, sexually transmitted infection (STI) caused by the invasive spirochete Treponema pallidum subspecies pallidum (herein referred to as T. pallidum). While penicillin is an effective clinical treatment, syphilis persists as a healthcare burden in low- and middle-income countries and is re-emerging as a public health threat in many areas around the world. The public health burden of syphilis infections is further compounded by the 3- to 5-fold increased susceptibility of individuals with syphilis to acquisition and transmission of HIV. Further, T. pallidum can be vertically transmitted from a mother to her developing fetus at any stage of pregnancy to cause congenital syphilis. Congenital syphilis is the leading cause of infectious stillbirth in low- and middle-income countries, and is re-emerging at an alarming rate as a cause of fetal loss and newborn death in high-income countries. The need for effective prophylactic-based clinical approaches, such as vaccines, to eliminate syphilis is urgent.

No commercially viable vaccine or vaccination approach exists for T. pallidum. Herein described is the design and development of a highly modular, multi-antigen, protein-based vaccine engineered using the T. pallidum protein Tp0751 (hereafter referred to as Tp0751) scaffold. Structural studies revealed that Tp0751 adopts a lipocalin fold, which consists of a core beta barrel where individual beta strands are connected by a network of flexible loops. With the lipocalin domain core possessing significant structural stability, it was determined if the inter-strand Tp0751 loops (FIG. 1C) would be amenable to substitution with immunogenic loops (B cell and/or T cell epitopes) from different proteins (e.g., T. pallidum Tp0326, Tp0346, Tp0856, Tp0483, TprC, and TprK proteins) to generate a Tp0751 chimera without compromising the physical characteristics (e.g., folding, thermostability, etc. . . . ) of the engineered protein. Thus, the Tp0751 scaffold (referred to as the chimeric Tp0751 (99-237 protein)) can support the development of a multi-antigen vaccine that is largely agnostic to the source of the engrafted epitope, hence the platform nature of the technology. This is core design feature of this modular Tp0751 vaccine platform where, for example, a chimeric Tp0751 (99-237) protein can be engineered with multiple T. pallidum epitopes (such as 2, 3, 4, 5, 6, 7, 8, 9, 10, 11, 12, 13, 14, 15, 16, 17, 18, 19, or 20 different epitopes), to generate an effective syphilis vaccine to stimulate multiple features of the host's immune response, or it can be engineered with epitopes from different STI bacteria to generate an effective pan-STI vaccine. The chimeric Tp0751 (99-237) proteins can serve as an effective vaccine platform can be used for communicable or non-communicable disease where a multi-antigen vaccination approach is needed. Extending the appeal of the chimeric Tp0751 (99-237) proteins as a highly efficacious vaccine platform are the findings that: (1) the chimeric Tp0751 (99-237) proteins are a naturally low-immunogenicity structure, that facilitates targeting of the immune response to the exogenous immunogenic epitopes and reduce the risk of vaccine-related autoimmune issues; and (2) the Tp0751 (99-237) amino acid sequence is unique and unrelated to any proteins present in current databases, including human proteins, reducing the risk of detrimental vaccine-related autoimmunity.

To identify and subsequently engineer Tp0751 (99-237) loops amenable for substitution with B cell and/or T cell epitopes (8 loops, FIG. 1C), structure-guided approaches were complemented with in silico protein stability and immunogenicity predictions to yield a proprietary engineering workflow. An additional feature of the chimeric Tp0751 (99-237) proteins is the ability to expand beyond simply decorating a core stable protein scaffold with multiple different antigens, but also to present epitopes in the appropriate structural context to elicit the necessary protective immune response. Many of the most desirable epitopes are canonically displayed as loops on outer membrane proteins (OMPs) presented at the interface between the pathogen and host. By targeting permissive loops in Tp0751 (99-237) for substitution with B cell and/or T cell epitopes, the structural context of these OMP derived epitopes was faithfully recapitulated, thereby improving the efficacy and potency of the engineered vaccine. The disclosed technology has also been designed to support downstream Chemistry, Manufacturing and Controls (CMC) processes. The engineered chimeric Tp0751 (99-237) proteins described herein are produced in high yield as single, multi-antigen, soluble recombinant proteins from E. coli that are easily purified, thermostable and proteolytically stable (similar to native Tp0751) and are amenable to endotoxin removal and sterile filtration.

This application describes the design and development of a novel platform-based vaccine technology using chimeric Tp0751 (99-237) proteins that incorporate multiple epitopes presented in the appropriate structural context to maximize the immune response and that is also amenable to large scale CMC production.

Despite the growing understanding of the types of immune responses required for treponemal clearance and protection, one barrier is the limited number of antigenic targets present on the bacterial surface (33, 34). The density of outer-membrane proteins on the surface of T. pallidum is approximately 100-fold less than that of Escherichia coli, resulting in a minimal immune response to natural infection (35). The paucity of outer-membrane antigens is a major contributing factor to the “stealth” nature of T. pallidum, making the outer-membrane proteins optimal targets for vaccine development (17, 36-38). Further compounding the vaccine development challenges associated with the minimalist surface protein repertoire is the fact that very few of the known surface proteins have been structurally characterized or epitope mapped. The lack of these experimental details offers little insight and few starting points into developing molecular strategies to drive a protective immune response.

### Chimeric Tp0751 (99-237) Peptides

Provided herein are chimeric Tp0751 (99-237) proteins, which can include one or more B- and/or T-cell epitopes from T. pallidum proteins to mount a protective immune response against T. pallidum. In some examples, a chimeric Tp0751 (99-237) protein includes 1 to 20 different B- and/or T-cell epitopes (such as 1, 2, 3, 4, 5, 6, 7, 8, 9, 10, 11, 12, 13, 14, 15, 16, 17, 18, 19, or 20 different epitopes) from T. pallidum protein(s), such as from Tp0326, Tp0346, Tp0856, Tp0483, TprC and/or TprK. In some examples, a chimeric Tp0751 (99-237) protein includes 1 to 20 different B- and/or T-cell epitopes (such as 1, 2, 3, 4, 5, 6, 7, 8, 9, 10, 11, 12, 13, 14, 15, 16, 17, 18, 19, or 20 different epitopes) from Chlamydia protein(s). In some examples, a chimeric Tp0751 (99-237) protein includes 1 to 20 different B- and/or T-cell epitopes (such as 1, 2, 3, 4, 5, 6, 7, 8, 9, 10, 11, 12, 13, 14, 15, 16, 17, 18, 19, or 20 different epitopes) from Neisseria gonorrhoeae protein(s). In one example, a chimeric Tp0751 (99-237) protein includes 1 to 20 different B- and/or T-cell epitopes from Treponema pallidum and 1 to 20 different B- and/or T-cell epitopes from Chlamydia. In one example, a chimeric Tp0751 (99-237) protein includes 1 to 20 different B- and/or T-cell epitopes from Treponema pallidum and 1 to 20 different B- and/or T-cell epitopes from Neisseria gonorrhoeae. In one example, a chimeric Tp0751 (99-237) protein includes 1 to 20 different B- and/or T-cell epitopes from Treponema pallidum, and 1 to 20 different B- and/or T-cell epitopes from Chlamydia, and 1 to 20 different B- and/or T-cell epitopes from Neisseria gonorrhoeae.

In one example, the chimeric Tp0751 (99-237) protein includes at least one non-native epitope peptide that replaces all or of part of one or more loops of a native Tp0751 (99-237) peptide. An exemplary native Tp0751 (99-237) peptide sequence is shown in SEQ ID NO: 3. The loops of the native Tp0751 (99-237) peptide are shown in FIG. 1C: loop 1 GEQGALQHLLA (SEQ ID NO: 4), loop 2 QTEISPNSGDIHP (SEQ ID NO: 5), loop 3 REHA (aa 147-150 of SEQ ID NO: 2), loop 4 AS (aa 157-158 of SEQ ID NO: 2), loop 5 RK (aa 172-173 of SEQ ID NO: 2), loop 6 NTAISSI (SEQ ID NO: 6), loop 7 HE (aa 198-199 of SEQ ID NO: 2), and loop 8 aa DVARLKIGSTSMWD (SEQ ID NO: 7). In some examples, the chimeric Tp0751 peptide further includes at least one non-native epitope peptide that replaces all or of part of the native C-terminus of the native Tp0751 (99-237) peptide, wherein the native C-terminus of the native Tp0751 (99-237) peptide is SEQ ID NO: 8. Thus, when referring to a non-native peptide or epitope herein (such as a T cell epitope or B cell epitope), it is relative to a Tp0751 (99-237) protein sequence (e.g., a non-native peptide or epitope is one not found in a native or wild-type a Tp0751 (99-237) protein sequence).

The at least one non-native epitope peptide “replaces” all of or part of one or more loops when at least 2 contiguous amino acids of the loop are removed and substituted with at least one non-native epitope peptide. Similarly, the at least one non-native epitope peptide “replaces” all or of part of the native C-terminus when at least 2 contiguous amino acids of the native C-terminus are removed and substituted with at least one non-native epitope peptide. In some examples, at least 3, at least 4, at least 5, at least 6, at least 7, at least 8, at least 9, at least 10, at least 11, at least 12, or at least 13 contiguous amino acids of a loop or the native C-terminus are removed and substituted with at least one non-native epitope peptide. In some examples, 2, 3, 4, 5, 6, 7, 8, 9, 10, 11, 12 or 13 contiguous amino acids of a loop or the native C-terminus are removed and substituted with at least one non-native epitope peptide. In some examples, 2, 3, 4, 5, 6, 7, or 8 of the loops of the native Tp0751 (99-237) peptide are replaced in all or part with the at least one non-native epitope peptide. In some examples, loop 8 of the native Tp0751 (99-237) peptide is replaced (such as 2, 3, 4, 5, 6, 7, 8, or 9 (such as 7-9) contiguous amino acids of loop 8) with at least one non-native epitope peptide. In some examples, (a) loops 2, 3, 6 and 8 loops 2, 3, and 8, (b) loops 2 and 8, loops 2, 3, and 8 and the C-terminus, or (c) loops 2 and 8 and the C-terminus are replaced in all or part with the at least one non-native epitope peptide.

In one example, 2, 3, 4, 5, or 6, contiguous amino acids of loop 1 are removed and substituted with at least one non-native epitope peptide. In one example, 2, 3, 4, 5, 6, 7, or 8 (such as 5-8) contiguous amino acids of loop 2 are removed and substituted with at least one non-native epitope peptide. In one example, 2, 3, or 4, (such as 2-4) contiguous amino acids of loop 3 are removed and substituted with at least one non-native epitope peptide. In one example, 2, 3, or 4, (such as 2-4) contiguous amino acids of loop 4 are removed and substituted with at least one non-native epitope peptide. In one example, 2 contiguous amino acids of loop 5 are removed and substituted with at least one non-native epitope peptide. In one example, 2, 3, 4, or 5 (such as 2-5) contiguous amino acids of loop 6 are removed and substituted with at least one non-native epitope peptide. In one example, 2 contiguous amino acids of loop 7 are removed and substituted with at least one non-native epitope peptide. In one example, 2, 3, 4, 5, 6, 7, 8, or 9 (such as 7-9) contiguous amino acids of loop 8 are removed and substituted with at least one non-native epitope peptide. In one example, 2, 3, 4, 5, 6, 7, 8, or 9 (such as 7-9) contiguous amino acids of the C-terminus are removed and substituted with at least one non-native epitope peptide. In some examples, combinations of these are generated, such as substitutions in (a) loops 2, 3, 6 and 8 loops 2, 3, and 8, (b) loops 2 and 8, loops 2, 3, and 8 and the C-terminus, or (c) loops 2 and 8 and the C-terminus.

The at least one non-native B cell and/or T cell epitope sequence is an epitope not found in a native Tp0751 (99-237) peptide, such as not found in SEQ ID NO: 3. However, the epitope may be from another T. pallidum protein, such as a B cell and/or T cell epitope from TprK (SEQ ID NO: 9) or TprC (SEQ ID NO: 94), Tp0326, Tp0346, Tp0856, and/or Tp0483. In one example, the at least one non-native epitope peptide is at least 2, at least 3, at least 4, at least 5, at least 6, at least 7, at least 8, at least 9, or at least 10 contiguous amino acids from SEQ ID NO: 9, such as 2-30, 2-15, 2-10, 3-15, 5-15, 5-20, or 5-10 contiguous amino acids from SEQ ID NO: 9, or such as 2, 3, 4, 5, 6, 7, 8, 9, 10, 11, 12, 13, 14, 15, 16, 17, 18, 19, 20, 21, 22, 23, 24, or 25 contiguous amino acids from SEQ ID NO: 9. In one example, the at least one non-native epitope peptide is at least 2, at least 3, at least 4, at least 5, at least 6, at least 7, at least 8, at least 9, or at least 10 contiguous amino acids from SEQ ID NO: 94, such as 2-30, 2-15, 2-10, 3-15, 5-15, 5-20, or 5-10 contiguous amino acids from SEQ ID NO: 94, or such as 2, 3, 4, 5, 6, 7, 8, 9, 10, 11, 12, 13, 14, 15, 16, 17, 18, 19, 20, 21, 22, 23, 24, or 25 contiguous amino acids from SEQ ID NO: 94. In some examples, the non-native epitope peptide is from a non-T. pallidum protein, such as Chlamydia or Neisseria gonorrhoeae.

In some examples, at least one non-native epitope peptide that replaces all or part of one or more loops of a native Tp0751 (99-237) peptide are from one or more pathogens, such as viral epitopes, bacterial epitopes, fungal epitopes and/or parasitic epitopes. In some examples, the at least one non-native epitope peptide protein found on the surface of a pathogen. In some examples, the at least one non-native epitope peptide protein is a bacterial surface protein, such as outer surface protein A (OspA) and/or outer surface protein C (OspC) of B. burgdorferi (such a peptide could be used to treat or prevent Borrelia/Lyme disease).

One or more one non-native epitope peptides from any pathogen or microbe can be included in the chimeric Tp0751 (99-237) peptides provided herein. Exemplary pathogens include, but are not limited to, viruses, bacteria, fungi, nematodes, and protozoa. A non-limiting list of pathogens that can be treated or prevented using the chimeric Tp0751 (99-237) peptides and methods provided herein are provided below.

For example, one or more non-native epitope peptides can be from a virus, such as positive-strand RNA viruses and negative-strand RNA viruses. Exemplary target positive-strand RNA viruses include, but are not limited to: Picornaviruses (such as Aphthoviridae [for example foot-and-mouth-disease virus (FMDV)]), Cardioviridae; Enteroviridae (such as Coxsackie viruses, Echoviruses, Enteroviruses, and Polioviruses); Rhinoviridae (Rhinoviruses)); Hepataviridae (Hepatitis A viruses); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); Calciviridae (which includes Norovirus and Sapovirus); and Coronaviruses (examples of which include SARS coronaviruses, such as the Urbani strain). Exemplary negative-strand RNA viruses include, but are not limited to: Orthomyxyoviruses (such as the influenza virus), Rhabdoviruses (such as Rabies virus), and Paramyxoviruses (examples of which include measles virus, respiratory syncytial virus, and parainfluenza viruses).

One or more non-native epitope peptides can be from a DNA virus. Exemplary DNA viruses include, but are not limited to: Herpesviruses (such as Varicella-zoster virus, for example the Oka strain; cytomegalovirus; and Herpes simplex virus (HSV) types 1 and 2), Adenoviruses (such as Adenovirus type 1 and Adenovirus type 41), Poxviruses (such as Vaccinia virus), and Parvoviruses (such as Parvovirus B19).

One or more non-native epitope peptides can be from a Retrovirus. Examples of retroviruses include, but are not limited to: human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); and avian sarcoma virus.

In one example, one or more non-native epitope peptides are from one or more of the following: HIV-1 (for example an HIV antibody, p24 antigen, or HIV genome); Hepatitis A virus (for example an Hepatitis A antibody, or Hepatitis A viral genome); Hepatitis B (HB) virus (for example an HB core antibody, HB surface antibody, HB surface antigen, or HB viral genome); Hepatitis C (HC) virus (for example an HC antibody, or HC viral genome); Hepatitis D (HD) virus (for example an HD antibody, or HD viral genome); Hepatitis E virus (for example a Hepatitis E antibody, or HE viral genome); a respiratory virus (such as influenza A & B, respiratory syncytial virus, human parainfluenza virus, or human metapneumovirus), or West Nile Virus. In one example, one or more non-native epitope peptides are from a SARS-CoV, such as SARS-CoV1 or SARS-CoV2.

In one example, one or more non-native epitope peptides are from bacteria. Bacteria can be classified as gram-negative or gram-positive. Exemplary target gram-negative bacteria include, but are not limited to: Escherichia coli (e.g., K-12 and O157:H7), Shigella dysenteriae, and Vibrio cholerae. Exemplary target gram-positive bacteria include, but are not limited to: Bacillus anthracis, Staphylococcus aureus, Listeria, pneumococcus, gonococcus, and streptococcal meningitis. In one example, I one or more non-native epitope peptides are from one or more of the following: Group A Streptococcus; Group B Streptococcus; Helicobacter pylori; Methicillin-resistant Staphylococcus aureus; vancomycin-resistant enterococci; Clostridium difficile; E. coli (e.g., Shiga toxin producing strains); Listeria; Salmonella; Campylobacter; B. anthracis, Chlamydia trachomatis; Ebola, and Neisseria gonorrhoeae.

Protozoa, nemotodes, and fungi are also types of pathogens whose epitopes can be part of a chimeric Tp0751 (99-237) peptide provided herein, and used with the disclosed methods. Exemplary protozoa include, but are not limited to, Plasmodium (e.g., Plasmodium falciparum to diagnose malaria), Leishmania, Acanthamoeba, Giardia, Entamoeba, Cryptosporidium, Isospora, Balantidium, Trichomonas, Trypanosoma (e.g., Trypanosoma brucei), Naegleria, and Toxoplasma. Exemplary target fungi include, but are not limited to, Coccidiodes immitis and Blastomyces dermatitidis.

The chimeric Tp0751 (99-237) peptides can include one or more non-native epitope peptides, such as 1-20, 1-10, 2-10, 5-10, or 2-8 (such as 2, 3, 4, 5, 6, 7, or 8) different non-native epitope peptides. In some examples, a single loop is replaced all or in part by one or more non-native epitope peptides, such as at least 2 non-native epitope peptides, at least 3 non-native epitope peptides, at least 4 non-native epitope peptides, such as 2, 3, 4, 5, 6, 7, 8, 9 or 10 non-native epitope peptides. In one example, all or a portion of loop 2 is replaced by one or more non-native epitope peptides of at least 5 amino acids (aa), such as at least 6, at least 7, at least 8, at least 9 or at least 10 aa, such as 5-10 or 5-12 aa. In one example, all or a portion of loop 3 is replaced by one or more non-native epitope peptides of at least 2, at least 4, at least 5, at least 6, or at least 7, such as 2-7, 2-5, or 3-8 aa. In one example, all or a portion of loop 6 is replaced by one or more non-native epitope peptides of at least 5 aa, such as at least 6, at least 7, at least 8, at least 9, at least 10 aa, or at least 13 aa, such as 5-10, 5-12, or 5-13 aa. In one example, all or a portion of loop 8 is replaced by one or more non-native epitope peptides of at least 5 aa, such as at least 6, at least 7, at least 8, at least 9, at least 10, at least 15, at least 20, or at least 22 aa such as 5-23, 7-25, 7-22, or 10-15 aa.

In one example, the chimeric Tp0751 (99-237) peptide includes at least one non-native epitope peptide (such as 1, 2, 3, 4, 5, 6, 7, or 8 epitopes) from T. pallidum TprC protein, such as one or more of SEQ ID NO: 17, 18, 24, 25, 26, 31, 32, 35, 36, or 37. In one example, the chimeric Tp0751 (99-237) peptide includes at least one non-native epitope peptide (such as 1, 2, 3, 4, 5, 6, 7, or 8 epitopes) from T. pallidum TprK protein, such as one or more of SEQ ID NO: 19, 20, 21, 22, 27, 28, 29, 30, 33, and 34. In one example, the chimeric Tp0751 (99-237) peptide includes at least one non-native B cell epitope (such as 1, 2, 3, 4, 5, 6, 7, or 8 epitopes) from a T. pallidum protein, such as one or more of SEQ ID NOs: 115, 116, 117, 118, 119, 120, 121, 122, 123, 124, 125, 126, 127, 128, 217, 218, 219, 220, 221, 222, 223, 224, 225, 226, 227, 228, 229, 230, 231, or 232 (for example alone or in combination with one or more epitopes from Tp0326, Tp0346, Tp0856, Tp0483, TprC and/or TprK peptides, such as one or more of those provided herein). In one example, the chimeric Tp0751 (99-237) peptide includes at least one non-native T cell epitope (such as 1, 2, 3, 4, 5, 6, 7, or 8 epitopes) from a T. pallidum protein, such as one or more of SEQ ID NOs: 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, or 312 (for example alone or in combination with one or more epitopes from Tp0326, Tp0346, Tp0856, Tp0483, TprC and/or TprK peptides, such as one or more of those provided herein). In one example, the chimeric Tp0751 (99-237) peptide includes at least one non-native epitope peptide (such as 1, 2, 3, 4, 5, 6, 7, or 8 epitopes) from T. pallidum Tp0326 protein, such as SEQ ID NO: 23. In one example, the chimeric Tp0751 (99-237) peptide includes at least one non-native epitope peptide (such as 1, 2, 3, 4, 5, 6, 7, or 8 epitopes) from T. pallidum Tp0856 protein, such as SEQ ID NO: 143 or 145. In one example, the chimeric Tp0751 (99-237) peptide includes at least one non-native epitope peptide (such as 1, 2, 3, 4, 5, 6, 7, or 8 epitopes) from T. pallidum Tp0346 protein, such as SEQ ID NO: 144. In one example, the chimeric Tp0751 (99-237) peptide includes at least one non-native epitope peptide (such as 1, 2, 3, 4, 5, 6, 7, or 8 epitopes) from T. pallidum Tp0483 protein, such as SEQ ID NO: 146. In one example, the chimeric Tp0751 (99-237) peptide includes at least one non-native epitope peptide (such as 1, 2, 3, 4, 5, 6, 7, or 8 epitopes) from T. pallidum TprC protein (such as one or more of SEQ ID NO: 17, 18, 24, 25, 26, 31, 32, 35, 36, 37) and at least one non-native epitope peptide (such as 1, 2, 3, 4, 5, 6, 7, or 8 epitopes) from T. pallidum TprK protein (such as one or more of SEQ ID NO: 19, 20, 21, 22, 23, 27, 28, 29, 30, 33, and 34). In one example, the chimeric Tp0751 (99-237) peptide includes at least one non-native B- or T-cell epitope peptide (such as 1, 2, 3, 4, 5, 6, 7, or 8 epitopes) from T. pallidum TprC protein (such as one or more of SEQ ID NO: 17, 18, 24, 25, 26, 31, 32, 35, 36, 37), at least one non-native epitope peptide (such as 1, 2, 3, 4, 5, 6, 7, or 8 epitopes) from T. pallidum TprK protein (such as one or more of SEQ ID NO: 19, 20, 21, 22, 27, 28, 29, 30, 33, and 34), and at least one non-native B cell epitope peptide (such as 1, 2, 3, 4, 5, 6, 7, or 8 epitopes) from a T. pallidum protein, such as one or more of SEQ ID NO: 115, 116, 117, 118, 119, 120, 121, 122, 123, 124, 125, 126, 127, 128, 217, 218, 219, 220, 221, 222, 223, 224, 225, 226, 227, 228, 229, 230, 231, or 232. In one example, the chimeric Tp0751 (99-237) peptide includes at least one non-native B- or T-cell epitope peptide (such as 1, 2, 3, 4, 5, 6, 7, or 8 epitopes) from T. pallidum TprC protein (such as one or more of SEQ ID NO: 17, 18, 24, 25, 26, 31, 32, 35, 36, 37), at least one non-native epitope peptide (such as 1, 2, 3, 4, 5, 6, 7, or 8 epitopes) from T. pallidum TprK protein (such as one or more of SEQ ID NO: 19, 20, 21, 22, 27, 28, 29, 30, 33, and 34), and at least one non-native T cell epitope peptide (such as 1, 2, 3, 4, 5, 6, 7, or 8 epitopes) from a T. pallidum protein, such as one or more of SEQ ID NO: 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, or 312. In one example, the chimeric Tp0751 (99-237) peptide includes at least one non-native epitope peptide (such as 1, 2, 3, 4, 5, 6, 7, or 8 epitopes) from T. pallidum Tp0326 protein, such as SEQ ID NO: 23, and at least one non-native B cell epitope peptide (such as 1, 2, 3, 4, 5, 6, 7, or 8 epitopes) from a T. pallidum protein, such as one or more of SEQ ID NO: 115, 116, 117, 118, 119, 120, 121, 122, 123, 124, 125, 126, 127, 128, 217, 218, 219, 220, 221, 222, 223, 224, 225, 226, 227, 228, 229, 230, 231, or 232.

In one example, the chimeric Tp0751 (99-237) peptide includes a sequence having at least 80%, at least 85%, at least 90%, at least 95%, at least 96%, at least 97%, at least 98%, at least 99%, or 100% sequence identity to SEQ ID NO 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114. In one example, the chimeric Tp0751 (99-237) peptide includes a sequence having at least 80%, at least 85%, at least 90%, at least 95%, at least 96%, at least 97%, at least 98%, at least 99%, or 100% sequence identity to SEQ ID NO: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or 91. In some examples, the sequence of the chimeric Tp0751 (99-237) peptide comprises SEQ ID NO: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114, and further includes 1, 2, 3, 4, 5, 6, 7, 8, 9, 10, 11, 12, 13, 14, 15, 16, 17, 18, 19, or 20 conservative amino acid substitutions. In some examples, such conservative amino acid substitutions are not made to a non-native epitope peptide sequence. One skilled in the art will appreciate that variations can be made to the disclosed chimeric Tp0751 (99-237) peptides, as long as the chimeric Tp0751 (99-237) peptide can retain its ability to stimulate an immune response to the one or more non-native epitope peptides in vivo. In some examples, a variant chimeric Tp0751 (99-237) peptide retains the one or more non-native epitope peptides, but has changes at one or more other amino acids in the Tp0751 (99-237) peptide.

The disclosed chimeric Tp0751 (99-237) peptide sequences can be joined at either end to other unrelated sequences (for example, a linker or adjuvant).

Several embodiments include a multimer of chimeric Tp0751 (99-237) peptides, for example, a multimer including 2, 3, 4, 5, 6, 7, 8, 9, or 10 or more of the disclosed chimeric Tp0751 (99-237) peptides. In some examples, any of the disclosed chimeric Tp0751 (99-237) peptides are linked to another of the disclosed chimeric Tp0751 (99-237) peptides to form the multimer, such as a dimer or trimer.

In some embodiments, a chimeric Tp0751 (99-237) peptide is linked to a heterologous scaffold derived from another protein of human, animal, vegetal or synthetic origin, for example to stabilize its structure, increase its potency, or improve its pharmacological properties such as plasma half-life or resistance to protease digestion. In other examples, a single scaffold can bind multiple copies of the peptide. Examples of scaffold proteins include (but are not limited to) tetanus toxoid, cholera toxin beta-subunit, albumin, or the Fe portion of human immunoglobulin (Ig)G or IgM.

In some embodiments, any of the chimeric Tp0751 (99-237) peptides are used to elicit an immune response to an STI in a subject. In some such embodiments, induction of the immune response includes production of protective antibodies to STI, such as T. pallidum. Methods to assay for protective activity are known to the person of ordinary skill in the art and are further described herein.

Any suitable method may be used to make the disclosed peptides. For example, recombinant DNA technology can be used to generate a nucleic acid encoding the disclosed peptides, and from which the peptide can be expressed and purified. In addition to recombinant methods, the chimeric Tp0751 (99-237) peptides that are disclosed herein can also be constructed in whole or in part using standard peptide synthesis. Solid phase synthesis of the peptides can be accomplished by attaching the C-terminal amino acid of the sequence to an insoluble support followed by sequential addition of the remaining amino acids in the sequence. Proteins of greater length may be synthesized by condensation of the amino and carboxyl termini of shorter fragments.

Methods of forming peptide bonds by activation of a carboxyl terminal end (such as by the use of the coupling reagent N, N′-dicylohexylcarbodimide) can be used.

### Conjugation to a Carrier

In some implementations, a disclosed chimeric Tp0751 (99-237) peptide can be linked to a carrier protein by a linker (such as a peptide linker) or can be directly linked to the carrier protein (for example, by conjugation, or synthesis as a fusion protein) to form an immunogenic conjugate.

Suitable linkers include, but are not limited to, straight or branched-chain carbon linkers, heterocyclic carbon linkers or peptide linkers. One skilled in the art will recognize, for an immunogenic conjugate from two or more constituents, each of the constituents will contain the necessary reactive groups. Representative combinations of such groups are amino with carboxyl to form amide linkages or carboxy with hydroxyl to form ester linkages or amino with alkyl halides to form alkylamino linkages or thiols with thiols to form disulfides or thiols with maleimides or alkylhalides to form thioethers. Hydroxyl, carboxyl, amino and other functionalities, where not present may be introduced by known methods. Likewise, as those skilled in the art will recognize, a wide variety of linking groups may be employed. In some cases, the linking group can be designed to be either hydrophilic or hydrophobic to enhance the desired binding characteristics of the chimeric Tp0751 (99-237) peptide and the carrier. The covalent linkages should be stable relative to the solution conditions under which the conjugate is subjected.

In some implementations, the linkers are joined to the constituent amino acids through their side groups (such as through a disulfide linkage to cysteine) or to the alpha carbon amino and carboxyl groups of the terminal amino acids. In some implementations, the chimeric Tp0751 (99-237) peptide, the linker, and the carrier can be encoded as a single fusion polypeptide such that the chimeric Tp0751 (99-237) peptide and the carrier are joined by peptide bonds.

The procedure for attaching a molecule to a polypeptide varies according to the chemical structure of the molecule. Polypeptides typically contain a variety of functional groups; for example, carboxylic acid (COOH), free amine (—NH₂) or sulfhydryl (—SH) groups, which are available for reaction with a suitable functional group on a polypeptide. Alternatively, the polypeptide is derivatized to expose or attach additional reactive functional groups. The derivatization may involve attachment of any number of linker molecules such as those available from Pierce Chemical Company, Rockford, IL.

Conjugates in which more than one chimeric Tp0751 (99-237) peptide is conjugated to a single carrier protein can be prepared. The conjugation of multiple chimeric Tp0751 (99-237) peptides to a single carrier protein is possible because the carrier protein has multiple lysine or cysteine side-chains that can serve as sites of attachment. The amount of chimeric Tp0751 (99-237) peptide reacted with the amount of carrier may vary depending upon the specific chimeric Tp0751 (99-237) peptide and the carrier protein. However, the respective amounts should be sufficient to introduce about 1-30 chains of chimeric Tp0751 (99-237) peptide onto the carrier protein. The resulting number of chimeric Tp0751 (99-237) peptide linked to a single carrier molecule may vary depending upon the specific chimeric Tp0751 (99-237) peptide and the carrier protein. In some implementations, from 1 to 30, such as about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 chimeric Tp0751 (99-237) peptide can be linked to each carrier protein molecule. “About” in this context refers to plus or minus 5% when measuring an average number of chimeric Tp0751 (99-237) peptide per carrier molecule in the conjugate. Thus, in some implementations, the average ratio of chimeric Tp0751 (99-237) peptide to carrier protein molecules is between about 1:1 and about 30:1, such as about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1, about 21:1, about 22:1, about 23:1, about 24:1, about 25:1, about 26:1, about 27:1, about 28:1, about 29:1, or about 30:1, for example, between about 1:1 and about 15:1, between about 5:1 and about 20:1, or between about 10:1 and about 30:1.

In some implementations (such as when KLH is used as a carrier), from 1 to 1000, such as about 50, about 100, about 200, about 300, about 400, about 500, about 700, about 1000, about 9, about 10, about 11, about 12, about 13, about 14, about 15, about 16, about 17, about 18, or about 19 chimeric Tp0751 (99-237) peptide molecules can be linked to each carrier protein. Thus, in some implementations, the average ratio of chimeric Tp0751 (99-237) peptide molecule to carrier protein is between about 1:1 and about 1000:1, such as between about 100:1 and about 500:1, between about 500:1 and about 10000:1, or between about 250:1 and about 750:1.

Examples of suitable carriers are those that can increase the immunogenicity of the conjugate and/or elicit antibodies against the carrier which are diagnostically, analytically, and/or therapeutically beneficial. Useful carriers include polymeric carriers, which can be natural, recombinantly produced, semi-synthetic or synthetic materials containing one or more amino groups, such as those present in a lysine amino acid residue present in the carrier, to which a reactant moiety can be attached. Carriers that fulfill these criteria are generally known in the art (see, for example, Fattom et al., Infect. Immun. 58:2309-12, 1990; Devi et al., PNAS 88:7175-79, 1991; Szu et al., Infect. Immun. 59:4555-61, 1991; Szu et al., J. Exp. Med. 166:1510-24, 1987; and Pavliakova et al., Infect. Immun. 68:2161-66, 2000). A carrier can be useful even if the antibody that it elicits is not of benefit by itself.

Specific, non-limiting examples of suitable polypeptide carriers include, but are not limited to, natural, semi-synthetic or synthetic polypeptides or proteins from bacteria or viruses. In one implementation, bacterial products for use as carriers include bacterial toxins. Bacterial toxins include bacterial products that mediate toxic effects, inflammatory responses, stress, shock, chronic sequelae, or mortality in a susceptible host. Specific, non-limiting examples of bacterial toxins include, but are not limited to: B. anthracis PA, B. anthracis LF, bacterial toxins and toxoids, such as tetanus toxin/toxoid, C. perfringens exotoxin/toxoid, diphtheria toxin/toxoid, P. aeruginosa exotoxin/toxoid, pertussis toxin/toxoid, and C. difficile toxin B or A, as well as fragments, analogs, and mimetics thereof, and combinations of two or more thereof. In some implementations, the carrier is tetanus toxin heavy chain C fragment, or a fragment, analog, or mimetic thereof. In some implementations, the carrier is cholera toxin B (CTB), or a fragment, analog, or mimetic thereof. Viral proteins, such as hepatitis B surface antigen (for example, as described in U.S. Pat. Nos. 5,151,023 and 6,013,264) and core antigen (for example, as described in U.S. Pat. Nos. 4,547,367 and 4,547,368) can also be used as carriers, as well as proteins from higher organisms such as keyhole limpet hemocyanin (KLH), horseshoe crab hemocyanin, Concholepas Concholepas Hemocyanin (CCH), Ovalbumin (OVA), edestin, mammalian serum albumins (such as bovine serum albumin), and mammalian immunoglobulins. In some examples, the carrier is bovine serum albumin.

In some implementations, the carrier is selected from one of: Keyhole Limpet Hemocyanin (KLH), tetanus toxoid, tetanus toxin heavy chain C fragment, diphtheria toxoid, diphtheria toxin variant CRM197, or H influenza protein D (HiD). CRM197 is a genetically detoxified form of diphtheria toxin; a single mutation at position 52, substituting glutamic acid for glycine, causes the ADP-ribosyltransferase activity of the native diphtheria toxin to be lost.

Following conjugation of a chimeric Tp0751 (99-237) peptide to the carrier protein, the conjugate can be purified. In one example, the purification step separates the unconjugated chimeric Tp0751 (99-237) peptide or carrier from the conjugate. The conjugates can be purified away from unconjugated chimeric Tp0751 (99-237) peptide or carrier by any number of standard techniques including, for example, size exclusion chromatography, density gradient centrifugation, hydrophobic interaction chromatography, or ammonium sulfate fractionation.

In several implementations, the disclosed immunogenic conjugates can be formulated into immunogenic composition (such as vaccines), for example by the addition of a pharmaceutically acceptable carrier and/or adjuvant.

### Polynucleotides and Expression

Polynucleotides encoding a disclosed chimeric Tp0751 (99-237) peptide are provided. These polynucleotides include DNA, cDNA and RNA sequences (such as mRNA) which encode the antigen. One of skill in the art can readily use the genetic code to construct a variety of functionally equivalent nucleic acids, such as nucleic acids which differ in sequence but which encode the same protein sequence, or encode a conjugate or fusion protein including the nucleic acid sequence.

The nucleic acid molecule encoding the chimeric Tp0751 (99-237) peptide can be any suitable type of nucleic acid molecule including DNA (such as cDNA) and RNA (such as mRNA, circular RNA), as well as modified forms thereof (such as but are not limited to modified mRNA with N1-methylpseudouridine in place of uridine), that encode the chimeric Tp0751 (99-237) peptide, as well as vectors including the DNA, cDNA and RNA sequences, such as a DNA or RNA vector used for immunization. The genetic code may be used to construct a variety of functionally equivalent nucleic acids, such as nucleic acids which differ in sequence but which encode the same fusion protein sequence.

Exemplary nucleic acid molecules can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Nucleic acid molecules can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

Nucleic acid molecules can be prepared by in vitro transcription. For example, cDNA encoding the polynucleotides described herein may be transcribed to mRNA using an in vitro transcription (IVT) system. A non-limiting example of in vitro transcription of RNA is described in WO2014/152027, which is incorporated by reference herein in its entirety.

Nucleic acid molecules can be prepared by chemical synthesis. For example, nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

The polynucleotides can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Nucleic acid molecules encoding the disclosed chimeric Tp0751 (99-237) peptides can be expressed in vitro by transfer into a suitable host cell. The cell may be prokaryotic or eukaryotic. In one example, the cell is E. coli. In one example, the cell is a mammalian cell. The term also includes any progeny of the subject host cell. It is understood that all progenies may not be identical to the parental cell since there may be mutations that occur during replication. Any methods of stable transfer, meaning that the foreign DNA is continuously maintained in the host, can be used.

In some implementations, a nucleic acid molecule encoding a disclosed chimeric Tp0751 (99-237) peptide can be included in a viral vector (such as an AAV or adenoviral vector), for example, for expression of the immunogen in a host cell, or for immunization of a subject. In some aspects, the viral vectors are administered to a subject as part of a prime-boost vaccination. In some aspects, the viral vectors are included in a vaccine, such as a primer vaccine or a booster vaccine for use in a prime-boost vaccination.

In some aspects, the viral vector is replication-competent. For example, the viral vector can have a mutation in the viral genome that does not inhibit viral replication in host cells. The viral vector also can be conditionally replication-competent. In other examples, the viral vector is replication-deficient in host cells.

Exemplary viral vectors that can be used to express the disclosed chimeric Tp0751 (99-237) peptides, include, but are not limited to, polyoma (e.g., SV40), adenovirus, adeno-associated virus, Sindbis viruses, alphaviruses, and retroviruses of avian, murine, and human origin. Baculovirus (Autographa californica multinuclear polyhedrosis virus; AcMNPV) vectors can be used.

In some aspects, the viral vector can include an adenoviral vector that expresses a disclosed chimeric Tp0751 (99-237) peptides. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Replication competent and deficient adenoviral vectors (including singly and multiply replication deficient adenoviral vectors) can be used. Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/02231 1.

In one example, the disclosed isolated nucleic acid molecules encode a chimeric Tp0751 (99-237) peptide having at least 80%, at least 85%, at least 90%, at least 95%, at least 96%, at least 97%, at least 98%, at least 99%, or 100% sequence identity to any one of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114. In one example, the disclosed isolated nucleic acid molecules encoding a chimeric Tp0751 (99-237) peptide have at least 80%, at least 85%, at least 90%, at least 95%, at least 96%, at least 97%, at least 98%, at least 99%, or 100% sequence identity to SEQ ID NO: 95, 96, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 201, 203, 204, 205, 206, 307, 208, 209, 210, 211, 212, 213, 214, 215, or 216. In one example, the isolated nucleic acid molecule is DNA, such as cDNA. In another example, the isolated nucleic acid molecule is RNA, such as mRNA. Such isolated nucleic acid molecules can be operably linked to a promotor, such as a constitutive or inducible promoter. In some examples, the isolated nucleic acid molecules are part of a vector.

### Compositions and Cells

Pharmaceutical compositions, including immunogenic compositions, that include one or more chimeric Tp0751 (99-237) peptides or isolated nucleic acid molecules that encode one or more chimeric Tp0751 (99-237) peptides, and a pharmaceutically acceptable carrier, are provided. Such compositions can be administered to subjects by a variety of administration modes, for example, intramuscular, subcutaneous, intravenous, intra-arterial, intra-articular, intraperitoneal, or parenteral routes. Methods for preparing administrable compositions are described in more detail in such publications as Remington: The Science and Practice of Pharmacy, 22^{nd} ed., London, UK: Pharmaceutical Press, 2013.

Pharmaceutical compositions, which include one or more chimeric Tp0751 (99-237) peptides, isolated nucleic acid molecules that encode one or more chimeric Tp0751 (99-237) peptides, such as one or more chimeric Tp0751 (99-237) peptides having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114, or one or more nucleic acid molecules (or vectors containing such) encoding one or more chimeric Tp0751 (99-237) peptides having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114 are provided. In some examples, the compositions include two or more chimeric Tp0751 (99-237) peptides, such as 2, 3, 4, 5, 6, 7, 8, 9 or 10 different one chimeric Tp0751 (99-237) peptides, such as 2, 3, 4, 5, 6, 7, 8, 9 or 10 different chimeric Tp0751 (99-237) peptides having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114. In some examples, the compositions include two or more chimeric Tp0751 (99-237) peptide coding sequences, such as 2, 3, 4, 5, 6, 7, 8, 9 or 10 different one chimeric Tp0751 (99-237) peptides coding sequences, such as 2, 3, 4, 5, 6, 7, 8, 9 or 10 different chimeric Tp0751 (99-237) coding sequences that encode 2, 3, 4, 5, 6, 7, 8, 9 or 10 different chimeric Tp0751 (99-237) proteins having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114. Such compositions include at least one pharmaceutically acceptable carrier, such as water or saline.

Also provided are liposomes, which may be present in a pharmaceutical composition, which include one or more chimeric Tp0751 (99-237) peptides, or isolated nucleic acid molecules that encode one or more chimeric Tp0751 (99-237) peptides, such as one or more chimeric Tp0751 (99-237) peptides having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114 or one or more nucleic acid molecules (or vectors containing such) encoding one or more chimeric Tp0751 (99-237) peptides having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114. For example, such a liposome can include a pharmaceutically acceptable carrier, such as water, oil, a water and oil emulsion, or saline. In one example the liposome is a neutral liposome or a liposome containing the one or more chimeric Tp0751 (99-237) peptides/nucleic acid molecules and one or more cytokines,

Also provided are cells, which may be present in a pharmaceutical composition, which include one or more chimeric Tp0751 (99-237) peptides, or isolated nucleic acid molecules that encode one or more chimeric Tp0751 (99-237) peptides, such as one or more chimeric Tp0751 (99-237) peptides having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114, or one or more nucleic acid molecules (or vectors containing such) encoding one or more chimeric Tp0751 (99-237) peptides having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114. Exemplary cells include bacterial cells (such as E. coli), mammalian cells, and yeast cells. Such cells can be present in a pharmaceutically acceptable carrier, such as water, oil, a water and oil emulsion, or saline.

Immunogenic compositions, such as vaccines, which include one or more of the chimeric Tp0751 (99-237) peptides, such as one or more chimeric Tp0751 (99-237) peptides having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114, one or more nucleic acid molecules (or vectors containing such) encoding one or more chimeric Tp0751 (99-237) peptides having at least 80%, at least 90%, at least 95%, at least 98%, at least 99%, or 100% sequence identity to any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114, are provided. For example, such a vaccine composition can include an adjuvant to enhance an immune response of the host, such as alum, aluminum hydroxide, aluminum phosphate, Freund incomplete adjuvant), immunostimulatory oligonucleotides (such as those including a CpG motif), costimulatory molecules, IL-2, RANTES, GM-CSF, TNF-α, IFN-γ, G-CSF, LFA-3, CD72, B7-1, B7-2, OX-40L, 4-1 BBL, immune stimulating complex (ISCOM) matrix, and toll-like receptor (TLR) agonists, such as TLR-9 agonists, Poly I:C, or PolyICLC. Additional adjuvants include monophosphoryl lipid A (MPL) and Alhydroxiquim-II. In one example the adjuvant includes chitosan, Bacillus-Calmette-Guerin adjuvant or RIBI adjuvant. In one example the adjuvant is TiterMax Gold (Sigma). In some examples, vaccines further include a pharmaceutically acceptable carrier, such as water, oil, a water and oil emulsion, or saline. Other exemplary adjuvants include alhydrogel, Lipid-A and derivatives or variants thereof, oil-emulsions, saponins, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12, may be used as an adjuvant. One exemplary adjuvant that can be used is LiT4Q (e.g., liposomal TLR4/7 adjuvant). In one example the adjuvant used is PAI-RIBI Natural. In one example the adjuvant used is PAI-RIBI synthetic. In one example the adjuvant used is PAI-sRI-T1/2. In one example the adjuvant used is alum. In one example the adjuvant used is AS04. Some adjuvants help to stimulate the immune system in a non-specific way, thus enhancing the immune response to a pharmaceutical product.

Thus, a composition described herein (including immunogenic compositions and vaccines) can be formulated with pharmaceutically acceptable carriers to help retain biological activity while also promoting increased stability during storage within an acceptable temperature range. Exemplary carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

In some aspects, the immunogenic composition can be provided as a sterile composition. The immunogenic composition typically contains an effective amount of a disclosed immunogen and can be prepared by conventional techniques. Typically, the amount of immunogen in each dose of the immunogenic composition is selected as an amount which elicits an immune response without significant, adverse side effects. In some aspects, the immunogenic composition can be provided in unit dosage form for use to elicit an immune response in a subject, for example, to prevent STI in the subject. A unit dosage form contains a suitable single preselected dosage for administration to a subject, or suitable marked or measured multiples of two or more preselected unit dosages, and/or a metering mechanism for administering the unit dose or multiples thereof. In other aspects, the composition further includes an adjuvant.

In some aspects, the composition comprises mRNA encoding a chimeric Tp0751 (99-237) peptide formulated in a lipid nanoparticle. Exemplary lipid nanoparticles typically include ionizable cationic lipid, non-cationic lipid, sterol and PEG lipid components along with the nucleic acid cargo of interest. The lipid nanoparticles can be generated using any suitable components, compositions, and methods, such as described in PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575, PCT/US2016/069491, and U.S. App. Publ. 2022/0241399, all of which are incorporated by reference herein in their entirety.

In several embodiments, the mRNA is formulated in a lipid nanoparticle; for example, comprising a PEG-modified lipid, a non-cationic lipid, a sterol, an ionizable lipid, or any combination thereof. In some embodiments, the lipid nanoparticle is composed of 50 mol % ionizable lipid ((2 hydroxyethyl)(6 oxo 6-(undecycloxy)hexyl)amino)octanoate, 10 mol % 1,2 distearoyl sn glycerol-3 phosphocholine (DSPC), 38.5 mol % cholesterol, and 1.5 mol % 1-monomethoxypolyethyleneglycol-2,3,dimyristylglycerol with polyethylene glycol of average molecular weight 2000 (PEG2000 DMG). The mRNA/lipid nanoparticle composition may be provided in any suitable carrier, such as a sterile liquid for injection at a concentration of 0.5 mg/mL in 20 mM trometamol (Tris) buffer containing 87 mg/mL sucrose and 10.7 mM sodium acetate, at pH 7.5 and with appropriate diluent.

### Methods of Inducing an Immune Response and Treatment

The disclosed compositions, chimeric Tp0751 (99-237) peptides/nucleic acids, cells, and vectors, and immunogenic compositions including same, can be used in methods of inducing an immune response to an STI to prevent, inhibit, and/or treat an STI, such as T. pallidum subsp. pallidum infection which causes syphilis, Chlamydia infection which causes chlamydia, and N. gonorrhoeae which causes gonorrhea. The non-native epitope peptide(s) in the chimeric Tp0751 (99-237) peptides/nucleic acids is selected based on the STI to be prevented, inhibited, and/or treated. For example, if the method is for preventing or inhibiting T. pallidum or treating syphilis, the non-native epitope peptide(s) is/are from a T. pallidum protein, such as TprC and/or TprK. For example, if the method is for preventing or inhibiting Chlamydia or treating chlamydia, the non-native epitope peptide(s) is/are from a Chlamydia protein. For example, if the method is for preventing or inhibiting N. gonorrhoeae or treating gonorrhea, the non-native epitope peptide(s) is/are from a Neisseria gonorrhoeae protein. In some examples, a chimeric Tp0751 (99-237) peptides/nucleic acid includes multiple epitope peptides from two or more different STIs, such as T. pallidum and Chlamydia, T. pallidum and Neisseria gonorrhoeae, T. pallidum, Chlamydia, and N. gonorrhoeae.

The disclosed chimeric Tp0751 (99-237) peptides/nucleic acid molecules can be used to create epitopes from an STIs grafted into one or more different loops 1-8 of the scaffold, which can then be used as an immunogen and administered as a subunit vaccine with a customized, disease relevant human-track adjuvant (i.e., adjuvant whose components are suitable for use in humans). For example, heterologous vaccine constructs can be created, which encompass the chimeric Tp0751 (99-237) peptide scaffold with different epitopes engrafted onto the scaffold. These homologous and heterologous constructs can be multimerized into dimers, trimers, or additional multimers using linker regions, to enhance immunogenicity while maintaining solubility and stability. Alternatively, homologous or heterologous chimera constructs can be expressed and displayed on outer membrane vesicles or contained within liposomes, either with or without a human-track adjuvant, to allow exposure of a multimerized vaccine construct to the immune system in a conformationally conserved manner. In one example, mRNA vaccines are generated using the chimeric Tp0751 (99-237) peptide coding sequence, thus taking advantage of the inherent adjuvant capacity, multivalency potential, and high immunogenicity associated with this mode of delivery.

When inhibiting, treating, or preventing STI, the methods can be used either to avoid infection in a seronegative subject (e.g., by inducing an immune response that protects against infection), or to treat existing infection in a seropositive subject. Hence in some implementations the methods involve selecting a subject at risk for contracting STI, or a subject at risk of developing STD (such as a subject with an STI), and administering a disclosed immunogen to the subject to elicit an immune response to the pathogen in the subject.

To identify subjects for prophylaxis or treatment according to the methods of the disclosure, screening methods can be employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize STI. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The disclosed immunogens can be used in coordinate (or prime-boost) immunization protocols or combinatorial formulations. In certain implementations, novel combinatorial immunogenic compositions and coordinate immunization protocols employ separate immunogens or formulations, each directed toward eliciting an anti-STI immune response, such as an immune response to an STI (such as the one or more of the epitopes in the disclosed chimeric Tp0751 (99-237) peptides). Separate immunogenic compositions (such as two or more different chimeric Tp0751 (99-237) peptides/nucleic acids) that elicit the anti-STI immune response can be combined in a polyvalent immunogenic composition administered to a subject in a single immunization step, or they can be administered separately (in monovalent immunogenic compositions) in a coordinate immunization protocol.

In one implementation, a suitable immunization regimen includes at least two separate inoculations with one or more immunogenic compositions including a disclosed immunogen, with a second inoculation being administered more than about two, about three to eight, or about four, weeks following the first inoculation. A third inoculation can be administered several months after the second inoculation, and in specific implementations, more than about five months after the first inoculation, more than about six months to about two years after the first inoculation, or about eight months to about one year after the first inoculation. Periodic inoculations beyond the third are also desirable to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect, e.g., prevention of T. pallidum infection or progression to syphilis, improvement in disease state (e.g., reduction in chancres), or reduction in transmission frequency to an uninfected partner. If such monitoring indicates that vaccination is sub-optimal, the subject can be boosted with an additional dose of immunogenic composition, and the vaccination parameters can be modified in a fashion expected to potentiate the immune response. Thus, for example, a dose of a disclosed immunogen can be increased or the route of administration can be changed.

There can be several boosts, and that each boost can be a different immunogen. It is also contemplated in some examples that the boost may be the same immunogen as another boost, or the prime.

In several implementations, a disclosed immunogen (chimeric Tp0751 peptides/nucleic acids) can be administered to the subject simultaneously with the administration of an adjuvant. In other implementations, the immunogen can be administered to the subject after the administration of an adjuvant and within a sufficient amount of time to elicit the immune response.

Determination of effective dosages is typically based on animal model studies followed up by human clinical trials and is guided by administration protocols that significantly reduce the occurrence or severity of targeted disease symptoms or conditions in the subject, or that elicit a desired response in the subject (such as a protective immune response). Suitable models in this regard include, for example, rabbits. Alternatively, effective dosages can be determined using in vitro models (for example, immunologic and histopathologic assays). Using such models, only ordinary calculations and adjustments are required to determine an appropriate concentration and dose to administer an effective amount of the composition (for example, amounts that are effective to elicit a desired immune response or alleviate one or more symptoms of a targeted disease). In alternative implementations, an effective amount or effective dose of the composition may simply inhibit or enhance one or more selected biological activities correlated with a disease or condition, as set forth herein, for either therapeutic or diagnostic purposes.

Dosage can be varied by the attending clinician to maintain a desired concentration at a target site (for example, systemic circulation). Higher or lower concentrations can be selected based on the mode of delivery, for example, trans-epidermal, rectal, oral, pulmonary, or intranasal delivery versus intravenous or subcutaneous delivery. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

A non-limiting range for an effective amount of the disclosed immunogen (such chimeric Tp0751 (99-237) peptides/nucleic acids) within the methods and immunogenic compositions of the disclosure is about 0.0001 mg/kg body weight to about 10 mg/kg body weight, such as about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example, 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight. In some implementations, the dosage includes a set amount of a disclosed immunogen such as from about 1-300 μg, for example, a dosage of about 1, 2, 3, 4, 5, 6, 7, 8, 9, 10, 15, 20, 30, 40, 50, 60, 70, 80, 90, 100, 150, 200, 250, or about 300 μg.

In some embodiments, a single dose of an mRNA vaccine (such as one encoding a chimeric Tp0751 (99-237) peptide of the present disclosure) comprises 100 μg to 1000 μg of mRNA. For example, a single dose (e.g., comprising mRNA encoding a disclosed chimeric Tp0751 (99-237) peptide formulated in a lipid nanoparticle) may be 100 μg to 900 μg, 100 μg to 800 μg, 100 μg to 700 μg, 100 μg to 600 μg, 100 μg to 500 μg, 200 μg to 900 μg, 200 μg to 800 μg, 200 μg to 700 μg, 200 μg to 600 μg, 200 μg to 500 μg, 300 μg to 900 μg, 300 μg to 800 μg, 300 μg to 700 μg, 300 g to 600 μg, or 300 μg to 600 μg of mRNA. In some embodiments, a single dose (e.g., comprising mRNA encoding a disclosed chimeric Tp0751 (99-237) peptide formulated in a lipid nanoparticle) is 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 300 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, or 400 μg of mRNA.

The dosage and number of doses will depend on the setting, for example, in an adult or anyone primed by prior STI infection or immunization, a single dose may be a sufficient booster. In naïve subjects, in some examples, at least two doses would be given, for example, at least three doses. In some implementations, an annual boost is given, for example, along with an annual influenza vaccination.

STI does not need to be completely inhibited for the methods to be effective. For example, elicitation of an immune response to the STI with one or more of the disclosed chimeric Tp0751 (99-237) peptides/nucleic acid molecules can reduce or inhibit STI by at least 10%, at least 20%, at least 50%, at least 60%, at least 70%, at least 80%, at least 90%, at least 95%, at least 98%, or even at least 100% (elimination or prevention of detectable infected cells), as compared to STI in the absence of the therapeutic agent.

Following immunization of a subject, serum can be collected from the subject at appropriate time points, frozen, and stored for testing. In some examples, the sample is assessed for the presence of the STI.

In some examples, a plasmid DNA vaccine is used to express a disclosed immunogen in a subject. For example, a nucleic acid molecule encoding a disclosed immunogen can be administered to a subject to elicit an immune response to one or more STIs. In some implementations, the nucleic acid molecule can be included on a plasmid vector for DNA immunization, such as the pVRC8400 vector (described in Barouch et al., J. Virol, 79, 8828-8834, 2005).

In another approach to using nucleic acids for immunization, a disclosed chimeric Tp0751 (99-237) peptide coding sequence can be expressed by attenuated viral hosts or vectors or bacterial vectors. Recombinant vaccinia virus, adeno-associated virus (AAV), herpes virus, retrovirus, or other viral vectors can be used to express the peptide thereby eliciting an immune response.

In one implementation, a nucleic acid encoding a disclosed chimeric Tp0751 (99-237) peptide is introduced directly into cells. For example, a nucleic acid encoding a chimeric Tp0751 (99-237) peptide can be loaded onto gold microspheres and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 g/kg to about 50 mg/kg, and typically are about 0.005 mg/kg to about 5 mg/kg (see, e.g., U.S. Pat. No. 5,589,466).

In one example, a vaccine encoding a disclosed chimeric Tp0751 (99-237) peptide includes outer membrane vesicles (OMV), such as Neisseria OMVs (NOMV), cationic nanocarriers, such as a Lipid InOrganic Nanoparticle (LION), or both. For example, mRNA encoding one or more disclosed chimeric Tp0751 (99-237) peptides can be synthesized and combined with Lipid InOrganic Nanoparticle (LION), for example at a nitrogen-to-phosphate ratio of 15 in a simple 1:1 volume mix and incubated on ice, for example for 30 minutes [see, for example, Hawman et al, EBioMedicine 83, 104196, doi:10.1016/j.ebiom.2022.104196 (2022)]. In another example, a NOMV-chimeric Tp0751 (99-237) peptide vaccine formulation can be produced using the methods provided in Beernink et al. J Infect Dis 219, 1130-1137, doi:10.1093/infdis/jiy609 (2019)].

Thus, in some examples, provided herein are methods of stimulating an immune response in a subject, for example against, T. pallidum by administering to the subject a therapeutically effective amount of one or more chimeric Tp0751 (99-237) proteins or nucleic acids (or composition, liposome, cell, or vector including such). In some examples, 2, 3, 4 or 5 different chimeric Tp0751 (99-237) proteins or nucleic acids (or composition, liposome, cell, or vector including such) are administered. In some examples, the methods elicit both a humoral and cell-mediated immune response in the treated subject. Also provided are methods of preventing or treating Treponema pallidum subsp. pallidum infection, such as treating syphilis, in a subject by administering to the subject a therapeutically effective amount of one or more chimeric Tp0751 (99-237) proteins or nucleic acids (or composition, liposome, cell, or vector including such).

In some examples, the disclosed methods reduce one or more symptoms of syphilis in the subject by at least 20%, at least 25%, at least 50%, at least 60%, at least 70%, at least 80%, at least 90%, at least 95%, at least 99% or even 100%, for example, relative to no administration of the chimeric Tp0751 (99-237) protein or nucleic acid (or composition, liposome, cell, or vector including such). In some examples, the one or more symptoms of syphilis include one or more of chancre(s), rash, gumma, lymphadenopathy, tabes dorsalis, general paresis, panuvetis, conjunctivitis, anterior uveitis, posterior interstitial keratitis, optic neuropathy, retinal vasculitis, and mucosal lesions.

In some examples, the disclosed methods induce an immune response (such as a B-cell and T cell response), such as increase production of Th1 cytokines (such as interferon-γ (IFN-γ), interleukin-2 (IL-2), and/or interleukin-12 (IL-12), in the treated subject by at least 20%, at least 25%, at least 50%, at least 60%, at least 70%, at least 80%, at least 90%, at least 95%, at least 99%, at least 100%, at least 200%, at least 300%, at least 400%, or at least 500%, for example, relative to no administration of the chimeric Tp0751 (99-237) protein or nucleic acid (or composition, liposome, cell, or vector including such).

In some examples, the disclosed methods increase antibody production against T. pallidum in the treated subject by at least 20%, at least 25%, at least 50%, at least 60%, at least 70%, at least 80%, at least 90%, at least 95%, at least 99%, at least 100%, at least 200%, at least 300%, at least 400%, or at least 500%, for example, relative to no administration of the chimeric Tp0751 (99-237) protein or nucleic acid (or composition, liposome, cell, or vector including such).

In some examples, the disclosed methods increase phagocytosis of opsonized T. pallidum in the treated subject by at least 20%, at least 25%, at least 50%, at least 60%, at least 70%, at least 80%, at least 90%, at least 95%, at least 99%, at least 100%, at least 200%, at least 300%, at least 400%, or at least 500%, for example, relative to no administration of the chimeric Tp0751 (99-237) protein or nucleic acid (or composition, liposome, cell, or vector including such).

In some examples, the disclosed methods reduce chancre volume and/or the number of chancres in the treated subject by at least 20%, at least 25%, at least 50%, at least 60%, at least 70%, at least 80%, at least 90%, at least 95%, at least 99%, at least 100%, at least 200%, at least 300%, at least 400%, or at least 500%, for example, relative to no administration of the chimeric Tp0751 (99-237) protein or nucleic acid (or composition, liposome, cell, or vector including such).

In some examples, the disclosed methods reduce the % of chancres that ulcerate in the treated subject by at least 20%, at least 25%, at least 50%, at least 60%, at least 70%, at least 80%, at least 90%, at least 95%, at least 99%, at least 100%, at least 200%, at least 300%, at least 400%, or at least 500%, for example, relative to no administration of the chimeric Tp0751 (99-237) protein or nucleic acid (or composition, liposome, cell, or vector including such).

In some examples, the disclosed methods reduce dissemination in the treated subject by at least 20%, at least 25%, at least 50%, at least 60%, at least 70%, at least 80%, at least 90%, at least 95%, at least 99%, at least 100%, at least 200%, at least 300%, at least 400%, or at least 500%, for example, relative to no administration of the chimeric Tp0751 (99-237) protein or nucleic acid (or composition, liposome, cell, or vector including such).

In some examples, the disclosed methods reduce dissemination of T. pallidum to lymph nodes and other tissues and organs in the treated subject by at least 20%, at least 25%, at least 50%, at least 60%, at least 70%, at least 80%, at least 90%, at least 95%, at least 99%, at least 100%, at least 200%, at least 300%, at least 400%, or at least 500%, for example, relative to no administration of the chimeric Tp0751 (99-237) protein or nucleic acid (or composition, liposome, cell, or vector including such).

In some examples, the disclosed methods reduce congenital syphilis in the treated subject by at least 20%, at least 25%, at least 50%, at least 60%, at least 70%, at least 80%, at least 90%, at least 95%, at least 99%, at least 100%, at least 200%, at least 300%, at least 400%, or at least 500%, for example, relative to no administration of the chimeric Tp0751 (99-237) protein or nucleic acid (or composition, liposome, cell, or vector including such).

In some examples, combinations of these effects are achieved.

In some examples, the disclosed methods include administering additional therapeutic agents to the subject, for example before, after, or concurrently with the disclosed chimeric Tp0751 (99-237) protein or nucleic acid. In one example, the subject is further treated with one or more antibiotics, such as a therapeutically effective amount of penicillin, doxycycline, tetracycline and/or ceftriaxone.

### EXAMPLES

The following examples are provided to illustrate particular features of certain aspects of the disclosure, but the scope of the claims should not be limited to those features exemplified.

### Example 1

### In Silico Construct Design

In-depth structural analysis of the Tp0751 (99-237) lipocalin-like domain (PDB: 5JK2) was performed to assess the engineering potential of each flexible region as defined by a region that is predicted to be dispensable to the core stability of the lipocalin-like domain. Structural data, including analysis of thermal motion information (b-factor), were used to generate a preliminary map of permissive regions.

For example, loop 8 (SEQ ID NO: 7), which is the longest loop in Tp0751, showed the highest b-factor overall (FIGS. 1A-1C) and was prioritized for substitution. Additional regions, including the C-terminus (SEQ ID NO: 8) and loop 2 (SEQ ID NO: 5), could not be fully modeled in the structure consistent with inherent flexibility and greater potential to accommodate substitutions.

Boundaries of each flexible region (loops and termini) of Tp0751 (99-237) were delineated using structural analysis. Each of the loop residues were assessed for their interactions with the core beta-barrel structure, ensuring that the residues chosen for removal were not participating in backbone hydrogen bonding between beta-strands, which would in turn affect structural stability.

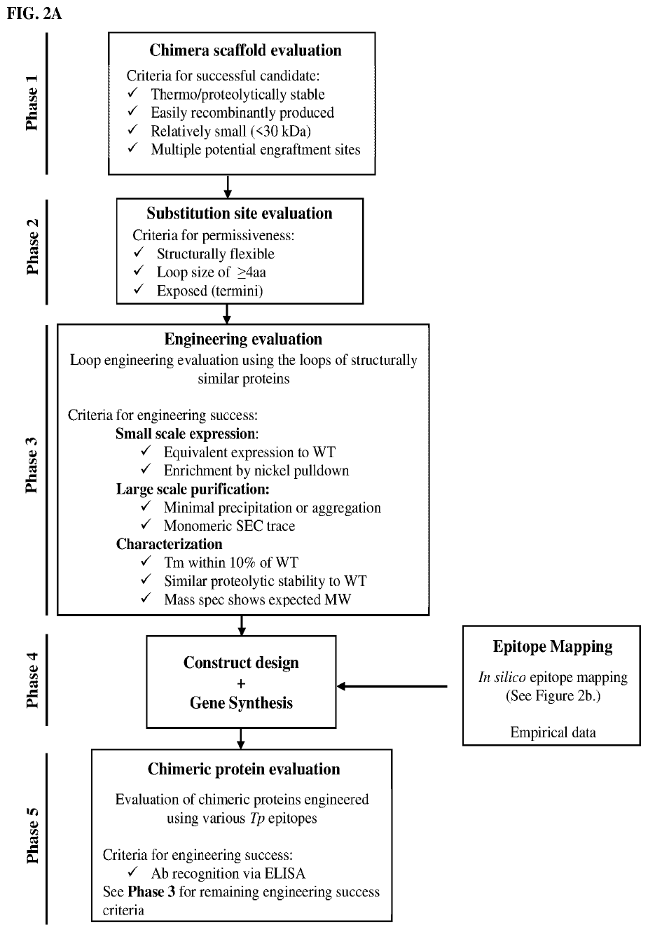

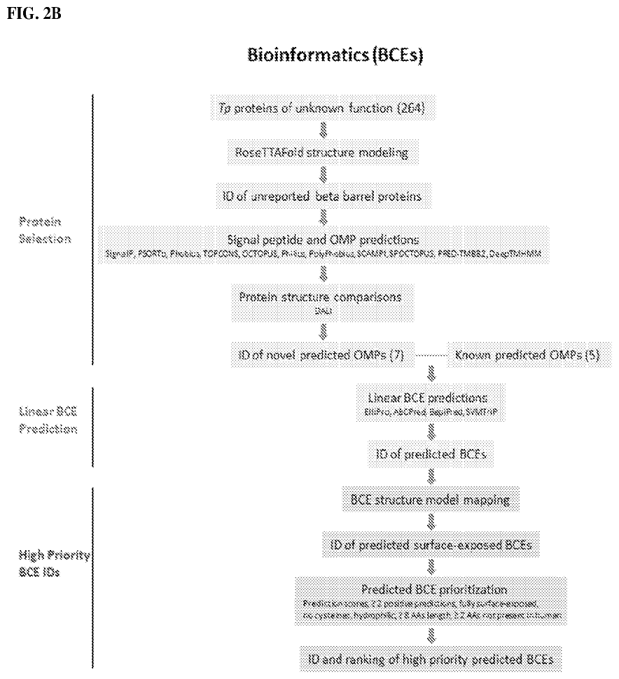

Each of the targeted loops of Tp0751 (99-237) were excised and replaced with antigenic loops from Neisseria meningitidis NmfHbp and NHBA (FIGS. 2A-2B; SEQ ID NOS: 14-16). Three loops from these two proteins, 2 loops from NmfHbp and 1 loop from NHBA, we placed into loops 2, 3, 6 and 8 of Tp0751. 11 constructs were designed (Tables 3 and 4; SEQ ID NOS: 39-48). In silico free energy calculations were performed for each theoretical construct as a way of rapidly prioritizing construct designs for advancement to expression trials. Synthetic genes encoding the engineered chimeras were codon optimized for E. coli and were evaluated in small scale expression trials.

### Example 2

### Protein Solubility and Nickel Affinity Enrichment

Expression trials were conducted to assess the engineering potential of each loop of Tp0751 (99-237). The native coding and amino acid sequence of full-length Tp0751 are shown in Table 1 (SEQ ID NOS: 1 and 2), with aa 99-237 underlined (SEQ ID NO: 3; FIG. 1C). The sequence of the 8 loops is shown in FIG. 1C (L1 through L8).

TABLE 1
Wild-type Tp0751 sequences

| | Genbank | | |
|---|---|---|---|
| | Accession | | |
| Name | ID | Nucleotide Sequence | Seq ID |
| Tp0751 | AAC65720.1 | GTGAATAGACCTTTACTGAGTGTGGCCGGATCGTT | 1 |
| (native | | GTTCGTCGCTGCGTGGGCTCTATATATTTTCTCCTG | |
| sequence) | | CTTTCAGCACGGTCACGTTCCTCCACGCAGAATTC | |
| | | CCCCGCATGATACCTTCGGCGCTCTACCCACTGCT | |
| | | GCACTTCCCAGCAACGCGCGGGACACCGCCGCAC | |
| | | ACCCGTCTGACACGGCAGACAATACGTCCGGCTCG | |
| | | TCCACCACGACAGACCCACGGTCACATGGAAACG | |
| | | CCCCGCCGGCCCCTGTAGGCGGAGCAGCACAGAC | |
| | | ACACACACAACCGCCTGTACAAACAGCAATGCGC | |
| | | ATAGCCCTTTGGAACCGTGCAACACATGGTGAACA | |
| | | GGGAGCACTCCAGCACCTCTTGGCAGGACTGTGG | |
| | | ATACAAACTGAAATCTCCCCGAACTCAGGCGATAT | |
| | | CCATCCTCTGCTGTTTTTTGACCGAGAACACGCGG | |
| | | AGATCACATTCTCACGCGCATCAGTCCAAGAAATC | |
| | | TTCCTGGTAGATAGCGCGCACACACACCGCAAGA | |
| | | CGGTGTCATTTCTCACGCGCAACACCGCAATTTCC | |
| | | AGCATCCGCCGCCGCCTTGAGGTAACATTTGAATC | |
| | | CCACGAGGTGATACACGTAAGGGCGGTTGAAGAC | |
| | | GTAGCACGGCTCAAAATTGGCAGCACGTCGATGT | |
| | | GGGACGGTCAATACACCAGATATCACGCCGGTCC | |
| | | GGCTAGTGCTCCTTCGCCCTGA | |
| Name | Uniprot ID | Peptide Sequence | |
| Tp0751 | O83732 | MNRPLLSVAGSLFVAAWALYIFSCFQHGHVPPRRIPP | 2 |
| 1-237 | | HDTFGALPTAALPSNARDTAAHPSDTADNTSGSSTT | (SEQ ID |
| | | TDPRSHGNAPPAPVGGAAQTHTQPPVQTAMRIALW | NO: 3 |
| | | NRATHGEQGALQHLLAGLWIQTEISPNSGDIHPLLFF | underlined) |
| | | DREHAEITFSRASVQEIFLVDSAHTHRKTVSFLTRNT | |
| | | AISSIRRRLEVTFESHEVIHVRAVEDVARLKIGSTSM | |
| | | WDGQYTRYHAGPASAPSP | |

The chimeric Tp0751 (99-237) constructs shown in Tables 2-4 were expressed in E. coli BL21 DE3 cells and grown in 5 mL 2×YT media overnight at 37° C. Briefly, synthesized genes were subcloned into an engineered vector which included a TEV protease cleavable N-terminal hexa-histidine tag.

TABLE 2
Tp0751 lipocalin containing domain sequences

| | Genbank | | |
|---|---|---|---|
| Domain | Accession ID | Nucleotide Sequence | Seq ID |
| Tp0751 | AAC65720.1 | GTACAAACAGCAATGCGCATAGCCCTTTGGAAC | 10 |
| (lipocalin | | CGTGCAACACATGGTGAACAGGGAGCACTCCAG | |
| containing | | CACCTCTTGGCAGGACTGTGGATACAAACTGAA | |
| domain, | | ATCTCCCCGAACTCAGGCGATATCCATCCTCTGC | |
| native | | TGTTTTTTGACCGAGAACACGCGGAGATCACAT | |
| sequence) | | TCTCACGCGCATCAGTCCAAGAAATCTTCCTGGT | |
| | | AGATAGCGCGCACACACACCGCAAGACGGTGTC | |
| | | ATTTCTCACGCGCAACACCGCAATTTCCAGCATC | |
| | | CGCCGCCGCCTTGAGGTAACATTTGAATCCCAC | |
| | | GAGGTGATACACGTAAGGGCGGTTGAAGACGTA | |
| | | GCACGGCTCAAAATTGGCAGCACGTCGATGTGG | |
| | | GACGGTCAATACACCAGATATCACGCCGGTCCG | |
| | | GCTAGTGCTCCTTCGCCCTGA | |
| Tp0751 | | GTGCAGACCGCGATGCGTATCGCGCTGTGGAAC | 11 |
| (lipocalin | | CGTGCGACCCACGGTGAACAAGGTGCGCTGCAA | |
| containing | | CACCTGCTGGCGGGTCTGTGGATTCAGACCGAA | |
| domain) | | ATTAGCCCGAACAGCGGCGACATTCACCCGCTG | |
| expression | | CTGTTCTTTGATCGTGAGCACGCGGAAATCACCT | |
| construct | | TCAGCCGTGCGAGCGTGCAGGAGATTTTTCTGG | |
| codon | | TTGACAGCGCGCACACCCACCGTAAGACCGTGA | |
| optimized | | GCTTCCTGACCCGTAACACCGCGATCAGCAGCA | |
| for E. coli | | TCCGTCGTCGTCTGGAAGTGACCTTTGAGAGCC | |
| | | ACGAAGTGATCCACGTTCGTGCGGTGGAAGACG | |
| | | TTGCGCGTCTGAAAATTGGTAGCACCAGCATGT | |
| | | GGGATGGCCAATACACCCGTTATCACGCGGGTC | |
| | | CGGCGAGCGCGCCGAGCCCG | |
| Domain | Uniprot ID | Peptide Sequence | |
| Tp0751 | O83732 | VQTAMRIALWNRATHGEQGALQHLLAGLWIQTEI | 3 |
| (lipocalin | | SPNSGDIHPLLFFDREHAEITFSRASVQEIFLVDSAH | |
| containing | | THRKTVSFLTRNTAISSIRRRLEVTFESHEVIHVRA | |
| domain) 99- | | VEDVARLKIGSTSMWDGQYTRYHAGPASAPSP | |
| 237 | | | |
| Tp0751 | | MGSTSGSGHHHHHHSSGRENLYFQGSMAVQTAM | 12 |
| (lipocalin | | RIALWNRATHGEQGALQHLLAGLWIQTEISPNSGD | |
| containing | | IHPLLFFDREHAEITFSRASVQEIFL VDSAHTHRKT | |
| domain) 99- | | VSFLTRNTAISSIRRRLEVTFESHEVIHVRAVEDVA | |
| 237 | | RLKIGSTSMWDGQYTRYHAGPASAPSP | |
| expression | | Note: This includes the native Tp0751(99-237) | |
| construct for | | sequence in a modified pET vector, with an | |
| E. coli | | expression tag (underlined) on the N terminus. In | |
| | | addition, the wild-type sequence further includes | |
| | | AAA (NotI restriction site) after PSP. | |
| Tp0751 | | GSMAVQTAMRIALWNRATHGEQGALQHLLAGL | 13 |
| (lipocalin | | WIQTEISPNSGDIHPLLFFDREHAEITFSRASVQEIFL | |
| containing | | VDSAHTHRKTVSFLTRNTAISSIRRRLEVTFESHEV | |
| domain) 99- | | IHVRAVEDVARLKIGSTSMWDGQYTRYHAGPASA | |
| 237 | | PSP | |
| expression | | Note: Post-TEV cleavage: Each protein sequence is | |
| construct for | | expressed in a vector encoding a TEV-cleavable hexa- | |
| E. coli, | | histidine tag. The TEV protease recognizes this | |
| post-TEV | | specific peptide sequence encoded in each protein, | |
| cleavage | | to allow for the N-terminal hexa-histidine tag to | |
| | | be cleaved during purification. This is the | |
| | | sequence of the protein once the expression tag has | |
| | | been proteolytically removed by TEV. The N-terminal | |
| | | “G” is a residual from the TEV site and the “SMA” | |
| | | residues are cloning artifacts. The native Tpo0751 | |
| | | lipocalin sequence starts with VQT and ends with | |
| | | PSP | |

TABLE 3
Tp0751 (99-237) mutant chimera IDs and sequence descriptions.

| Construct | |
|---|---|
| ID | Sequence Description |
| 0.1 | Tp0751 ΔLoop 2 ISPNS→GDDLHMG (SEQ ID NO: 14) |
| 0.2 | Tp0751 ΔLoop 2 ISPNS→KTVNGIRH (SEQ ID NO: 15) |
| 0.3 | Tp0751 ΔLoop 2 ISPNS→VLYNQAEKGS (SEQ ID NO: 16) |
| 0.4 | Tp0751 ΔLoop 3 EH→GDDLHMG (SEQ ID NO: 14) |
| 0.5 | Tp0751 ΔLoop 3 EH→VLYNQAEKGS (SEQ ID NO: 16) |
| 0.6 | Tp0751 ΔLoop 6 IS→GDDLHMG (SEQ ID NO: 14) |
| 0.7 | Tp0751 ΔLoop 6 IS→KTVNGIRH (SEQ ID NO: 15) |
| 0.8 | Tp0751 ΔLoop 6 IS→VLYNQAEKGS (SEQ ID NO: 16) |
| 0.9 | Tp0751 ΔLoop 8 LKIGSTS→GDDLHMG (SEQ ID NO: 14) |
| 0.10 | Tp0751 ΔLoop 8 LKIGSTS→KTVNGIRH (SEQ ID NO: 15) |
| 0.11 | Tp0751 ΔLoop 8 LKIGSTS→VLYNQAEKGS (SEQ ID NO: 16) |
| 1.1 | Tp0751 Expanded ΔLoop 2 EISPNSGDI→TprC Loop 3 RAYSEKDTRYA |
| | (SEQ ID NO: 17) |
| 1.2 | Tp0751 Expanded ΔLoop 2 EISPNSGDI→TprC Loop 6 |
| | GKLVQNLPNIMMPPGIT (SEQ ID NO: 18) |
| 1.3 | Tp0751 ΔLoop 2 ISPNS→TprK Loop 2 AKTIEATLH (SEQ ID NO: 19) |
| 1.4 | Tp0751 Expanded ΔLoop 2 EISPNSGDI→TprK Loop 2 AKTIEATLH |
| | (SEQ ID NO: 19) |
| 1.5 | Tp0751 ΔLoop 2 ISPNS→TprK New Loop 2 ESNGGAK (SEQ ID NO: 20) |
| 1.6 | Tp0751 New Expanded ΔLoop 2 ISPNSGD→TprK Loop 3 Truncation 1 |
| | DYKSKGDKPVYE (SEQ ID NO: 21) |
| 1.7 | Tp0751 New Expanded ΔLoop 2 ISPNSGD→TprK Loop 10 |
| | LQENSNVVIEKNV (SEQ ID NO: 22) |
| 1.8 | Tp0751 Expanded ΔLoop 3 REHA→BamA1 DNNQPFDLTV (SEQ ID NO: |
| | 23) |
| 1.9 | Tp0751 Expanded ΔLoop 3 REHA→TprK New Loop 2 ESNGGAK (SEQ |
| | ID NO: 20) |
| 1.10 | Tp0751 Expanded ΔLoop 3 REHA→TprK Loop 10 LQENSNVVIEKNV |
| | (SEQ ID NO: 22) |
| 1.11 | Tp0751 Expanded ΔLoop 6 TAISS→BamA1 DNNQPFDLTV (SEQ ID NO: |
| | 23) |
| 1.12 | Tp0751 Expanded ΔLoop 6 TAISS→TprC Loop 3 RAYSEKDTRYA (SEQ |
| | ID NO: 17) |
| 1.13 | Tp0751 Expanded ΔLoop 6 TAISS→TprK New Loop 2 ESNGGAK (SEQ |
| | ID NO: 20) |
| 1.14 | Tp0751 Expanded ΔLoop 6 TAISS→TprK Loop 10 LQENSNVVIEKNV |
| | (SEQ ID NO: 22) |
| 1.15 | Tp0751 New Expanded ΔLoop 8 RLKIGSTSM→BamA1 DNNQPFDLTV |
| | (SEQ ID NO: 23) |
| 1.16 | Tp0751 Expanded ΔLoop 8 LKIGSTSM→TprC Loop 1 |
| | FQKNPRTGPGKHTHGFRTTN (SEQ ID NO: 24) |
| 1.17 | Tp0751 Expanded ΔLoop 8 LKIGSTSM→TprC Loop 2 |
| | LAVELASSKSSTALSFTKPTAS (SEQ ID NO: 25) |
| 1.18 | Tp0751 New Expanded ΔLoop 8 RLKIGSTSM→TprC Loop 3 |
| | RAYSEKDTRYA (SEQ ID NO: 17) |
| 1.19 | Tp0751 New Expanded ΔLoop 8 RLKIGSTSM→TprC Loop 10 |
| | GTTNRFNIINAAGNLLN (SEQ ID NO: 26) |
| 1.20 | Tp0751 Expanded ΔLoop 8 LKIGSTSM→TprK Loop 3 |
| | NGDYKSKGDKPVYEPG (SEQ ID NO: 27) |
| 1.21 | Tp0751 Expanded ΔLoop 8 LKIGSTSM→TprK Loop 3 Truncation 1 |
| | DYKSKGDKPVYE (SEQ ID NO: 21) |
| 1.22 | Tp0751 New Expanded ΔLoop 8 RLKIGSTSM→TprK Loop 3 Truncation 1 |
| | DYKSKGDKPVYE (SEQ ID NO: 21) |
| 1.23 | Tp0751 Expanded ΔLoop 8 LKIGSTSM→TprK Loop 3 Truncation 2 |
| | DYKSKGDKP (SEQ ID NO: 28) |
| 1.24 | Tp0751 Expanded ΔLoop 8 LKIGSTSM→TprK Loop 3 Truncation 3 |
| | SKGDKPVYE (SEQ ID NO: 29) |
| 1.25 | Tp0751 Expanded ΔLoop 8 LKIGSTSM→TprK Loop 3 Truncation 4 |
| | KSKGDKPVY (SEQ ID NO: 30) |
| 2.1 | Tp0751 ΔLoop 2 ISPNS→TprC Loop 3 RAYSEKDTRYA (SEQ ID NO: 17) |
| | Tp0751 Expanded ΔLoop 8 LKIGSTSM→TprK Loop 3 Truncation 1 |
| | DYKSKGDKPVYE (SEQ ID NO: 21) |
| 2.2 | Tp0751 ΔLoop 2 ISPNS→TprC Loop 6 GKLVQNLPNIMMPPGIT (SEQ |
| | ID NO: 18) |
| | Tp0751 Expanded ΔLoop 8 LKIGSTSM→TprK Loop 3 Truncation 1 |
| | DYKSKGDKPVYE (SEQ ID NO: 21) |
| 2.3 | Tp0751 ΔLoop 2 ISPNS→TprC Loop 10 GTTNRFNIINAAGNLLN (SEQ |
| | ID NO: 26) |
| | Tp0751 Expanded ΔLoop 8 LKIGSTSM→TprK Loop 3 Truncation 1 |
| | DYKSKGDKPVYE (SEQ ID NO: 21) |
| 2.4 | Tp0751 ΔLoop 2 ISPNS→TprC Loop 10 (Nichols Strain) |
| | GTTNRFNIINPAGNLLN (SEQ ID NO: 31) |
| | Tp0751 Expanded ΔLoop 8 LKIGSTSM→TprK Loop 3 Truncation 1 |
| | DYKSKGDKPVYE (SEQ ID NO: 21) |
| 2.5 | Tp0751 ΔLoop 2 ISPNS→TprC Loop 11 QGVLADAPYMGIAESIW (SEQ |
| | ID NO: 32) |
| | Tp0751 Expanded ΔLoop 8 LKIGSTSM→TprK Loop 3 Truncation 1 |
| | DYKSKGDKPVYE (SEQ ID NO: 21) |
| 2.6 | Tp0751 ΔLoop 2 ISPNS→TprK Loop 1 KYRGEGNVYA (SEQ ID NO: 33) |
| | Tp0751 Expanded ΔLoop 8 LKIGSTSM→TprK Loop 3 Truncation 1 |
| | DYKSKGDKPVYE (SEQ ID NO: 21) |
| 2.7 | Tp0751 ΔLoop 2 ISPNS→TprK Loop 2 AKTIEATLH (SEQ ID NO: 19) |
| | Tp0751 Expanded ΔLoop 8 LKIGSTSM→TprK Loop 3 Truncation 1 |
| | DYKSKGDKPVYE (SEQ ID NO: 21) |
| 2.8 | Tp0751 ΔLoop 2 ISPNS→TprK Loop 10 Truncation 1 GKLQENSNVV |
| | (SEQ ID NO: 34) |
| | Tp0751 Expanded ΔLoop 8 LKIGSTSM→TprK Loop 3 Truncation 1 |
| | DYKSKGDKPVYE (SEQ ID NO: 21) |
| 3.1 | Tp0751 ΔLoop 2 ISPNS→TprK Loop 2 AKTIEATLH (SEQ ID NO: 19) |
| | Tp0751 Expanded ΔLoop 8 LKIGSTSM→TprK Loop 3 Truncation 1 |
| | DYKSKGDKPVYE (SEQ ID NO: 21) |
| | Tp0751 C-term + TprC Loop 6 GKLVQNLPNIMMPPGIT (SEQ ID NO: 18) |
| 3.2 | Tp0751 ΔLoop 2 ISPNS→TprK Loop 2 AKTIEATLH (SEQ ID NO: 19) |
| | Tp0751 Expanded ΔLoop 8 LKIGSTSM→TprK Loop 3 Truncation 1 |
| | DYKSKGDKPVYE (SEQ ID NO: 21) |
| | Tp0751 AC-term AGPASAPSP→TprC Loop 6 GKLVQNLPNIMMPPGIT |
| | (SEQ ID NO: 18) |
| 3.3 | Tp0751 ΔLoop 2 ISPNS→TprK Loop 2 AKTIEATLH (SEQ ID NO: 19) |
| | Tp0751 Expanded ΔLoop 8 LKIGSTSM→TprK Loop 3 Truncation 1 |
| | DYKSKGDKPVYE (SEQ ID NO: 21) |
| | Tp0751 AC-term AGPASAPSP→TprC Loop 6 Truncation 1 LVQNLPNIM |
| | (SEQ ID NO: 35) |
| 3.4 | Tp0751 ΔLoop 2 ISPNS→TprK Loop 2 AKTIEATLH (SEQ ID NO: 19) |
| | Tp0751 Expanded ΔLoop 8 LKIGSTSM→TprK Loop 3 Truncation 1 |
| | DYKSKGDKPVYE (SEQ ID NO: 21) |
| | Tp0751 AC-term AGPASAPSP→TprC Loop 6 Truncation 2 NLPNIMMPP |
| | (SEQ ID NO: 36) |
| 3.5 | Tp0751 ΔLoop 2 ISPNS→TprK Loop 2 AKTIEATLH (SEQ ID NO: 19) |
| | Tp0751 Expanded ΔLoop 8 LKIGSTSM→TprK Loop 3 Truncation 1 |
| | DYKSKGDKPVYE (SEQ ID NO: 21) |
| | Tp0751 AC-term AGPASAPSP→TprC Loop 6 Truncation 3 NIMMPPGIT |
| | (SEQ ID NO: 37) |
| 3.6 | Tp0751 ΔLoop 2 ISPNS→TprK New Loop 2 ESNGGAK (SEQ ID NO: 20) |
| | Tp0751 Expanded ΔLoop 8 LKIGSTSM→TprK Loop 3 Truncation 1 |
| | DYKSKGDKPVYE (SEQ ID NO: 21) |
| | Tp0751 AC-term AGPASAPSP→TprC Loop 6 GKLVQNLPNIMMPPGIT |
| | (SEQ ID NO: 18) |
| 3.7 | Tp0751 ΔLoop 2 ISPNS→TprK Loop 10 Truncation 1 GKLQENSNVV |
| | (SEQ ID NO: 34) |
| | Tp0751 Expanded ΔLoop 8 LKIGSTSM→TprK Loop 3 Truncation 1 |
| | DYKSKGDKPVYE (SEQ ID NO: 21) |
| | Tp0751 AC-term AGPASAPSP→TprC Loop 6 GKLVQNLPNIMMPPGIT |
| | (SEQ ID NO: 18) |
| 4.1 | Tp0751 New Expanded ΔLoop 2 ISPNSGD→TprK Loop 3 Truncation 1 |
| | DYKSKGDKPVYE (SEQ ID NO: 21) |
| | Tp0751 Expanded ΔLoop 3 REHA→TprK New Loop 2 ESNGGAK (SEQ |
| | ID NO: 20) |
| | Tp0751 New Expanded ΔLoop 8 RLKIGSTSM→TprC Loop 3 |
| | RAYSEKDTRYA (SEQ ID NO: 17) |
| | Tp0751 AC-term AGPASAPSP→TprC Loop 6 GKLVQNLPNIMMPPGIT |
| | (SEQ ID NO: 18) |
| 4.2 | Tp0751 New Expanded ΔLoop 2 ISPNSGD→TprK Loop 3 Truncation 1 |
| | DYKSKGDKPVYE (SEQ ID NO: 21) |
| | Tp0751 Expanded ΔLoop 3 REHA→TprK Loop 10 LQENSNVVIEKNV |
| | (SEQ ID NO: 22) |
| | Tp0751 New Expanded ΔLoop 8 RLKIGSTSM→TprC Loop 3 |
| | RAYSEKDTRYA (SEQ ID NO: 17) |
| | Tp0751 AC-term AGPASAPSP→TprC Loop 6 GKLVQNLPNIMMPPGIT |
| | (SEQ ID NO: 18) |
| 4.3 | Tp0751 New Expanded ΔLoop 2 ISPNSGD→TprK Loop 10 |
| | LQENSNVVIEKNV (SEQ ID NO: 22) |
| | Tp0751 Expanded ΔLoop 3 REHA→TprK New Loop 2 ESNGGAK (SEQ |
| | ID NO: 20) |
| | Tp0751 New Expanded ΔLoop 8 RLKIGSTSM→TprK Loop 3 Truncation 1 |
| | DYKSKGDKPVYE (SEQ ID NO: 21) |
| | Tp0751 AC-term AGPASAPSP→TprC Loop 6 GKLVQNLPNIMMPPGIT |
| | (SEQ ID NO: 18) |
| 4.4 | Tp0751 ΔLoop 2 ISPNSGD→TprK Loop 3 Truncation 1 |
| (4B1) | DYKSKGDKPVYE (SEQ ID NO: 21) |
| | Tp0751 ΔLoop 3 REHA→Tp0483C VKPSFTGV (SEQ ID NO: 118) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 4.5 | Tp0751 ΔLoop 2 ISPNSGD→TprK Loop 3 Truncation 1 |
| (4B2) | DYKSKGDKPVYE (SEQ ID NO: 21) |
| | Tp0751 ΔLoop 3 REHA→Tp0856A ESGFNFS (SEQ ID NO: 143) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 4.6 | Tp0751 ΔLoop 2 ISPNSGD→TprK Loop 3 Truncation 1 |
| (4B3) | DYKSKGDKPVYE (SEQ ID NO: 21) |
| | Tp0751 ΔLoop 3 REHA→Tp0346A GNEIKDS (SEQ ID NO: 144) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.1 | Tp0751 AN-term VQTAMRIALWNRATHGE→Tp0483A |
| (5A1) | APVYRPQDHAATINGVSD (SEQ ID NO: 116) |
| | Tp0751 ΔLoop 2 ISPNSGD→TprK Loop 3 Truncation 1 |
| | DYKSKGDKPVYE (SEQ ID NO: 21) |
| | Tp0751 ΔLoop 3 REHA→TprK New Loop 2 ESNGGAK (SEQ ID NO: 20) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.2 | Tp0751 AN-term VQTAMRIALWNRATHGE→Tp0856B |
| (5A2) | VNVSNLQVDHLWKR (SEQ ID NO: 120) |
| | Tp0751 ΔLoop 2 ISPNSGD→TprK Loop 3 Truncation 1 |
| | DYKSKGDKPVYE (SEQ ID NO: 21) |
| | Tp0751 ΔLoop 3 REHA→TprK New Loop 2 ESNGGAK (SEQ ID NO: 20) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.3 | Tp0751 AN-term VQTAMRIALWNRATHGE→TpRS01700B |
| (5A3) | VDFPLNPNTRFLAGKLPAET (SEQ ID NO: 124) |
| | Tp0751 ΔLoop 2 ISPNSGD→TprK Loop 3 Truncation 1 |
| | DYKSKGDKPVYE (SEQ ID NO: 21) |
| | Tp0751 ΔLoop 3 REHA→TprK New Loop 2 ESNGGAK (SEQ ID NO: 20) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.4 | Tp0751 AN-term VQTAMRIALWNRATHGE→TpRS03465B |
| (5A4) | KNGAGSTDLKWEQFLQKY (SEQ ID NO: 128) |
| | Tp0751 ΔLoop 2 ISPNSGD→TprK Loop 3 Truncation 1 |
| | DYKSKGDKPVYE (SEQ ID NO: 21) |
| | Tp0751 ΔLoop 3 REHA→TprK New Loop 2 ESNGGAK (SEQ ID NO: 20) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.5 | Tp0751 AN-term VQTAMRIALWNRATHGE→Tp0346B |
| (5A5) | ANRWISGPHWWNSLSS (SEQ ID NO: 126) |
| | Tp0751 ΔLoop 2 ISPNSGD→TprK Loop 3 Truncation 1 |
| | DYKSKGDKPVYE (SEQ ID NO: 21) |
| | Tp0751 ΔLoop 3 REHA→TprK New Loop 2 ESNGGAK (SEQ ID NO: 20) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.6 | Tp0751 ΔLoop 1 EQGALQHL→Tp0856 SSEAAAKT |
| (5B1) | Tp0751 ΔLoop 2 ISPNSGD→TprK Loop 3 Truncation 1 |
| | DYKSKGDKPVYE (SEQ ID NO: 21) |
| | Tp0751 ΔLoop 3 REHA→TprK New Loop 2 ESNGGAK (SEQ ID NO: 20) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.7 | Tp0751 ΔLoop 1 EQGALQHL→Tp0483C VKPSFTGV (SEQ ID NO: 118) |
| (5B2) | Tp0751 ΔLoop 2 ISPNSGD→TprK Loop 3 Truncation 1 |
| | DYKSKGDKPVYE (SEQ ID NO: 21) |
| | Tp0751 ΔLoop 3 REHA→TprK New Loop 2 ESNGGAK (SEQ ID NO: 20) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.8 | Tp0751 ΔLoop 1 EQGALQHL→Tp0856A PESGENFSP (SEQ ID NO: |
| (5B3) | 119) |
| | Tp0751 ΔLoop 2 ISPNSGD→TprK Loop 3 Truncation 1 |
| | DYKSKGDKPVYE (SEQ ID NO: 21) |
| | Tp0751 ΔLoop 3 REHA→TprK New Loop 2 ESNGGAK (SEQ ID NO: 20) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.9 | Tp0751 ΔLoop 2 ISPNSGD→TprK Loop 3 Truncation 1 |
| (5C1) | DYKSKGDKPVYE (SEQ ID NO: 21) |
| | Tp0751 ΔLoop 3 REHA→TprK New Loop 2 ESNGGAK (SEQ ID NO: 20) |
| | Tp0751 ΔLoop 4 AS→Tp0346A GNEIKDS (SEQ ID NO: 144) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.10 | Tp0751 ΔLoop 2 ISPNSGD→TprK Loop 3 Truncation 1 |
| (5D1) | DYKSKGDKPVYE (SEQ ID NO: 21) |
| | Tp0751 ΔLoop 3 REHA→TprK New Loop 2 ESNGGAK (SEQ ID NO: 20) |
| | Tp0751 ΔLoop 5 RK→Tp0346A GNEIKDS (SEQ ID NO: 144) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.11 | Tp0751 ΔLoop 2 ISPNSGD→TprK Loop 3 Truncation 1 |
| (5E1) | DYKSKGDKPVYE (SEQ ID NO: 21) |
| | Tp0751 ΔLoop 3 REHA→TprK New Loop 2 ESNGGAK (SEQ ID NO: 20) |
| | Tp0751 ΔLoop 6 TAISS→Tp0856C STGDEQQGT (SEQ ID NO: 145) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.12 | Tp0751 ΔLoop 2 ISPNSGD→TprK Loop 3 Truncation 1 |
| (5E2) | DYKSKGDKPVYE (SEQ ID NO: 21) |
| | Tp0751 ΔLoop 3 REHA→TprK New Loop 2 ESNGGAK (SEQ ID NO: 20) |
| | Tp0751 ΔLoop 6 TAISS→TpRS01700A AVGKDIEGQ (SEQ ID NO: 123) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.13 | Tp0751 ΔLoop 2 ISPNSGD→TprK Loop 3 Truncation 1 |
| (5E3) | DYKSKGDKPVYE (SEQ ID NO: 21) |
| | Tp0751 ΔLoop 3 REHA→TprK New Loop 2 ESNGGAK (SEQ ID NO: 20) |
| | Tp0751 ΔLoop 6 TAISS→Tp0483B FGQRKED (SEQ ID NO: 146) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.14 | Tp0751 ΔLoop 2 ISPNSGD→TprK Loop 3 Truncation 1 |
| (5E4) | DYKSKGDKPVYE (SEQ ID NO: 21) |
| | Tp0751 ΔLoop 3 REHA→TprK New Loop 2 ESNGGAK (SEQ ID NO: 20) |
| | Tp0751 ΔLoop 6 TAISS→Tp0483C VKPSFTGV (SEQ ID NO: 118) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |
| 5.15 | Tp0751 ΔLoop 2 ISPNSGD→TprK Loop 3 Truncation 1 |
| (5E5) | DYKSKGDKPVYE (SEQ ID NO: 21) |
| | Tp0751 ΔLoop 3 REHA→TprK New Loop 2 ESNGGAK (SEQ ID NO: 20) |
| | Tp0751 ΔLoop 6 TAISS→Tp0346A GNEIKDS (SEQ ID NO: 144) |
| | Tp0751 Expanded ΔLoop 8 RLKIGSTSM→TprC Loop 3 RAYSEKDTRYA |
| | (SEQ ID NO: 17) |
| | Tp0751 AC-term AGPASAPSP→TprC Loop 6 GKLVQNLPNIMMPPGIT |
| | (SEQ ID NO: 18) |

Construct IDs with the 0.X (X = any #) designation refers to chimeras with one loop substitution from the structurally homologous proteins NHBA (SEQ ID NO: 14) or NmfHbp (SEQ ID NO: 15 and 16). The 0.X designated constructs represent the original engineering of chimeras where substituted sequences were selected from lipocalin-domain containing proteins. Constructs designated as 1.X, 2.X, 3.X and 4.X refer to chimeras with one, two, three or four loop engraftments from either Treponema pallidum TprC and/or Treponema pallidum TprK, respectively. These latter constructs were selected for maximum immunogenicity potential rather than structural homology. Sequences of replaced Tp0751 (99-237) loops and subsequently engrafted epitopes shown in bold.
The nomenclature under the “Sequence Description” column is as follows: Tp0751 sequence removed → engrafted sequence. See Table 4 for full protein sequence of each chimera.

TABLE 4
Tp0751 (99-237) mutant chimera designations, construct sequences, and sequence
IDs. See Table 3 for construct description legend.

| Construct | | |
|---|---|---|
| ID | Peptide Sequence | Seq ID |
| 0.1 | VQTAMRIALWNRATHGEQGALQHLLAGLWIQTEGDDLHMGGDIHPLLFFD | 91 |
| | REHAEITFSRASVQEIFLVDSAHTHRKTVSFLTRNTAISSIRRRLEVTFESHEVI | |
| | HVRAVEDVARLKIGSTSMWDGQYTRYHAGPASAPSP | |
| 0.2 | VQTAMRIALWNRATHGEQGALQHLLAGLWIQTEKTVNGIRHGDIHPLLFFD | 38 |
| | REHAEITFSRASVQEIFLVDSAHTHRKTVSFLTRNTAISSIRRRLEVTFESHEVI | |
| | HVRAVEDVARLKIGSTSMWDGQYTRYHAGPASAPSP | |
| 0.3 | VQTAMRIALWNRATHGEQGALQHLLAGLWIQTEVLYNQAEKGSGDIHPLLF | 39 |
| | FDREHAEITFSRASVQEIFLVDSAHTHRKTVSFLTRNTAISSIRRRLEVTFESHE | |
| | VIHVRAVEDVARLKIGSTSMWDGQYTRYHAGPASAPSP | |
| 0.4 | VQTAMRIALWNRATHGEQGALQHLLAGLWIQTEISPNSGDIHPLLFFDRGDD | 40 |
| | LHMGAEITFSRASVQEIFLVDSAHTHRKTVSFLTRNTAISSIRRRLEVTFESHE | |
| | VIHVRAVEDVARLKIGSTSMWDGQYTRYHAGPASAPSP | |
| 0.5 | VQTAMRIALWNRATHGEQGALQHLLAGLWIQTEISPNSGDIHPLLFFDRVLY | 41 |
| | NQAEKGSAEITFSRASVQEIFLVDSAHTHRKTVSFLTRNTAISSIRRRLEVTFES | |
| | HEVIHVRAVEDVARLKIGSTSMWDGQYTRYHAGPASAPSP | |
| 0.6 | VQTAMRIALWNRATHGEQGALQHLLAGLWIQTEISPNSGDIHPLLFFDREHA | 42 |
| | EITFSRASVQEIFLVDSAHTHRKTVSFLTRNTAGDDLHMGSIRRRLEVTFESH | |
| | EVIHVRAVEDVARLKIGSTSMWDGQYTRYHAGPASAPSP | |
| 0.7 | VQTAMRIALWNRATHGEQGALQHLLAGLWIQTEISPNSGDIHPLLFFDREHA | 43 |
| | EITFSRASVQEIFLVDSAHTHRKTVSFLTRNTAKTVNGIRHSIRRRLEVTFESH | |
| | EVIHVRAVEDVARLKIGSTSMWDGQYTRYHAGPASAPSP | |
| 0.8 | VQTAMRIALWNRATHGEQGALQHLLAGLWIQTEISPNSGDIHPLLFFDREHA | 44 |
| | EITFSRASVQEIFLVDSAHTHRKTVSFLTRNTAVLYNQAEKGSSIRRRLEVTFE | |
| | SHEVIHVRAVEDVARLKIGSTSMWDGQYTRYHAGPASAPSP | |
| 0.9 | VQTAMRIALWNRATHGEQGALQHLLAGLWIQTEISPNSGDIHPLLFFDREHA | 45 |
| | EITFSRASVQEIFLVDSAHTHRKTVSFLTRNTAISSIRRRLEVTFESHEVIHVRA | |
| | VEDVARGDDLHMGMWDGQYTRYHAGPASAPSP | |
| 0.10 | VQTAMRIALWNRATHGEQGALQHLLAGLWIQTEISPNSGDIHPLLFFDREHA | 46 |
| | EITFSRASVQEIFLVDSAHTHRKTVSFLTRNTAISSIRRRLEVTFESHEVIHVRA | |
| | VEDVARKTVNGIRHMWDGQYTRYHAGPASAPSP | |
| 0.11 | VQTAMRIALWNRATHGEQGALQHLLAGLWIQTEISPNSGDIHPLLFFDREHA | 47 |
| | EITFSRASVQEIFLVDSAHTHRKTVSFLTRNTAISSIRRRLEVTFESHEVIHVRA | |
| | VEDVARVLYNQAEKGSMWDGQYTRYHAGPASAPSP | |
| 1.1 | VQTAMRIALWNRATHGEQGALQHLLAGLWIQTRAYSEKDTRYAHPLLFFDR | 48 |
| | EHAEITFSRASVQEIFLVDSAHTHRKTVSFLTRNTAISSIRRRLEVTFESHEVIH | |
| | VRAVEDVARLKIGSTSMWDGQYTRYHAGPASAPSP | |
| 1.2 | VQTAMRIALWNRATHGEQGALQHLLAGLWIQTGKLVQNLPNIMMPPGITH | 49 |
| | PLLFFDREHAEITFSRASVQEIFLVDSAHTHRKTVSFLTRNTAISSIRRRLEVTFE | |
| | SHEVIHVRAVEDVARLKIGSTSMWDGQYTRYHAGPASAPSP | |
| 1.3 | VQTAMRIALWNRATHGEQGALQHLLAGLWIQTEAKTIEATLHGDIHPLLFF | 50 |
| | DREHAEITFSRASVQEIFLVDSAHTHRKTVSFLTRNTAISSIRRRLEVTFESHEV | |
| | IHVRAVEDVARLKIGSTSMWDGQYTRYHAGPASAPSP | |
| 1.4 | VQTAMRIALWNRATHGEQGALQHLLAGLWIQTAKTIEATLHHPLLFFDREH | 51 |
| | AEITFSRASVQEIFLVDSAHTHRKTVSFLTRNTAISSIRRRLEVTFESHEVIHVR | |
| | AVEDVARLKIGSTSMWDGQYTRYHAGPASAPSP | |
| 1.5 | VQTAMRIALWNRATHGEQGALQHLLAGLWIQTEESNGGAKGDIHPLLFFDR | 52 |
| | EHAEITFSRASVQEIFLVDSAHTHRKTVSFLTRNTAISSIRRRLEVTFESHEVIH | |
| | VRAVEDVARLKIGSTSMWDGQYTRYHAGPASAPSP | |
| 1.6 | VQTAMRIALWNRATHGEQGALQHLLAGLWIQTEDYKSKGDKPVYEIHPLLF | 53 |
| | FDREHAEITFSRASVQEIFLVDSAHTHRKTVSFLTRNTAISSIRRRLEVTFESHE | |
| | VIHVRAVEDVARLKIGSTSMWDGQYTRYHAGPASAPSP | |
| 1.7 | VQTAMRIALWNRATHGEQGALQHLLAGLWIQTELQENSNVVIEKNVIHPLL | 54 |
| | FFDREHAEITFSRASVQEIFLVDSAHTHRKTVSFLTRNTAISSIRRRLEVTFESH | |
| | EVIHVRAVEDVARLKIGSTSMWDGQYTRYHAGPASAPSP | |
| 1.8 | VQTAMRIALWNRATHGEQGALQHLLAGLWIQTEISPNSGDIHPLLFFDDNNQ | 55 |
| | PFDLTVEITFSRASVQEIFLVDSAHTHRKTVSFLTRNTAISSIRRRLEVTFESHE | |
| | VIHVRAVEDVARLKIGSTSMWDGQYTRYHAGPASAPSP | |
| 1.9 | VQTAMRIALWNRATHGEQGALQHLLAGLWIQTEISPNSGDIHPLLFFDESNG | 56 |
| | GAKEITFSRASVQEIFLVDSAHTHRKTVSFLTRNTAISSIRRRLEVTFESHEVIH | |
| | VRAVEDVARLKIGSTSMWDGQYTRYHAGPASAPSP | |
| 1.10 | VQTAMRIALWNRATHGEQGALQHLLAGLWIQTEISPNSGDIHPLLFFDLQEN | 57 |
| | SNVVIEKNVEITFSRASVQEIFLVDSAHTHRKTVSFLTRNTAISSIRRRLEVTFE | |
| | SHEVIHVRAVEDVARLKIGSTSMWDGQYTRYHAGPASAPSP | |
| 1.11 | VQTAMRIALWNRATHGEQGALQHLLAGLWIQTEISPNSGDIHPLLFFDREHA | 58 |
| | EITFSRASVQEIFLVDSAHTHRKTVSFLTRNDNNQPFDLTVIRRRLEVTFESHE | |
| | VIHVRAVEDVARLKIGSTSMWDGQYTRYHAGPASAPSP | |
| 1.12 | VQTAMRIALWNRATHGEQGALQHLLAGLWIQTEISPNSGDIHPLLFFDREHA | 59 |
| | EITFSRASVQEIFLVDSAHTHRKTVSFLTRNRAYSEKDTRYAIRRRLEVTFESH | |
| | EVIHVRAVEDVARLKIGSTSMWDGQYTRYHAGPASAPSP | |
| 1.13 | VQTAMRIALWNRATHGEQGALQHLLAGLWIQTEISPNSGDIHPLLFFDREHA | 60 |
| | EITFSRASVQEIFLVDSAHTHRKTVSFLTRNESNGGAKIRRRLEVTFESHEVIH | |
| | VRAVEDVARLKIGSTSMWDGQYTRYHAGPASAPSP | |
| 1.14 | VQTAMRIALWNRATHGEQGALQHLLAGLWIQTEISPNSGDIHPLLFFDREHA | 61 |
| | EITFSRASVQEIFLVDSAHTHRKTVSFLTRNLQENSNVVIEKNVIRRRLEVTFE | |
| | SHEVIHVRAVEDVARLKIGSTSMWDGQYTRYHAGPASAPSP | |
| 1.15 | VQTAMRIALWNRATHGEQGALQHLLAGLWIQTEISPNSGDIHPLLFFDREHA | 62 |
| | EITFSRASVQEIFLVDSAHTHRKTVSFLTRNTAISSIRRRLEVTFESHEVIHVRA | |
| | VEDVADNNQPFDLTVWDGQYTRYHAGPASAPSP | |
| 1.16 | VQTAMRIALWNRATHGEQGALQHLLAGLWIQTEISPNSGDIHPLLFFDREHA | 63 |
| | EITFSRASVQEIFLVDSAHTHRKTVSFLTRNTAISSIRRRLEVTFESHEVIHVRA | |
| | VEDVARFQKNPRTGPGKHTHGFRTTNWDGQYTRYHAGPASAPSP | |
| 1.17 | VQTAMRIALWNRATHGEQGALQHLLAGLWIQTEISPNSGDIHPLLFFDREHA | 64 |
| | EITFSRASVQEIFLVDSAHTHRKTVSFLTRNTAISSIRRRLEVTFESHEVIHVRA | |
| | VEDVARLAVELASSKSSTALSFTKPTASWDGQYTRYHAGPASAPSP | |
| 1.18 | VQTAMRIALWNRATHGEQGALQHLLAGLWIQTEISPNSGDIHPLLFFDREHA | 65 |
| | EITFSRASVQEIFLVDSAHTHRKTVSFLTRNTAISSIRRRLEVTFESHEVIHVRA | |
| | VEDVARAYSEKDTRYAWDGQYTRYHAGPASAPSP | |
| 1.19 | VQTAMRIALWNRATHGEQGALQHLLAGLWIQTEISPNSGDIHPLLFFDREHA | 66 |
| | EITFSRASVQEIFLVDSAHTHRKTVSFLTRNTAISSIRRRLEVTFESHEVIHVRA | |
| | VEDVAGTTNRFNIINAAGNLLNWDGQYTRYHAGPASAPSP | |
| 1.20 | VQTAMRIALWNRATHGEQGALQHLLAGLWIQTEISPNSGDIHPLLFFDREHA | 67 |
| | EITFSRASVQEIFLVDSAHTHRKTVSFLTRNTAISSIRRRLEVTFESHEVIHVRA | |
| | VEDVARNGDYKSKGDKPVYEPGWDGQYTRYHAGPASAPSP | |
| 1.21 | VQTAMRIALWNRATHGEQGALQHLLAGLWIQTEISPNSGDIHPLLFFDREHA | 68 |
| | EITFSRASVQEIFLVDSAHTHRKTVSFLTRNTAISSIRRRLEVTFESHEVIHVRA | |
| | VEDVARDYKSKGDKPVYEWDGQYTRYHAGPASAPSP | |
| 1.22 | VQTAMRIALWNRATHGEQGALQHLLAGLWIQTEISPNSGDIHPLLFFDREHA | 69 |
| | EITFSRASVQEIFLVDSAHTHRKTVSFLTRNTAISSIRRRLEVTFESHEVIHVRA | |
| | VEDVADYKSKGDKPVYEWDGQYTRYHAGPASAPSP | |
| 1.23 | VQTAMRIALWNRATHGEQGALQHLLAGLWIQTEISPNSGDIHPLLFFDREHA | 70 |
| | EITFSRASVQEIFLVDSAHTHRKTVSFLTRNTAISSIRRRLEVTFESHEVIHVRA | |
| | VEDVARDYKSKGDKPWDGQYTRYHAGPASAPSP | |
| 1.24 | VQTAMRIALWNRATHGEQGALQHLLAGLWIQTEISPNSGDIHPLLFFDREHA | 71 |
| | EITFSRASVQEIFLVDSAHTHRKTVSFLTRNTAISSIRRRLEVTFESHEVIHVRA | |
| | VEDVARSKGDKPVYEWDGQYTRYHAGPASAPSP | |
| 1.25 | VQTAMRIALWNRATHGEQGALQHLLAGLWIQTEISPNSGDIHPLLFFDREHA | 72 |
| | EITFSRASVQEIFLVDSAHTHRKTVSFLTRNTAISSIRRRLEVTFESHEVIHVRA | |
| | VEDVARKSKGDKPVYWDGQYTRYHAGPASAPSP | |
| 2.1 | VQTAMRIALWNRATHGEQGALQHLLAGLWIQTERAYSEKDTRYAGDIHPLL | 73 |
| | FFDREHAEITFSRASVQEIFLVDSAHTHRKTVSFLTRNTAISSIRRRLEVTFESH | |
| | EVIHVRAVEDVARDYKSKGDKPVYEWDGQYTRYHAGPASAPSP | |
| 2.2 | VQTAMRIALWNRATHGEQGALQHLLAGLWIQTEGKLVQNLPNIMMPPGIT | 74 |
| | GDIHPLLFFDREHAEITFSRASVQEIFLVDSAHTHRKTVSFLTRNTAISSIRRRLE | |
| | VTFESHEVIHVRAVEDVARDYKSKGDKPVYEWDGQYTRYHAGPASAPSP | |
| 2.3 | VQTAMRIALWNRATHGEQGALQHLLAGLWIQTEGTTNRFNIINAAGNLLNG | 75 |
| | DIHPLLFFDREHAEITFSRASVQEIFLVDSAHTHRKTVSFLTRNTAISSIRRRLEV | |
| | TFESHEVIHVRAVEDVARDYKSKGDKPVYEWDGQYTRYHAGPASAPSP | |
| 2.4 | VQTAMRIALWNRATHGEQGALQHLLAGLWIQTEGTTNRFNIINPAGNLLNG | 76 |
| | DIHPLLFFDREHAEITFSRASVQEIFLVDSAHTHRKTVSFLTRNTAISSIRRRLEV | |
| | TFESHEVIHVRAVEDVARDYKSKGDKPVYEWDGQYTRYHAGPASAPSP | |
| 2.5 | VQTAMRIALWNRATHGEQGALQHLLAGLWIQTEQGVLADAPYMGIAESIW | 77 |
| | GDIHPLLFFDREHAEITFSRASVQEIFLVDSAHTHRKTVSFLTRNTAISSIRRRLE | |
| | VTFESHEVIHVRAVEDVARDYKSKGDKPVYEWDGQYTRYHAGPASAPSP | |
| 2.6 | VQTAMRIALWNRATHGEQGALQHLLAGLWIQTEKYRGEGNVYAGDIHPLL | 78 |
| | FFDREHAEITFSRASVQEIFLVDSAHTHRKTVSFLTRNTAISSIRRRLEVTFESH | |
| | EVIHVRAVEDVARDYKSKGDKPVYEWDGQYTRYHAGPASAPSP | |
| 2.7 | VQTAMRIALWNRATHGEQGALQHLLAGLWIQTEAKTIEATLHGDIHPLLFF | 79 |
| | DREHAEITFSRASVQEIFLVDSAHTHRKTVSFLTRNTAISSIRRRLEVTFESHEV | |
| | IHVRAVEDVARDYKSKGDKPVYEWDGQYTRYHAGPASAPSP | |
| 2.8 | VQTAMRIALWNRATHGEQGALQHLLAGLWIQTEGKLQENSNVVGDIHPLLF | 80 |
| | FDREHAEITFSRASVQEIFLVDSAHTHRKTVSFLTRNTAISSIRRRLEVTFESHE | |
| | VIHVRAVEDVARDYKSKGDKPVYEWDGQYTRYHAGPASAPSP | |
| 3.1 | VQTAMRIALWNRATHGEQGALQHLLAGLWIQTEAKTIEATLHGDIHPLLFF | 81 |
| | DREHAEITFSRASVQEIFLVDSAHTHRKTVSFLTRNTAISSIRRRLEVTFESHEV | |
| | IHVRAVEDVARDYKSKGDKPVYEWDGQYTRYHAGPASAPSPGKLVQNLPN | |
| | IMMPPGIT | |
| 3.2 | VQTAMRIALWNRATHGEQGALQHLLAGLWIQTEAKTIEATLHGDIHPLLFF | 82 |
| | DREHAEITFSRASVQEIFLVDSAHTHRKTVSFLTRNTAISSIRRRLEVTFESHEV | |
| | IHVRAVEDVARDYKSKGDKPVYEWDGQYTRYHGKLVQNLPNIMMPPGIT | |
| 3.3 | VQTAMRIALWNRATHGEQGALQHLLAGLWIQTEAKTIEATLHGDIHPLLFF | 83 |
| | DREHAEITFSRASVQEIFLVDSAHTHRKTVSFLTRNTAISSIRRRLEVTFESHEV | |
| | IHVRAVEDVARDYKSKGDKPVYEWDGQYTRYHLVQNLPNIM | |
| 3.4 | VQTAMRIALWNRATHGEQGALQHLLAGLWIQTEAKTIEATLHGDIHPLLFF | 84 |
| | DREHAEITFSRASVQEIFLVDSAHTHRKTVSFLTRNTAISSIRRRLEVTFESHEV | |
| | IHVRAVEDVARDYKSKGDKPVYEWDGQYTRYHNLPNIMMPP | |
| 3.5 | VQTAMRIALWNRATHGEQGALQHLLAGLWIQTEAKTIEATLHGDIHPLLFF | 85 |
| | DREHAEITFSRASVQEIFLVDSAHTHRKTVSFLTRNTAISSIRRRLEVTFESHEV | |
| | IHVRAVEDVARDYKSKGDKPVYEWDGQYTRYHNIMMPPGIT | |
| 3.6 | VQTAMRIALWNRATHGEQGALQHLLAGLWIQTEESNGGAKGDIHPLLFFDR | 86 |
| | EHAEITFSRASVQEIFLVDSAHTHRKTVSFLTRNTAISSIRRRLEVTFESHEVIH | |
| | VRAVEDVARDYKSKGDKPVYEWDGQYTRYHGKLVQNLPNIMMPPGIT | |
| 3.7 | VQTAMRIALWNRATHGEQGALQHLLAGLWIQTEGKLQENSNVVGDIHPLLF | 87 |
| | FDREHAEITFSRASVQEIFLVDSAHTHRKTVSFLTRNTAISSIRRRLEVTFESHE | |
| | VIHVRAVEDVARDYKSKGDKPVYEWDGQYTRYHGKLVQNLPNIMMPPGI | |
| | T | |
| 4.1 | VQTAMRIALWNRATHGEQGALQHLLAGLWIQTEDYKSKGDKPVYEIHPLLF | 88 |
| | FDESNGGAKEITFSRASVQEIFLVDSAHTHRKTVSFLTRNTAISSIRRRLEVTFE | |
| | SHEVIHVRAVEDVARAYSEKDTRYAWDGQYTRYHGKLVQNLPNIMMPPG | |
| | IT | |
| 4.2 | VQTAMRIALWNRATHGEQGALQHLLAGLWIQTEDYKSKGDKPVYEIHPLLF | 89 |
| | FDLQENSNVVIEKNVEITFSRASVQEIFLVDSAHTHRKTVSFLTRNTAISSIRR | |
| | RLEVTFESHEVIHVRAVEDVARAYSEKDTRYAWDGQYTRYHGKLVQNLPN | |
| | IMMPPGIT | |
| 4.3 | VQTAMRIALWNRATHGEQGALQHLLAGLWIQTELQENSNVVIEKNVIHPLL | 90 |
| | FFDESNGGAKEITFSRASVQEIFLVDSAHTHRKTVSFLTRNTAISSIRRRLEVTF | |
| | ESHEVIHVRAVEDVADYKSKGDKPVYEWDGQYTRYHGKLVQNLPNIMMP | |
| | PGIT | |
| 4.4 | VQTAMRIALWNRATHGEQGALQHLLAGLWIQTEDYKSKGDKPVYEIHPLLF | 97 |
| (4B1) | FDVKPSFTGVEITFSRASVQEIFLVDSAHTHRKTVSFLTRNTAISSIRRRLEVTF | |
| | ESHEVIHVRAVEDVARAYSEKDTRYAWDGQYTRYHGKLVQNLPNIMMPP | |
| | GIT | |
| 4.5 | VQTAMRIALWNRATHGEQGALQHLLAGLWIQTEDYKSKGDKPVYEIHPLLF | 98 |
| (4B2) | FDESGFNFSEITFSRASVQEIFLVDSAHTHRKTVSFLTRNTAISSIRRRLEVTFE | |
| | SHEVIHVRAVEDVARAYSEKDTRYAWDGQYTRYHGKLVQNLPNIMMPPG | |
| | IT | |
| 4.6 | VQTAMRIALWNRATHGEQGALQHLLAGLWIQTEDYKSKGDKPVYEIHPLLF | 99 |
| (4B3) | FDGNEIKDSEITFSRASVQEIFLVDSAHTHRKTVSFLTRNTAISSIRRRLEVTFE | |
| | SHEVIHVRAVEDVARAYSEKDTRYAWDGQYTRYHGKLVQNLPNIMMPPG | |
| | IT | |
| 5.1 | APVYRPQDHAATINGVSDQGALQHLLAGLWIQTEDYKSKGDKPVYEIHPLL | 100 |
| (5A1) | FFDESNGGAKEITFSRASVQEIFLVDSAHTHRKTVSFLTRNTAISSIRRRLEVTF | |
| | ESHEVIHVRAVEDVARAYSEKDTRYAWDGQYTRYHGKLVQNLPNIMMPP | |
| | GIT | |
| 5.2 | VNVSNLQVDHLWKRQGALQHLLAGLWIQTEDYKSKGDKPVYEIHPLLFFD | 101 |
| (5A2) | ESNGGAKEITFSRASVQEIFLVDSAHTHRKTVSFLTRNTAISSIRRRLEVTFESH | |
| | EVIHVRAVEDVARAYSEKDTRYAWDGQYTRYHGKLVQNLPNIMMPPGIT | |
| 5.3 | VDFPLNPNTRFLAGKLPAETQGALQHLLAGLWIQTEDYKSKGDKPVYEIHP | 102 |
| (5A3) | LLFFDESNGGAKEITFSRASVQEIFLVDSAHTHRKTVSFLTRNTAISSIRRRLEV | |
| | TFESHEVIHVRAVEDVARAYSEKDTRYAWDGQYTRYHGKLVQNLPNIMM | |
| | PPGIT | |
| 5.4 | KNGAGSTDLKWEQFLQKYQGALQHLLAGLWIQTEDYKSKGDKPVYEIHP | 103 |
| (5A4) | LLFFDESNGGAKEITFSRASVQEIFLVDSAHTHRKTVSFLTRNTAISSIRRRLEV | |
| | TFESHEVIHVRAVEDVARAYSEKDTRYAWDGQYTRYHGKLVQNLPNIMM | |
| | PPGIT | |
| 5.5 | ANRWISGPHWWNSLSSQGALQHLLAGLWIQTEDYKSKGDKPVYEIHPLLFF | 104 |
| (5A5) | DESNGGAKEITFSRASVQEIFLVDSAHTHRKTVSFLTRNTAISSIRRRLEVTFES | |
| | HEVIHVRAVEDVARAYSEKDTRYAWDGQYTRYHGKLVQNLPNIMMPPGI | |
| | T | |
| 5.6 | VQTAMRIALWNRATHGSSEAAAKTLAGLWIQTEDYKSKGDKPVYEIHPLLF | 105 |
| (5B1) | FDESNGGAKEITFSRASVQEIFLVDSAHTHRKTVSFLTRNTAISSIRRRLEVTFE | |
| | SHEVIHVRAVEDVARAYSEKDTRYAWDGQYTRYHGKLVQNLPNIMMPPG | |
| | IT | |
| 5.7 | VQTAMRIALWNRATHGVKPSFTGVLAGLWIQTEDYKSKGDKPVYEIHPLLF | 106 |
| (5B2) | FDESNGGAKEITFSRASVQEIFLVDSAHTHRKTVSFLTRNTAISSIRRRLEVTFE | |
| | SHEVIHVRAVEDVARAYSEKDTRYAWDGQYTRYHGKLVQNLPNIMMPPG | |
| | IT | |
| 5.8 | VQTAMRIALWNRATHGPESGFNFSPLAGLWIQTEDYKSKGDKPVYEIHPLL | 107 |
| (5B3) | FFDESNGGAKEITFSRASVQEIFLVDSAHTHRKTVSFLTRNTAISSIRRRLEVTF | |
| | ESHEVIHVRAVEDVARAYSEKDTRYAWDGQYTRYHGKLVQNLPNIMMPP | |
| | GIT | |
| 5.9 | VQTAMRIALWNRATHGEQGALQHLLAGLWIQTEDYKSKGDKPVYEIHPLLF | 108 |
| (5C1) | FDESNGGAKEITFSRGNEIKDSVQEIFLVDSAHTHRKTVSFLTRNTAISSIRRR | |
| | LEVTFESHEVIHVRAVEDVARAYSEKDTRYAWDGQYTRYHGKLVQNLPNI | |
| | MMPPGIT | |
| 5.10 | VQTAMRIALWNRATHGEQGALQHLLAGLWIQTEDYKSKGDKPVYEIHPLLF | 109 |
| (5D1) | FDESNGGAKEITFSRASVQEIFLVDSAHTHGNEIKDSTVSFLTRNTAISSIRRR | |
| | LEVTFESHEVIHVRAVEDVARAYSEKDTRYAWDGQYTRYHGKLVQNLPNI | |
| | MMPPGIT | |
| 5.11 | VQTAMRIALWNRATHGEQGALQHLLAGLWIQTEDYKSKGDKPVYEIHPLLF | 110 |
| (5E1) | FDESNGGAKEITFSRASVQEIFLVDSAHTHRKTVSFLTRNSTGDEQQGTIRRR | |
| | LEVTFESHEVIHVRAVEDVARAYSEKDTRYAWDGQYTRYHGKLVQNLPNI | |
| | MMPPGIT | |
| 5.12 | VQTAMRIALWNRATHGEQGALQHLLAGLWIQTEDYKSKGDKPVYEIHPLLF | 111 |
| (5E2) | FDESNGGAKEITFSRASVQEIFLVDSAHTHRKTVSFLTRNAVGKDIEGQIRRR | |
| | LEVTFESHEVIHVRAVEDVARAYSEKDTRYAWDGQYTRYHGKLVQNLPNI | |
| | MMPPGIT | |
| 5.13 | VQTAMRIALWNRATHGEQGALQHLLAGLWIQTEDYKSKGDKPVYEIHPLLF | 112 |
| (5E3) | FDESNGGAKEITFSRASVQEIFLVDSAHTHRKTVSFLTRNFGQRKEDIRRRLE | |
| | VTFESHEVIHVRAVEDVARAYSEKDTRYAWDGQYTRYHGKLVQNLPNIM | |
| | MPPGIT | |
| 5.14 | VQTAMRIALWNRATHGEQGALQHLLAGLWIQTEDYKSKGDKPVYEIHPLLF | 113 |
| (5E4) | FDESNGGAKEITFSRASVQEIFLVDSAHTHRKTVSFLTRNVKPSFTGVIRRRL | |
| | EVTFESHEVIHVRAVEDVARAYSEKDTRYAWDGQYTRYHGKLVQNLPNIM | |
| | MPPGIT | |
| 5.15 | VQTAMRIALWNRATHGEQGALQHLLAGLWIQTEDYKSKGDKPVYEIHPLLF | 114 |
| (5E5) | FDESNGGAKEITFSRASVQEIFLVDSAHTHRKTVSFLTRNGNEIKDSIRRRLE | |
| | VTFESHEVIHVRAVEDVARAYSEKDTRYAWDGQYTRYHGKLVQNLPNIM | |
| | MPPGIT | |

TABLE 5
Tp0751 (99-237) mutant chimera designations, coding sequences, and sequence IDs.
See Table 3 for construct description legend.

| Construct | | SEQ |
|---|---|---|
| ID | Sequence Description ad Nucleotide Sequence | ID NO: |
| 0.1 | Tp0751 ΔLoop 2 ISPNS→GDDLHMG | |
| | GTGCAGACCGCGATGCGTATCGCGCTGTGGAACCGTGCGACCCACGGT | 147 |
| | GAACAAGGTGCGCTGCAACACCTGCTGGCGGGTCTGTGGATTCAGACC | |
| | GAAGGCGATGATCTGCACATGGGCGGCGACATTCACCCGCTGCTGTTC | |
| | TTTGATCGTGAGCACGCGGAAATCACCTTCAGCCGTGCGAGCGTGCAG | |
| | GAGATTTTTCTGGTTGACAGCGCGCACACCCACCGTAAGACCGTGAGC | |
| | TTCCTGACCCGTAACACCGCGATCAGCAGCATCCGTCGTCGTCTGGAA | |
| | GTGACCTTTGAGAGCCACGAAGTGATCCACGTTCGTGCGGTGGAAGAC | |
| | GTTGCGCGTCTGAAAATTGGTAGCACCAGCATGTGGGATGGCCAATAC | |
| | ACCCGTTATCACGCGGGTCCGGCGAGCGCGCCGAGCCCGGCGGCCGC | |
| 0.2 | Tp0751 ΔLoop 2 ISPNS→KTVNGIRH | |
| | GTGCAGACCGCGATGCGTATCGCGCTGTGGAACCGTGCGACCCACGGT | 148 |
| | GAACAAGGTGCGCTGCAACACCTGCTGGCGGGTCTGTGGATTCAGACC | |
| | GAAAAAACCGTGAACGGCATTCGTCATGGCGACATTCACCCGCTGCTG | |
| | TTCTTTGATCGTGAGCACGCGGAAATCACCTTCAGCCGTGCGAGCGTG | |
| | CAGGAGATTTTTCTGGTTGACAGCGCGCACACCCACCGTAAGACCGTG | |
| | AGCTTCCTGACCCGTAACACCGCGATCAGCAGCATCCGTCGTCGTCTG | |
| | GAAGTGACCTTTGAGAGCCACGAAGTGATCCACGTTCGTGCGGTGGAA | |
| | GACGTTGCGCGTCTGAAAATTGGTAGCACCAGCATGTGGGATGGCCAA | |
| | TACACCCGTTATCACGCGGGTCCGGCGAGCGCGCCGAGCCCGGCGGCC | |
| | GC | |
| 0.3 | Tp0751 ΔLoop 2 ISPNS→VLYNQAEKGS | |
| | GTGCAGACCGCGATGCGTATCGCGCTGTGGAACCGTGCGACCCACGGT | 149 |
| | GAACAAGGTGCGCTGCAACACCTGCTGGCGGGTCTGTGGATTCAGACC | |
| | GAAGTGCTGTATAACCAGGCGGAAAAAGGCAGCGGCGACATTCACCC | |
| | GCTGCTGTTCTTTGATCGTGAGCACGCGG | |
| | AAATCACCTTCAGCCGTGCGAGCGTGCAGGAGATTTTTCTGGTTGACA | |
| | GCGCGCACACCCACCGTAAGACCGTGAGCTTCCTGACCCGTAACACCG | |
| | CGATCAGCAGCATCCGTCGTCGTCTGGAAGTGACCTTTGAGAGCCACG | |
| | AAGTGATCCACGTTCGTGCGGTGGAAGACGTTGCGC | |
| | GTCTGAAAATTGGTAGCACCAGCATGTGGGATGGCCAATACACCCGTT | |
| | ATCACGCGGGTCCGGCGAGCGCGCCGAGCCCGGCGGCCGC | |
| 0.4 | Tp0751 ΔLoop 3 EH→GDDLHMG | |
| | GTGCAGACCGCGATGCGTATCGCGCTGTGGAACCGTGCGACCCACGGT | 150 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGGCGATGATCTGCACATGGGCGCGGAAATCACCTTCAGCCGTGCG | |
| | AGCGTGCAGGAGATTTTTCTGGTTGACAGCGCGCACACCCACCGTAAG | |
| | ACCGTGAGCTTCCTGACCCGTAACACCGCGATCAGCAGCATCCGTCGT | |
| | CGTCTGGAAGTGACCTTTGAGAGCCACGAAGTGATCCACGTTCGTGCG | |
| | GTGGAAGACGTTGCGCGTCTGAAAATTGGTAGCACCAGCATGTGGGAT | |
| | GGCCAATACACCCGTTATCACGCGGGTCCGGCGAGCGCGCCGAGCCCG | |
| | GCGGCCGC | |
| 0.5 | Tp0751 ΔLoop 3 EH→VLYNQAEKGS | |
| | GTGCAGACCGCGATGCGTATCGCGCTGTGGAACCGTGCGACCCACGGT | 151 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGTGCTGTATAACCAGGCGGAAAAAGGCAGCGCGGAAATCACCTTC | |
| | AGCCGTGCGAGCGTGCAGGAGATTTTTCTGGTTGACAGCGCGCACACC | |
| | CACCGTAAGACCGTGAGCTTCCTGACCCGTAACACCGCGATCAGCAGC | |
| | ATCCGTCGTCGTCTGGAAGTGACCTTTGAGAGCCACGAAGTGATCCAC | |
| | GTTCGTGCGGTGGAAGACGTTGCGCGTCTGAAAATTGGTAGCACCAGC | |
| | ATGTGGGATGGCCAATACACCCGTTATCACGCGGGTCCGGCGAGCGCG | |
| | CCGAGCCCGGCGGCCGC | |
| 0.6 | Tp0751 ΔLoop 6 IS→GDDLHMG | |
| | GTGCAGACCGCGATGCGTATCGCGCTGTGGAACCGTGCGACCCACGGT | 152 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGGGCGATGATCTGCACATGGGCAGCATCCGTCGT | |
| | CGTCTGGAAGTGACCTTTGAGAGCCACGAAGTGATCCACGTTCGTGCG | |
| | GTGGAAGACGTTGCGCGTCTGAAAATTGGTAGCACCAGCATGTGGGAT | |
| | GGCCAATACACCCGTTATCACGCGGGTCCGGCGAGCGCGCCGAGCCCG | |
| | GCGGCCGC | |
| 0.7 | Tp0751 ΔLoop 6 IS→KTVNGIRH | |
| | GTGCAGACCGCGATGCGTATCGCGCTGTGGAACCGTGCGACCCACGGT | 153 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGAAAACCGTGAACGGCATTCGTCATAGCATCCGT | |
| | CGTCGTCTGGAAGTGACCTTTGAGAGCCACGAAGTGATCCACGTTCGT | |
| | GCGGTGGAAGACGTTGCGCGTCTGAAAATTGGTAGCACCAGCATGTGG | |
| | GATGGCCAATACACCCGTTATCACGCGGGTCCGGCGAGCGCGCCGAGC | |
| | CCGGCGGCCGC | |
| 0.8 | Tp0751 ΔLoop 6 IS→VLYNQAEKGS | |
| | GTGCAGACCGCGATGCGTATCGCGCTGTGGAACCGTGCGACCCACGGT | 154 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGGTGCTGTATAACCAGGCGGAAAAAGGCAGCAG | |
| | CATCCGTCGTCGTCTGGAAGTGACCTTTGAGAGCCACGAAGTGATCCA | |
| | CGTTCGTGCGGTGGAAGACGTTGCGCGTCTGAAAATTGGTAGCACCAG | |
| | CATGTGGGATGGCCAATACACCCGTTATCACGCGGGTCCGGCGAGCGC | |
| | GCCGAGCCCGGCGGCCGC | |
| 0.9 | Tp0751 ΔLoop 8 LKIGSTS→GDDLHMG | |
| | GTGCAGACCGCGATGCGTATCGCGCTGTGGAACCGTGCGACCCACGGT | 155 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGATCAGCAGCATCCGTCGTCGTCTGGAAGTGACC | |
| | TTTGAGAGCCACGAAGTGATCCACGTTCGTGCGGTGGAAGACGTTGCG | |
| | CGTGGCGATGATCTGCACATGGGTATGTGGGATGGCCAATACACCCGT | |
| | TATCACGCGGGTCCGGCGAGCGCGCCGAGCCCGGCGGCCGC | |
| 0.10 | Tp0751 ΔLoop 8 LKIGSTS→KTVNGIRH | |
| | GTGCAGACCGCGATGCGTATCGCGCTGTGGAACCGTGCGACCCACGGT | 156 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGATCAGCAGCATCCGTCGTCGTCTGGAAGTGACC | |
| | TTTGAGAGCCACGAAGTGATCCACGTTCGTGCGGTGGAAGACGTTGCG | |
| | CGTAAAACCGTGAACGGTATTCGTCAcATGTGGGATGGCCAATACACC | |
| | CGTTATCACGCGGGTCCGGCGAGCGCGCCGAGCCCGGCGGCCGC | |
| 0.11 | Tp0751 ΔLoop 8 LKIGSTS→VLYNQAEKGS | |
| | GTGCAGACCGCGATGCGTATCGCGCTGTGGAACCGTGCGACCCACGGT | 157 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGATCAGCAGCATCCGTCGTCGTCTGGAAGTGACC | |
| | TTTGAGAGCCACGAAGTGATCCACGTTCGTGCGGTGGAAGACGTTGCG | |
| | CGTGTGCTGTATAACCAGGCGGAAAAAGGCAGCATGTGGGATGGCCA | |
| | ATACACCCGTTATCACGCGGGTCCGGCGAGCGCGCCGAGCCCGGCGGC | |
| | CGC | |
| 1.1 | Tp0751 Expanded ΔLoop 2 EISPNSGDI→TprC Loop 3 RAYSEKDTRYA | |
| | GTACAAACAGCAATGAGGATAGCTCTATGGAACCGTGCTACCCACGGC | 158 |
| | GAACAGGGTGCGCTGCAGCACCTGTTAGCAGGCCTTTGGATTCAAACC | |
| | CGTGCGTATAGCGAAAAAGATACCCGTTACGCGCACCCGCTGTTGTTT | |
| | TTCGATCGTGAGCACGCGGAAATTACCTTCAGCCGCGCGTCTGTTCAA | |
| | GAGATCTTTCTGGTGGACAGCGCCCATACGCATCGTAAGACCGTTAGC | |
| | TTTCTGACCAGAAATACTGCCATTAGCTCGATCCGTCGTCGCTTGGAGG | |
| | TTACCTTCGAGTCCCATGAAGTAATCCACGTGCGCGCTGTGGAGGACG | |
| | TCGCGCGTCTGAAAATCGGCAGCACGTCCATGTGGGACGGTCAGTATA | |
| | CGCGCTACCATGCTGGTCCGGCATCCGCGCCATCTCCG | |
| 1.2 | Tp0751 Expanded ΔLoop 2 EISPNSGDI→TprC Loop 6 | |
| | GKLVQNLPNIMMPPGIT | |
| | GTACAAACAGCTATGAGGATAGCACTATGGAACCGTGCTACCCACGGT | 159 |
| | GAGCAGGGTGCGCTGCAGCACCTGTTGGCGGGCCTGTGGATTCAAACG | |
| | GGTAAACTTGTTCAGAATCTGCCGAACATCATGATGCCGCCAGGCATC | |
| | ACCCATCCGCTGTTGTTTTTCGACCGTGAGCACGCCGAAATCACCTTCT | |
| | CTCGTGCTTCCGTGCAAGAGATTTTTCTGGTGGACAGCGCGCACACCC | |
| | ACCGTAAGACGGTTTCGTTTTTAACGCGTAACACCGCGATTAGCAGCA | |
| | TCCGTCGCCGTTTGGAAGTCACTTTCGAGAGCCATGAAGTGATTCACG | |
| | TGCGCGCGGTTGAAGATGTTGCACGCCTGAAAATCGGCTCCACCAGCA | |
| | TGTGGGATGGCCAATATACCCGCTACCATGCGGGTCCGGCAAGCGCCC | |
| | CGTCTCCG | |
| 1.3 | Tp0751 ΔLoop 2 ISPNS→TprK Loop 2 AKTIEATLH | |
| | GTACAAACAGCTATGAGGATAGCACTATGGAATCGTGCCACGCACGGC | 160 |
| | GAACAGGGTGCTCTGCAACATCTGCTGGCAGGTTTATGGATTCAGACC | |
| | GAGGCTAAAACCATTGAAGCGACTCTGCATGGCGACATCCACCCGCTG | |
| | TTGTTTTTTGATCGTGAGCACGCGGAAATCACCTTCAGCCGTGCGTCGG | |
| | TGCAAGAGATCTTCCTGGTGGACAGCGCGCACACCCACCGCAAAACCG | |
| | TTAGCTTTCTCACGCGCAACACTGCGATTAGCTCTATCCGCCGTCGTTT | |
| | GGAGGTCACCTTCGAGAGCCACGAAGTTATTCATGTTCGTGCAGTAGA | |
| | AGACGTGGCCCGTCTGAAGATCGGCTCCACCTCCATGTGGGATGGTCA | |
| | GTATACCAGATACCATGCGGGTCCAGCATCTGCTCCGAGCCCG | |
| 1.4 | Tp0751 Expanded ΔLoop 2 EISPNSGDI→TprK Loop 2 AKTIEATLH | |
| | GTACAAACAGCAATGAGGATAGCTCTATGGAATCGTGCTACCCACGGT | 161 |
| | GAACAAGGTGCGCTGCAGCACTTACTGGCAGGCCTGTGGATTCAGACC | |
| | GCGAAAACGATCGAAGCAACGCTGCATCATCCGCTGTTGTTTTTTGAT | |
| | CGTGAGCACGCGGAAATCACCTTCAGCCGTGCCTCTGTTCAAGAGATC | |
| | TTCCTGGTGGACAGCGCGCACACCCACCGCAAAACGGTGTCGTTCCTG | |
| | ACTCGCAACACCGCGATTTCCAGCATTCGTCGCCGTTTGGAGGTCACCT | |
| | TTGAGAGCCACGAGGTGATTCATGTTAGAGCCGTTGAAGATGTAGCGC | |
| | GTCTTAAGATCGGCTCCACCAGCATGTGGGACGGCCAGTACACCCGTT | |
| | ATCATGCTGGTCCGGCATCCGCGCCAAGCCCG | |
| 1.5 | Tp0751 ΔLoop 2 ISPNS→TprK New Loop 2 ESNGGAK | |
| | GTACAAACAGCAATGAGGATAGCTCTATGGAACCGCGCTACCCACGGC | 162 |
| | GAACAGGGTGCGCTGCAACATCTGTTGGCAGGCCTGTGGATTCAGACT | |
| | GAGGAGTCTAACGGTGGTGCCAAGGGTGATATTCACCCACTGTTGTTT | |
| | TTCGACCGCGAGCACGCCGAAATCACCTTCAGCCGTGCGTCCGTGCAG | |
| | GAGATCTTTCTTGTAGACAGCGCGCACACCCATCGTAAAACCGTTAGC | |
| | TTTTTGACCCGTAATACCGCAATTAGCTCCATTCGTCGTCGCCTGGAAG | |
| | TTACGTTCGAGTCCCATGAAGTCATCCACGTGCGTGCGGTGGAAGATG | |
| | TTGCGCGTCTGAAAATCGGCAGCACCTCTATGTGGGACGGCCAATATA | |
| | CGAGATACCATGCGGGTCCGGCAAGCGCTCCGTCGCCG | |
| 1.6 | Tp0751 ΔLoop 2 ISPNS→TprK Loop 3 Truncation 1 DYKSKGDKPVYE | |
| | GTACAAACAGCTATGAGGATAGCACTATGGAACCGCGCTACCCACGGT | 163 |
| | GAACAGGGTGCGCTGCAGCATCTGCTGGCAGGCCTGTGGATTCAGACG | |
| | GAGGACTACAAAAGCAAAGGTGATAAACCGGTTTATGAAATTCACCC | |
| | GCTGTTATTTTTTGATCGTGAGCACGCCGAGATCACCTTCAGCCGTGCT | |
| | TCTGTTCAAGAGATCTTTTTGGTTGATAGCGCGCACACCCACCGCAAG | |
| | ACCGTTTCGTTCTTGACCCGTAATACCGCAATTAGCAGCATTCGTAGAC | |
| | GCCTGGAAGTGACGTTCGAGTCCCATGAAGTCATCCACGTGCGTGCGG | |
| | TGGAAGACGTGGCCCGTCTCAAGATCGGCAGCACTTCAATGTGGGACG | |
| | GCCAATATACCCGCTACCATGCGGGTCCGGCGTCTGCGCCATCCCCG | |
| 1.7 | Tp0751 New Expanded ΔLoop 2 ISPNSGD→TprK Loop 10 | |
| | LQENSNVVIEKNV | |
| | GTACAAACAGCTATGAGGATAGCACTATGGAACCGTGCCACCCACGGT | 164 |
| | GAGCAGGGCGCGCTGCAACACCTGCTGGCGGGTCTGTGGATTCAGACC | |
| | GAACTGCAAGAGAATAGCAATGTTGTTATTGAAAAAAACGTGATCCAC | |
| | CCACTGTTGTTTTTCGACCGTGAGCACGCCGAAATCACGTTCAGCCGTG | |
| | CGTCCGTGCAAGAGATCTTCCTCGTCGATAGCGCGCACACCCACCGCA | |
| | AAACCGTTAGCTTTCTGACCCGCAACACCGCAATTTCTAGCATTCGTCG | |
| | TCGCTTGGAGGTGACGTTTGAATCCCATGAAGTCATCCATGTTCGTGCT | |
| | GTGGAAGATGTTGCGCGTCTTAAGATCGGCAGCACTTCAATGTGGGAC | |
| | GGCCAGTATACCAGATACCATGCGGGTCCGGCATCTGCTCCGTCGCCG | |
| 1.8 | Tp0751 Expanded ΔLoop 3 REHA→BamA1 DNNQPFDLTV | |
| | GTACAAACAGCTATGAGGATAGCACTATGGAACCGTGCGACCCACGG | 165 |
| | CGAACAGGGCGCACTGCAACACCTGTTAGCGGGTCTGTGGATTCAAAC | |
| | CGAAATCTCCCCGAATAGCGGTGACATCCACCCGCTGCTGTTCTTCGA | |
| | CGACAACAACCAACCGTTTGACCTGACGGTGGAGATCACTTTCAGCCG | |
| | TGCGTCTGTTCAGGAGATCTTTCTGGTTGATAGCGCGCATACCCACCGC | |
| | AAAACCGTGAGCTTTTTGACGCGTAATACCGCAATTTCGAGCATTCGT | |
| | CGCCGCTTGGAAGTGACTTTCGAGTCCCATGAAGTTATTCACGTGCGT | |
| | GCTGTCGAGGATGTTGCGCGTCTTAAGATCGGCAGCACCAGCATGTGG | |
| | GATGGTCAGTATACCAGATACCATGCAGGTCCGGCTTCTGCCCCATCC | |
| | CCG | |
| 1.9 | Tp0751 Expanded ΔLoop 3 REHA→TprK New Loop 2 ESNGGAK | |
| | GTACAAACAGCAATGAGGATAGCTCTATGGAACCGCGCGACCCACGG | 166 |
| | CGAGCAGGGTGCGCTGCAGCATCTGCTGGCAGGCCTGTGGATTCAAAC | |
| | GGAAATCTCACCGAACAGCGGTGACATCCACCCGCTGTTGTTTTTCGA | |
| | TGAATCCAACGGCGGTGCCAAGGAGATCACCTTCAGCCGTGCCTCCGT | |
| | GCAAGAGATCTTTCTTGTGGACAGCGCGCACACCCACCGTAAAACCGT | |
| | TTCTTTTCTCACGCGTAATACCGCAATTTCTTCCATTCGCAGACGTCTG | |
| | GAAGTCACCTTCGAGAGCCATGAAGTGATTCACGTTCGTGCTGTTGAG | |
| | GACGTTGCGCGTTTGAAGATCGGCAGCACCAGCATGTGGGATGGTCAG | |
| | TACACTCGCTATCATGCGGGTCCAGCTTCGGCTCCGAGCCCG | |
| 1.10 | Tp0751 Expanded ΔLoop 3 REHA→TprK Loop 10 LQENSNVVIEKNV | |
| | GTACAAACAGCAATGAGGATAGCTCTATGGAACCGTGCGACGCATGGT | 167 |
| | GAACAGGGCGCGCTGCAACACCTGTTGGCTGGCCTGTGGATTCAGACC | |
| | GAAATCTCCCCGAATTCTGGTGATATCCACCCGCTGCTCTTCTTTGACC | |
| | TGCAAGAAAATTCCAACGTCGTGATTGAAAAAAACGTGGAGATCACCT | |
| | TCAGCCGTGCGAGCGTTCAAGAGATCTTTTTAGTTGATTCTGCCCACAC | |
| | GCACCGTAAAACCGTTAGCTTTCTGACTCGCAACACCGCAATTTCGAG | |
| | CATTCGTCGCCGTTTGGAGGTCACCTTCGAGAGCCATGAAGTGATCCA | |
| | CGTTAGAGCTGTGGAGGACGTTGCGCGTCTGAAGATCGGTTCCACCAG | |
| | CATGTGGGACGGCCAGTATACCCGCTACCATGCCGGTCCGGCAAGCGC | |
| | GCCATCTCCG | |
| 1.11 | Tp0751 Expanded ΔLoop 6 TAISS→BamA1 DNNQPFDLTV | |
| | GTACAAACAGCTATGAGGATAGCACTATGGAACCGTGCTACGCATGGT | 168 |
| | GAACAGGGCGCGCTGCAACACCTGTTAGCGGGCCTGTGGATTCAGACC | |
| | GAAATCAGCCCAAATTCTGGTGACATCCACCCGCTGCTCTTTTTCGACC | |
| | GTGAGCACGCCGAGATTACCTTCAGCCGTGCGTCCGTGCAAGAGATCT | |
| | TTCTGGTTGATAGCGCGCATACCCACCGCAAGACCGTGAGCTTTTTGA | |
| | CTCGCAATGACAACAACCAACCGTTCGACCTGACGGTTATTCGTAGAC | |
| | GCTTGGAAGTCACCTTCGAGTCGCACGAGGTGATTCATGTTCGTGCTGT | |
| | GGAAGATGTTGCGCGTCTGAAAATCGGCAGCACCAGCATGTGGGATG | |
| | GTCAGTATACCCGTTACCATGCCGGTCCGGCATCTGCACCGTCCCCG | |
| 1.12 | Tp0751 Expanded ΔLoop 6 TAISS→TprC Loop 3 RAYSEKDTRYA | |
| | GTACAAACAGCAATGAGGATAGCTCTATGGAATCGTGCCACGCATGGT | 169 |
| | GAGCAGGGTGCTCTGCAGCACTTGCTGGCAGGCCTGTGGATTCAAACC | |
| | GAAATCAGCCCGAACAGCGGTGATATTCACCCGCTGCTTTTTTTTGATC | |
| | GTGAACATGCGGAAATTACGTTCAGCCGTGCTTCCGTGCAGGAGATCT | |
| | TCTTGGTTGACTCCGCGCACACCCATCGTAAAACCGTTAGCTTTCTGAC | |
| | TCGCAACCGCGCGTATTCCGAAAAAGATACCAGATACGCAATTCGTCG | |
| | TCGCCTGGAAGTTACCTTCGAGAGCCACGAGGTCATCCACGTTCGTGC | |
| | GGTGGAGGACGTGGCGCGTCTCAAGATCGGCTCGACCAGCATGTGGG | |
| | ACGGCCAATATACCCGCTACCATGCCGGTCCGGCGTCTGCACCGTCTC | |
| | CA | |
| 1.13 | Tp0751 Expanded ΔLoop 6 TAISS→TprK New Loop 2 ESNGGAK | |
| | GTACAAACAGCTATGAGGATAGCACTATGGAACCGCGCTACTCACGGC | 170 |
| | GAACAGGGTGCGCTGCAACACTTGCTGGCAGGCCTGTGGATTCAGACC | |
| | GAGATCTCCCCGAATAGCGGTGACATTCATCCGCTGTTGTTTTTCGACC | |
| | GCGAGCACGCCGAGATCACGTTCAGCCGTGCGAGCGTTCAAGAGATTT | |
| | TTCTGGTGGACAGCGCGCATACCCACAGAAAGACCGTTAGCTTTCTCA | |
| | CGCGTAACGAATCTAACGGCGGTGCCAAGATTCGTCGTCGCCTGGAAG | |
| | TTACCTTCGAGTCCCATGAAGTCATCCACGTGCGTGCTGTGGAAGATG | |
| | TAGCACGTTTAAAAATCGGCTCGACCAGCATGTGGGATGGTCAGTACA | |
| | CCCGTTATCATGCGGGTCCGGCGTCTGCGCCATCCCCG | |
| 1.14 | Tp0751 Expanded ΔLoop 6 TAISS→TprK Loop 10 LQENSNVVIEKNV | |
| | GTACAAACAGCAATGAGGATAGCTCTATGGAACCGTGCAACGCACGG | 171 |
| | CGAACAGGGTGCTTTGCAACATCTGTTGGCGGGTCTGTGGATTCAGAC | |
| | CGAAATCTCGCCGAATTCTGGTGACATCCACCCGCTGCTGTTTTTTGAT | |
| | CGTGAGCACGCCGAAATTACCTTCAGCCGTGCGTCCGTGCAGGAGATC | |
| | TTCCTGGTTGACAGCGCGCATACCCACCGTAAGACCGTTTCCTTTCTGA | |
| | CTCGTAATCTGCAAGAGAACTCTAACGTAGTTATTGAGAAAAACGTGA | |
| | TTCGCCGTCGCTTAGAGGTTACCTTCGAGAGCCATGAAGTCATCCACG | |
| | TGCGCGCTGTGGAAGATGTCGCCCGTCTCAAGATCGGCAGCACGAGCA | |
| | TGTGGGACGGCCAATATACCCGCTACCATGCGGGTCCGGCATCCGCGC | |
| | CAAGCCCG | |
| 1.15 | Tp0751 New Expanded ΔLoop 8 RLKIGSTSM→BamA1 DNNQPFDLTV | |
| | GTACAAACAGCTATGAGGATAGCACTATGGAACCGCGCCACCCACGG | 172 |
| | CGAGCAGGGCGCGCTGCAACATCTCTTGGCTGGCCTGTGGATTCAGAC | |
| | CGAAATCTCTCCGAATAGCGGTGACATCCACCCGCTGTTGTTTTTCGAC | |
| | CGTGAACACGCGGAGATCACCTTTAGCCGTGCGTCCGTGCAAGAGATC | |
| | TTCTTAGTTGACAGCGCGCATACCCATAGAAAGACCGTGTCGTTCCTG | |
| | ACGCGTAATACCGCAATTTCTAGCATTCGTCGCCGTCTGGAAGTGACC | |
| | TTCGAGAGCCATGAGGTTATTCACGTGCGTGCCGTTGAAGATGTCGCA | |
| | GATAACAACCAGCCATTTGACCTGACGGTTTGGGATGGTCAATATACT | |
| | CGCTACCACGCGGGTCCGGCGTCCGCTCCGAGCCCG | |
| 1.16 | Tp0751 Expanded ΔLoop 8 LKIGSTSM→TprC Loop 1 | |
| | FQKNPRTGPGKHTHGFRTTN | |
| | GTGCAGACCGCGATGCGTATCGCGCTGTGGAACCGTGCGACCCACGGT | 173 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGATCAGCAGCATCCGTCGTCGTCTGGAAGTGACC | |
| | TTTGAGAGCCACGAAGTGATCCACGTTCGTGCGGTGGAAGACGTTGCG | |
| | CGTTTCCAGAAGAACCCGCGTACCGGTCCGGGCAAACACACCCACGGT | |
| | TTTCGTACCACCAACTGGGATGGCCAATACACCCGTTATCACGCGGGT | |
| | CCGGCGAGCGCGCCGAGCCCGGCGGCCGCCTAAGTAACTAAACTCGA | |
| | GCACCACCACCACCACCACTAATGTTAATTAAGTTGGGCGTTCCTAGG | |
| | CTGATAAAACAGAATTTGCCTGGCGGCAGTAGCGCGGTGGTCCCACCT | |
| | GACCCCATGCCGAACTCAGAAGTGAAACGCCGTAGCGCCGATGGTAGT | |
| | GTGGGGTCTCCCCATGCGAGAGTAGGGAACTGCCAGGCATCAAATAA | |
| | AACGAAAGGCTCAGTCGAAAGACTGGGCCTTTCGTTTTATCTGTTGTTT | |
| | GTCGGTGAACGCTCTCCTGAGTAGGACAAATCCGCCGGGAGCGGATTT | |
| | GAACGTTGCGAAGCAACGGCCCGGAGGGTGGCGGGCAGGACGCCCGC | |
| | CATAAACTGCCAGGCATCAAATTAAGCAGAAGGCCATCCTGACGGATG | |
| | GCCTTTTTGCGTTTCTACAAACTCTTTTGTTTATTTTTCTAAATACATTC | |
| | AAATATGTATCCGCTGAGCAATAACTAGCATAACCCCTTGGGGCCTCT | |
| | AAACGGGTCTTGAGGGGTTTTTTGCTGAAAGGAGGAACTATATCCGGA | |
| 1.17 | Tp0751 Expanded ΔLoop 8 LKIGSTSM→TprC Loop 2 | |
| | LAVELASSKSSTALSFTKPTAS | |
| | GTGCAGACCGCGATGCGTATCGCGCTGTGGAACCGTGCGACCCACGGT | 174 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGATCAGCAGCATCCGTCGTCGTCTGGAAGTGACC | |
| | TTTGAGAGCCACGAAGTGATCCACGTTCGTGCGGTGGAAGACGTTGCG | |
| | CGTCTGGCGGTGGAGCTGGCGAGCAGCAAGAGCAGCACCGCGCTGAG | |
| | CTTCACCAAACCGACCGCGAGCTGGGATGGCCAATACACCCGTTATCA | |
| | CGCGGGTCCGGCGAGCGCGCCGAGCCCGGCGGCCGCCTAAGTAACTA | |
| | AACTCGAGCACCACCACCACCACCACTAATGTTAATTAAGTTGGGCGT | |
| | TCCTAGGCTGATAAAACAGAATTTGCCTGGCGGCAGTAGCGCGGTGGT | |
| | CCCACCTGACCCCATGCCGAACTCAGAAGTGAAACGCCGTAGCGCCGA | |
| | TGGTAGTGTGGGGTCTCCCCATGCGAGAGTAGGGAACTGCCAGGCATC | |
| | AAATAAAACGAAAGGCTCAGTCGAAAGACTGGGCCTTTCGTTTTATCT | |
| | GTTGTTTGTCGGTGAACGCTCTCCTGAGTAGGACAAATCCGCCGGGAG | |
| | CGGATTTGAACGTTGCGAAGCAACGGCCCGGAGGGTGGCGGGCAGGA | |
| | CGCCCGCCATAAACTGCCAGGCATCAAATTAAGCAGAAGGCCATCCTG | |
| | ACGGATGGCCTTTTTGCGTTTCTACAAACTCTTTTGTTTATTTTTCTAAA | |
| | TACATTCAAATATGTATCCGCTGAGCAATAACTAGCATAACCCCTTGG | |
| | GGCCTCTAAACGGGTCTTGAGGGGTTTTTTGCTGAAAGGAGGAACTAT | |
| | ATCCGGA | |
| 1.18 | Tp0751 New Expanded ΔLoop 8 RLKIGSTSM→TprC Loop 3 | |
| | RAYSEKDTRYA | |
| | GTACAAACAGCTATGAGGATAGCACTATGGAACCGTGCGACCCACGGT | 175 |
| | GAACAGGGTGCACTCCAACACCTGCTGGCTGGCCTGTGGATTCAAACC | |
| | GAAATCTCCCCGAACAGCGGCGACATCCACCCGCTGTTATTTTTCGATC | |
| | GTGAGCACGCCGAGATCACGTTCAGCCGTGCTTCGGTGCAGGAGATCT | |
| | TTTTGGTTGATAGCGCGCATACCCACAGAAAGACCGTAAGCTTTTTGA | |
| | CCCGTAATACGGCGATTTCTTCCATTCGTCGCCGCCTGGAAGTGACCTT | |
| | CGAGTCCCATGAAGTTATTCACGTGCGTGCGGTCGAGGACGTTGCGCG | |
| | TGCGTATAGCGAAAAAGACACTCGCTACGCCTGGGATGGTCAGTATAC | |
| | CCGCTACCATGCGGGCCCAGCAAGCGCACCGTCTCCG | |
| 1.19 | Tp0751 New Expanded ΔLoop 8 RLKIGSTSM→TprC Loop 10 | |
| | GTTNRFNIINAAGNLLN | |
| | GTACAAACAGCAATGAGGATAGCTCTATGGAACCGTGCTACCCACGGC | 176 |
| | GAGCAGGGTGCGCTGCAACACCTGCTTGCCGGCCTGTGGATTCAGACG | |
| | GAAATTTCTCCGAACAGCGGTGACATCCACCCGCTGTTGTTTTTCGACC | |
| | GCGAGCACGCCGAAATCACGTTCTCTAGAGCGAGCGTTCAAGAGATCT | |
| | TTTTGGTGGACAGCGCGCATACCCATCGTAAAACCGTAAGCTTTCTGA | |
| | CCCGTAATACCGCAATTTCGAGCATTCGTCGTCGCCTGGAAGTCACCTT | |
| | CGAGTCCCATGAGGTGATTCACGTTCGTGCTGTGGAAGATGTTGCAGG | |
| | CACTACAAATCGCTTCAACATCATCAACGCGGCGGGAAACCTGTTGAA | |
| | TTGGGATGGTCAGTACACCCGTTATCATGCGGGTCCGGCAAGCGCGCC | |
| | GTCCCCA | |
| 1.20 | Tp0751 Expanded ΔLoop 8 LKIGSTSM→TprK Loop 3 | |
| | NGDYKSKGDKPVYEPG | |
| | GTGCAGACCGCGATGCGTATCGCGCTGTGGAACCGTGCGACCCACGGT | 177 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGATCAGCAGCATCCGTCGTCGTCTGGAAGTGACC | |
| | TTTGAGAGCCACGAAGTGATCCACGTTCGTGCGGTGGAAGACGTTGCG | |
| | CGTAACGGTGACTACAAGAGCAAAGGCGATAAGCCGGTGTATGAGCC | |
| | GGGTTGGGATGGCCAATACACCCGTTATCACGCGGGTCCGGCGAGCGC | |
| | GCCGAGCCCGGCGGCCGCCTAAGTAACTAAACTCGAGCACCACCACCA | |
| | CCACCACTAATGTTAATTAAGTTGGGCGTTCCTAGGCTGATAAAACAG | |
| | AATTTGCCTGGCGGCAGTAGCGCGGTGGTCCCACCTGACCCCATGCCG | |
| | AACTCAGAAGTGAAACGCCGTAGCGCCGATGGTAGTGTGGGGTCTCCC | |
| | CATGCGAGAGTAGGGAACTGCCAGGCATCAAATAAAACGAAAGGCTC | |
| | AGTCGAAAGACTGGGCCTTTCGTTTTATCTGTTGTTTGTCGGTGAACGC | |
| | TCTCCTGAGTAGGACAAATCCGCCGGGAGCGGATTTGAACGTTGCGAA | |
| | GCAACGGCCCGGAGGGTGGCGGGCAGGACGCCCGCCATAAACTGCCA | |
| | GGCATCAAATTAAGCAGAAGGCCATCCTGACGGATGGCCTTTTTGCGT | |
| | TTCTACAAACTCTTTTGTTTATTTTTCTAAATACATTCAAATATGTATCC | |
| | GCTGAGCAATAACTAGCATAACCCCTTGGGGCCTCTAAACGGGTCTTG | |
| | AGGGGTTTTTTGCTGAAAGGAGGAACTATATCCGGA | |
| 1.21 | Tp0751 Expanded ΔLoop 8 LKIGSTSM→TprK Loop 3 Truncation 1 | |
| | DYKSKGDKPVYE | |
| | GTGCAGACCGCGATGCGTATCGCGCTGTGGAACCGTGCGACCCACGGT | 178 |
| | GAACAAGGTGCGCTGCAACACCTGCTGGCGGGTCTGTGGATTCAGACC | |
| | GAAATTAGCCCGAACAGCGGCGACATTCACCCGCTGCTGTTCTTTGAT | |
| | CGTGAGCACGCGGAAATCACCTTCAGCCGTGCGAGCGTGCAGGAGATT | |
| | TTTCTGGTTGACAGCGCGCACACCCACCGTAAGACCGTGAGCTTCCTG | |
| | ACCCGTAACACCGCGATCAGCAGCATCCGTCGTCGTCTGGAAGTGACC | |
| | TTTGAGAGCCACGAAGTGATCCACGTTCGTGCGGTGGAAGACGTTGCG | |
| | CGTGACTACAAGAGCAAAGGTGATAAGCCGGTGTATGAGTGGGATGG | |
| | CCAATACACCCGTTATCACGCGGGTCCGGCGAGCGCGCCGAGCCCGGC | |
| | GGCCGCCTAAGTAACTAAACTCGAGCACCACCACCACCACCACTAATG | |
| | TTAATTAAGTTGGGCGTTCCTAGGCTGATAAAACAGAATTTGCCTGGC | |
| | GGCAGTAGCGCGGTGGTCCCACCTGACCCCATGCCGAACTCAGAAGTG | |
| | AAACGCCGTAGCGCCGATGGTAGTGTGGGGTCTCCCCATGCGAGAGTA | |
| | GGGAACTGCCAGGCATCAAATAAAACGAAAGGCTCAGTCGAAAGACT | |
| | GGGCCTTTCGTTTTATCTGTTGTTTGTCGGTGAACGCTCTCCTGAGTAG | |
| | GACAAATCCGCCGGGAGCGGATTTGAACGTTGCGAAGCAACGGCCCG | |
| | GAGGGTGGCGGGCAGGACGCCCGCCATAAACTGCCAGGCATCAAATT | |
| | AAGCAGAAGGCCATCCTGACGGATGGCCTTTTTGCGTTTCTACAAACT | |
| | CTTTTGTTTATTTTTCTAAATACATTCAAATATGTATCCGCTGAGCAAT | |
| | AACTAGCATAACCCCTTGGGGCCTCTAAACGGGTCTTGAGGGGTTTTTT | |
| | GCTGAAAGGAGGAACTATATCCGGA | |
| 1.22 | Tp0751 New Expanded ΔLoop 8 RLKIGSTSM→TprK Loop 3 | |
| | Truncation 1 DYKSKGDKPVYE | |
| | GTACAAACAGCAATGAGGATAGCTCTATGGAACCGTGCGACTCATGGT | 179 |
| | GAACAAGGTGCCTTACAGCACCTGTTGGCTGGCCTGTGGATTCAGACG | |
| | GAAATCTCCCCGAACAGCGGCGACATCCACCCGCTGTTGTTCTTTGATC | |
| | GTGAGCACGCGGAGATCACCTTTTCTCGTGCGTCGGTTCAGGAGATCT | |
| | TCCTTGTTGACAGCGCCCACACGCATCGTAAGACCGTTAGCTTTCTGAC | |
| | CCGCAATACCGCGATTTCTAGCATTAGACGTCGCCTGGAAGTCACCTT | |
| | CGAGAGCCATGAAGTGATTCACGTGCGTGCTGTGGAAGATGTAGCAGA | |
| | CTACAAATCCAAGGGTGATAAACCAGTTTATGAGTGGGACGGCCAATA | |
| | TACCCGCTACCATGCGGGTCCGGCATCCGCGCCGAGCCCG | |
| 1.23 | Tp0751 Expanded ΔLoop 8 LKIGSTSM→TprK Loop 3 Truncation 2 | |
| | DYKSKGDKP | |
| | GTACAAACAGCAATGAGGATAGCTCTATGGAACAGAGCTACTCACGG | 180 |
| | CGAACAAGGTGCGCTGCAACATCTGCTGGCTGGCCTGTGGATTCAGAC | |
| | CGAAATCTCCCCGAACAGCGGTGACATCCACCCGCTCTTGTTTTTCGAT | |
| | CGTGAGCACGCGGAAATCACGTTCAGCCGTGCGAGCGTACAGGAGAT | |
| | CTTTTTAGTTGATTCTGCCCACACCCATCGTAAGACGGTGAGCTTTTTG | |
| | ACCCGTAATACCGCGATTTCTAGCATTCGTCGCCGCCTGGAGGTCACCT | |
| | TCGAGAGCCATGAAGTTATTCACGTGCGCGCGGTTGAGGACGTGGCAC | |
| | GTGATTATAAATCCAAAGGCGACAAGCCGTGGGACGGTCAGTACACCC | |
| | GTTACCATGCCGGTCCGGCATCCGCGCCATCGCCG | |
| 1.24 | Tp0751 Expanded ΔLoop 8 LKIGSTSM→TprK Loop 3 Truncation 3 | |
| | SKGDKPVYE | |
| | GTACAAACAGCAATGAGGATAGCTCTATGGAACAGAGCCACCCACGG | 181 |
| | CGAGCAGGGTGCACTGCAGCATCTGCTCGCTGGCCTGTGGATTCAGAC | |
| | GGAAATCTCTCCGAATAGCGGTGACATCCACCCGCTGTTGTTTTTCGAT | |
| | CGTGAGCACGCGGAAATCACGTTCAGCCGTGCGTCCGTGCAAGAGATC | |
| | TTTTTGGTTGATAGCGCGCATACCCACCGCAAGACCGTTAGCTTTCTGA | |
| | CTCGCAACACCGCAATTTCTAGCATTCGTCGTCGCCTGGAAGTCACCTT | |
| | CGAGAGCCATGAAGTGATTCATGTTCGTGCTGTGGAAGATGTTGCGCG | |
| | TTCCAAAGGTGACAAACCGGTGTACGAGTGGGACGGCCAATACACCC | |
| | GTTATCACGCCGGTCCGGCATCGGCGCCAAGTCCG | |
| 1.25 | Tp0751 Expanded ΔLoop 8 LKIGSTSM→TprK Loop 3 Truncation 4 | |
| | KSKGDKPVY | |
| | GTACAAACAGCTATGAGGATAGCACTATGGAACCGCGCGACCCACGG | 182 |
| | CGAACAGGGTGCACTGCAACATCTGTTAGCCGGTCTGTGGATTCAGAC | |
| | CGAGATTAGCCCAAACAGCGGCGACATCCATCCGCTGCTGTTTTTCGA | |
| | CCGCGAGCACGCCGAAATCACCTTTAGCCGCGCGTCCGTGCAAGAGAT | |
| | CTTCCTGGTCGATAGCGCGCACACGCACCGTAAGACGGTTTCCTTCTTG | |
| | ACCCGTAATACCGCAATTTCTAGCATTAGACGCCGTTTGGAAGTGACC | |
| | TTTGAGAGCCATGAGGTGATCCACGTTCGTGCTGTGGAAGATGTTGCG | |
| | CGTAAGTCGAAAGGTGATAAACCGGTTTACTGGGACGGCCAGTATACT | |
| | CGTTACCATGCGGGTCCGGCATCTGCTCCGTCCCCG | |
| 2.1 | Tp0751 ΔLoop 2 ISPNS→TprC Loop 3 RAYSEKDTRYA (SEQ ID NO: 17) | |
| | Tp0751 Expanded ΔLoop 8 LKIGSTSM→TprK Loop 3 Truncation 1 | |
| | DYKSKGDKPVYE (SEQ ID NO: 21) | |
| | GTGCAGACCGCGATGCGTATCGCGCTGTGGAACCGTGCGACCCACGGT | 183 |
| | GAACAAGGTGCGCTGCAACACCTGCTGGCGGGTCTGTGGATTCAGACC | |
| | GAACGTGCGTATAGCGAAAAAGATACCCGTTATGCGGGCGACATTCAC | |
| | CCGCTGCTGTTCTTTGATCGTGAGCACGCGGAAATCACCTTCAGCCGTG | |
| | CGAGCGTGCAGGAGATTTTTCTGGTTGACAGCGCGCACACCCACCGTA | |
| | AGACCGTGAGCTTCCTGACCCGTAACACCGCGATCAGCAGCATCCGTC | |
| | GTCGTCTGGAAGTGACCTTTGAGAGCCACGAAGTGATCCACGTTCGTG | |
| | CGGTGGAAGACGTTGCGCGTGATTATAAAAGCAAAGGTGATAAACCG | |
| | GTGTATGAATGGGATGGCCAATACACCCGTTATCACGCGGGTCCGGCG | |
| | AGCGCGCCGAGCCCGGCGGCCGC | |
| 2.2 | Tp0751 ΔLoop 2 ISPNS→TprC Loop 6 GKLVQNLPNIMMPPGIT (SEQ ID | |
| | NO: 18) | |
| | Tp0751 Expanded ΔLoop 8 LKIGSTSM→TprK Loop 3 Truncation 1 | |
| | DYKSKGDKPVYE (SEQ ID NO: 21) | |
| | GTGCAGACCGCGATGCGTATCGCGCTGTGGAACCGTGCGACCCACGGT | 184 |
| | GAACAAGGTGCGCTGCAACACCTGCTGGCGGGTCTGTGGATTCAGACC | |
| | GAAGGCAAACTGGTGCAGAACCTGCCGAACATTATGATGCCGCCGGG | |
| | CATTACCGGCGACATTCACCCGCTGCTGTTCTTTGATCGTGAGCACGCG | |
| | GAAATCACCTTCAGCCGTGCGAGCGTGCAGGAGATTTTTCTGGTTGAC | |
| | AGCGCGCACACCCACCGTAAGACCGTGAGCTTCCTGACCCGTAACACC | |
| | GCGATCAGCAGCATCCGTCGTCGTCTGGAAGTGACCTTTGAGAGCCAC | |
| | GAAGTGATCCACGTTCGTGCGGTGGAAGACGTTGCGCGTGATTATAAA | |
| | AGCAAAGGTGATAAACCGGTGTATGAATGGGATGGCCAATACACCCG | |
| | TTATCACGCGGGTCCGGCGAGCGCGCCGAGCCCGGCGGCCGC | |
| 2.3 | Tp0751 ΔLoop 2 ISPNS→TprC Loop 10 GTTNRFNIINAAGNLLN (SEQ ID | |
| | NO: 26) | |
| | Tp0751 Expanded ΔLoop 8 LKIGSTSM→TprK Loop 3 Truncation 1 | |
| | DYKSKGDKPVYE (SEQ ID NO: 21) | |
| | GTGCAGACCGCGATGCGTATCGCGCTGTGGAACCGTGCGACCCACGGT | 185 |
| | GAACAAGGTGCGCTGCAACACCTGCTGGCGGGTCTGTGGATTCAGACC | |
| | GAAGGCACCACCAACCGTTTTAACATTATTAACGCGGCGGGCAACCTG | |
| | CTGAACGGCGACATTCACCCGCTGCTGTTCTTTGATCGTGAGCACGCG | |
| | GAAATCACCTTCAGCCGTGCGAGCGTGCAGGAGATTTTTCTGGTTGAC | |
| | AGCGCGCACACCCACCGTAAGACCGTGAGCTTCCTGACCCGTAACACC | |
| | GCGATCAGCAGCATCCGTCGTCGTCTGGAAGTGACCTTTGAGAGCCAC | |
| | GAAGTGATCCACGTTCGTGCGGTGGAAGACGTTGCGCGTGATTATAAA | |
| | AGCAAAGGTGATAAACCGGTGTATGAATGGGATGGCCAATACACCCG | |
| | TTATCACGCGGGTCCGGCGAGCGCGCCGAGCCCGGCGGCCGC | |
| 2.4 | Tp0751 ΔLoop 2 ISPNS→TprC Loop 10 (Nichols Strain) | |
| | GTTNRFNIINPAGNLLN (SEQ ID NO: 31) | |
| | Tp0751 Expanded ΔLoop 8 LKIGSTSM→TprK Loop 3 Truncation 1 | |
| | DYKSKGDKPVYE (SEQ ID NO: 21) | |
| | GTGCAGACCGCGATGCGTATCGCGCTGTGGAACCGTGCGACCCACGGT | 186 |
| | GAACAAGGTGCGCTGCAACACCTGCTGGCGGGTCTGTGGATTCAGACC | |
| | GAAGGCACCACCAACCGTTTTAACATTATTAACCCGGCGGGCAACCTG | |
| | CTGAACGGCGACATTCACCCGCTGCTGTTCTTTGATCGTGAGCACGCG | |
| | GAAATCACCTTCAGCCGTGCGAGCGTGCAGGAGATTTTTCTGGTTGAC | |
| | AGCGCGCACACCCACCGTAAGACCGTGAGCTTCCTGACCCGTAACACC | |
| | GCGATCAGCAGCATCCGTCGTCGTCTGGAAGTGACCTTTGAGAGCCAC | |
| | GAAGTGATCCACGTTCGTGCGGTGGAAGACGTTGCGCGTGATTATAAA | |
| | AGCAAAGGTGATAAACCGGTGTATGAATGGGATGGCCAATACACCCG | |
| | TTATCACGCGGGTCCGGCGAGCGCGCCGAGCCCGGCGGCCGC | |
| 2.5 | Tp0751 ΔLoop 2 ISPNS→TprC Loop 11 QGVLADAPYMGIAESIW (SEQ | |
| | ID NO: 32) | |
| | Tp0751 Expanded ΔLoop 8 LKIGSTSM→TprK Loop 3 Truncation 1 | |
| | DYKSKGDKPVYE (SEQ ID NO: 21) | |
| | CCATGGGAGTGCAGACCGCGATGCGTATCGCGCTGTGGAACCGTGCGA | 187 |
| | CCCACGGTGAACAAGGTGCGCTGCAACACCTGCTGGCGGGTCTGTGGA | |
| | TTCAGACCGAACAGGGCGTGCTGGCGGATGCGCCGTATATGGGCATTG | |
| | CGGAAAGCATTTGGGGCGACATTCACCCGCTGCTGTTCTTTGATCGTG | |
| | AGCACGCGGAAATCACCTTCAGCCGTGCGAGCGTGCAGGAGATTTTTC | |
| | TGGTTGACAGCGCGCACACCCACCGTAAGACCGTGAGCTTCCTGACCC | |
| | GTAACACCGCGATCAGCAGCATCCGTCGTCGTCTGGAAGTGACCTTTG | |
| | AGAGCCACGAAGTGATCCACGTTCGTGCGGTGGAAGACGTTGCGCGTG | |
| | ATTATAAAAGCAAAGGTGATAAACCGGTGTATGAATGGGATGGCCAA | |
| | TACACCCGTTATCACGCGGGTCCGGCGAGCGCGCCGAGCCCGGCGGCC | |
| | GC | |
| 2.6 | Tp0751 ΔLoop 2 ISPNS→TprK Loop 1 KYRGEGNVYA (SEQ ID NO: 33) | |
| | Tp0751 Expanded ΔLoop 8 LKIGSTSM→TprK Loop 3 Truncation 1 | |
| | DYKSKGDKPVYE (SEQ ID NO: 21) | |
| | GTGCAGACCGCGATGCGTATCGCGCTGTGGAACCGTGCGACCCACGGT | 188 |
| | GAACAAGGTGCGCTGCAACACCTGCTGGCGGGTCTGTGGATTCAGACC | |
| | GAAAAATATCGTGGCGAAGGCAACGTGTATGCGGGCGACATTCACCC | |
| | GCTGCTGTTCTTTGATCGTGAGCACGCGGAAATCACCTTCAGCCGTGC | |
| | GAGCGTGCAGGAGATTTTTCTGGTTGACAGCGCGCACACCCACCGTAA | |
| | GACCGTGAGCTTCCTGACCCGTAACACCGCGATCAGCAGCATCCGTCG | |
| | TCGTCTGGAAGTGACCTTTGAGAGCCACGAAGTGATCCACGTTCGTGC | |
| | GGTGGAAGACGTTGCGCGTGATTATAAAAGCAAAGGTGATAAACCGG | |
| | TGTATGAATGGGATGGCCAATACACCCGTTATCACGCGGGTCCGGCGA | |
| | GCGCGCCGAGCCCGGCGGCCGC | |
| 2.7 | Tp0751 ΔLoop 2 ISPNS→TprK Loop 2 AKTIEATLH (SEQ ID NO: 19) | |
| | Tp0751 Expanded ΔLoop 8 LKIGSTSM→TprK Loop 3 Truncation 1 | |
| | DYKSKGDKPVYE (SEQ ID NO: 21) | |
| | GTGCAGACCGCGATGCGTATCGCGCTGTGGAACCGTGCGACCCACGGT | 189 |
| | GAACAAGGTGCGCTGCAACACCTGCTGGCGGGTCTGTGGATTCAGACC | |
| | GAAGCGAAAACCATTGAAGCGACCCTGCATGGCGACATTCACCCGCTG | |
| | CTGTTCTTTGATCGTGAGCACGCGGAAATCACCTTCAGCCGTGCGAGC | |
| | GTGCAGGAGATTTTTCTGGTTGACAGCGCGCACACCCACCGTAAGACC | |
| | GTGAGCTTCCTGACCCGTAACACCGCGATCAGCAGCATCCGTCGTCGT | |
| | CTGGAAGTGACCTTTGAGAGCCACGAAGTGATCCACGTTCGTGCGGTG | |
| | GAAGACGTTGCGCGTGATTATAAAAGCAAAGGTGATAAACCGGTGTAT | |
| | GAATGGGATGGCCAATACACCCGTTATCACGCGGGTCCGGCGAGCGCG | |
| | CCGAGCCCGGCGGCCGC | |
| 2.8 | Tp0751 ΔLoop 2 ISPNS→TprK Loop 10 Truncation 1 GKLQENSNVV (SEQ | |
| | ID NO: 34) | |
| | Tp0751 Expanded ΔLoop 8 LKIGSTSM→TprK Loop 3 Truncation 1 | |
| | DYKSKGDKPVYE (SEQ ID NO: 21) | |
| | GTGCAGACCGCGATGCGTATCGCGCTGTGGAACCGTGCGACCCACGGT | 190 |
| | GAACAAGGTGCGCTGCAACACCTGCTGGCGGGTCTGTGGATTCAGACC | |
| | GAAGGCAAACTGCAGGAAAACAGCAACGTGGTGGGCGACATTCACCC | |
| | GCTGCTGTTCTTTGATCGTGAGCACGCGGAAATCACCTTCAGCCGTGC | |
| | GAGCGTGCAGGAGATTTTTCTGGTTGACAGCGCGCACACCCACCGTAA | |
| | GACCGTGAGCTTCCTGACCCGTAACACCGCGATCAGCAGCATCCGTCG | |
| | TCGTCTGGAAGTGACCTTTGAGAGCCACGAAGTGATCCACGTTCGTGC | |
| | GGTGGAAGACGTTGCGCGTGATTATAAAAGCAAAGGTGATAAACCGG | |
| | TGTATGAATGGGATGGCCAATACACCCGTTATCACGCGGGTCCGGCGA | |
| | GCGCGCCGAGCCCGGCGGCCGC | |
| 3.1 | Tp0751 ΔLoop 2 ISPNS→TprK Loop 2 AKTIEATLH (SEQ ID NO: 19) | |
| | Tp0751 Expanded ΔLoop 8 LKIGSTSM→TprK Loop 3 Truncation 1 | |
| | DYKSKGDKPVYE (SEQ ID NO: 21) | |
| | Tp0751 C-term + TprC Loop 6 GKLVQNLPNIMMPPGIT (SEQ ID NO: 18) | |
| | GTACAAACAGCAATGAGGATAGCTCTATGGAACCGTGCGACCCACGG | 191 |
| | CGAACAGGGTGCGCTGCAACATCTGTTGGCTGGCCTGTGGATTCAGAC | |
| | CGAAGCCAAAACGATTGAGGCCACGCTGCATGGTGACATCCATCCGCT | |
| | GTTGTTTTTCGACCGTGAGCACGCGGAAATCACTTTCAGCCGTGCGTCC | |
| | GTGCAAGAGATCTTTTTAGTTGATAGCGCGCACACCCACCGCAAGACG | |
| | GTGAGCTTTTTGACCCGTAATACCGCGATCTCCAGCATTCGTCGTCGCC | |
| | TGGAAGTCACCTTCGAGAGCCACGAGGTCATCCACGTTCGTGCAGTTG | |
| | AAGACGTTGCGAGAGATTATAAAAGTAAAGGCGACAAGCCGGTGTAC | |
| | GAGTGGGATGGCCAGTATACCCGCTACCATGCTGGTCCGGCATCTGCT | |
| | CCGTCGCCAGGTAAGCTGGTGCAAAACCTGCCGAACATCATGATGCCG | |
| | CCTGGTATTACC | |
| 3.2 | Tp0751 ΔLoop 2 ISPNS→TprK Loop 2 AKTIEATLH (SEQ ID NO: 19) | |
| | Tp0751 Expanded ΔLoop 8 LKIGSTSM→TprK Loop 3 Truncation 1 | |
| | DYKSKGDKPVYE (SEQ ID NO: 21) | |
| | Tp0751 AC-term AGPASAPSP→TprC Loop 6 GKLVQNLPNIMMPPGIT | |
| | (SEQ ID NO: 18) | |
| | GTACAAACAGCTATGAGGATAGCACTATGGAACCGTGCGACCCACGGT | 95 |
| | GAGCAGGGTGCTTTGCAGCATCTGTTAGCAGGCCTGTGGATTCAGACC | |
| | GAGGCGAAAACCATTGAAGCAACCCTGCATGGCGACATCCACCCGCTC | |
| | CTGTTTTTCGATCGTGAGCACGCCGAAATCACCTTCAGCCGTGCTTCGG | |
| | TGCAAGAGATCTTCTTGGTCGACAGCGCGCATACCCACCGCAAGACGG | |
| | TTTCCTTTCTGACCCGTAATACCGCGATTAGCTCTATCAGACGTCGCCT | |
| | GGAGGTGACGTTTGAGAGCCATGAAGTAATCCACGTGCGCGCCGTGGA | |
| | AGACGTTGCGCGTGATTACAAATCCAAGGGCGACAAACCGGTTTATGA | |
| | ATGGGATGGTCAATATACTCGCTACCACGGCAAGTTGGTTCAAAACCT | |
| | GCCGAACATCATGATGCCACCGGGTATTACT | |
| 3.3 | Tp0751 ΔLoop 2 ISPNS→TprK Loop 2 AKTIEATLH (SEQ ID NO: 19) | |
| | Tp0751 Expanded ΔLoop 8 LKIGSTSM→TprK Loop 3 Truncation 1 | |
| | DYKSKGDKPVYE (SEQ ID NO: 21) | |
| | Tp0751 AC-term AGPASAPSP→TprC Loop 6 Truncation 1 LVQNLPNIM | |
| | (SEQ ID NO: 35) | |
| | GTACAAACAGCTATGAGGATAGCACTATGGAACCGCGCCACGCACGG | 192 |
| | CGAGCAGGGTGCGCTGCAACATTTGCTGGCAGGCCTGTGGATCCAGAC | |
| | CGAAGCGAAAACCATTGAGGCGACTCTGCACGGCGACATCCATCCGCT | |
| | CCTGTTCTTTGATCGTGAACACGCCGAAATCACCTTCAGCCGTGCGAG | |
| | CGTTCAAGAGATCTTTCTTGTGGACAGCGCTCACACCCACCGCAAAAC | |
| | GGTGAGCTTTTTGACCAGAAACACCGCAATTTCTTCGATTCGTCGTCGC | |
| | CTGGAGGTGACCTTCGAGTCCCATGAAGTTATCCACGTTCGTGCTGTG | |
| | GAAGATGTAGCGCGTGACTACAAGTCCAAAGGTGACAAGCCGGTTTAT | |
| | GAGTGGGATGGTCAGTACACCCGTTATCATTTGGTCCAAAATCTGCCG | |
| | AACATTATG | |
| 3.4 | Tp0751 ΔLoop 2 ISPNS→TprK Loop 2 AKTIEATLH (SEQ ID NO: 19) | |
| | Tp0751 Expanded ΔLoop 8 LKIGSTSM→TprK Loop 3 Truncation 1 | |
| | DYKSKGDKPVYE (SEQ ID NO: 21) | |
| | Tp0751 AC-term AGPASAPSP→TprC Loop 6 Truncation 2 NLPNIMMPP | |
| | (SEQ ID NO: 36) | |
| | GTACAAACAGCTATGAGGATAGCACTATGGAACCGTGCGACGCACGG | 193 |
| | CGAACAGGGTGCGCTGCAACACTTGCTGGCGGGTCTGTGGATTCAGAC | |
| | CGAAGCAAAAACCATTGAGGCTACCCTGCATGGCGACATCCACCCGCT | |
| | GCTCTTCTTTGACCGCGAACATGCGGAAATTACCTTTTCTCGTGCTAGC | |
| | GTTCAAGAGATCTTCCTGGTTGATAGCGCACACACCCACCGCAAGACG | |
| | GTCAGCTTTTTGACTCGCAACACCGCCATCTCGAGCATTCGTAGACGTT | |
| | TGGAGGTTACCTTCGAGTCCCATGAAGTGATCCACGTGCGTGCGGTGG | |
| | AAGATGTGGCCCGTGACTATAAATCCAAAGGCGACAAGCCGGTTTACG | |
| | AGTGGGATGGTCAGTATACCCGTTACCATAACCTGCCGAATATCATGA | |
| | TGCCGCCA | |
| 3.5 | Tp0751 ΔLoop 2 ISPNS→TprK Loop 2 AKTIEATLH (SEQ ID NO: 19) | |
| | Tp0751 Expanded ΔLoop 8 LKIGSTSM→TprK Loop 3 Truncation 1 | |
| | DYKSKGDKPVYE (SEQ ID NO: 21) | |
| | Tp0751 AC-term AGPASAPSP→TprC Loop 6 Truncation 3 NIMMPPGIT | |
| | (SEQ ID NO: 37) | |
| | GTACAAACAGCTATGAGGATAGCACTATGGAATCGCGCGACCCACGG | 194 |
| | CGAGCAAGGTGCGCTGCAACACCTGCTCGCGGGCCTGTGGATTCAGAC | |
| | CGAGGCCAAAACCATTGAAGCGACGCTGCATGGCGACATCCACCCGCT | |
| | GTTGTTTTTCGATCGTGAGCACGCTGAAATCACCTTCTCTCGTGCTAGC | |
| | GTTCAGGAGATCTTTTTGGTGGATAGCGCACATACCCATCGTAAAACG | |
| | GTTAGCTTTCTGACCCGCAACACCGCAATTTCGAGCATCCGTCGTCGCC | |
| | TGGAAGTCACCTTCGAGTCCCATGAAGTGATCCACGTTCGTGCCGTTG | |
| | AAGACGTGGCGCGTGACTACAAGTCCAAGGGTGACAAACCGGTGTAC | |
| | GAGTGGGATGGTCAGTATACTCGCTATCACAACATCATGATGCCGCCA | |
| | GGTATTACT | |
| 3.6 | Tp0751 ΔLoop 2 ISPNS→TprK New Loop 2 ESNGGAK (SEQ ID NO: 20) | |
| | Tp0751 Expanded ΔLoop 8 LKIGSTSM→TprK Loop 3 Truncation 1 | |
| | DYKSKGDKPVYE (SEQ ID NO: 21) | |
| | Tp0751 AC-term AGPASAPSP→TprC Loop 6 GKLVQNLPNIMMPPGIT | |
| | (SEQ ID NO: 18) | |
| | GTACAAACAGCTATGAGGATAGCACTATGGAACCGTGCTACCCACGGC | 195 |
| | GAACAAGGTGCGCTGCAGCACCTGCTCGCGGGTCTGTGGATTCAGACC | |
| | GAGGAATCCAATGGTGGTGCGAAAGGCGACATCCATCCGCTGTTGTTT | |
| | TTTGATCGTGAACATGCAGAAATCACGTTCAGCCGTGCGAGCGTTCAA | |
| | GAGATCTTCTTAGTGGACAGCGCACACACCCACCGCAAGACCGTGAGC | |
| | TTTTTGACTCGCAACACCGCGATTTCGAGCATTCGTCGCCGTCTGGAGG | |
| | TTACCTTCGAGTCCCATGAAGTCATCCACGTCCGTGCTGTGGAGGACG | |
| | TGGCCCGTGATTACAAATCTAAGGGCGACAAACCGGTTTATGAGTGGG | |
| | ATGGCCAATATACCCGCTACCACGGTAAGCTGGTTCAGAATCTGCCGA | |
| | ACATCATGATGCCGCCAGGTATTACG | |
| 3.7 | Tp0751 ΔLoop 2 ISPNS→TprK Loop 10 Truncation 1 GKLQENSNVV (SEQ | |
| | ID NO: 34) | |
| | Tp0751 Expanded ΔLoop 8 LKIGSTSM→TprK Loop 3 Truncation 1 | |
| | DYKSKGDKPVYE (SEQ ID NO: 21) | |
| | Tp0751 AC-term AGPASAPSP→TprC Loop 6 GKLVQNLPNIMMPPGIT | |
| | (SEQ ID NO: 18) | |
| | GTACAAACAGCAATGAGGATAGCTCTATGGAACCGCGCGACCCACGG | 196 |
| | CGAACAGGGTGCGCTGCAGCACCTGCTTGCCGGTCTGTGGATCCAGAC | |
| | CGAAGGTAAGCTGCAAGAGAACTCCAATGTCGTGGGTGACATCCACCC | |
| | GCTCTTGTTTTTCGACCGTGAACATGCCGAGATCACGTTCAGCCGTGCT | |
| | AGCGTTCAAGAGATCTTTCTGGTGGACAGCGCACATACTCACCGTAAA | |
| | ACCGTCAGCTTTTTGACCCGCAACACCGCAATTTCTTCGATTAGACGTC | |
| | GCTTAGAGGTGACCTTCGAGAGCCATGAAGTTATTCATGTTCGTGCGG | |
| | TGGAAGATGTTGCGCGTGATTATAAATCCAAGGGCGACAAACCGGTTT | |
| | ATGAGTGGGATGGCCAGTACACCCGTTACCACGGCAAGCTGGTGCAAA | |
| | ACCTGCCGAATATCATGATGCCACCGGGTATTACG | |
| 4.1 | Tp0751 New Expanded ΔLoop 2 ISPNSGD→TprK Loop 3 Truncation 1 | |
| | DYKSKGDKPVYE (SEQ ID NO: 21) | |
| | Tp0751 Expanded ΔLoop 3 REHA→TprK New Loop 2 ESNGGAK (SEQ ID | |
| | NO: 20) | |
| | Tp0751 New Expanded ΔLoop 8 RLKIGSTSM→TprC Loop 3 | |
| | RAYSEKDTRYA (SEQ ID NO: 17) | |
| | Tp0751 AC-term AGPASAPSP→TprC Loop 6 GKLVQNLPNIMMPPGIT | |
| | (SEQ ID NO: 18) | |
| | GTACAAACAGCAATGAGGATAGCTCTATGGAACCGTGCTACCCACGGC | 96 |
| | GAACAAGGTGCGCTGCAGCACCTGCTGGCTGGCCTGTGGATTCAGACC | |
| | GAAGACTACAAGTCCAAAGGTGATAAGCCGGTGTATGAGATCCACCC | |
| | GCTGTTGTTTTTCGACGAATCTAATGGTGGTGCGAAAGAAATCACGTT | |
| | CAGCAGAGCGTCCGTACAAGAGATCTTTTTGGTTGATAGCGCACATAC | |
| | CCATCGTAAAACCGTGAGCTTTTTGACCCGCAACACCGCAATTTCTAG | |
| | CATTCGTCGCCGGCTGGAGGTGACCTTCGAGTCGCACGAGGTCATCCA | |
| | CGTTCGTGCCGTTGAGGACGTGGCCCGTGCGTATAGCGAAAAGGACAC | |
| | GCGTTACGCGTGGGATGGTCAGTATACGCGCTACCACGGTAAGCTCGT | |
| | TCAAAACCTGCCGAATATTATGATGCCACCGGGCATCACT | |
| 4.2 | Tp0751 New Expanded ΔLoop 2 ISPNSGD→TprK Loop 3 Truncation 1 | |
| | DYKSKGDKPVYE (SEQ ID NO: 21) | |
| | Tp0751 Expanded ΔLoop 3 REHA→TprK Loop 10 LQENSNVVIEKNV | |
| | (SEQ ID NO: 22) | |
| | Tp0751 New Expanded ΔLoop 8 RLKIGSTSM→TprC Loop 3 | |
| | RAYSEKDTRYA (SEQ ID NO: 17) | |
| | Tp0751 AC-term AGPASAPSP→TprC Loop 6 GKLVQNLPNIMMPPGIT | |
| | (SEQ ID NO: 18) | |
| | GTACAAACAGCTATGAGGATAGCACTATGGAACCGTGCAACGCACGG | 197 |
| | CGAGCAGGGTGCGCTGCAACATTTGCTGGCGGGTCTGTGGATTCAAAC | |
| | CGAAGACTATAAATCGAAGGGTGATAAGCCGGTCTATGAGATCCATCC | |
| | GCTGTTATTCTTTGACCTCCAAGAGAATTCTAACGTTGTGATTGAAAAG | |
| | AACGTGGAAATCACCTTTAGCCGTGCGAGCGTTCAGGAGATCTTTTTG | |
| | GTTGATAGCGCGCACACCCATCGTAAAACCGTTTCCTTCTTGACGCGTA | |
| | ACACTGCGATTTCTTCCATCCGTAGACGCCTGGAGGTGACCTTCGAAA | |
| | GCCACGAAGTCATCCACGTGCGTGCCGTTGAGGACGTAGCTCGTGCAT | |
| | ACAGCGAAAAGGACACCCGCTACGCTTGGGATGGTCAGTATACGCGCT | |
| | ACCACGGCAAACTGGTGCAGAATCTGCCGAATATCATGATGCCGCCAG | |
| | GCATTACC | |
| 4.3 | Tp0751 New Expanded ΔLoop 2 ISPNSGD→TprK Loop 10 | |
| | LQENSNVVIEKNV (SEQ ID NO: 22) | |
| | Tp0751 Expanded ΔLoop 3 REHA→TprK New Loop 2 ESNGGAK (SEQ ID | |
| | NO: 20) | |
| | Tp0751 New Expanded ΔLoop 8 RLKIGSTSM→TprK Loop 3 Truncation 1 | |
| | DYKSKGDKPVYE (SEQ ID NO: 21) | |
| | Tp0751 AC-term AGPASAPSP→TprC Loop 6 GKLVQNLPNIMMPPGIT | |
| | (SEQ ID NO: 18) | |
| | GTACAAACAGCTATGAGGATAGCACTATGGAACCGTGCGACCCACGG | 198 |
| | CGAACAGGGTGCGCTGCAACACCTGCTCGCCGGTCTGTGGATTCAGAC | |
| | GGAACTGCAAGAGAACAGCAACGTCGTGATCGAGAAGAACGTGATCC | |
| | ACCCGTTGTTGTTTTTCGACGAGTCCAATGGCGGTGCGAAAGAAATCA | |
| | CCTTCAGCCGTGCTAGCGTTCAAGAGATCTTTCTTGTGGACTCCGCGCA | |
| | CACGCATCGTAAGACCGTATCTTTTTTGACCCGCAACACCGCAATTAG | |
| | CAGCATCCGTCGCCGTCTGGAAGTGACCTTCGAGTCACATGAAGTCAT | |
| | CCACGTGCGTGCCGTTGAAGATGTTGCAGACTACAAGTCGAAAGGTGA | |
| | TAAGCCGGTTTATGAGTGGGATGGCCAGTATACCCGCTACCACGGTAA | |
| | ACTGGTTCAGAATCTGCCGAATATTATGATGCCGCCAGGTATTACT | |
| 4.4 | GTACAAACAGCAATGAGGATAGCTCTATGGAACCGCGCTACCCACGGC | 199 |
| (4B1) | GAACAGGGCGCACTGCAGCACCTCTTAGCCGGTCTGTGGATTCAAACC | |
| | GAAGACTACAAGTCGAAAGGCGATAAGCCGGTTTATGAGATCCACCC | |
| | GCTGCTGTTTTTTGACGTTAAACCGTCCTTCACCGGTGTCGAGATTACG | |
| | TTCTCTCGTGCGAGCGTGCAAGAAATCTTCTTGGTCGATAGCGCGCAC | |
| | ACCCATCGTAAGACGGTTAGCTTTTTGACCCGCAACACCGCGATTTCC | |
| | AGCATCCGTCGTCGCCTGGAAGTTACTTTCGAGTCTCACGAGGTGATC | |
| | CATGTTCGTGCTGTGGAGGACGTGGCGCGTGCGTATAGCGAAAAGGAC | |
| | ACCCGTTACGCATGGGATGGTCAGTATACCAGATACCACGGTAAACTG | |
| | GTGCAAAACCTGCCAAAT | |
| | ATCATGATGCCGCCGGGTATTACG | |
| 4.5 | GTACAAACAGCTATGAGGATAGCACTATGGAACCGTGCCACCCACGGC | 200 |
| (4B2) | GAACAGGGCGCTCTGCAGCATCTCCTGGCAGGCCTGTGGATTCAAACC | |
| | GAAGACTACAAAAGCAAGGGTGATAAACCGGTTTATGAAATCCACCC | |
| | GCTGTTGTTTTTCGACGAGTCTGGTTTTAACTTCTCGGAGATCACGTTC | |
| | TCTCGTGCTAGCGTTCAAGAGATCTTTTTAGTCGACAGCGCACATACCC | |
| | ACCGTAAAACCGTTAGCTTTTTGACCCGTAATACCGCGATTAGCAGCA | |
| | TTCGTCGCCGTCTGGAAGTAACGTTCGAGTCCCATGAAGTGATCCACG | |
| | TGAGAGCGGTGGAAGATGTTGCGCGTGCGTATTCCGAGAAGGACACTC | |
| | GCTACGCGTGGGATGGTCAGTATACCCGCTACCACGGCAAGCTGGTGC | |
| | AAAACCTGCCGAATATCATGATGCCGCCAGGTATTACC | |
| 4.6 | GTACAAACAGCTATGAGGATAGCACTATGGAACCGTGCGACGCACGG | 201 |
| (4B3) | CGAACAAGGTGCTCTGCAGCACCTGCTGGCAGGCCTCTGGATCCAGAC | |
| | CGAAGACTACAAATCGAAGGGTGATAAACCGGTTTACGAGATCCACCC | |
| | GCTGTTATTTTTCGACGGCAACGAAATCAAGGACTCTGAGATCACTTTC | |
| | AGCCGTGCCTCTGTGCAAGAGATCTTTTTGGTCGATAGCGCACATACG | |
| | CATCGTAAGACCGTGTCCTTTTTGACCCGTAATACCGCTATCAGCTCCA | |
| | TTCGTCGTCGCCTGGAGGTGACCTTCGAGAGCCATGAGGTTATTCACG | |
| | TTCGTGCGGTTGAAGACGTCGCGAGAGCGTATAGCGAAAAAGATACCC | |
| | GCTACGCGTGGGATGGCCAGTATACCCGCTATCACGGTAAACTGGTGC | |
| | AAAACCTGCCGAATATTATGATGCCGCCAGGTATTACC | |
| 5.1 | GCTCCAGTATATAGGCCCCAAGATCACGCAGCGACCATTAACGGCGTT | 202 |
| (5A1) | TCGGACCAGGGTGCGCTGCAACACCTGCTGGCAGGCTTATGGATTCAA | |
| | ACGGAAGACTACAAGTCCAAAGGTGATAAACCGGTTTATGAAATCCAC | |
| | CCGCTGCTGTTTTTCGACGAGTCTAACGGCGGCGCTAAAGAGATCACG | |
| | TTCAGCCGTGCGAGCGTTCAAGAGATCTTTTTGGTAGATAGCGCCCAC | |
| | ACCCATCGTAAGACCGTGTCCTTTTTGACCCGCAACACCGCGATCTCTT | |
| | CCATTCGTAGACGCCTGGAAGTCACCTTCGAGAGCCATGAAGTGATCC | |
| | ACGTTCGTGCTGTGGAAGATGTCGCACGTGCGTATAGCGAGAAGGACA | |
| | CTCGCTACGCCTGGGATGGTCAGTATACCCGTTACCACGGTAAACTCG | |
| | TGCAGAATCTGCCGAATATTATGATGCCACCGGGTATTACT | |
| 5.2 | GTAAATGTTTCAAACCTACAAGTCGATCACCTGTGGAAACGTCAGGGT | 203 |
| (5A2) | GCTCTGCAACACCTGCTCGCAGGTCTGTGGATTCAAACCGAAGACTAC | |
| | AAGTCGAAAGGCGACAAGCCGGTTTATGAGATCCACCCGCTGTTGTTT | |
| | TTCGATGAGTCTAATGGCGGTGCGAAAGAAATCACGTTCAGCCGTGCC | |
| | AGTGTTCAGGAGATCTTTTTAGTTGATAGCGCGCATACCCATCGTAAG | |
| | ACCGTCTCCTTTTTGACTCGTAACACCGCAATTAGCAGCATTCGTCGCC | |
| | GTCTGGAGGTGACCTTCGAGTCCCATGAAGTGATCCACGTACGCGCGG | |
| | TGGAAGACGTGGCGCGTGCGTATAGCGAAAAGGACACCCGCTACGCTT | |
| | GGGATGGTCAGTATACCAGATACCACGGCAAATTGGTTCAAAACCTGC | |
| | CGAACATTATGATGCCGCCAGGCATCACG | |
| 5.3 | GTAGATTTTCCACTAAATCCCAACACAAGGTTCCTGGCAGGCAAGCTG | 204 |
| (5A3) | CCGGCTGAAACGCAGGGTGCGCTGCAGCACCTGCTGGCGGGCCTCTGG | |
| | ATTCAGACCGAAGACTACAAATCTAAGGGTGATAAGCCGGTTTATGAG | |
| | ATCCACCCGCTGTTGTTTTTCGACGAGAGCAATGGCGGTGCGAAAGAA | |
| | ATCACTTTCAGCCGTGCTTCCGTGCAAGAAATCTTTTTAGTAGACTCTG | |
| | CGCATACCCATCGTAAAACCGTTAGCTTTTTGACTAGAAACACCGCAA | |
| | TTAGCTCCATCCGTCGCCGTTTGGAGGTCACCTTCGAGTCGCACGAAGT | |
| | GATTCACGTGCGCGCAGTTGAGGACGTGGCCCGTGCGTATAGCGAGAA | |
| | AGATACCCGCTACGCGTGGGATGGTCAATATACGCGTTACCACGGTAA | |
| | GCTGGTTCAAAACCTGCCGAACATCATGATGCCACCGGGCATTACC | |
| 5.4 | AAAAATGGGGCTGGATCAACAGATCTAAAGTGGGAACAGTTCTTACA | 205 |
| (5A4) | GAAATACCAGGGTGCGCTGCAACACCTGCTCGCGGGTCTGTGGATTCA | |
| | GACCGAAGACTACAAATCCAAAGGCGATAAGCCGGTTTATGAGATCC | |
| | ACCCGCTGTTGTTTTTCGATGAATCCAACGGCGGCGCGAAAGAGATCA | |
| | CGTTCAGCAGAGCCTCGGTGCAAGAGATCTTTCTGGTGGATAGCGCAC | |
| | ACACCCATCGTAAGACCGTGAGCTTCTTGACCCGCAACACCGCTATTT | |
| | CTAGCATTCGTCGTCGCTTGGAGGTCACCTTTGAAAGTCATGAAGTCAT | |
| | CCACGTTCGTGCTGTGGAGGACGTTGCGCGTGCGTATAGCGAAAAGGA | |
| | CACCCGTTACGCATGGGACGGCCAGTATACGCGCTACCACGGTAAGCT | |
| | GGTTCAAAACCTGCCGAATATCATGATGCCGCCAGGTATTACT | |
| 5.5 | GCTAATAGGTGGATAAGTGGACCCCACTGGTGGAACAGCCTGAGCTCT | 206 |
| (5A5) | CAGGGCGCGCTGCAACACCTGCTGGCAGGCTTATGGATTCAGACCGAA | |
| | GACTACAAAAGCAAAGGTGATAAACCGGTTTATGAGATCCACCCGCTG | |
| | TTGTTTTTCGATGAAAGTAATGGCGGTGCGAAAGAGATCACGTTCAGC | |
| | CGTGCGTCCGTGCAAGAAATCTTTCTCGTGGATAGCGCTCACACCCAT | |
| | CGTAAGACTGTAAGCTTTTTGACCCGCAACACCGCAATTAGCTCCATTC | |
| | GTCGCCGTCTGGAAGTTACCTTCGAGTCCCATGAGGTGATCCACGTGC | |
| | GTGCCGTTGAAGACGTTGCGCGTGCGTATTCTGAGAAGGACACCCGCT | |
| | ACGCTTGGGACGGCCAGTATACCAGATACCACGGTAAGCTGGTCCAAA | |
| | ACCTGCCGAATATCATGATGCCGCCAGGTATTACG | |
| 5.6 | GTACAAACAGCTATGAGGATAGCACTATGGAACCGTGCTACCCACGGC | 207 |
| (5B1) | AGCTCGGAGGCGGCGGCTAAGACGCTGGCGGGTCTGTGGATTCAGACC | |
| | GAAGACTACAAGTCCAAAGGTGATAAACCGGTTTATGAAATCCACCCG | |
| | CTGTTGTTTTTCGACGAAAGCAATGGTGGCGCAAAGGAAATCACGTTC | |
| | TCTCGTGCGTCCGTGCAAGAGATCTTCTTAGTGGACTCCGCACACACCC | |
| | ATCGTAAAACCGTTAGCTTTTTGACCCGTAATACCGCAATTAGCAGCA | |
| | TCAGACGTCGCCTGGAGGTCACCTTTGAATCTCATGAGGTTATTCACGT | |
| | GCGTGCCGTCGAGGACGTGGCGCGTGCGTATAGCGAGAAAGATACGC | |
| | GCTACGCCTGGGATGGCCAGTATACTCGCTACCACGGTAAGCTGGTTC | |
| | AAAACCTGCCGAACATCATGATGCCACCGGGTATTACC | |
| 5.7 | GTACAAACAGCTATGAGGATAGCACTATGGAACCGCGCTACCCACGGC | 208 |
| (5B2) | GTGAAGCCGAGCTTCACCGGTGTTCTGGCGGGTCTGTGGATTCAGACC | |
| | GAGGACTATAAGTCTAAAGGTGATAAACCGGTGTATGAAATCCACCCG | |
| | CTGCTGTTTTTTGACGAATCTAATGGCGGCGCGAAAGAGATCACGTTC | |
| | AGCCGTGCGAGCGTTCAAGAGATCTTTTTGGTTGATAGCGCACATACT | |
| | CACCGTAAGACTGTATCGTTCTTGACCAGAAACACCGCAATTTCCAGC | |
| | ATTCGTCGCCGTCTGGAGGTGACCTTCGAGTCCCATGAAGTCATCCAC | |
| | GTGCGTGCTGTGGAAGACGTTGCGCGTGCCTACAGCGAAAAGGACACC | |
| | CGTTACGCGTGGGATGGTCAGTATACCCGCTACCACGGTAAACTGGTT | |
| | CAAAACTTACCGAATATCATGATGCCACCGGGTATTACG | |
| 5.8 | GTACAAACAGCTATGAGGATAGCACTATGGAACCGTGCTACCCACGGC | 209 |
| (5B3) | CCGGAATCCGGTTTTAACTTCTCTCCGCTGGCAGGCCTGTGGATTCAGA | |
| | CCGAGGACTACAAATCCAAGGGTGATAAACCGGTTTATGAAATCCACC | |
| | CGCTCTTGTTTTTCGATGAGTCTAATGGTGGCGCAAAAGAGATCACGTT | |
| | CAGCCGTGCGAGCGTTCAAGAGATCTTTCTGGTCGACAGCGCGCATAC | |
| | CCATCGTAAAACCGTTAGCTTTTTGACCCGCAACACCGCGATTAGCTC | |
| | GATCCGTCGTAGACTGGAAGTCACCTTCGAGTCCCACGAGGTGATCCA | |
| | CGTGCGTGCGGTTGAAGACGTGGCCCGTGCGTATAGCGAAAAGGACA | |
| | CGCGCTACGCTTGGGATGGTCAGTATACCCGCTACCACGGTAAGCTGG | |
| | TGCAAAACCTGCCGAATATTATGATGCCACCGGGTATTACT | |
| 5.9 | GTACAAACAGCAATGAGGATAGCTCTATGGAACCGCGCTACGCACGG | 210 |
| (5C1) | CGAGCAGGGTGCTCTGCAACACCTGTTGGCGGGTCTGTGGATTCAAAC | |
| | CGAAGATTATAAATCTAAGGGTGATAAACCGGTTTATGAAATCCACCC | |
| | GTTGTTGTTTTTCGACGAAAGCAACGGCGGCGCGAAAGAGATCACGTT | |
| | CAGCCGTGGTAACGAAATCAAGGACTCTGTACAAGAGATCTTCCTGGT | |
| | GGATTCCGCGCACACCCACCGTAAGACGGTGAGCTTTCTGACCCGTAA | |
| | TACCGCAATTAGCTCCATTCGTAGACGTCTCGAGGTGACCTTTGAGAG | |
| | CCATGAAGTGATTCATGTTCGTGCCGTCGAGGACGTTGCACGTGCGTA | |
| | TTCGGAAAAGGACACCCGCTACGCGTGGGATGGTCAGTACACCCGCTA | |
| | CCACGGTAAACTGGTTCAGAATCTGCCGAACATCATGATGCCACCGGG | |
| | TATTACT | |
| 5.10 | GTACAAACAGCAATGAGGATAGCTCTATGGAACCGTGCTACCCACGGC | 211 |
| (5D1) | GAACAGGGTGCTCTGCAGCACCTGCTTGCCGGTCTGTGGATCCAAACC | |
| | GAGGACTACAAAAGCAAAGGTGACAAACCGGTTTATGAAATCCACCC | |
| | GCTGTTGTTTTTCGACGAGTCTAACGGCGGTGCGAAAGAGATCACGTT | |
| | CAGCCGTGCGTCTGTTCAAGAGATCTTCTTGGTGGATAGCGCGCATAC | |
| | TCACGGCAATGAAATCAAGGACAGCACCGTTTCCTTTTTGACCCGCAA | |
| | CACCGCAATTAGCTCCATTCGTCGCCGCCTGGAGGTCACCTTTGAAAG | |
| | TCATGAAGTGATCCACGTGCGTGCCGTCGAGGATGTTGCACGTGCGTA | |
| | TTCGGAAAAGGATACCAGATACGCGTGGGATGGTCAGTATACCCGTTA | |
| | CCACGGTAAGCTGGTGCAAAACCTGCCGAATATTATGATGCCGCCAGG | |
| | TATTACG | |
| 5.11 | GTACAAACAGCTATGAGGATAGCACTATGGAACCGCGCGACCCACGG | 212 |
| (5E1) | CGAACAGGGCGCGCTGCAGCACCTGCTGGCAGGTCTGTGGATTCAGAC | |
| | CGAAGACTACAAGTCGAAAGGTGACAAACCGGTTTATGAAATCCACCC | |
| | GCTCTTATTTTTCGATGAGTCCAACGGCGGTGCGAAAGAGATCACTTTC | |
| | TCTCGTGCGAGCGTTCAAGAGATCTTTTTGGTCGATAGCGCACATACCC | |
| | ACCGTAAGACCGTGAGCTTTTTGACCCGCAACAGCACGGGTGACGAGC | |
| | AACAAGGTACAATTCGTAGACGTCTGGAGGTGACCTTCGAGTCCCATG | |
| | AAGTCATCCACGTTCGTGCCGTTGAAGACGTGGCTCGTGCGTATAGCG | |
| | AAAAAGATACCCGCTACGCTTGGGATGGCCAGTATACCCGTTACCACG | |
| | GTAAGCTGGTGCAGAATCTGCCGAATATTATGATGCCGCCAGGTATTA | |
| | CG | |
| 5.12 | GTACAAACAGCAATGAGGATAGCTCTATGGAACCGTGCGACCCACGG | 213 |
| (5E2) | CGAACAAGGTGCGCTGCAGCATCTGCTGGCAGGTCTGTGGATTCAGAC | |
| | CGAAGACTACAAGAGTAAAGGCGATAAGCCGGTGTATGAAATCCACC | |
| | CGTTGCTCTTTTTTGATGAATCCAACGGCGGTGCTAAAGAGATCACGTT | |
| | CAGCCGTGCGAGCGTTCAAGAGATCTTCTTAGTCGATAGCGCACACAC | |
| | CCATCGTAAAACTGTAAGCTTTTTGACCCGCAACGCTGTCGGTAAGGA | |
| | CATCGAGGGTCAGATTCGTCGCCGTCTGGAAGTTACCTTCGAGTCTCA | |
| | CGAGGTTATCCATGTTCGTGCGGTGGAGGACGTGGCCCGTGCGTATTC | |
| | GGAAAAAGACACCCGCTACGCCTGGGATGGCCAATATACCAGATACC | |
| | ACGGCAAGCTGGTGCAGAATCTGCCGAATATTATGATGCCGCCAGGTA | |
| | TTACG | |
| 5.13 | GTACAAACAGCTATGAGGATAGCACTATGGAACCGTGCTACCCACGGC | 214 |
| (5E3) | GAACAGGGTGCGCTGCAGCATCTGCTGGCGGGTCTGTGGATTCAGACC | |
| | GAAGACTACAAGTCCAAGGGCGATAAACCGGTTTATGAAATCCACCCG | |
| | CTGTTGTTTTTCGACGAGTCGAACGGCGGTGCGAAAGAGATCACGTTC | |
| | AGCCGTGCTTCTGTTCAAGAGATCTTTCTTGTGGATAGCGCACATACCC | |
| | ACCGCAAGACGGTCAGCTTTTTGACCCGTAATTTCGGCCAACGTAAGG | |
| | AGGACATCCGTCGCCGTCTGGAAGTTACCTTCGAGAGCCACGAAGTGA | |
| | TTCACGTGCGTGCCGTTGAAGACGTCGCGAGAGCGTATTCCGAGAAAG | |
| | ATACCCGCTATGCATGGGATGGTCAGTACACCCGCTACCACGGTAAAC | |
| | TGGTGCAAAACCTCCCGAATATTATGATGCCGCCAGGTATTACT | |
| 5.14 | GTACAAACAGCAATGAGGATAGCTCTATGGAACAGAGCTACCCACGG | 215 |
| (5E4) | CGAACAGGGCGCGCTGCAACATCTGCTTGCCGGTCTGTGGATTCAAAC | |
| | GGAGGACTACAAGTCCAAAGGTGATAAACCGGTTTATGAAATCCACCC | |
| | GCTGCTGTTTTTCGATGAAAGCAATGGCGGTGCAAAAGAGATCACGTT | |
| | TAGCCGTGCGTCTGTTCAAGAGATCTTTCTCGTGGACAGCGCACACAC | |
| | CCACCGTAAAACCGTTAGCTTCTTGACCCGCAACGTGAAGCCGTCGTT | |
| | CACCGGTGTTATTCGTCGTCGCTTGGAGGTGACCTTCGAGTCCCATGAA | |
| | GTGATCCACGTTCGTGCTGTGGAAGACGTCGCGCGTGCGTATAGCGAA | |
| | AAGGACACTCGCTACGCGTGGGATGGTCAGTATACCCGCTACCACGGT | |
| | AAGCTGGTCCAGAATCTGCCGAACATTATGATGCCGCCAGGTATTACC | |
| 5.15 | GTACAAACAGCTATGAGGATAGCACTATGGAACAGAGCTACCCACGG | 216 |
| (5E5) | CGAACAAGGTGCGCTGCAACATCTGTTGGCGGGTCTGTGGATCCAGAC | |
| | CGAGGACTACAAAAGCAAAGGTGATAAGCCGGTCTATGAAATCCACC | |
| | CGCTGCTCTTCTTTGATGAATCCAACGGTGGCGCGAAAGAGATCACGT | |
| | TCAGCCGTGCGTCGGTTCAAGAGATCTTTTTGGTGGACAGCGCACACA | |
| | CCCACCGTAAGACCGTTAGCTTTTTGACCCGTAACGGCAACGAGATCA | |
| | AAGATTCTATTCGTCGTCGCCTGGAAGTCACCTTCGAGTCCCATGAGGT | |
| | GATTCATGTTCGTGCTGTGGAAGACGTGGCCCGTGCGTATAGCGAAAA | |
| | GGACACTCGCTACGCATGGGATGGCCAGTACACCCGCTATCACGGCAA | |
| | GCTGGTTCAGAATCTGCCGAATATTATGATGCCGCCAGGTATTACG | |

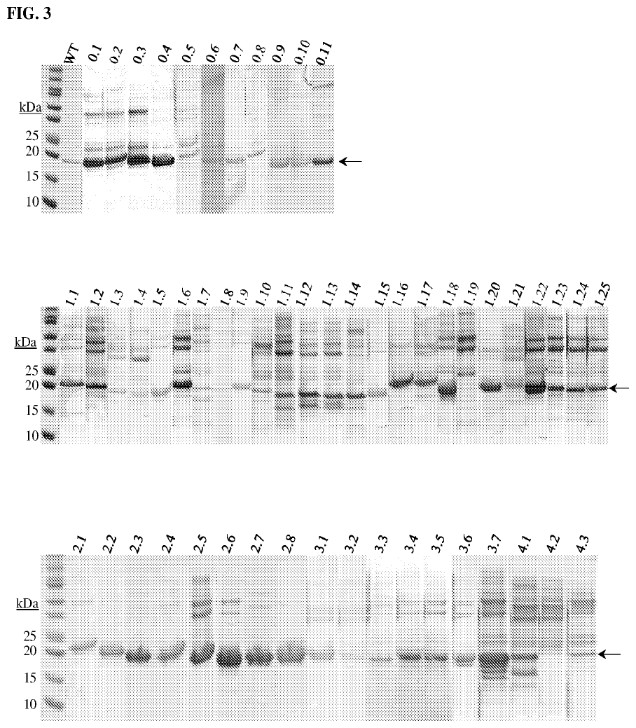

Following overnight growth, 50 mL of 2×YT media was inoculated with 0.5 mL of starter culture and grown at 37° C. until an OD₆₀₀ of 1.6-1.8 was reached. 2 mL of culture was removed as a negative control, and cultures were then induced with 0.4 mM IPTG and left to express overnight at 16° C. Following expression, 2 mL of culture was removed and purified using Ni-affinity chromatography. Samples taken from multiple stages of purification (insoluble pellet, lysis supernatant, purified sample) were evaluated on a 12% Bolt 12% Bis-Tris Plus gel at 200 V for 30 minutes, and expression was assessed by the presence or absence of a strong band at the expected molecular weight (FIG. 3). Chimeric constructs that showed expression equivalent to wild type Tp0751 (99-237) and that could be enriched by nickel pulldown indicated correctly folded protein and were advanced to large scale expression trials.

### Example 4

### Protein Stability

Following small-scale expression trials, Tp0751 (99-237) constructs (Tables 3-4; SEQ ID NOS: 38-91 and 97-114) were expressed in E. coli BL21 DE3 cells and grown in 50 mL of 2×YT media overnight at 37° C. Following overnight growth, 2 L of 2×YT media was inoculated with 2 mL of starter culture and grown at 37° C. until an OD₆₀₀ of 1.6-1.8 was reached. Cultures were then induced with 0.4 mM IPTG and grown overnight at 16° C. Following overnight expression, cells were lysed using lysis buffer (10 mM Tris-HCl pH 8.8, 130 mM NaCl, 10 mM NaF, 0.01% Triton-X-100) and protein was purified using Ni-affinity chromatography in 20 mM HEPES pH 8.0, 1.0 M NaCl, 30 mM imidazole+1% glycerol. The proteins were further purified using SEC on either an ENrich™ SEC 70 or a Superdex™ 75 column in 20 mM HEPES pH 8.0, 150 mM NaCl+1% glycerol. The final samples were flash frozen using liquid nitrogen and stored at −80° C. for future use.

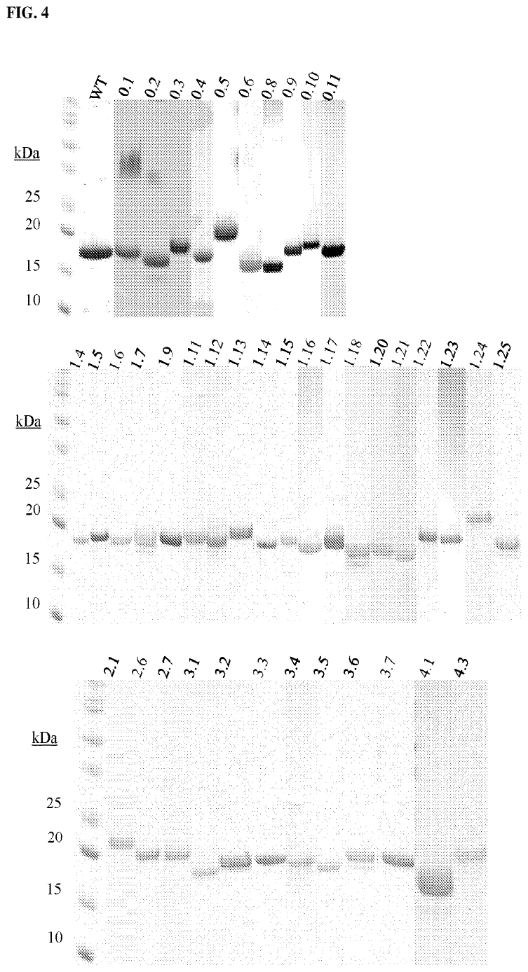

Throughout the large-scale purification, four metrics (benchmarked to wild type Tp0751 (99-237)) were used to measure prioritize chimeras; minimal protein precipitation and aggregation during concentration, remaining soluble following TEV cleavage, a monodispersed and monomeric SEC trace, and sufficient yield and purity following SEC (FIG. 4). Marked amounts of precipitation during purification indicate protein instability, signaling that the engineering strategy used for that loop was not sufficient, or that the epitope sequence used for engraftment was unstable in that specific Tp0751 loop. TEV cut proteins were monitored to ensure that solubility was retained following tag-removal. Next, each construct was assessed following SEC to ensure that a monodispersed and monomeric peak was obtained, indicating that each protein expressed as a monomer, and exhibited correct folding. The final metric of a successful purification is the purity gel following SEC (FIG. 4). High purity and minimal protein degradation are indicated when a strong single band at the expected MW is observed.

### Example 5

### Protein Characterization

Following successful large-scale purification, each purified construct was assessed for thermal stability using a thermal shift assay. Samples of each construct (Tables 3-4; SEQ ID NOS: 38-91 and 97-114) were prepared to a final concentration of 0.125 mg/mL protein and 5× SYPRO Orange dye. 20 μL of each sample was ran in triplicate using an Applied Biosystems™ StepOnePlus™ Real-Time PCR System. Protein denaturation was measured using a melt curve with a range from 25° C. to 99° C. As a general guideline, to be considered thermally stable each protein would need to display a Tm within 10% from the WT Tp0751 (99-237) Tm. This ensures that the protein modifications made during engineering did not significantly alter the structure or stability of the protein compared to WT. Tₘ data for each construct is provided in Table 6.

TABLE 6
Melting temperature (Tₘ) results for each chimeric protein purified
from large scale E. coli preps (FIG. 4). For Tₘ analysis, each protein
was prepared in a reaction mixture to a final concentration of 0.125
mg/mL protein and 5X SYPRO Orange hydrophobic binding dye.
Samples were run in triplicate on a CFX Opus 96 Real-Time PCR
machine for 60 minutes on a continuous temperature ramp
(1%) from 25 to 99° C.

| Construct Design | Purification successful? | Tₘ ± std dev (° C.) (° C.) |
|---|---|---|
| WT | Yes | 51.5 ± 0.0 |
| 0.1 | Yes | 52.3 ± 0.0 |
| 0.2 | Yes | 52.9 ± 0.2 |
| 0.3 | Yes | 50.2 ± 0.1 |
| 0.4 | Yes | 42.9 ± 0.3 |
| 0.5 | No | N/A |
| 0.6 | Yes | 49.9 ± 0.1 |
| 0.7 | Yes | 49.9 ± 0.0 |
| 0.8 | Yes | 48.0 ± 0.2 |
| 0.9 | Yes | 52.5 ± 0.0 |
| 0.10 | Yes | 52.2 ± 0.2 |
| 0.11 | Yes | 52.9 ± 0.1 |
| 1.1 | No | N/A |
| 1.2 | No | N/A |
| 1.3 | N/A | N/A |
| 1.4 | Yes | 51.7 ± 0.1 |
| 1.5 | Yes | 53.0 ± 0.1 |
| 1.6 | Yes | 51.1 ± 0.1 |
| 1.7 | Yes | 50.3 ± 0.1 |
| 1.8 | No | N/A |
| 1.9 | Yes | 49.8 ± 0.0 |
| 1.10 | No | N/A |
| 1.11 | Yes | 45.7 ± 0.2 |
| 1.12 | Yes | 51.9 ± 0.1 |
| 1.13 | Yes | 52.6 ± 0.0 |
| 1.14 | Yes | 49.9 ± 0.1 |
| 1.15 | Yes | 53.4 ± 0.1 |
| 1.16 | Yes | 48.3 ± 0.0 |
| 1.17 | Yes | 47.7 ± 0.0 |
| 1.18 | Yes | 49.8 ± 0.0 |
| 1.19 | N/A | N/A |
| 1.20 | Yes | 49.6 ± 0.0 |
| 1.21 | Yes | 51.3 ± 0.1 |
| 1.22 | Yes | 52.2 ± 0.1 |
| 1.23 | Yes | 50.3 ± 0.1 |
| 1.24 | Yes | 51.5 ± 0.0 |
| 1.25 | Yes | 50.3 ± 0.1 |
| 2.1 | Yes | 52.5 ± 0.2 |
| 2.2 | No | N/A |
| 2.3 | No | N/A |
| 2.4 | No | N/A |
| 2.5 | No | N/A |
| 2.6 | No | N/A |
| 2.7 | Yes | 50.2 ± 0.1 |
| 2.8 | Yes | 52.2 ± 0.1 |
| 3.1 | Yes | 50.7 ± 0.0 |
| 3.2 | Yes | 51.9 ± 0.1 |
| 3.3 | Yes | 52.4 ± 0.0 |
| 3.4 | Yes | 52.1 ± 0.1 |
| 3.5 | Yes | 51.4 ± 0.0 |
| 3.6 | Yes | 54.5 ± 0.1 |
| 3.7 | Yes | 53.5 ± 0.5 |
| 4.1 | Yes | 47.3 ± 0.1 |
| 4.2 | No | N/A |
| 4.3 | Yes | 48.2 ± 0.5 |

When possible, chimeras that showed sufficient thermostability were tested for antibody recognition via ELISA. Using sera raised against either N. meningitidis or T. pallidum, whether the newly engrafted loops were recognized by antibodies in sera was tested. This can allow for differentiation between candidates when deciding between proteins to be used in protection trials.

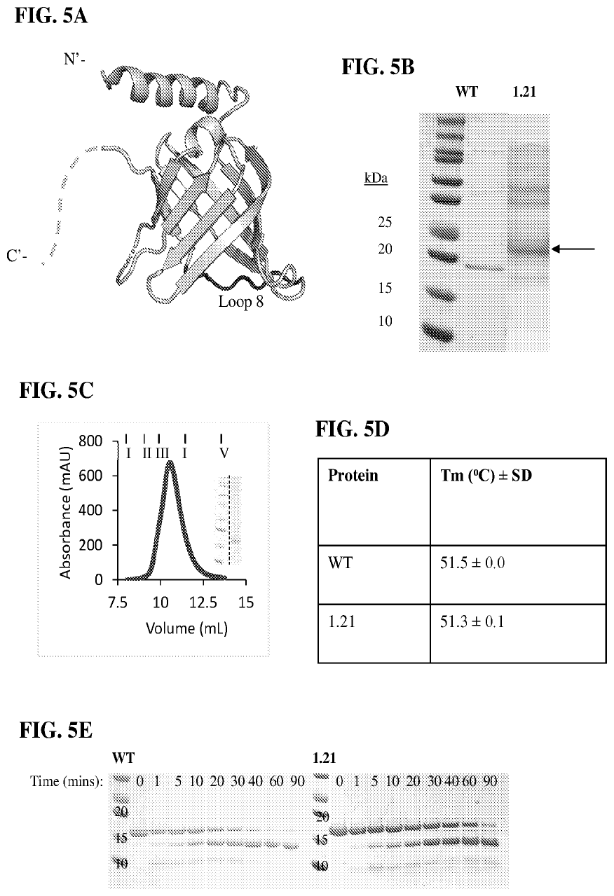

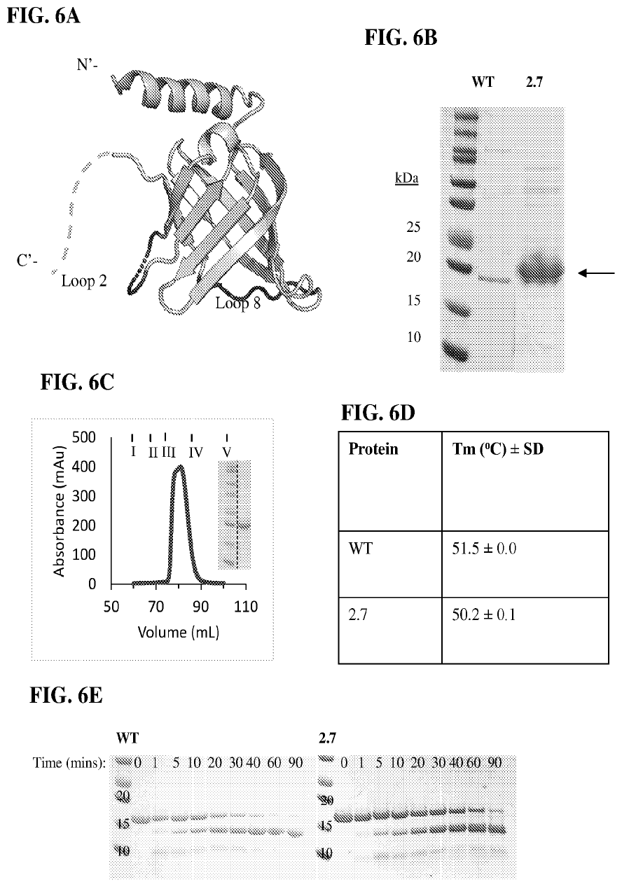

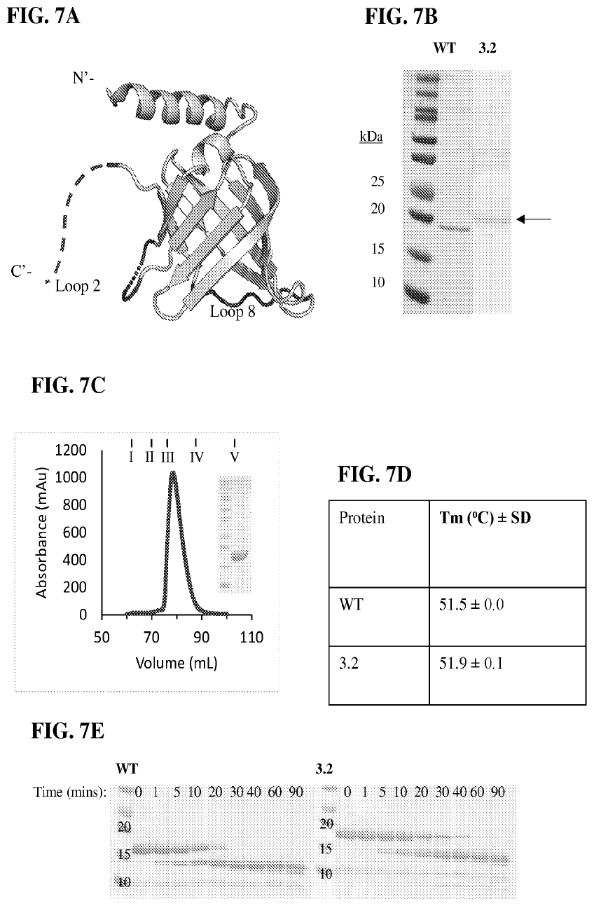

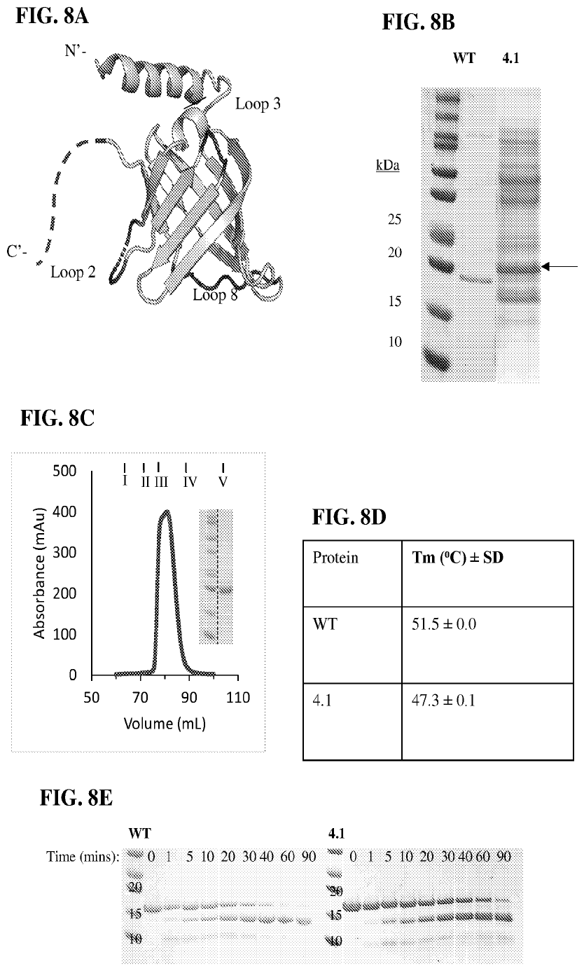

The workflow described herein reflects an iterative process where learnings that underpin prioritization of targeted loops and engrafted sequences are fed back into the engineering process to refine a Tp0751 chimera that satisfies biophysical criteria. FIGS. 5-8 show purification data sets for four engineered chimera constructs, including structural schematics of the replaced loop regions, small scale expression gels, SEC profile and purity gel, melting temperature analyses, and proteolytic stability profiles.

### Example 6

### Protective Immune Response Against T. pallidum

Using the Tp0751-TprK-TprC chimera (construct 3.2; SEQ ID NO: 82), a protection experiment was performed by immunizing 8 rabbits 5 times at three week intervals with 125 μg of the chimera emulsified in adjuvant. Three weeks following the final boost, 8 immunized rabbits and 8 unimmunized control rabbits were challenged at each of 10 sites on their shaved back with 1×10⁵ T. pallidum, Nichols strain/site. Lesion development was monitored daily, and on day 22, the challenged rabbits were euthanized, testes were collected from 3 random animals per group, and popliteal lymph nodes were transferred to the testes of naïve animals to conduct the RIT portion of the protection experiment.

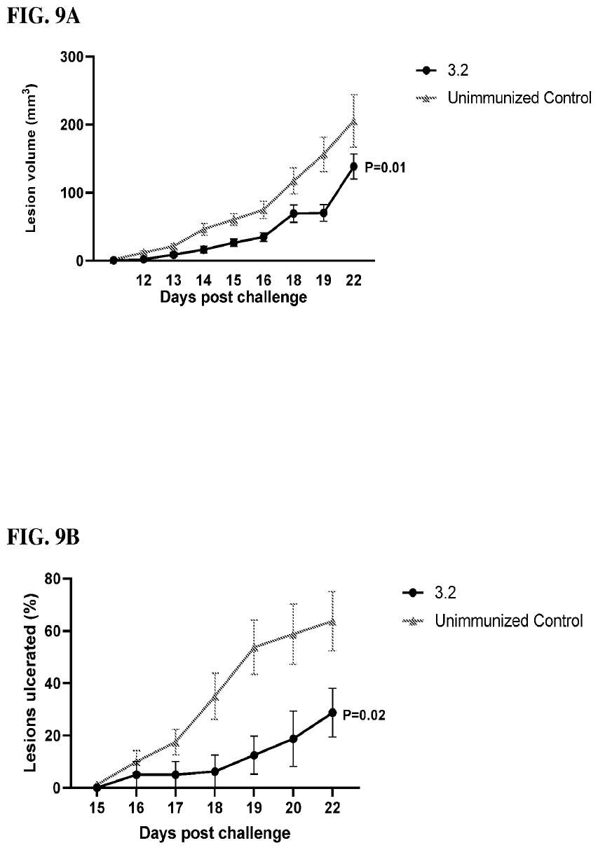

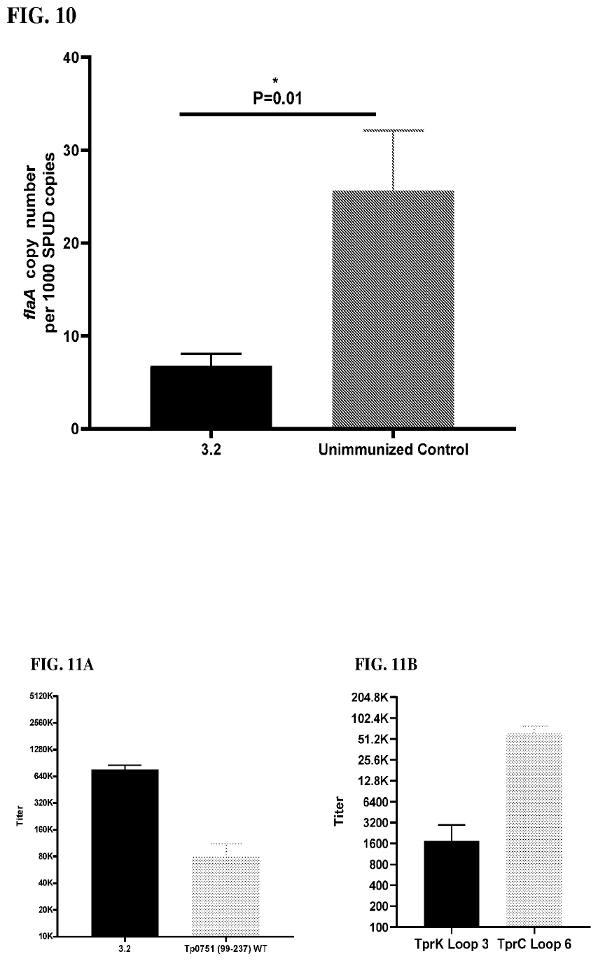

Rabbits immunized with the Tp0751-TprK-TprC chimera (construct 3.2; SEQ ID NO: 82) had significantly attenuated lesion volume (FIG. 9A) and ulceration (FIG. 9B) compared to the unimmunized control animals. Immunized animals also had a lower T. pallidum burden at the local infection site compared to unimmunized controls, as measured by qPCR analysis of lesion aspirates collected on day 19 post challenge (FIG. 10). Immunization induced a high antibody titer against Tp0751 (FIG. 11A), as well as against the engineered TprK and TprC epitopes (FIG. 11B).

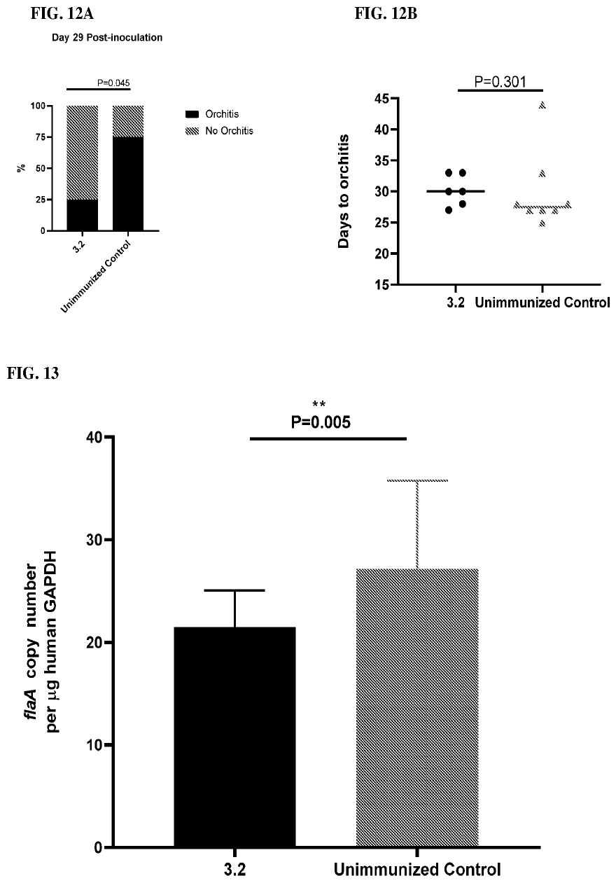

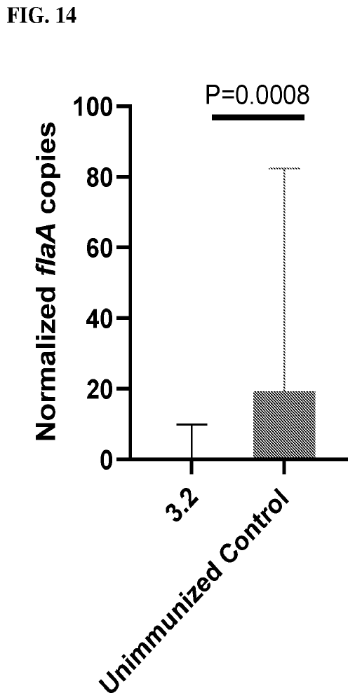

The RIT portion of the protection experiment demonstrated that 2 rabbits receiving lymph nodes from immunized animals exhibited no evidence of infection, while the remaining 6 rabbits had a median 3 day delay in orchitis development compared to rabbits receiving lymph nodes from unimmunized controls (FIGS. 12A, 12B). The testes of the immunized animals (n=3) had significantly lower burden of T. pallidum compared to the unimmunized control animals (n=3) (FIG. 14). Thus, there is a reduction of T. pallidum dissemination from the site of infection when immunizing using the disclosed chimeric proteins. Serum from chimera-immunized animals decreased attachment of T. pallidum to endothelial cells compared to serum from unimmunized controls (FIG. 13).

These results show that immunization with the Tp0751 chimeras disclosed herein partially protect rabbits from T. pallidum infection. A high titer antibody response was generated against the chimera through immunization, antibodies against the Tp0751 scaffold and the inserted epitopes was raised, and the antibodies raised are functional, as they partially inhibiting T. pallidum dissemination to distant organ sites.

### Example 7

### Immunization of Mice with New Chimera Constructs Induced an Antibody Response

To access chimera construct immunization of mice, constructs 3.2 (SEQ ID NO: 82), 3.7 (SEQ ID NO: 87), and 4.1 (SEQ ID NO: 88) were tested with adjuvants_LiT4Q, PAI-sRI-T1/2, and PAI-RIBI (PAI, Seattle). Groups of mice were immunized twice subcutaneously (4-week interval) with 20 μg/immunization of construct 3.2, 3.7 or 4.1, emulsified in either PAI-RIBI Natural, LiT4Q or PAI-sRI-T1/2 adjuvants [n=3 per construct+adjuvant or n=3 adjuvant only (control)]. The latter two adjuvants are human-track adjuvants. Antibody titers were measured using serum collected 4 weeks after the primary immunization (W4) and 2 weeks after the final immunization (W8).

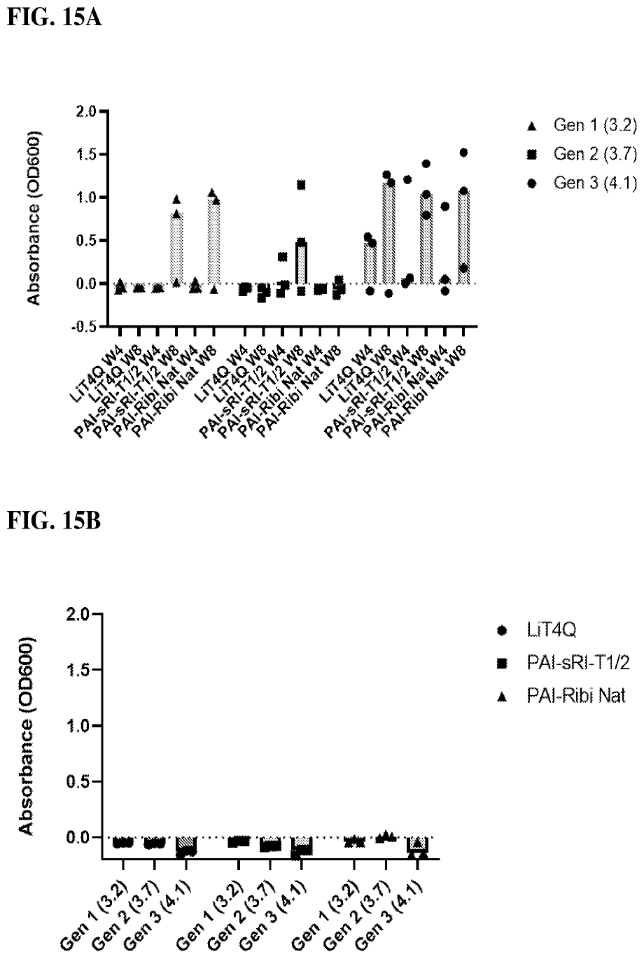

All three constructs tested induced an antibody response. Mice immunized with Construct 4.1 had the highest antibody titers compared to mice immunized with Constructs 3.2 and 3.7 (FIG. 15A). Sham-immunized (adjuvant only) mice did not produce antibodies that recognized all three constructs (FIG. 15B).

### Example 8

### New B Cell Epitopes

To extend the Tp0751 chimera design provided herein, a pool of six predicted T. pallidum OMPs that can be used as additional sources of epitopes in the chimera design were identified. All of these T. pallidum proteins were confirmed to be expressed by T. pallidum via mass spectrometry analyses of the T. pallidum proteome.

Three predicted T. pallidum OMPs demonstrated to have a pathogenesis-related function were compiled including TPANIC_0326^{36,41,42}, TPANIC_0483⁴³, and TPANIC_0856¹⁴.

Structural modeling of 264 T. pallidum proteins of unknown function (i.e., proteins with no known annotation in the NCBI database) was performed, identifying three novel potential OMPs that have molecular signatures compatible with a surface-exposed outer membrane locale (i.e., SP1 signal sequence, beta-barrel structure, and structural similarity to OMPs in other bacterial pathogens). This included TPANIC_RS01700, TPANIC_0346, and TPANIC-RS03465.

These six potential OMPs were then subjected to B cell prediction programs (ElliPro⁴⁴, ABCPred⁴⁵, BepiPred⁴⁶, SVMTriP⁴⁷) to identify predicted epitopes. From these analyses, 14 B cell epitopes from the six proteins were identified as ones that can be engineered into the Tp0751 scaffold (Table 7). That is, one or more of these 14 epitopes can be used to generate a disclosed chimeric Tp0751 (99-237) peptide, wherein one or more of SEQ ID NOS: 115-128 or 217-232 replaces all of or part of one or more loops of a native Tp0751 (99-237) peptide. Any of SEQ ID NOS: 115-128 and 217-232 can be used alone or in combination with the TprC and/or TprK epitopes provided herein.

TABLE 7
B cell epitopes for inclusion in chimeric Tp0751 (99-237) peptides.

| Protein | Predicted B Cell Epitope | Nucleotide Sequences |
|---|---|---|
| TPANIC_0483 | APVYRPQDHAATINGVSD | geg ccg gtg tat egg ceg cag gac cac gcc geg aca atc aac |
| | (SEQ ID NO: 116) | ggt gtt tct gac (SEQ ID NO: 130) |
| | PFGQRKED (SEQ ID NO: 117) | cca ttc gga cag cgc aag gaa gac (SEQ ID NO: 131) |
| | VKPSFTGV (SEQ ID NO: 118) | gta aag ccg agt ttc aca gga gtg (SEQ ID NO: 132) |
| TPANIC_RS01700 | AVGKDIEGQ (SEQ ID NO: | gcc gtg ggg aaa gac att gag ggg cag (SEQ ID NO: 137) |
| | 123) | |
| | VDFPLNPNTRFLAGKLPAET | gtg gac ttt ccg ctc aac cct aac acc cgc ttt tta gca ggt aag |
| | (SEQ ID NO: 124) | ctg cct gca gaa acg (SEQ ID NO: 138) |
| TPANIC_0346 | YGNEIKDS (SEQ ID NO: 125) | tat ggg aac gag ata aag gac tcc (SEQ ID NO: 139) |
| | ANRWISGPHWWNSLSS | gct aat cgc tgg ata tca ggt ccc cac tgg tgg aat tct ctt tct tcg |
| | (SEQ ID NO: 126) | (SEQ ID NO: 140) |
| TPANIC_RS03465 | KGANGEGGTQGTKRTDIL | aag ggc gcg aac ggt gag ggc gga acg caa ggt acg aaa cgc |
| | (SEQ ID NO: 127) | acc gat ata ctg (SEQ ID NO: 141) |
| | KNGAGSTDLKWEQFLQKY | aag aac ggc gca ggc tcc act gat ttg aaa tgg gaa cag ttt ttg |
| | (SEQ ID NO: 128) | caa aag tat (SEQ ID NO: 142) |
| TPANIC_0304 | GANGPHYYKSLESP (SEQ ID | ggc gca aac gga cca cat tat tac aag age ctt gaa age ccc |
| | NO: 217) | (SEQ ID NO: 233) |
| | PVYGFPGPTYLPKLAG (SEQ | cca gtg tac ggc ttt ccc ggt cct acg tat tta ccc aag ctc gca |
| | ID NO: 218) | ggg (SEQ ID NO: 234) |
| | PDFSSPKHL (SEQ ID NO: | cca gac ttt tcc tea ccc aaa cac ctg (SEQ ID NO: 235) |
| | 219) | |
| | QLAETQSATT (SEQ ID NO: | caa ctt gca gag acg cag age gcc aca acc (SEQ ID NO: |
| | 220) | 236) |
| TPANIC_0619 | PTMPDTENQAPP (SEQ ID | ccc aca atg cca gat aca gag aac cag geg cet ccg (SEQ ID |
| | NO: 221) | NO: 237) |
| | SSSYSAGNWHRPRSLL (SEQ | agc agc tcg tct tat tca gcc ggt aac tgg cac cgc cca cgt tca |
| | ID NO: 222) | ttg ctt (SEQ ID NO: 238) |
| | TKLAKFRI (SEQ ID NO: 223) | acc aag ctg get aag ttc cgc atc (SEQ ID NO: 239) |
| TPANIC_RS05525 | GNTYRDTNSARARVVE | gga aat acc tac cgg gat cg aac agt geg aga gca cgt gtc gtc |
| | (SEQ ID NO: 224) | gaa (SEQ ID NO: 240) |
| | SVFGSGPFVKSTFQY (SEQ | tct gtt ttt ggg agt ggt ccc ttt gta aag tca act ttt caa tac (SEQ |
| | ID NO: 225) | ID NO: 241) |
| | NFVARAAGLPADAIATGAAS | aac ttt gta gca cgc gog gca ggg tta ccg gca gac gog ata gca |
| | (SEQ ID NO: 226) | aca ggc gca gct tca (SEQ ID NO: 242) |
| | RFNFNGFKGTRNLKG (SEQ | cga ttc aat ttc aat ggg ttc aaa gga acg cgc aat tta aag gga |
| | ID NO: 227) | (SEQ ID NO: 243) |
| | NWSWPGYT (SEQ ID NO: | aac tgg agc tgg cct gga tac acc (SEQ ID NO: 244) |
| | 228) | |
| TPANIC_RS05460 | GLVQYRPRGSGGVAA (SEQ | gga ctg gtg caa tac cgc ccg cgg gga tct ggg ggc gtt gca gcc |
| | ID NO: 229) | (SEQ ID NO: 245) |
| | SYPFYYEKERL (SEQ ID NO: | agt tat ccg ttc tat tac gag aag gag cgg ctg (SEQ ID NO: |
| | 230) | 246) |
| | EKVVEEEREQTNTR (SEQ ID | gaa aaa gtg gtt gaa gaa gag aga gag cag act aac acg cgc |
| | NO: 231) | (SEQ ID NO: 247) |
| TPANIC_0326 | KQTVVSRIQFKGNKAF (SEQ | aag cag act gtt gtc tcg cgg ata cag ttt aag gga aat aag gcg |
| | ID NO: 115) | ttt (SEQ ID NO: 129) |
| | DNNQPFDLTV (SEQ ID NO: | gat aac aat cag ccc ttc gac ctg acc gta (SEQ ID NO: 248) |
| | 232) | |

### Example 9

### New T Cell Epitopes

To extend the Tp0751 chimera design provided herein, a pool of eight predicted T. pallidum OMPs that can be used as additional sources of epitopes in the chimera design were identified. Six of these T. pallidum proteins were confirmed to be expressed by T. pallidum via mass spectrometry analyses of the T. pallidum proteome.

One predicted T. pallidum OMP demonstrated to have a pathogenesis-related function was compiled (TPANIC_0483, Cameron et al., J Bacteriol 2004, 186, 7019-22).

Structural modeling of 264 T. pallidum proteins of unknown function (i.e., proteins with no known annotation in the NCBI database) was performed, identifying eight novel potential OMPs that have molecular signatures compatible with a surface-exposed outer membrane locale (i.e., SP1 signal sequence, beta-barrel structure, and structural similarity to OMPs in other bacterial pathogens). This included TPANIC_0304, TPANIC_0346, TPANIC_0483, TPANIC_0619, TPANIC_RS01700, TPANIC_RS03465, TPANIC_RS05460, and TPANIC_RS05525.

These eight potential OMPs were then subjected to T cell prediction (IEDB TepiTool—MHC Class II Predictions, see Paul et al., Curr Protoc Immunol 2016, 114, 18 19 1-18 19 24) to identify predicted epitopes. From these analyses, 64 T cell epitopes from the eight proteins were identified as ones that can be engineered into the Tp751 scaffold (Table 8). That is, one or more of these 64 epitopes can be used to generate a disclosed chimeric Tp0751 (99-237) peptide, wherein one or more of SEQ ID NOS: 249-312 replaces all of or part of one or more loops of a native Tp0751 (99-237) peptide. Any of SEQ ID NOS: 249-32 can be used alone or in combination with the TprC and/or TprK peptides provided herein.

TABLE 8
T cell epitopes for inclusion in chimeric Tp0751 (99-237) peptides.

| Protein | Predicted T Cell Epitope | SEQ ID NO: |
|---|---|---|
| Tp_0304 Loop 3 (G900-S939) 40 amino acids | AEHPVYGFPGPTYLP | 249 |
| Tp_0346 Loop 4 (G139-P220) 82 amino acids | AGFVARLIANYLLKG | 250 |
| Tp_0346 Loop 4 (G139-P220) 82 amino acids | KVAGFVARLIANYLL | 251 |
| Tp_0346 Loop 4 (G139-P220) 82 amino acids | LSSWVGNTAGKVAGF | 252 |
| Tp_0346 Loop 4 (G139-P220) 82 amino acids | PELAKILITHTVTHG | 253 |
| Tp_0346 Loop 4 (G139-P220) 82 amino acids | LAKILITHTVTHGLA | 254 |
| Tp_0346 Loop 4 (G139-P220) 82 amino acids | FVARLIANYLLKGSQ | 255 |
| Tp_0346 Loop 4 (G139-P220) 82 amino acids | NSNLRYLMRVGTPEL | 256 |
| Tp_0346 Loop 4 (G139-P220) 82 amino acids | RYLMRVGTPELAKIL | 257 |
| Tp_0346 Loop 4 (G139-P220) 82 amino acids | AKILITHTVTHGLAN | 258 |
| Tp_0346 Loop 4 (G139-P220) 82 amino acids | RLIANYLLKGSQYSM | 259 |
| Tp_0346 Loop 4 (G139-P220) 82 amino acids | WISGPHWWNSLSSWV | 260 |
| Tp_0346 Loop 4 (G139-P220) 82 amino acids | ANYLLKGSQYSMLVP | 261 |
| Tp_0346 Loop 4 (G139-P220) 82 amino acids | LRYLMRVGTPELAKI | 262 |
| Tp_0346 Loop 4 (G139-P220) 82 amino acids | NSLSSWVGNTAGKVA | 263 |
| Tp_0346 Loop 4 (G139-P220) 82 amino acids | WVGNTAGKVAGFVAR | 264 |
| Tp_0346 Loop 4 (G139-P220) 82 amino acids | GKVAGFVARLIANYL | 265 |
| Tp_0346 Loop 4 (G139-P220) 82 amino acids | GNTAGKVAGFVARLI | 266 |
| Tp_0346 Loop 4 (G139-P220) 82 amino acids | TPELAKILITHTVTH | 267 |
| Tp_0346 Loop 4 (G139-P220) 82 amino acids | GVDVPVNSNLRYLMR | 268 |
| Tp_0346 Loop 4 (G139-P220) 82 amino acids | AGKVAGFVARLIANY | 269 |
| Tp_0346 Loop 4 (G139-P220) 82 amino acids | IANYLLKGSQYSMLV | 270 |
| Tp_0346 Loop 4 (G139-P220) 82 amino acids | VAGFVARLIANYLLK | 271 |
| Tp_0346 Loop 4 (G139-P220) 82 amino acids | SNLRYLMRVGTPELA | 272 |
| Tp_0483 Loop 1 (A224-G250) 27 amino acids | RPQDHAATINGVSDF | 273 |
| Tp_0483 Loop 1 (A224-G250) 27 amino acids | INGVSDFFKICSPIG | 274 |
| Tp_0619 Loop 1 (A21-E51) 31 amino acids | RGGSDLWGICIQAPT | 275 |
| Tp_0619 Loop 1 (A21-E51) 31 amino acids | SDLWGICIQAPTMPD | 276 |
| Tp_0619 Loop 3 (Y114-G168) 55 amino acids | DSQHFLSVLGQGLLQ | 277 |
| Tp_0619 Loop 3 (Y114-G168) 55 amino acids | LGQGLLQPSSSSYSA | 278 |
| Tp_0619 Loop 3 (Y114-G168) 55 amino acids | GQGLLQPSSSSYSAG | 279 |
| Tp_0619 Loop 3 (Y114-G168) 55 amino acids | QHFLSVLGQGLLQPS | 280 |
| Tp_0619 Loop 3 (Y114-G168) 55 amino acids | YDSQHFLSVLGQGLL | 281 |
| Tp_0619 Loop 3 (Y114-G168) 55 amino acids | SQHFLSVLGQGLLQP | 282 |
| Tp_0619 Loop 3 (Y114-G168) 55 amino acids | AGNWHRPRSLLGVLT | 283 |
| Tp_0619 Loop 3 (Y114-G168) 55 amino acids | LTCTAKEVGAIHEES | 284 |
| Tp_RS01700 Loop 4 (V138-D180) 43 amino acids | VDFPLNPNTRFLAGK | 285 |
| Tp_RS01700 Loop 4 (V138-D180) 43 amino acids | AETVQRVALVALRQK | 286 |
| Tp_RS01700 Loop 4 (V138-D180) 43 amino acids | QRVALVALRQKLISE | 287 |
| Tp_RS01700 Loop 4 (V138-D180) 43 amino acids | VALVALRQKLISEGI | 288 |
| Tp_RS01700 Loop 4 (V138-D180) 43 amino acids | VQRVALVALRQKLIS | 289 |
| Tp_RS01700 Loop 4 (V138-D180) 43 amino acids | PNTRFLAGKLPAETV | 290 |
| Tp_RS01700 Loop 4 (V138-D180) 43 amino acids | NTRFLAGKLPAETVQ | 291 |
| Tp_RS01700 Loop 4 (V138-D180) 43 amino acids | LRQKLISEGIIKALD | 292 |
| Tp_RS03465 Loop 4 (V170-P200) 31 amino acids | STDLKWEQFLQKYCL | 293 |
| Tp_RS03465 Loop 4 (V170-P200) 31 amino acids | LKWEQFLQKYCLECP | 294 |
| Tp_RS05460 Loop 1 (G25-A48) 24 amino acids | LVQYRPRGSGGVAAN | 295 |
| Tp_RS05460 Loop 1 (G25-A48) 24 amino acids | RGSGGVAANGNKEVY | 296 |
| Tp_RS05460 Loop 4 (R159-R175) 17 amino acids | RSCEKVVEEEREQTN | 297 |
| Tp_RS05525 Loop 1 (D13-162) 50 amino acids | LRTVRRIFSWMVQHV | 298 |
| Tp_RS05525 Loop 1 (D13-162) 50 amino acids | YPSLRTVRRIFSWMV | 299 |
| Tp_RS05525 Loop 1 (D13-162) 50 amino acids | VRRIFSWMVQHVDSL | 300 |
| Tp_RS05525 Loop 1 (D13-162) 50 amino acids | PSLRTVRRIFSWMVQ | 301 |
| Tp_RS05525 Loop 1 (D13-162) 50 amino acids | GNTYRDTNSARARVV | 302 |
| Tp_RS05525 Loop 1 (D13-162) 50 amino acids | NSARARVVEQALELA | 303 |
| Tp_RS05525 Loop 1 (D13-162) 50 amino acids | RTVRRIFSWMVQHVD | 304 |
| Tp_RS05525 Loop 1 (D13-162) 50 amino acids | ARARVVEQALELAEK | 305 |
| Tp_RS05525 Loop 1 (D13-162) 50 amino acids | SARARVVEQALELAE | 306 |
| Tp_RS05525 Loop 1 (D13-162) 50 amino acids | RIFSWMVQHVDSLGI | 307 |
| Tp_RS05525 Loop 1 (D13-162) 50 amino acids | EKTYPSLRTVRRIFS | 308 |
| Tp_RS05525 Loop 1 (D13-162) 50 amino acids | YRDTNSARARVVEQA | 309 |
| Tp_RS05525 Loop 2 (G82-Y99) 18 amino acids | GAASVFGSGPFVKST | 310 |
| Tp_RS05525 Loop 2 (G82-Y99) 18 amino acids | SVFGSGPFVKSTFQY | 311 |
| Tp_RS05525 Loop 2 (G82-Y99) 18 amino acids | AASVFGSGPFVKSTF | 312 |

### REFERENCES

1. Institute for Health Metrics and Evaluation. http://www.healthdata.org/.
2. Ghanem et al., 2020. N Engl J Med 382:845-854.
3. Choudhri et al., 2018. Infectious and congenital syphilis in Canada, 2010-2015. Canada Commun Dis Rep 44:43-48.
4. Spiteri et al., 2019. Epidemiol Infect 147:e143.
5. Thng C C M. 2018. Acad Forensic Pathol 8:938-946.
6. Chen X S. 2017. Syphilis trends in China: biased by screening and reporting? Lancet Infect Dis 17.
7. Dyer O. 2022. Syphilis and gonorrhoea cases soar in US. BMJ o2355.
8. Government of Canada. 2020. Infectious Syphilis and Congenital Syphilis in Canada, 2020.
9. Korenromp et al., 2019. PLoS One 14:e0211720.
10. Mabey et al., 2017. Lancet 390:2036.
11. Newman et al., 2013. PLoS Med 10:e1001396.
12. Douglas J M. 2009. Penicillin Treatment of Syphilis. JAMA 301:769.
13. Chesson et al., 1999. AIDS 13:1387-1396.
14. Centres for Disease Control and Prevention. 2006. The national plan to eliminate syphilis from the United States.
15. Centres for Disease Control and Prevention. 2000. Syphilis Elimination Communication Plan.
16. World Health Organization. 2007. The global elimination of congenital syphilis: rationale and strategy for action.
17. LaFond R E, Lukehart S A. 2006. Biological Basis for Syphilis. Clin Microbiol Rev 19:29-49.
18. Lukehart S A. 1988. Ann Intern Med 109:855.
19. Raiziss G, Severac M. 1937. ArchDermatolSyphilol 35:1101-1109.
20. Lukehart et al., 1981. J Immunol 127:1361-1368.
21. Tosca et al., 1988. ex Transm Infect 64:289-293.
22. Arroll et al., 1999. Infect Immun 67:4757-4763.
23. Sell et al., 1980. Sex Transm Dis 7:74-84.
24. van Voorhis et al., 1996. Infect Immun 64:1048-1050.
25. Lukehart et al., 1980. J Immunol 124:461-467.
26. Carlson et al., 2011. Am J Dermatopathol 33:433-460.
27. Podwinska et al., 2000. FEMS Immunol Med Microbiol 28:1-14.
28. Moore et al., 2007. Infect Immun 75:2046-2062.
29. Leader et al., 2007. CD4⁺ Infect Immun 75:3021-3026.
30. Van Voorhis et al., 1996. J Infect Dis 173:491-495.
31. Lukehart S A. 1982. Infect Immun 37:64-69.
32. Cruz et al., 2012. PLoS Negl Trop Dis 6:e1717.
33. Izard et al., 2009. J Bacteriol 191:7566-7580.
34. Liu et al., 2010. Cellular Architecture of Treponema pallidum: Novel Flagellum, Periplasmic Cone, and Cell Envelope as Revealed by Cryo Electron Tomography. J Mol Biol 403:546-561.
35. Radolf et al., 1988. Infect Immun 56:490-498.
36. Radolf J, Lukehart S. 2006. Pathogenic Treponema: molecular and cellular biology. Caister Academic Press, Norfolk, England.
37. Lithgow K V., Cameron C E. 2017. Expert Rev Vaccines 16:37-44.
38. Radolf J D. 1995. Mol Microbiol 16:1067-1073.
39. Cameron C E. 2003. Identification of a Treponema pallidum Laminin-Binding Protein. Infect Immun 71:2525-2533.
40. Cameron et al., 2005. Defining the Interaction of the Treponema pallidum Adhesin Tp0751 with Laminin. Infect Immun 73:7485-7494.
41. Houston et al., 2011. Bifunctional Role of the Treponema pallidum Extracellular Matrix Binding Adhesin Tp0751. Infect Immun 79.
42. Houston et al., 2012. PLoS Pathog 8:e1002822.
43. Cameron et al., 2008. J Bacteriol 190:2565-2571.
44. Parker et al., 2016. PLOS Pathog 12:e1005919.
45. Lithgow et al., 2017. A defined syphilis vaccine candidate inhibits dissemination of Treponema pallidum subspecies pallidum. Nat Commun 8.
46. Skerra A. 2000. Lipocalins as a scaffold. Biochim Biophys Acta—Protein Struct Mol Enzymol 1482:337-350.
47. FLOWER D R. 1996. Biochem J 318:1-14.
48. Bishop R E, Weiner J H. 1996. Trends Biochem Sci 21:127.
49. Newcomer M E, Jones T A, Aqvist J, Sundelin J, Eriksson U, Rask L, Peterson P A. 1984. The three-dimensional structure of retinol-binding protein. EMBO J 3:1451-1454.
50. Salier et al., 2004. BioEssays 26:456-458.
51. Schlehuber S, Skerra A. 2002. Biophys Chem 96:213-228.
52. Breustedt et al., 2006 Biochim Biophys Acta—Proteins Proteomics 1764:161-173.
53. Skerra A. 2003. Imitating the humoral immune response. Curr Opin Chem Biol 7:683-693.
54. Beste G, et al., 1999. Proc Natl Acad Sci 96:1898-1903.
55. Skerra A. 2001 Rev Mol Biotechnol 74:257-275.
56. Schönfeld et al., 2009. Proc Natl Acad Sci 106:8198-8203.
57. Gille et al., 2016. Functional characterization of a VEGF-A-targeting Anticalin, prototype of a novel therapeutic human protein class. Angiogenesis 19:79-94.
58. Masuda et al., 2018. J Pharmacol Exp Ther 365:368-378.
59. Anderson et al., 2015. Discovery of PRS-060, an inhalable CD123/IL4Ra/TH2 blocking anti-asthmatic anticalin protein re-engineered from endogenous lipocalin-1, p. OA3256. In 5.1 Airway Pharmacology and Treatment. European Respiratory Society.

In view of the many possible embodiments to which the principles of the disclosure may be applied, it should be recognized that the illustrated embodiments are only examples of the invention and should not be taken as limiting the scope of the invention.

1. A chimeric Tp0751 (99-237) peptide, comprising:
at least one non-native epitope peptide that replaces all of or part of one or more loops of a native Tp0751 (99-237) peptide,
wherein the native Tp0751 (99-237) peptide sequence is SEQ ID NO: 3, and
wherein the one or more loops of the native Tp0751 (99-237) peptide are
loop 1 GEQGALQHLLA (SEQ ID NO: 4),
loop 2 QTEISPNSGDIHP (SEQ ID NO: 5),
loop 3 REHA (aa 147-150 of SEQ ID NO: 2),
loop 4 AS (aa 157-158 of SEQ ID NO: 2),
loop 5 RK (aa 172-173 of SEQ ID NO: 2),
loop 6 NTAISSI (SEQ ID NO: 6),
loop 7 HE (aa 198-199 of SEQ ID NO: 2), and
loop 8 aa DVARLKIGSTSMWD (SEQ ID NO: 7).

2. (canceled)

3. The chimeric Tp0751 (99-237) peptide of claim 1, further comprising at least one non-native epitope peptide that replaces all or of part of the native C-terminus of the native Tp0751 (99-237) peptide, wherein the native C-terminus of the native Tp0751 (99-237) peptide is SEQ ID NO: 8.

4. The chimeric Tp0751 (99-237) peptide of claim 1, wherein:
2, 3, 4, 5, 6, 7, or 8 of the loops of the native Tp0751 (99-237) peptide are replaced in all or part with the at least one non-native epitope peptide
loop 8 of the native Tp0751 (99-237) peptide is replaced in all or part with the at least one non-native epitope peptide, or
loops 2, 3, 6 and 8 loops 2, 3, and 8, loops 2 and 8, loops 2, 3, and 8 and the C-terminus or loops 2 and 8 and the C-terminus of the native Tp0751 (99-237) peptide are replaced with the at least one non-native epitope peptide.

5. (canceled)

6. The chimeric Tp0751 (99-237) peptide of claim 1, wherein the at least one non-native epitope peptide comprises 2, 3, 4, 5, 6, 7, or 8 different non-native epitope peptides.

7. The chimeric Tp0751 (99-237) peptide of claim 1, wherein the at least one non-native epitope peptide comprises one or more antigenic sequences from T. pallidum TprC protein, T. pallidum TprK protein, T. pallidum Tp0326 protein, T. pallidum Tp0856 protein, T. pallidum Tp0346 protein, and/or T. pallidum Tp0483 protein.

8. The chimeric Tp0751 (99-237) peptide of claim 7, wherein:
the one or more antigenic sequences from T. pallidum TprC protein is one or more of SEQ ID NO: 17, 18, 24, 25, 26, 31, 32, 35, 36 or 37;
the one or more antigenic sequences from T. pallidum TprK protein is one or more of SEQ ID NO: 19, 20, 21, 22, 27, 28, 29, 30, 33, or 34;
the one or more antigenic sequences from T. pallidum Tp0326 protein is SEQ ID NO: 23;
the one or more antigenic sequences from T. pallidum Tp0856 protein is SEQ ID NO: 143 or 145;
the one or more antigenic sequences from T. pallidum Tp0346 protein is SEQ ID NO: 144; and/or
the one or more antigenic sequences from T. pallidum Tp0483 protein is SEQ ID NO: 146.

9. The chimeric Tp0751 (99-237) peptide of claim 1, wherein the sequence of the chimeric Tp0751 peptide comprises at least 80%, at least 85%, at least 90%, at least 95%, at least 96%, at least 97%, at least 98%, at least 99%, or 100% sequence identity to any one of SEQ ID NOS: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114, such as at least 80%, at least 85%, at least 90%, at least 95%, at least 96%, at least 97%, at least 98%, at least 99%, or 100% sequence identity to SEQ ID NO: 82 or 88.

10. The chimeric Tp0751 (99-237) peptide of claim 1, wherein the sequence of the chimeric Tp0751 peptide comprises any one of SEQ ID NOS: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 97, 98, 99, 100, 101, 102, 103, 104, 105, 106, 107, 108, 109, 110, 111, 112, 113, or 114 with 1, 2, 3, 4, 5, 6, 7, 8, 9, 10, 11, 12, 13, 14, 15, 16, 17, 18, 19, or 20 conservative amino acid substitutions.

11. The chimeric Tp0751 (99-237) peptide of claim 1, wherein the at least one non-native epitope peptide comprises one or more B cell epitopes from T. pallidum proteins, such as one or more of SEQ ID NO: 115, 116, 117, 118, 119, 120, 121, 122, 123, 124, 125, 126, 127, 128, 217, 218, 219, 220, 221, 222, 223, 224, 225, 226, 227, 228, 229, 230, 231, or 232.

12. The chimeric Tp0751 (99-237) peptide of claim 11, wherein the at least one non-native epitope peptide is encoded by a sequence having at least 80%, at least 85%, at least 90%, at least 95%, at least 96%, at least 97%, at least 98%, at least 99%, or 100% sequence identity to any one of SEQ ID NOS: 130-142 or 233-248.

13. The chimeric Tp0751 (99-237) peptide of claim 1, wherein the one or more T cell epitopes from T. pallidum proteins, comprise one or more of SEQ ID NO: 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, or 312.

14. An isolated nucleic acid molecule encoding the chimeric Tp0751 (99-237) peptide of claim 1.

15. The isolated nucleic acid molecule of claim 12, wherein the isolated nucleic acid molecule comprises at least 80%, at least 85%, at least 90%, at least 95%, at least 96%, at least 97%, at least 98%, at least 99%, or 100% sequence identity to SEQ ID NO: 95, 96, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 201, 203, 204, 205, 206, 307, 208, 209, 210, 211, 212, 213, 214, 215, or 216.

16. (canceled)

17. A vector comprising the isolated nucleic acid molecule of claim 14.

18. An isolated cell, comprising;
the chimeric Tp0751 (99-237) peptide of claim 1.

19. (canceled)

20. A liposome, comprising,
the chimeric Tp0751 (99-237) peptide of claim 1.

21. A composition, comprising:
the chimeric Tp0751 (99-237) peptide of claim 1, and
a pharmaceutically acceptable carrier and/or an adjuvant.

22. A method of stimulating an immune response in a subject, comprising:
administering to the subject a therapeutically effective amount of
the chimeric Tp0751 (99-237) peptide of claim 1,
thereby stimulating the immune response.

23. A method of treating or preventing Treponema pallidum subsp. pallidum infection in a subject, comprising:
administering to the subject a therapeutically effective amount of
the chimeric Tp0751 (99-237) peptide of claim 1,
thereby treating or preventing Treponema pallidum subsp. pallidum infection.

24.-25. (canceled)

26. The method of claim 22, further comprising administering penicillin, doxycycline, tetracycline and/or ceftriaxone to the subject.
